(19) United States
(12) Patent Application Publication
(10) Pub. No.: US 2025/0236915 A1
(43) Pub. Date: Jul. 24, 2025

(54) CD4+ T CELL MARKERS, COMPOSITIONS, AND METHODS FOR CANCER

(71) Applicant: Fred Hutchinson Cancer Center, Seattle, WA (US)

(72) Inventors: Joshua VEATCH, Seattle, WA (US); Stanley R. RIDDELL, Sammamish, WA (US)

(21) Appl. No.: 18/840,039

(22) PCT Filed: Feb. 21, 2023
(86) PCT No.: PCT/US2023/062964

§ 371 Date: Aug. 20, 2024

Related U.S. Application Data
(60) Provisional application No. 63/314,294, filed on Feb. 25, 2022.

(51) Int. Cl. C12Q 1/6886; G01N 33/569; G01N 33/574

(52) U.S. Cl. CPC C12Q 1/6886; G01N 33/56972; G01N 33/57492; C12Q 2600/112; C12Q 2600/158

(57) ABSTRACT

The present disclosure relates, in part, to the discovery that CD4+ T cells characterized by certain markers are over-represented in solid tumors. Markers include, for example, CD200, CXCL13, and PD-1, as well as combinations of these, and other markers. The markers can identify CD4+ T cells having certain phenotypes. Identifying such cells enables a variety of therapeutic and prognostic applications. Such CD4+ T cells will typically include those specific for a tumor neoantigen or antigen, and may be enriched for and/or expanded and used in a T cell therapy. TCRs from such CD4+ T cells may be used in therapy (e.g., comprising T cells recombinantly expressing such a TCR or an engineered TCR or binding domain derived therefrom). The relative presence or absence of such CD4+ T cells in a tumor sample can provide information regarding the immune state of the subject and the likely prognosis of disease.

### STATEMENT OF GOVERNMENT INTEREST

This invention was made with government support under CA241523, and CA114536 awarded by the National Institutes of Health. The government has certain rights in the invention.

### BACKGROUND

Clinical responses to immune checkpoint blockade (ICB) in solid tumors have been proposed to be mediated in part by T-cell recognition of neoantigens that are derived from cancer-specific mutations and presented on major histocompatibility complex (MHC) molecules (Gubin et al., 2014; Le et al., 2015; Rizvi et al., 2015). Work characterizing the antigen specificity and transcriptional states of T cells in human tumors has primarily focused on class I MHC-restricted CD8⁺ cytotoxic T cells. Tumor infiltrates contain heterogeneous populations of CD8⁺ T cells, some of which are specific for tumor antigens and exist in varying states of functional exhaustion (Caushi et al., 2021; Oliveira et al., 2021; Siddiqui et al., 2019), and many “bystander” CD8⁺ T cells that are phenotypically distinct and recognize viral or other antigen specificities (Duhen et al., 2018; Scheper et al., 2019; Simoni et al., 2018). CD4⁺ T cells specific for neoantigens including oncogenic driver mutations presented by class II MHC have also been identified in cancer patients (Linnemann et al., 2015; Ott et al., 2017; Sahin et al., 2017; Cafri et al., 2019; Veatch et al., 2019; Veatch et al., 2018; Yossef et al., 2018). In murine models, CD4+ neoantigen-specific T cells residing within tumors are required for response to ICB (Alspach et al., 2019), and may mediate tumor rejection through direct destruction of tumor cells (Quezada et al., 2010), activation of innate immune cells (Mumberg et al., 1999; Tveita et al., 2016), and stimulation of CD8⁺ T cells (Ossendorp et al., 1998). Anecdotal reports in patients have shown that the adoptive transfer of CD4⁺ T cells alone can have marked antitumor effects (Hunder et al., 2008; Tran et al., 2014), and CD4⁺ T cells expanded from bladder cancers are capable of antigen-specific lysis of autologous tumor, suggesting that direct recognition can be a mechanism in human cancer. (Oh et al., 2020)

Single cell sequencing of CD4⁺ T cells from primary human samples has identified multiple subpopulations of cells, including regulatory T cells, T cells expressing cytolytic proteins, and cells sharing markers with T follicular helper cells (T_{FH}) (Bassez et al., 2021; Bonnal et al., 2021; Oh et al., 2020). Tertiary lymphoid structures containing B cells and CD4⁺ T cells of unknown specificity, and a B cell transcriptional signature have been associated with response to ICB in melanoma, suggesting functional contributions of some subsets of CD4⁺ T cells or B cells (Cabrita et al., 2020; Helmink et al., 2020; Voabil et al., 2021). However, the phenotype, heterogeneity, and functional roles of the CD4⁺ T cells that infiltrate tumors and are specific for tumor antigens has not been well defined beyond enrichment in fractions of cells that express PD-1 or CD39 (Balança et al., 2021; Kortekaas et al., 2020; Yossef et al., 2018).

### BRIEF DESCRIPTION OF THE DRAWINGS

FIG. 1. Identification of neoantigen-specific TCRVB clonotypes from melanoma patients. A-B. IFN-γ secretion by T cell lines expanded in limiting dilution cultures from intratumoral PD-1-high CD4⁺ T cells from patients X205 (A) and X422 (B) after incubation with 1 μM 27-mer peptide containing the mutant or wildtype amino acid at position +14. The dominant TCR VB clonotype present in each culture was identified by TCR Vβ sequencing. C. T cell cultures expanded in bulk from intratumoral PD-1-high CD4⁺ T cells from patient X205 were incubated with indicated pools of peptides (WT=wildtype peptide) and CD40L-expressing cells were isolated by FACS. TCRVβ genes in sorted CD40L-positive T cells were identified by sequencing, and the percentage of selected clonotypes (rows) found in replicate assays relative to other peptide stimulations (columns) are shown. D. PBMC from patient X198 were stimulated with a pool of mutant peptides and restimulated on day +13 with individual 27-mer peptides containing the wildtype or mutated amino acid at position +14 and the frequency of IFN-γ secreting CD4⁺ T cells was measured by Elispot. E. Multiple parallel cultures of PBMC from patient X198 were stimulated with individual mutant peptides in duplicate and cultured for 13 days followed by TCRVβ sequencing of enriched CD4⁺ T cells. The percentage of TCRVβ templates (rows) seen in replicate cultures stimulated with a single mutant peptide (columns) relative to other stimulations are shown. F. Summary of the total number of TCRVβ clonotypes specific for neoantigens, tumor self antigens and viral antigens that were identified from each of the 4 patients. N/A indicates not evaluated. It will be understood that TCRVβ and “TCRVb” (as shown in certain of the figures herein) have the same meaning, unless otherwise indicated.

FIG. 2. Tumor antigen-specific conventional CD4⁺ T cells exhibit transcriptional signatures distinct from Treg and bystander conventional CD4⁺ T cells. A-B. Unsupervised clustering of 10,186 CD4⁺ T cells from primary tumor samples of 4 patients visualized by UMAP. C. Expression of FoxP3, CXCL13, TCF7, GZMA, TYMS, IL7R and IFNG mRNA, and PD-1/CD279, Tim3, CXCR5, CD103 and CD39 surface protein in tumor-infiltrating CD4⁺ T cells. D. Summary of antigen-specific CD4⁺ T cells identified in the tumor infiltrate by TCR Vβ sequence. E. Cells with TCRVβ clonotypes specific for neoantigens and self-antigens are largely restricted to CXCL13⁺ clusters (cells with TCRVβ clonotypes for viral antigens are indicated in the left and upper left portions of the plot; approx. 9 dots). F. Proportion of neoantigen-, self-antigen-and viral antigen-specific cells in CXCL13⁺, CXCL13⁺, and Foxp3⁺ clusters. G-H. The number of cells with each clonotype (G) and the relative enrichment of TCR clonotypes detected in tumor relative to peripheral blood (H). I. mRNA expression of immune-related genes defining each cluster of CD4⁺ T cells in the tumor.

FIG. 3: The fraction of CXCL13⁺ CD4⁺ T cells correlates with overall survival in melanoma. A-C. Data from 26773 sorted CD4⁺ T cells from 20 patients were visualized by UMAP with (A) patients indicated by shading or (B) cells clustered by phenotype and (C) gene expression of the indicated mRNA or surface markers (cell clusters in (C) are generally according to the numbers shown). D. Heatmap showing expression of mRNA that define phenotypic clusters of CD4⁺ T cells. E. Overall survival of 20 melanoma patients stratified by greater or less than 30% of conventional CD4⁺ T cells belonging to CXCL13⁺ clusters. F. Overall survival of 471 patients in the cancer genome atlas based on the median of CXCL13 expression normalized to CD4 expression.

FIG. 4. Tumor-infiltrating CD8⁺ T cells share gene expression signatures and their frequency and proliferative state correlate with tumor antigen-specific CD4⁺ T cells. A. 2585 CD8⁺ T cells from 2 melanoma patients were visualized by UMAP and clustered by phenotype. Cell clusters in (A) are generally according to the numbers shown. B. Presence of tumor antigen-specific TCRVβ in the UMAP plots. C. 15332 CD8⁺ T cells from 20 patients visualized by UMAP showing mRNA expression of CXCL13, TYMS, IL7R, TCF7, Tox, and GZMB, and surface expression of CD279/PD-1, CD39, Tim3 and CD103. D. Phenotypic categories of CD8⁺ T cells are indicated. Cell clusters in (D) are generally according to the numbers shown. E. Jaccard similarity index of mRNA expressed genes upregulated at least 2-fold in phenotypic categories of CD4⁺ and CD8⁺ T cells. F. Spearman correlation of different CD4⁺ T cell populations as a fraction of CD4⁺ T cells compared to CD8⁺ T cell populations as a fraction of CD45⁺ cells across the 20-patient cohort are shown. G. Spearman correlation of CXCL13 expression with CD8a expression in the 471 patients with melanoma. H. Spearman correlations of CXCL13, BTLA and IL-21 expression to CD8a expression, with and without normalization to CD4⁺ expression. * FDR <. 05 ** FDR<0.01 *** p<10⁻⁴. p<10⁻⁶ for all correlations in H.

FIG. 5. The presence of CXCL13⁺ CD4⁺ T cells correlates with an immune stimulatory phenotype in macrophages. A-B. 12977 CD45⁺ CD19-CD3-cells from 20 melanoma patients were separated into phenotypic clusters with differentially expressed genes shown (A) and phenotypic clusters visualized by UMAP (B). Cell clusters in (B) are generally according to the numbers shown. C. Spearman correlations of expression of the indicated mRNA in macrophages in the 20-patient cohort with the fraction of CD4⁺ T cells in CXCL13⁺ clusters. D. Correlation of the fraction of CXCL9⁺ macrophages and the fraction of CXCL13⁺ CD4⁺ cells of total CD4⁺ T cells. E-F. Expression of CXCL13 compared to CXCL9 (E) or the indicated chemokines (F) in 471 melanoma patients was compared to expression of CXCL13, BTLA, and IL21, with or without normalization of these to CD4, with Spearman correlations shown. *significant with FDR <0.05 ** significant with FDR <0.01. All correlations in (F) p<10-10 for correlations with CXCL9, CXCL10, CXCL11 and IL15.

FIG. 6. TFH-like CD4⁺ T cells correlate with B cell infiltration and colocalize with B cells in melanoma. A-B. 9428 CD19⁺ B lineage cells from 20 patients were visualized by UMAP, and cell differentiation states assigned by gene expression. C-D. Correlation of memory B cells as a percentage of CD45⁺ cells and CXCL13⁺, TCF7⁺, CD4⁺ T cells as a percentage of CD4⁺ T cells (C) and Spearman correlation with B cells subsets with CXCL13+CD4⁺ T cell subsets in 20 melanoma patients (D). E-F. Spearman correlations of CXCL13 versus IGHG1 (E) or CXCL13, BTLA, and IL21 expression versus B cell markers in 471 melanoma patients (F). G. Multiplex immunohistochemistry of a tertiary lymphoid structure in patient X198, with CD4 T cells inferred from cells staining for CD3 but not CD8. The text above the images repeats the text within the images (see lines associating text above images with corresponding text within images) for clarity, and is not intended to indicate specific cells/tissues. H-I. Proximity analysis of the number of CD79a⁺ b lineage cells within 100 microns of each CD4⁺ or CD8⁺ T cell based on CXCL13 staining in patient X197 (H) and X198 (I). * FDR<0.05 ** p<0.0001

FIG. 7. Infiltrating CD4⁺ T cells in breast cancer treated with immune checkpoint inhibitors exhibit similar phenotypes and correlate to activation of CD8⁺ T cells and myeloid cells. A. Single cell RNA sequencing of CD4⁺ T cells from 31 patients with breast cancer visualized by UMAP for the expression of the indicated genes, with cells clustered by phenotype (right lower panel). Cell clusters in the right lower panel are generally according to the numbers shown. B. Correlation between CXCL13⁺ cell populations as a fraction of CD4⁺ T cells and proliferating CD8⁺ T cells as a fraction of all cells. C. Spearman correlations of the indicated cell subsets. D-F. Correlation between CXCL13⁺ CD4⁺ T cells as a fraction of CD4⁺ T cells and the fraction of myeloid cells expressing CXCL9 (D), IL15 (E), and a panel of genes (F).

FIGS. 8A-8B. Schematic of methods for identification of tumor antigen-specific TCRVβ clonotypes. A. Determination of TCRVβ clonotypes expressed by tumor antigen-specific CD4⁺ T cells by analysis of neoantigen reactivity of PD-1-high CD4⁺ T cells from tumors of melanoma patients. B. Determination of TCRVβ clonotypes expressed by tumor antigen-specific CD4⁺ T cells isolated from the peripheral blood of melanoma patients previously treated with tumor infiltrating lymphocytes.

FIG. 9. Identification of neoantigen-specific T cells using peptide stimulation of PD-1-high CD4⁺ T cells expanded from melanoma tumors. A-B. Oligoclonal or monoclonal T cell lines were derived from CD4⁺ PD-1-high T cells from patient X205 and incubated with overlapping pools of peptides indicated in the columns and rows in A. IFN-γ secretion was measured with absorbance indicated in B to identify reactivity with individual pools. C-D. T cell responses in patient X422 were analyzed as in A and B. E. T cell cultures expanded in bulk from intratumoral PD-1-high CD4⁺ T cells from patient X205 were incubated with indicated pools of peptides, stained for expression of CD40L and CD4, and CD40L⁺ CD4⁺ cells were isolated by FACS. F. Percentage of CD40L⁺ CD4⁺ T cells after stimulation of cells expanded from PD-1-low,-intermediate, and -high intratumoral T cells from patient X205 with neoantigen, wild type, tumor associated self peptides, and a panel of viral peptides. G. TCRVβ clonotypes enriched in sorted CD40L⁺ cells after restimulation with viral antigens. H. PD-1-low or PD-1-high intratumoral CD4⁺ T cells from patient X422 were expanded and incubated with peptides encompassing neoantigens or viral antigens and IFN-γ secretion measured by FACS. I. PBMCs from patient X198 were stimulated with a pool of mutant peptides and cultured for 13 days followed by restimulation with peptides encompassing individual mutations, and quantitation of IFN-γ secreting cells by Elispot.

FIG. 10. Identification of viral-specific TCR Vβ clonotypes from patients X197 and X198. A-B. Aliquots of PBMC from patients X197 (A) and X198 (B) were stimulated in duplicate with pools of peptides corresponding to viral antigens, cancer testes antigens, and neoantigens and the percentage of each TCRVβ template (rows) observed in each stimulation condition (columns) relative to other stimulations for selected TCRVβ clonotypes is shown. C-D. The frequency of TCRVβ clonotypes identified to be viral-or neoantigen-specific in the peripheral blood and tumor from patients X197 (C) and X198 (D).

FIG. 11: Gene expression and phenotypic signatures of neoantigen-specific CD4+ T cells. A-B. Data from 10,186 sorted CD4⁺ T cells from 4 patients visualized by UMAP with each patient (A) and the antigen specificity for each patient (B). C. Jaccard similarity based on TCRVβ clonotypes between clusters of CD4⁺ T cells, normalized by number of cells in each cluster. D. Individual neoantigen-specific TCRVβ clonotypes are indicated and visualized by UMAP. E. CD25^{low} PD-1 ^{high} CXCR6-T_{FH} cells, CD25^{low} PD-1^{high} CXCR6⁺ effector cells and CD25_{low} PD-1_{low} CD127^{High} bystander cells were sorted from primary tumor samples and rested overnight prior to labeling with CFSE and stimulation with anti-CD3/anti-CD28 beads. Proliferation was measured 5 days later by CFSE dye dilution. * p=0.02 by paired t test.

FIG. 12: CXCL13⁺ CD4⁺ T cells and transcripts enriched in this subset correlate with survival in melanoma. A. Cox proportional hazard model for survival based on CXCL13+CD4⁺ T cells as a fraction of conventional T cells. B. Survival of 20 melanoma patients based on CD4⁺ T cells as a fraction of CD45⁺ cells, or CD4⁺ FoxP3⁺ regulatory T cells as a fraction of CD4⁺ T cells. C. Expression of CXCL 13 across different clusters of CD4⁺ and CD8⁺ T cells. D. Expression of BTLA across different populations of T cells and myeloid cells. E. Percentage of cells in different clusters expressing IL21 mRNA. F-H. Overall survival of 471 patients with melanoma based on expression of mRNA above the median for CXCL13 (F), BTLA (G), BTLA normalized to CD4 (H), and detectable IL21 expression (I).

FIG. 13: Identification of neoantigens recognized by CD8⁺ T cells after culture of tumor-infiltrating lymphocytes from a patient with melanoma. A. 169 9 and 10-mer peptides predicted to bind to HLA A01 A23 and B08 molecules expressed by the patient were combined into 26 pools represented by rows and columns. B-C. Therapeutic TIL product from patient X198 was restimulated with each neoantigen peptide pool and peptide pools corresponding to self antigens and IFN-γ secretion was measured by Elispot (B) and by intracellular staining (C). D. Candidate individual peptides were then used to restimulate the TIL product and IFN-γ release was measured by Elispot. E. Purified mutant and corresponding wildtype peptides were used to restimulate the therapeutic TIL product and IFN-γ release was measured by Elispot.

FIG. 14: Identification of TCR Vβ sequences of tumor antigen-specific CD8⁺ T cells and phenotype of tumor-infiltrating CD8⁺ T cells. A. Purified mutant and corresponding wildtype peptides were used to restimulate the therapeutic TIL product of patient X198 and IFN-y release was measured by Elispot. B. Pool of 5 mutant peptides from (A) were used to stimulate therapeutic TIL product in the presence of brefeldin A and IFN-γ secreting cells were sorted by FACS. C. 3 TCRVβ clones were identified that were enriched in IFN-γ secreted cells in two replicates. D. 2585 CD8⁺ T cells from tumors of patients X197 and X198 were visualized by UMAP and expression of the indicated mRNA genes and surface proteins are indicated. E-G. 15332 CD8⁺ T cells from 20 patients visualized by UMAP with patients (E) indicated and unsupervised cell clusters indicated (F) and genes associated with individual clusters are shown in (G).

FIG. 15: Myeloid cell gene expression and correlation of myeloid subsets to CXCL13⁺ CD4⁺ T ell populations in melanoma. A. UMAP of 12977 sorted CD45⁺ CD19-CD3-cells from 20 patients and expression of selected genes. B. Spearman correlation of CXCL13⁺ CD4⁺ T cell subsets with myeloid subsets are shown, none reaching significance at FDR of 0.05.

FIG. 16: Expanded B cell clones in patients with melanoma. A. CD45⁺ CD19⁺ cells were from 20 patients were visualized by UMAP of mRNA with individual patients indicated by color. B. Differentially expressed genes that distinguish phenotypic categories of B cells. C. CD25^{low} PD-1 ^{high} CXCR6⁺ T_{FH} cells, CD25^{low} PD-1 ^{high} CXCR6⁺ effector cells were sorted from patient X431, rested overnight, and incubated with cell trace violet-labelled allogeneic peripheral blood B cells in the presence of Staphylococcal enterotoxin B. B cell proliferation was measured 5 days later. Data is representative of three independent experiments. D. Expansion of B cell receptor clonotypes overlayed onto the UMAP plots. E. Expanded individual B cell receptor clonotypes from patients X198 and X431 are shown.

FIG. 17: Multiplex immunohistochemistry showing localization of CXCL13⁺ CD4⁺ and CD8⁺ T cells relative to TLS. A. Single colors from immunohistochemistry at 20× magnification are shown from patient X198. B. Multiplex immunohistochemistry of the tumor from patient X197. CD3⁺ CD8⁺ T cells are labelled as “CD4”.

FIGS. 18A, 18B, and 19 show characteristics of patients in a melanoma patient cohort, as described in Example 3.

FIG. 20: Tumor antigen-specific CD4⁺ T cells are lost during conventional TIL culture. A. TCRVβ clonotypes with at least 2 cells in the CXCL 13⁺ cluster from the tumor from two patients, as well as clonotypes with confirmed neoantigen specificity from patient X198 were quantitated in tumor and in therapeutic tumor infiltrating lymphocyte products (TIL product) by TCR sequencing. B. Individual neoantigen-specific clones were quantitated in the tumor and therapeutic product from patient X198. These indicate that the number and diversity of tumor antigen-specific clones are lost during the production of therapeutic TIL products.

FIG. 21: Flow panel schema for isolating CXCL13⁺ conventional CD4 T cells. Cells were gated for CD3⁺ CD4⁺ T cells and then CD25-high CD127-low regulatory T cells were gated out. The resulting conventional CD4⁺ T cells were sorted on the basis of CD200 and PD-1 expression. Note that in 2 patients who had been treated with PD-1 blocking antibodies, this population could still be identified with CD200 staining.

FIG. 22: CD25^{low} PD-1^{high} CD200^{high} cells from tumors of 4 patients were sorted based on CXCR6 expression.

FIG. 23: A. CD25^{low} PD-1^{high} CD200^{high} cells were sorted into CXCR6⁺ and CXCR6-fractions and TCF7 and CXCL13 expression was reassured by RNA sequencing (n=3 patients). This indicates that CD200 and PD-1 sorting isolates CXCL 13⁺ cells from CXCL 13-cells, and that CXCR6 negative cells define the TCF7⁺ population. B. CD25^{low} PD-1^{high} CD200^{high} cells were sorted into CXCR6⁺ (“TEFF”), CXCR6-(“T_{FH}”) and PD-11ow CD200^{low} (“bystander”) fractions. Cell were rested overnight in IL-2 50U/ml and IL-7 5 ng/ml and stimulated with anti CD3/anti CD28 beads for 4 days a proliferation was measured by dye dilution and flow cytometry (n=4 patients). This data suggests that the TCF7⁺ population isolated by CXCR6-negative staining is the proliferative stem/progenitor population.

FIG. 24 provides schematic diagrams showing (left) certain marker signatures of CD4⁺ T cells infiltrating human melanoma and (right) correlation of CXCL13⁺ CD4⁺ T cells with survival and state of other immune cells.

FIGS. 25A-25E show certain non-limiting examples of reagents and resources that may be useful in practicing embodiments of the present disclosure.

### DETAILED DESCRIPTION

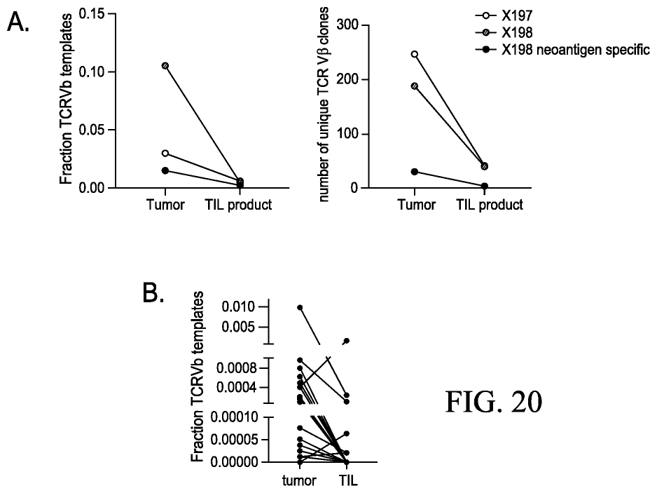

Tumor antigen-specific CD4+ T cells are required for the efficacy of immune checkpoint inhibitors in murine models but their contributions in human cancer are less understood. For example, phenotypic states and role(s) of tumor antigen-specific CD4+ T cells in the tumor microenvironment are poorly understood. Moreover, as shown in FIG. 20, tumor antigen-specific CD4+ T cells can be lost during conventional tumor infiltrating lymphocyte (TIL) culture.

The present disclosure relates, in part, to the discovery that CD4+ T cells characterized by expression of certain markers are over-represented in solid tumor samples. Identifying such cells enables a variety of therapeutic and prognostic applications. For example, such CD4+ T cells will typically include those expressing a TCR specific for a tumor neoantigen or tumor antigen, and these cells may be enriched for and/or expanded and used in a T cell (e.g., polyclonal) therapy. Alternatively or additionally, TCRs from such tumor-infiltrating CD4+ T cells may be sequenced and the sequence information can inform other therapies (e.g., comprising T cells recombinantly expressing such a TCR or an engineered TCR derived from such a TCR, or expressing a chimeric antigen receptor or single-chain TCR in which the antigen-binding domain is derived from such a TCR or engineered TCR, or even soluble molecules comprising a binding domain from such a TCR or engineered TCR). Furthermore, the relative presence or absence of such CD4+ T cells in a tumor sample can provide information regarding the immune state of the subject and the likely prognosis of disease.

Disclosed markers also improve the ability to detect and expand such tumor-infiltrating CD4+ T cells. Briefly, tumor samples will typically also contain non-tumor-antigen-specific “bystander” cells (e.g., T cells that happen to be present but are specific for non-tumor antigens) that are less affected or are unaffected by the tumor microenvironment and, consequently, are more robust than tumor antigen-specific CD4+ T cells, which can display an exhausted phenotype. Using conventional tumor-infiltrating lymphocyte (TIL) culture methods, bystander cells are present and can often outcompete the tumor antigen-specific CD4+ T cells for resources, significantly reducing the likelihood of identifying and successfully expanding the CD4+ T cells of interest. Thus, the disclosed markers permit selecting for CD4+ T cells of interest and against bystander cells, improving the ability to enrich for, maintain, and expand CD4+ T cells of interest. In some embodiments, CD4+ T cells of interest may be enriched for from about 2-fold to about 20-fold or even more, relative to their presence in the tumor sample, whereas conventional (e.g. TIL) culture methods can risk never identifying such CD4+ T cells and/or failing to effectively expand or enrich for them.

The present disclosure shows, in part, that targeted single cell RNA sequencing and matching of T cell receptor sequences can identify signatures and functional correlates of tumor antigen-specific CD4+ T cells infiltrating human solid cancers (e.g. melanoma tumors, breast cancer tumors). CD4+ T cells that recognize tumor-specific neoantigens and express CXCL13 can be subdivided into clusters expressing memory and T follicular helper markers, and those expressing cytolytic markers, exhaustion markers and IFN-γ. In a cohort of melanoma patients, the frequency of CXCL13+CD4+ T cells in the tumor correlated with the transcriptional states of CD8+ T cells and macrophages, maturation of B cells, and patient survival, and similar patterns were seen in a breast cancer cohort. These results identify distinct phenotypes and functional correlates of tumor antigen-specific CD4+ T cells in solid tumors such as melanoma and support use of such cells to modify the tumor microenvironment.

In accordance with the present disclosure, CD4+ T cells of interest from a tumor sample can be characterized by, for example, increased expression of CXCL13. CXCL13+CD4+ T cells can be further characterized using certain markers, discussed herein, as having various phenotypes, including, for example, increased proliferative ability.

CXCL13 is a chemokine and is secreted by cells. Thus, CXCL13 expression may be assessed by, for example, intracellular staining, mRNA expression levels, or secretion into cell supernatant or cell culture, but is not readily amenable to assessment by, for example, flow cytometry. As taught herein, CD200 expression can be more readily assessed by flow cytometry or the like than CXCL13, and CD200 expression, optionally further in combination with PD-1 expression (a marker of T cell exhaustion), can serve as a surrogate to isolate CXCL13+ from CXCL13-CD4+ T cells.

CD200 can be particularly useful for isolating CXCL13+CD4+ T cells from subjects who have received anti-PD-1 antibodies (e.g. as a cancer therapy) wherein there is a reduced or even abrogated the ability to stain these T cells for PD-1 expression (e.g. because PD-1 expressed on the T cells is already bound by a previously administered therapeutic anti-PD-1 antibody). Alternatively, CD4+ T cells from such subjects can be stained for CD200 and, indirectly, for PD-1 using an antibody or antigen-binding fragment thereof that binds to the antigen-bound PD-1-specific antibody (e.g., when a subject has previously received a PD-1-specific antibody of the IgG4 isotype, such as pembrolizumab (Keytruda®) or nivolumab (Opdivo®), detecting the antigen-bound PD-1-specific antibody by use of an anti-IgG4 antibody or antigen-binding fragment thereof). See, e.g., Zelba et al., Cancer Immunology, Immunotherapy 67:1845-1851 (2018) doi.org/10.1007/s00262-018-2244-7, which detection techniques and reagents, including the therapeutic and diagnostic anti-PD-1 antibodies described therein, are incorporated herein by reference in their entireties.

The present disclosure further teaches that CXCR6 expression can delineate CD4+ T cells having a proliferative stem/progenitor population from CD4+ T cells having an effector phenotype.

Moreover, the disclosure also teaches that the prevalence of CXCL13+CD4+ T cells in solid tumor correlates with a desired status of other tumor-infiltrating immune cells and with outcome.

Accordingly, the present disclosure provides, in-part, markers, methods, and compositions for identifying a CD4+ T cell (or T cell receptor genes thereof) with tumor antigen-specificity, tumor neoantigen-specificity, and/or tumor-infiltrating capacity.

Disclosed embodiments include methods for sorting, enriching for, activating, isolating, and/or expanding a CD4+ T cell of interest. Also provided are methods for generating recombinant host cells (e.g. T cells) that comprise introducing polynucleotides (or a single polynucleotide molecule) encoding a T cell receptor (or an antigen-binding fragment thereof) from a CD4+ T cell characterized in accordance with the present disclosure. Also provided are methods for administering a CD4+ T cell or host cell to a subject in need thereof; e.g. to treat a subject having a solid cancer. Also provided are CD4+ T cells, populations thereof, and compositions characterized in accordance with the present disclosure. In some embodiments, CD4+ T cells express certain markers, or CD4+ T cell populations or compositions are enriched for such CD4+ T cells.

Certain examples of such markers are shown in Table 1.

TABLE 1

| Marker | Surrogate Marker | Description (of the Marker) |
|---|---|---|
| CXCL13⁺ | CD200⁺, preferably | Chemokine |
| (secreted) | CD200⁺PD-1⁺ | |
| CD200 | | Gene associated with T follicular |
| | | helper cells |
| BCL6 | | Gene associated with T follicular |
| | | helper cells |
| TCF7 (nuclear | CD200⁺PD- | Memory gene |
| protein) | 1⁺CXCR6⁻ for TCF7⁺ | |
| IL-7R | | Memory gene |
| Tim-3 | | Coinhibitory protein |
| Lag-3 | | Coinhibitory protein |
| CCL3 | | Inflammatory marker |
| CCL4 | | Inflammatory marker |
| IFN-γ | | Inflammatory marker |
| GZMA/K | | Cytolytic marker |
| PRF1 | | Cytolytic marker |
| CD103 | | Tissue resident marker |
| TYMS | | Gene involved in proliferation |
| TOP2A | | Gene involved in proliferation |
| MCM2/4 | | Gene involved in proliferation |
| BTLA | | |

It will be understood that if a cell to be identified, selected, or sorted according to the present disclosure is desired for further use, such as expansion and/or use in therapy, the cell will be kept intact (e.g. not lysed) and preferably markers measurable external to the cell (e.g., cell surface markers) will be assessed, such as by, for example, flow cytometry. In some contexts, cell surface markers can be used as surrogates for secreted or intracellular markers. Secreted markers may be assessed by, for example, measuring in cell supernatants. In some preferred embodiments, one or more cell surface markers is assessed, such as by using flow cytometry. In some embodiments, flow cytometry can comprise use of a labelled internalizing antibody or antigen-binding fragment thereof, see e.g. Li et al. J Biomol Screen. 20 (7): 869-875 (2015), which techniques and reagents are incorporated herein by reference.

If a cell is to be lysed (e.g., if the cell's TCR nucleotide sequences are of interest), internally expressed markers may be directly assessed, e.g. using oligonucleotide probes or antibodies or antigen-binding fragments. In some cases, internally expressed markers may be measured using an internalizing antibody or antigen-binding fragment. For internally expressed markers, mRNA, protein, or both may be measured.

Also provided are methods of identifying prognosis, or identifying a risk profile, or reducing the risk of relapse and/or progression, or selecting or adjusting therapy, for a subject having or having been diagnosed with a solid cancer, wherein the methods comprise identifying or assessing the prevalence of CD4+ T cells characterized in accordance to the present disclosure.

Also provided are kits that comprise one or more reagent for sorting or for identifying CD4+ T cells in accordance with the teachings of the present disclosure.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps that do not materially affect the basic characteristics of a claimed subject-matter. For example, a protein domain, region, or module (e.g., a binding domain, hinge region, or linker) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid and non-naturally occurring amino acid polymers.

As used herein, “fusion protein” refers to a protein that, in a single chain, has at least two distinct domains, wherein the domains are not naturally found together in a protein. A polynucleotide encoding a fusion protein may be constructed using PCR, recombinantly engineered, or the like, or such fusion proteins can be synthesized. A fusion protein may further contain other components, such as a tag, a linker, or a transduction marker. In certain embodiments, a fusion protein comprises a binding protein that is expressed or produced by a host cell (e.g., a T cell) locates to a cell surface, where the binding protein is anchored to the cell membrane (e.g., via a transmembrane domain) and comprises an extracellular portion (e.g., containing a binding domain) and an intracellular portion (e.g., containing a effector domain, effector domain, co-stimulatory domain or combinations thereof).

“Nucleic acid molecule” or “polynucleotide” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), polydeoxyribonucleic acid (DNA), which includes cDNA, genomic DNA, and synthetic DNA, either of which may be single or double stranded. If single stranded, the nucleic acid molecule may be the coding strand or non-coding (anti-sense) strand. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

“Percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure, it will be understood that where sequence analysis software is used for analysis, the results of the analysis are based on the “default values” of the program referenced. “Default values” mean any set of values or parameters which originally load with the software when first initialized.

Variants of nucleic acid molecules of this disclosure are also contemplated. Variant nucleic acid molecules include those that are at least 70%, 75%, 80%, 85%, 90%, and preferably 95%, 96%, 97%, 98%, 99%, or 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a binding protein or a binding domain thereof having a functionality described herein, such as specifically binding a target molecule.

A “functional variant” refers to a polypeptide or polynucleotide that is structurally similar or substantially structurally similar to a parent or reference compound of this disclosure, but differs slightly in composition (e.g., one base, atom or functional group is different, added, or removed), such that the polypeptide or encoded polypeptide is capable of performing at least one function of the encoded parent polypeptide with at least 50% efficiency, preferably at least 55%, 60%, 70%, 75%, 80%, 85%, 90%, 95% or 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Ka) or a dissociation (K_{D}) constant).

As used herein, a “functional portion” or “functional fragment” refers to a polypeptide or polynucleotide that comprises only a domain, portion or fragment of a parent or reference compound, and the polypeptide or encoded polypeptide retains at least 50% activity associated with the domain, portion or fragment of the parent or reference compound, preferably at least 55%, 60%, 70%, 75%, 80%, 85%, 90%, 95% or 100% level of activity of the parent polypeptide, or provides a biological benefit (e.g., effector function). A “functional portion” or “functional fragment” of a polypeptide or encoded polypeptide of this disclosure has “similar binding” or “similar activity” when the functional portion or fragment displays no more than a 50% reduction in performance in a selected assay as compared to the parent or reference polypeptide (preferably no more than 20% or 10%, or no more than a log difference as compared to the parent or reference with regard to affinity), such as an assay for measuring binding affinity or measuring effector function (e.g., cytokine release).

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region (“leader and trailer”) as well as intervening sequences (introns) between individual coding segments (exons).

As used herein, the term “endogenous” or “native” refers to a polynucleotide, gene, protein, compound, molecule, or activity that is normally present in a host cell or a subject.

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As used herein, “expression vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. In the present specification, “plasmid,” “expression plasmid,” “virus” and “vector” are often used interchangeably.

The term “introduced” in the context of inserting a nucleic acid molecule into a cell, means “transfection”, or “transformation” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA). As used herein, the term “engineered,” “recombinant” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering (i.e., human intervention). Genetic alterations include, for example, modifications introducing expressible nucleic acid molecules encoding proteins, binding proteins or enzymes, or other nucleic acid molecule additions, deletions, substitutions or other functional disruption of a cell's genetic material. Additional modifications include, for example, non-coding regulatory regions in which the modifications alter expression of a polynucleotide, gene or operon.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a binding protein, or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

The term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g, Geurts et al., Mol. Ther. 8:108, 2003: Matés et al., Nat. Genet. 41:753 (2009)). Exemplary vectors are those capable of autonomous replication (episomal vector) or expression of nucleic acid molecules to which they are linked (expression vectors).

Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses.

“Lentiviral vector,” as used herein, means HIV-based lentiviral vectors for gene delivery, which can be integrative or non-integrative, have relatively large packaging capacity, and can transduce a range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells. Additional vectors useful for practicing embodiments of the present disclosure are described herein.

As used herein, the term “host” refers to a cell (e.g., T cell) or microorganism targeted for genetic modification with a heterologous nucleic acid molecule to produce a polypeptide of interest (e.g., a binding protein of the present disclosure). In certain embodiments, a host cell may optionally already possess or be modified to include other genetic modifications that confer desired properties related or unrelated to, e.g., biosynthesis of the heterologous protein (e.g., inclusion of a detectable marker; deleted, altered or truncated endogenous TCR; or increased co-stimulatory factor expression).

As used herein, a “hematopoietic progenitor cell” is a cell that can be derived from hematopoietic stem cells or fetal tissue and is capable of further differentiation into mature cells types (e.g., immune system cells). Exemplary hematopoietic progenitor cells include those with a CD24^{Lo} Lin-CD117′ phenotype or those found in the thymus (referred to as progenitor thymocytes).

As used herein, an “immune system cell” or “immune cell” means any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells, natural killer (NK) cells, and NK-T cells). Exemplary immune system cells include a CD4⁺ T cell, a CD8⁺ T cell, a CD4 CD8⁺ double negative T cell, a γδ T cell, a regulatory T cell, a natural killer cell (e.g., a NK cell or a NK-T cell), and a dendritic cell. Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

A “T cell” or “T lymphocyte” is an immune system cell that matures in the thymus and produces T cell receptors (TCRs). T cells can be naïve (not exposed to antigen; increased expression of CD62L, CCR7, CD28, CD3, CD127, and CD45RA, and decreased expression of CD45RO as compared to T_{CM}), memory T cells (T_{M}) (antigen-experienced and long-lived), and effector cells (antigen-experienced, cytotoxic). TM can be further divided into subsets of central memory T cells (T_{CM}, increased expression of CD62L, CCR7, CD28, CD127, CD45RO, and CD95, and decreased expression of CD54RA as compared to naïve T cells) and effector memory T cells (T_{EM}, decreased expression of CD62L, CCR7, CD28, CD45RA, and increased expression of CD127 as compared to naïve T cells or T_{CM}).

Effector T cells (T_{E}) refers to antigen-experienced CD8+ cytotoxic T lymphocytes that has decreased expression of CD62L, CCR7, CD28, and are positive for granzyme and perforin as compared to T_{CM}. Helper T cells (T_{H}) are CD4+ cells that influence the activity of other immune cells by releasing cytokines. CD4+ T cells can activate and suppress an adaptive immune response, and which of those two functions is induced can depend on presence of other cells and signals. Some CD4+ T cells can direct exert cytotoxic and/or cytolytic activity on a target cell, such as a tumor cell. T cells can be collected using known techniques, and the various subpopulations or combinations thereof can be enriched or depleted by known techniques, such as by affinity binding to antibodies, flow cytometry, or immunomagnetic selection. Other exemplary T cells include regulatory T cells, such as CD4⁺ CD25⁺ (Foxp3⁺) regulatory T cells, stem cell memory T cells, and Treg17 cells, as well as Tr1, Th3, CD8+CD28;, and Qa-1 restricted T cells.

“Cells of T cell lineage” refer to cells that show at least one phenotypic characteristic of a T cell, or a precursor or progenitor thereof that distinguishes the cells from other lymphoid cells, and cells of the erythroid or myeloid lineages. Such phenotypic characteristics can include expression of one or more proteins specific for T cells (e.g., CD3⁺, CD4⁺, CD8⁺), or a physiological, morphological, functional, or immunological feature specific for a T cell. For example, cells of the T cell lineage may be progenitor or precursor cells committed to the T cell lineage; CD25⁺ immature and inactivated T cells; cells that have undergone CD4 or CD8 linage commitment; thymocyte progenitor cells that are CD4 CD8+ double positive; single positive CD4⁺ or CD8⁺; TCRαβ or TCR γδ; or mature and functional or activated T cells. Additional description of T cells and certain T cell proteins is provided herein.As used herein, “hyperproliferative disease” refers to excessive growth or proliferation as compared to a normal or undiseased cell. Exemplary hyperproliferative disorders include tumors, cancers, neoplastic tissue, carcinoma, sarcoma, malignant cells, pre-malignant cells, as well as non-neoplastic or non-malignant hyperproliferative disorders (e.g., adenoma, fibroma, lipoma, leiomyoma, hemangioma, fibrosis, restenosis, as well as autoimmune diseases such as rheumatoid arthritis, osteoarthritis, psoriasis, inflammatory bowel disease, or the like). Certain diseases that involve abnormal or excessive growth that occurs more slowly than in the context of a hyperproliferative disease can be referred to as “proliferative diseases”, and include certain tumors, cancers, neoplastic tissue, carcinoma, sarcoma, malignant cells, pre-malignant cells, as well as non-neoplastic or non-malignant disorders “Treat” or “treatment” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising a host cell expressing a binding protein of the present disclosure, and optionally an adjuvant, is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; prolonged survival; or any combination thereof.

As used herein, “statistically significant” refers to a p value of 0.050 or less when calculated using the Students t-test and indicates that it is unlikely that a particular event or result being measured has arisen by chance.

As used herein, the term “adoptive immune therapy” or “adoptive immunotherapy” refers to administration of naturally occurring or genetically engineered, disease target (e.g. antigen)-specific immune cells (e.g., T cells). Adoptive cellular immunotherapy may be autologous (immune cells are from the recipient), allogeneic (immune cells are from a donor of the same species) or syngeneic (immune cells are from a donor genetically identical to the recipient).

### Cells, Markers, and Compositions

In certain aspects, the present disclosure provides CD4+ T cells characterized by or identifiable by expression of one or more markers. In any of the presently disclosed embodiments, a CD4+ T cell or CD4+ T cells may be human. CD4+ T cells can be, for example, isolated or present in a sample or can be derived or obtained from a sample, or can be comprised in a cell composition. A sample includes any biological sample from a subject that comprises CD4+ T cells. For example, a sample can comprise lymphocytes, blood, tumor, tumor infiltrated by lymphocytes, tumor-infiltrating lymphocytes, non-tumor-infiltrating lymphocytes, healthy tissue, or any combination thereof, provided that one or more CD4+ T cells are present. CD4+ T cells can be identified using, e.g., an antibody or antibodies specific for CD4. A sample can be from a subject having or previously diagnosed with a cancer, such as a solid cancer. Non-limiting examples of solid cancers include melanomas and breast cancers; other solid cancers are known in the art and described herein.

In some embodiments, a sample comprises material from two or more (e.g. human) subjects. In other embodiments, a sample comprises material from only one (e.g. human) subject. If the sample is from one or more subject having one or more tumors, the sample can comprise material from only one tumor, or from multiple tumors. A sample can include cells from one type of cancer (e.g., a solid cancer such as breast cancer or melanoma) or from two or more types of cancer (e.g., breast cancer and melanoma).

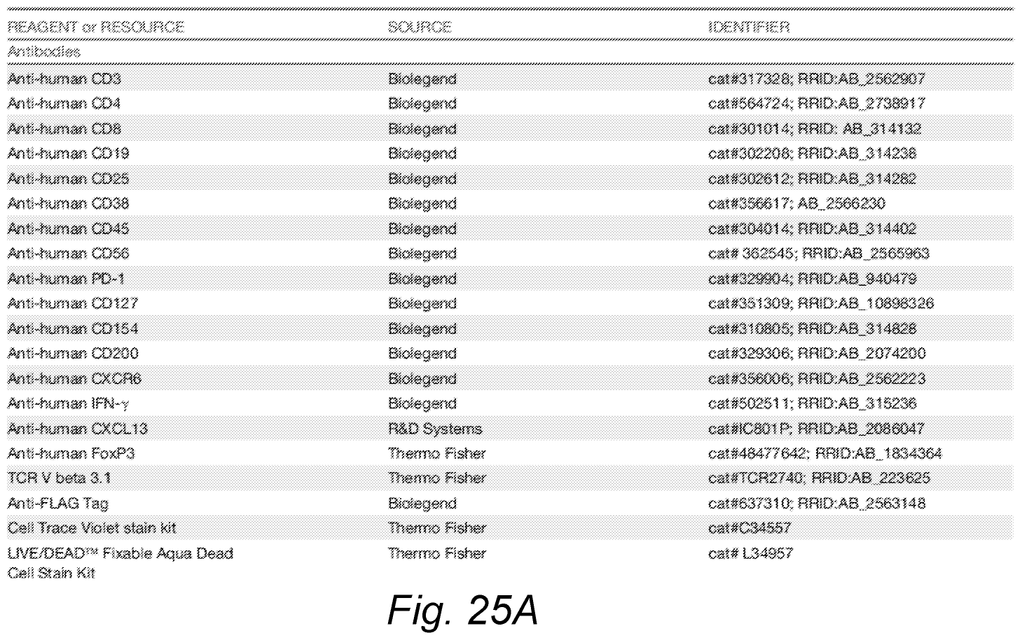

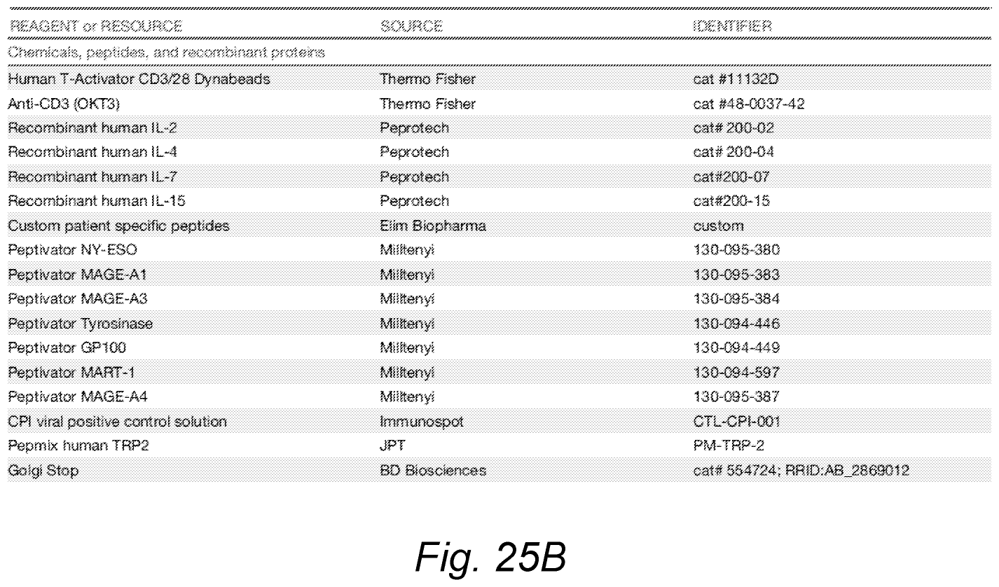

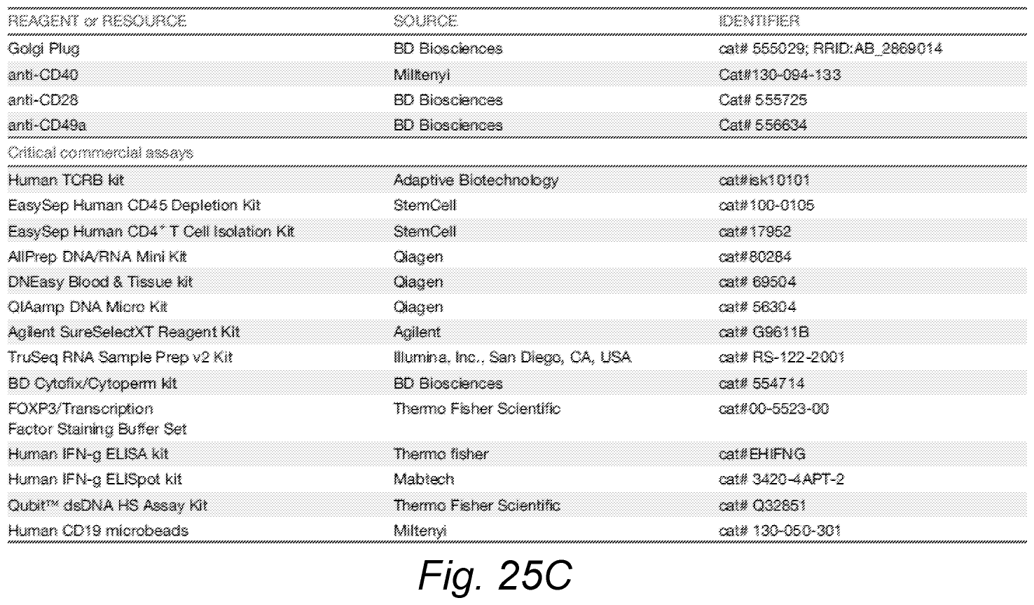

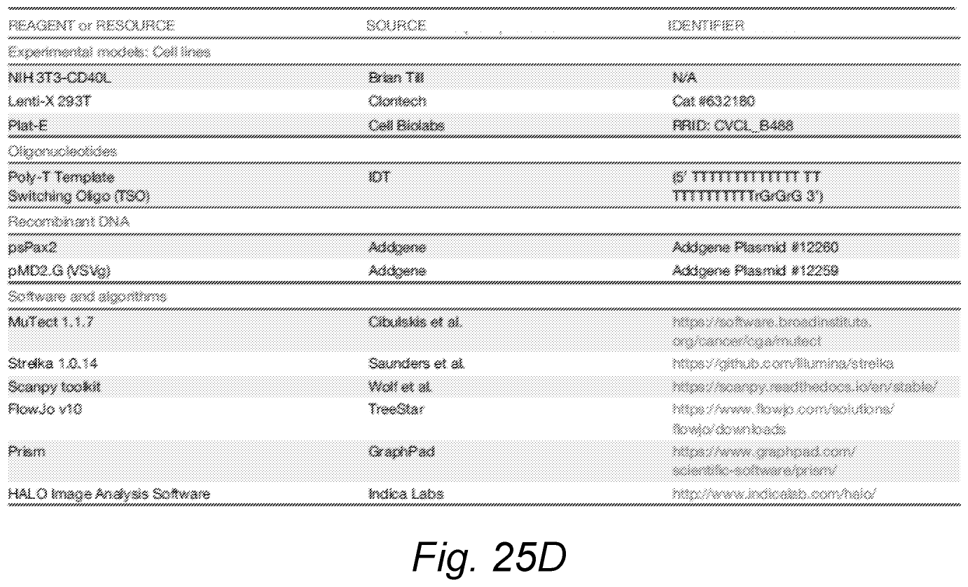

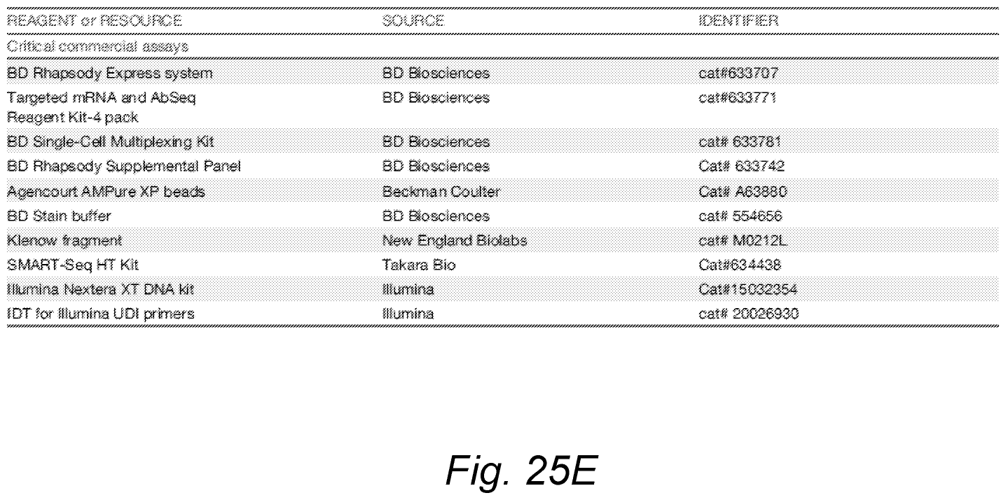

Expression of a marker can be identified using any appropriate reagent or technique. For example, expression of cell-surface markers can be identified using flow cytometry or immunohistochemistry, such as comprising use of an antibody or antigen-binding fragment (e.g., that is labelled, for example using a fluorescent label) specific for the cell-surface marker. Intracellular markers can be assessed using an antibody or antigen-binding fragment, using a labeled oligonucleotide probe, or the like. Binding agents such as antibodies or antigen-binding fragments can also be used to detect soluble or secreted markers. Non-limiting examples of reagents and resources that may be useful in the practice of embodiments of the present disclosure are provided in FIGS. 25A-25E.

Non-limiting examples of antibodies specific for certain T cell proteins, including CXCL13 and CD4, are shown in Table 2 of Example 11.

Non-limiting examples of anti-CD200 antibodies are provided in PCT Publication Nos.: WO2011100538; WO2007084321; WO2012106634; and WO2018102594, and in Rastogi et al., Br J Haematol. 193 (1): 155-159 (2021) doi: 10.1111/bjh.17125, Kretz-Rommel et al. J Immunol. 180 (2): 699-705 (2008) doi.org/10.4049/jimmunol.180.2.699, and Chen and Gorczynski Translpantation 79 (3): 282-8 (2005) doi: 10.1097/01.tp.0000149506.61000.86. These anti-CD200 antibodies are incorporated herein by reference. Anti-CD200 antibodies are also available from commercial suppliers such as BioLegend, ThermoFisher, and Abcam. These antibodies are incorporated herein by reference.

Non-limiting examples of anti-CXCL13 antibodies are provided in PCT Publication Nos.: WO2014003742; WO2014121053; and WO2014121053, and in Klimatcheva et al. BMC Immunol. 2015 Feb. 12;16 (1): 6. doi: 10.1186/s12865-015-0068-1, Yamamoto et al. Mucosal Immunol. 2014 September;7 (5): 1244-54. doi: 10.1038/mi.2014.14, Zheng et al. Arthritis Rheum. 2005 February;52 (2): 620-6. doi: 10.1002/art.20768, and Huang et al. Front Immunol. 2021 Nov. 12; 12:763065. doi: 10.3389/fimmu.2021.763065. These anti-CXCL13 antibodies are incorporated herein by reference. Anti-CXCL13 antibodies are also available from commercial suppliers such as RNDSystems. These antibodies are incorporated herein by reference.

Non-limiting examples of anti-PD-1 antibodies are provided in PCT Publication Nos.: WO/2022/141378; WO/2022/012428; WO/2022/124866; WO/2023/284741; WO/2023/011654; WO/2018/217227; WO/2021/263166; WO/2020/156509; WO/2021/000813; WO/2018/137576; WO/2018/034226; WO/2019/096136; WO/2021/063201; WO/2018/050039; WO/2022/258011; WO/2017/071625; WO/2016/197497; and WO/2017/166804. These anti-PD-1 antibodies are incorporated herein by reference. Anti-PD-1 antibodies also include, without limitation, nivolumab (OPDIVO®); pembrolizumab (KEYTRUDA®); cemiplimab (LIBTAYO®); IBI-308; BCD-100; avelumab (BAVENCIO®); camrelizumab; JS-001; spartalizumab; tislelizumab; AGEN-2034; CBT-501; dostarlimab; JNJ-3283; ABBV-181; AK-104; AK-105; AMP-224; BAT-1306; BI-754091; CC-90006; CS-1003; GLS-010; LZM-009; MEDI-5752; MGD-013; PF-06801591; Sym-021; XmAb-20717; AK-112; ALPN-202; AM-0001; BH-2922; BH-2941; BH-2950; BH-2954; BLSM-101; CB-201; CB-213; CBT-103; CBT-107; CX-188; HAB-21; HEISCOIII-003; IKT-202; JTX-4014; MCLA-134; MD-402; mDX-400; MGD-019; PEGMP-7; PRS-332; RXI-762; STIA-1110; TSR-075; XmAb-23104; AT-16201; IMM-1802; SNA-01; SSI-361; YBL-006; AK-103; JY-034; AUR-012; BGB-108; ENUM-244C8; ENUM-388D4; MEDI-0680; Sym-016; Retifanlimab; and MEDI-0680. These antibodies are incorporated herein by reference.

Non-limiting examples of anti-TCF7 antibodies include: C.725.7 (ThermoFisher, Cat. No. MA5-14965); 18H15L9 (ThermoFisher, Cat No. 702957); G.425.9 (ThermoFisher, Cat No. MA5-14972); TCF7 Polyclonal Antibody (ThermoFisher, Cat. Nos. 14464-1-AP, PA5-78191, PA5-116013, PA5-84842, PA5-102691, PA5-77813, 61579, and PA5-91923); 2E9 (ThermoFisher, Cat. No. H00006932-M02); and 7F11A10 (BioLegend, Cat No. 655207); and the lik. These antibodies are incorporated herein by reference.

Non-limiting examples of anti-BCL6 antibodies include: BCL6 Monoclonal Antibody (BL6.02 (PG-B6p)) (ThermoFisher, Cat No. MA5-11493); BCL6 Monoclonal Antibody (BCL-UP), APC, eBioscience™ (ThermoFisher, Cat. No. 17-9880-42); BCL6 Monoclonal Antibody (BCL-UP), PerCP-eFluor™ 710, eBioscience™ (ThermoFisher, Cat. No. 46-9880-42); BCL6 Polyclonal Antibody (ThermoFisher, Cat. No. 21187-1-AP); BCL6 Recombinant Rabbit Monoclonal Antibody (JB18-48) (ThermoFisher, Cat. No. MA5-34712); and BCL6 Monoclonal Antibody (GI191E), eBioscience™ (ThermoFisher, Cat. No. 14-9887-82); and the like. These antibodies are incorporated herein by reference.

Non-limiting examples of anti-CXCR5 antibodies include: CD185 (CXCR5) Monoclonal Antibody (MUSUBEE), PE, eBioscience™ (ThermoFisher, Cat. No. 12-9185-42); CD185 (CXCR5) Monoclonal Antibody (MUSUBEE), PE-Cyanine7, eBioscience™ (ThermoFisher, Cat. No. 25-9185-42); CD185 (CXCR5) Monoclonal Antibody (MUSUBEE), eBioscience™ (ThermoFisher, Cat. No. 14-9185-82); CD185 (CXCR5) Monoclonal Antibody (MUSUBEE), PE-Cyanine5, eBioscience™ (ThermoFisher, Cat. No. 15-9185-42); and the like. These antibodies are incorporated herein by reference.

Non-limiting examples of anti-TIM3 antibodies include: CD366 (TIM3) Monoclonal Antibody (F38-2E2), Super Bright™ 780, eBioscience™ (ThermoFisher, Cat. No. 78-3109-42); CD366 (TIM3) Monoclonal Antibody (F38-2E2), APC, eBioscience™ (ThermoFisher, Cat. No. 17-3109-42); CD366 (TIM3) Monoclonal Antibody (F38-2E2), Functional Grade, eBioscience™ (ThermoFisher, Cat. No. 16-3109-85); F38-2E2; TIM3 Monoclonal Antibody (4C4G3) (ThermoFisher, Cat. No. 60355-1-IG); TIM-3 Monoclonal Antibody (1E5) (ThermoFisher, Cat. No. MA5-32841); and the like. These antibodies are incorporated herein by reference.

Non-limiting examples of anti-LAG3 antibodies include: 3DS223H; CD223 (LAG-3) Monoclonal Antibody (3DS223H), PerCP-eFluor™ 710, eBioscience™ (ThermoFisher, Cat. No. 46-2239-42); LAG525 (IMP701); Relatlimab; TSR-033; Sym022; MK-4280; REGN3767; GSK2831781; INCAGN02385; and BMS-986016. These antibodies are incorporated herein by reference.

Non-limiting examples of anti-CCL3 antibodies include: CCL3 (MIP-1 alpha) Polyclonal Antibody (ThermoFisher, Cat. No. PA5-86025); CCL3 Polyclonal Antibody (ThermoFisher, Cat. No. PA5-96722); CCL3 (MIP-1 alpha) Monoclonal Antibody (CR3M), PE, eBioscience™ (ThermoFisher, Cat. No. 12-9706-42); CCL3 (MIP-1 alpha) Monoclonal Antibody (CR3M), FITC, eBioscience™ (ThermoFisher, Cat. No. 11-9706-42); CCL3 Polyclonal Antibody (ThermoFisher, Cat. No. PA5-86585); and CCL3 Monoclonal Antibody (14D7 1G7) (ThermoFisher, Cat. No. AHC6034). These antibodies are incorporated herein by reference.

Non-limiting examples of anti-CCL4 antibodies include: CCL4 Monoclonal Antibody (24006) (ThermoFisher, Cat. No. MA5-23713); FL34Z3L; CCL4 (MIP-1 beta) Monoclonal Antibody (FL34Z3L), APC, eBioscience™ (ThermoFisher, Cat. No. 17-7540-42); CCL4 Recombinant Polyclonal Antibody (6HCLC) (ThermoFisher, Cat. No. 710391); and CCL4 Polyclonal Antibody (ThermoFisher, Cat. No. PA5-34509). These antibodies are incorporated herein by reference.

Non-limiting examples of anti-CD103 antibodies include: B-Ly6; CD103 (Integrin alpha E) Monoclonal Antibody (B-Ly7), PerCP-eFluor™ 710, eBioscience™ (ThermoFisher, Cat. No. 46-1038-42); CD103 (Integrin alpha E) Monoclonal Antibody (Ber-ACT8), APC, eBioscience™ (ThermoFisher, Cat. No. 17-1037-42); and CD103 Polyclonal Antibody (ThermoFisher, Cat. No. PA5-80744). These antibodies are incorporated herein by reference.

Cells can also be evaluated using microarray, immunomagnetic selection, RNA sequencing (e.g., single-cell RNA sequencing), qPCR, or intracellular staining using an antibody or other binding agent, to confirm the presence or absence of cells expressing a marker or markers as disclosed herein. These and other techniques disclosed in the present Examples may be used. If cells in a composition are known to be clonally related (e.g., derived from a single ancestor), a representative parental or progeny cell can be analyzed and the results may be extrapolated to the number of known or estimated progeny in the sample.

Flow cytometry or the like can be used to, for example, identify, sort, or analyze cells of interest. Expression of a marker can also be assessed using, for example, mRNA sequencing, quantitative reverse-transcriptase PCR, or the like. Differential expression among cells or within or between populations or groups of cells can be assessed using, for example, MAST (Finak, G., McDavid, A., Yajima, M., Deng, J., Gersuk, V., Shalek, A. K., Slichter, C. K., Miller, H. W., McElrath, M. J., and Prlic, M. (2015). MAST: a flexible statistical framework for assessing transcriptional changes and characterizing heterogeneity in single-cell RNA sequencing data. Genome biology 16, 1-13).

Sorting can include, for example, removing cells of interest from a sample (and leaving therein cells not of interest), removing cells that are not of interest from a sample (and leaving therein cells of interest), or separating cells of interest from cells not of interest within the sample.

In some embodiments, a cell is said to be “positive” or “negative” for expression of a marker. Expression of a marker may vary between subjects or samples; e.g. in one subject or sample, an expression level of a marker may be lower in comparison to the expression level of the marker in another subject or sample, but both subjects or samples may be still be “positive” for expression. In certain preferred embodiments, expression is described with reference to expression by other cells in the sample or from the subject. An appropriate control may be used to determine positive or negative expression and degrees of positive expression (e.g., high, intermediate or low positive expression). For example, positive or negative staining for expression using an antibody, and relative degrees of positive staining, can be assessed versus an isotype control—e.g. an antibody that is of the same isotype as the marker-specific antibody but that is not specific for the marker (i.e. is specific for a negative control marker or altogether lacks specific target binding). Such a control provides a reference point from which to identify expression of (e.g. specific staining for) the marker.

For example, with reference to a sample comprising cells, expression of a marker by cells may be assessed using an antibody or antigen-binding fragment thereof that is specific for the marker, and an isotype control antibody or antigen-binding fragment. In such a setting, a (n e.g. CD4+ T) cell that is “positive” for expression of the marker has a detected level of the marker (e.g. as detected via staining using a labelled anti-marker antibody or antigen-binding fragment, such as by flow cytometry) that is preferably greater than staining on 99% of CD4+ T cells (preferably CD3+CD4+ T cells) in the sample when interrogated using the isotype control. For example, when staining cells using isotype control and e.g. anti-CD200, the cells of interest stain for CD200 with an intensity that is greater than the intensity with which 99% of the cells stain using isotype control. It will be understood that less than all CD4+ T cells in a sample may be analyzed, and in such a context “positive” refers to a detected level of the marker that is greater than the level of 99% of analyzed CD4+ T cells using a negative control (e.g. isotype control). In certain preferred embodiments, most if not all or substantially all CD4+ T cells in the sample may be analyzed.

“Negative” or “low” expression of the marker is expression equivalent to or below the threshold defined by the isotype control. “Intermediate” expression of a marker and “high” expression of a marker are relative to the given sample or subject and are defined relative to low/negative cells from the sample or subject. It will also be understood that a cell can be positive for expression, but at an intensity or level that is lower than an “intermediate” level of expression; e.g. as defined by other positive cells from the sample.

In some embodiments, expression of a marker by a CD4+ T cell is assessed or described relative to, or with reference to, expression of the marker by another cell or cells, e.g. other CD4+ T cells. Relative expression of a marker or markers as between different cells includes circumstances in which one cell (e.g. a CD4+ T cell of interest) expresses a marker and another cell (e.g. a CD4+ T cell not possessing one or more herein-described phenotype of interest) does not express, or does not detectably express, the marker using the same assay. Relative expression of a marker or markers also includes circumstances in which a cell has increased expression of the marker as compared to another cell or cells that also express the marker.

In some embodiments, expression of a marker or markers by a subject cell or cells is described with reference to expression of the marker by one or more other cells of the same sample or subset. For example, expression or a marker by a subject CD4+ T cell or cells can be “increased” or “elevated” or “reduced” or “decreased” as compared to CD4+ other cells from the sample (e.g., tumor, tumor infiltrate, tumor suspension, blood, or the like), and optionally to an isotype control. In some embodiments, a subject CD4+ T cell is from tumor infiltrate and a comparator cell comprises another CD4+ T cell from the tumor infiltrate or from infiltrate of another tumor of the (e.g. human) subject. In some embodiments, a subject CD4+ T cell is from tumor infiltrate and a comparator cell comprises a CD4+ T cell that is not from tumor infiltrate. In some embodiments, a subject CD4+ T cell does not express one or more markers of exhaustion and a comparator cell expresses one or more markers of exhaustion.

In some embodiments, expression of a marker by a subject CD4+ T cell or cells is described relative to: the average, median, typical, or most frequently observed or occurring level of expression by all, or all assessed, CD4+ T cells in the sample, or by other or all CD4+ T cells from tumor infiltrate (if, for example, the subject CD4+ T cell or cells is from tumor infiltrate), or by one or more CD4+ T cells not from tumor infiltrate (e.g. from blood or healthy tissue if, for example, the subject CD4+ T cell or cells is/are from tumor infiltrate), or the like.

In some embodiments, a flow cytometry method is provided that comprises identifying, from a (n e.g. tumor, such as a solid tumor) sample comprising CD4+ T cells, one or more CD4+ T cells that are positive for expression of CD200, or that express CD200. In some embodiments, a flow cytometry method is provided that comprises identifying, from a (n e.g. tumor, such as a solid tumor) sample comprising CD4+ T cells, one or more CD4+ T cells that are positive for expression of CD200 and for PD-1. In some embodiments, a flow cytometry method is provided that comprises identifying, from a (n e.g. tumor, such as a solid tumor) sample comprising CD4+ T cells, one or more CD4+ T cells that express CD200 and PD-1.

In some embodiments, a flow cytometry method is provided that comprises identifying, from a (n e.g. tumor, such as a solid tumor) sample comprising CD4+ T cells, one or more CD4+ T cells that have express CD200 and PD-1 with a greater intensity as compared to one or more other CD4+ T cells of the sample. In some embodiments, the one or more other CD4+ T cells comprise 99% or more of analyzed CD4+ T cells and the analyzed CD4+ T cells are stained using an isotype control.

In some embodiments, a flow cytometry method is provided that comprises identifying, from a (n e.g. tumor, such as a solid tumor) sample comprising CD4+ T cells, one or more CD4+ T cells that are positive for expression of CXCL13, or that express CXCL13.

In certain embodiments, the flow cytometry method further comprises sorting CD4+ T cells that express CXCR6 from CD4+ T cells that do not express CXCR6. In some embodiments, a method comprises identifying or sorting for one or more (e.g. CD4+) T cells positive for expression of PD-1, positive for expression of CD200, and negative for expression of CXCR6. In some embodiments, a method comprises identifying or sorting for one or more (e.g. CD4+) T cells expressing PD-1, expressing CD200, and not expressing CXCR6. In some embodiments, a method comprises identifying or sorting for one or more (e.g. CD4+) T cells positive for expression of PD-1, positive for expression of CD200, and positive for expression of CXCR6. In some embodiments, a method comprises identifying or sorting for one or more (e.g. CD4+) T cells expressing PD-1, expressing CD200, and expressing CXCR6. In some embodiments, a method comprises sorting away, from other CD4+ T cells (of e.g. a tumor sample), one more CD4+ T cells not expressing CD200.

In certain embodiments, a CD4+ T cell is provided, wherein the CD4+ T cell was obtained from a sample comprising a plurality of CD4+ T cells and: (i) expresses CXCL13, optionally having increased expression of CXCL13 as compared to other CD4+ T cells of the plurality; (ii) expresses PD-1 and CXCL13, optionally having increased expression of PD-1 and/or of CXCL13 as compared to other CD4+ T cells of the plurality; (iii) expresses CD200, optionally having increased expression of CXCL 13 as compared to other CD4+ T cells of the plurality; or (iv) expresses PD-1 and CD200, optionally having increased expression of PD-1 and/or of CXCL13 as compared to other CD4+ T cells of the plurality, wherein, optionally, the CD4+ T cell: (a) is negative for CD25 expression or has reduced expression of CD25 as compared to other CD4+ T cells of the plurality; (b) expresses CXCR6, optionally at an increased level as compared to other CD4+ T cells in the plurality; and/or (c) is negative for expression of CXCR6 or has reduced expression of CXCR6 as compared to other CD4+ T cells of the plurality. The CD4+ T cell may additionally: (i) have increased expression of (1) one or more memory gene and/or (2) one or more gene or surface marker associated with T follicular helper (T_{FH}) cells, as compared to other CD4+ T cells in the sample; (ii) (1) be negative for TCF7 expression or have reduced TCF7 expression, as compared to other CD4+ T cells in the sample; and/or (2) express one or more coinhibitory marker, one or more inflammatory marker, one or more cytolytic marker, and or one or more tissue resident memory marker; and/or (iii) express one or more gene involved in proliferation. The one or more memory gene can comprise TCF7, IL-7R, or both. The one or more gene or surface marker associated with T follicular helper (T_{FH}) cells can comprise BCL6, CD200, CXCR5, or any combination thereof. The one or more coinhibitory marker can comprise TIM-3, LAG-3, or both. The one or more inflammatory marker can comprise CCL3, CCL4, IFNγ, IFN-γ mRNA, or any combination thereof. The one or more cytolytic marker can comprise GZMA/K, PRF1 mRNA, or both. The one or more gene involved in proliferation can comprise TYMS, TOP2A, MCM2/4 mRNA, or any combination thereof. The one or more tissue resident memory marker can comprise CD103.

In certain embodiments, the CD4+ T cell: (i) has increased expression of CXCL13 and any one or more of TCF7, IL7R, BCL6, and CD200, as compared to other CD4+ T cells from the sample; (ii) has increased expression of CXCL13 as compared to other CD4+ T cells from the sample, are negative for TCF7 expression or have reduced TCF7 expression, as compared to other CD4+ T cells in the sample, and express one or more of TIM-3, LAG-3, IFN-γ mRNA, GZMA/K, PRF1 mRNA, and CD103; or (iii) has increased expression of CXCL13 as compared to other CD4+ T cells from the sample and express one or more of TYMS, TOP2A, and MCM2/4 mRNA, and optionally, expresses BTLA.

In certain embodiments, expression of a cell surface protein (e.g. CXCL13, CD4, CD200, PD-1, CXCR6) comprises expression of the cell surface protein at the cell surface.

In some embodiments, a CD4+ T cell is provided that expresses: (i) CXCL13; (ii) PD-1 and CXCL13; (iii) CD200; and/or (iv) PD-1 and CD200. In some embodiments, the CD4+ T cell (i) expresses (1) one or more memory gene and/or (2) one or more gene or surface marker associated with T follicular helper (TFH) cells; (ii) (1) is negative for TCF7 expression; and/or (2) expresses one or more coinhibitory marker, one or more inflammatory marker, one or more cytolytic marker, and or one or more tissue resident memory marker; (iii) expresses one or more gene involved in proliferation; (iv) expresses BTLA; (v) expresses CXCR6; and/or (vi) is negative for expression of CXCR6, and, optionally, is negative for CD25 expression.

The one or more memory gene can comprise TCF7, IL-7R, or both. The one or more gene or surface marker associated with T follicular helper (TFH) cells can comprise BCL6, CD200, CXCR5, or any combination thereof. The one or more coinhibitory marker can comprise TIM-3, LAG-3, or both. The one or more inflammatory marker can comprise CCL3, CCL4, IFNγ, IFN-γ mRNA, or any combination thereof. The one or more cytolytic marker can comprise GZMA/K, PRF1 mRNA, or both. The one or more gene involved in proliferation can comprise TYMS, TOP2A, MCM2/4 mRNA, or any combination thereof. The one or more tissue resident memory marker can comprise CD103.

In certain embodiments, a CD4+ T cell expresses (i) CXCL13, (ii) PD-1 and CXCL13, (iii) CD200; and/or (iv) PD-1 and CD200, and further expresses: (a) TCF7, IL7R, BCL6, CD200, CXCR5, or any combination thereof; (b) TIM-3, LAG-3, IFN-γ mRNA, GZMA/K, PRF1 mRNA, CD103, or any combination thereof; (c) TYMS, TOP1A, MCM2/4 mRNA, or any combination thereof; and/or (d) BTLA.

A CD4+ T cell or CD4+ T cells of the present disclosure can be from a sample: (i) comprising tumor, tumor infiltrated by lymphocytes, tumor infiltrating lymphocytes, and/or blood; (ii) from a subject having, or having previously been diagnosed with, a cancer, such as a solid cancer (e.g., melanoma or breast cancer); or (iii) both (i) and (ii). In some embodiments, the subject has previously received an anti-PD-1 antibody or antigen-binding fragment thereof.

A CD4+ T cell or CD4+ T cells of the present disclosure can be specific against a tumor antigen or a tumor neoantigen, optionally an antigen or neoantigen from a solid tumor. For example, tumor antigens or tumor neoantigens (e.g. from the same tumor from which a subject CD4+ T cell or CD4+ T cells were obtained or derived) can be identified using any suitable technique. For example, whole exome sequencing can be performed on tumor and normal cells, or RNA sequencing can be performed on tumor cells, to identify known antigenic mutations or nonsynonymous mutations that could serve as neoantigens. Highly expressed mutations can be screened for reactivity by a CD4+ T cell or CD4+ T cells of the subject. For candidate neoantigens, 20-mer peptides with the variant amino acid at either position 7 or 13 may be synthesized for screening. Screening can be performed by, for example, culturing the CD4+ T cell or CD4+ T cells in the presence of a single peptide (i.e. all peptides in the culture comprise the same amino acid sequence) or a in the presence of a pool of peptides comprising two or more different peptides and determining expression of, for example, one or more cytokines (e.g., IFN-Y), activation markers, or other biomolecules by the CD4+ T cell or CD4+ T cells.

Also provided are compositions comprising a plurality of a CD4+ T cell according to the present disclosure and one or both of: (i) a plurality of tumor antigen-specific or tumor neoantigen-specific CD8+ T cells; and (ii) a pharmaceutically acceptable carrier, excipient, or diluent. Cell compositions and pharmaceutically acceptable carriers, excipients, and diluents are described further herein.

Also provided is a population of CD4+ T cells or a composition comprising CD4+ T cells, wherein 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, or 100% of the CD4+ T cells in the population or composition, respectively, are CD4+ T cells according to the present disclosure.

Also provided is a CD4+ T cell population enriched for CD4+ T cells according to the present disclosure. Enrichment includes a relative increase in the number of CD4+ T cells as compared to a non-enriched sample. For example, a sample comprising cells of interest and cells that are not of interest can be enriched for the cells of interest by: (i) increasing the number of cells of interest (e.g. by selectively stimulating the cells of interest to proliferate) while not correspondingly increasing the number of cells not of interest; (ii) removing or depleting cells not of interest while not correspondingly removing or depleting cells of interest; or (iii) both (i) and (ii).

In certain embodiments, enrichment can occur prior to any one or both of an activation or expansion step. In certain embodiments, enrichment occurs after any one or both of an activation or expansion step (e.g., enrichment of cells that have been activated and thereafter expanded). Activation, transduction, and enrichment techniques are known in the art and include those disclosed herein and in U.S. Pat. No. 6,040,177.

In some contexts, cells not of interest comprise “bystander” cells that may be present in tumor infiltrate but are specific for non-tumor (e.g. viral or bacterial) antigens, Treg cells, or both. In some embodiments, bystander cells are PD-1 low, CXCL13 low, PD-1 low/CXCL13 low, PD-1 low/CD127+ high, CD25 low/PD-1 low/CD127 high, or any combination thereof. Bystander cells can be identified by expression of one or more markers (e.g. as described above), reactivity against a non-tumor antigen, or the like.

In some embodiments, a sample enriched for a cell or cells of interest (e.g. having an expression profile as described herein) has from about a 2-fold to about a 20-fold increase in the number of cells of interest as compared to a non-enriched sample (e.g. as compared to tumor infiltrate from which the cell or cells of interest were obtained or derived). In some embodiments, a sample enriched for a cell or cells of interest (e.g. having an expression profile as described herein) has at least about 1.5-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 10.5-fold, at least about 11-fold, at least about 11.5-fold, at least about 12-fold, at least about 12.5-fold, about 13-fold, about 13.5-fold, about 14-fold, about 14.5-fold, about 15-fold, about 15.5-fold, at least about 16-fold, at least about 16.5-fold, at least about 17-fold, at least about 17.5-fold, at least about 18-fold, at least about 18.5-fold, at least about 19-fold, at least about 19.5-fold, or at least about 20-fold the number of cells of interest as compared to a non-enriched sample. In some contexts, enrichment may vary depending on how common the cell of interest is in the sample or subject. For example, fewer cells of interest in a tumor infiltrate sample may result in a lower enrichment. Furthermore, in some contexts, cells not of interest (e.g. bystander cells) may be more likely to grow well in culture and isolation of the cells of interest (e.g. by deleting or depleting cells no of interest) prior to expansion of a cell sample can result in even further enrichment, after expansion, of the cells of interest, such as for CXCR6+CD4+ T cells.

Also provided is a population of CD4+ T cells or a composition comprising CD4+ T cells, wherein 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, or 100% of the CD4+ T cells in the population or composition are: (i) CXCL13+; (ii) CXCL13+PD-1+; (iii) CD200+; (iv) CD200+PD-1+; (v) CD200+PD-1+ CXCR6-; (vi) CD200+PD-1+CXCR6+; (vii) TCF7+; (vii) TCF7-; or (viii) any combination of (i)-(viii). In some embodiments, the CD4+ T cells were obtained from a sample and have increased expression of CXCL13, PD-1, and/or CD200 as compared to other CD4+ T cells from the sample.

Also provided is a population of CD4+ T cells or a composition comprising CD4+ T cells, wherein the CD4+ T cells are: (i) CXCL13+; (ii) CXCL13+PD-1+; (iii) CD200+; (iv) CD200+PD-1+; (v) CD200+PD-1+ CXCR6-; (vi) CD200+PD-1+ CXCR6+; (vii) TCF7+; (vii) TCF7-; or (viii) any combination of (i)-(viii), and are present in an amount that is at least about 1.5-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 10.5-fold, at least about 11-fold, at least about 11.5-fold, at least about 12-fold, at least about 12.5-fold, about 13-fold, about 13.5-fold, about 14-fold, about 14.5-fold, about 15-fold, about 15.5-fold, at least about 16-fold, at least about 16.5-fold, at least about 17-fold, at least about 17.5-fold, at least about 18-fold, at least about 18.5-fold, at least about 19-fold, at least about 19.5-fold, or at least about 20-fold the amount in a sample (e.g. comprising tumor, tumor infiltrate, or the like) from which the CD4+ T cells were obtained or derived.

Also provided herein are cells (e.g. CD4+ T cells, host cells) made in accordance with a disclosed method, as well as compositions and populations that comprise the cells, and methods of using the cells to, for example, treat cancer.

Also provided are kits for identifying or sorting CD4+ T cells, wherein the kits comprise: (i) a reagent for detecting expression of CXCL13; (ii) a reagent for detecting expression of CD200; or both of (i) and (ii). A kit can further comprise (iii) a reagent for or detecting expression of PD-1, (iv) a reagent for detecting expression of CXCR6, (v) a reagent for detecting expression of TCF7, or any combination of (iii)-(v). A kit can further comprise a reagent for detecting expression of CD25, a reagent for detecting expression of CD127, a reagent for detecting expression of CD4, a reagent for detecting expression of CD3, a reagent for detecting expression of CD40L, or any combination thereof. In some embodiments, a reagent for determining expression comprises an antibody or an antigen-binding fragment thereof. In some embodiments, the kit comprises an antibody or antigen-binding fragment that binds specifically to: CXCL13; CD200; PD-1; CXCR6; TCF7; CD25; CD127; CD4; CD3; CD40L. In some embodiments, the kit comprises more antibodies or antigen-binding fragments for detecting any two or more of: CXCL13; CD200; PD-1; CXCR6; TCF7; CD25; CD127; CD4; CD3; and CD40L. In some embodiments, a reagent for detecting expression comprises a labeled nucleic acid probe specific for mRNA. In some embodiments, the mRNA comprises mRNA from CXCL13; CD200; PD-1; CXCR6; TCF7; CD25; CD127; CD4; CD3; or CD40L. In some embodiments, a kit comprises two or more labeled nucleic acid probes for detecting mRNA from any two or more of CXCL13; CD200; PD-1; CXCR6; TCF7; CD25; CD127; CD4; CD3; and CD40L. In certain embodiments, the kits further comprise instructions for using the reagent(s) to detect expression of the one or more markers, to sort CD4+ T cells, or both.

Also provided is a method comprising identifying, selecting, and/or sorting, from a sample comprising CD4+ T cells, one or more CD4+ T cells positive for expression of CD200, wherein, optionally, the sample is a tumor sample.

Also provided is a method comprising identifying, selecting, and/or sorting, from a sample comprising CD4+ T cells, CD4+ T cells that: (i) express CD200, wherein, optionally, the sample comprises a tumor sample; or (ii) have increased expression of CD200 relative to one or more other CD4+ T cells of the sample, wherein, optionally, the sample comprises a tumor sample.

Also provided is a method comprising identifying, selecting, and/or sorting, from a sample comprising CD4+ T cells, one or more CD4+ T cells positive for expression of CXCL13, wherein, optionally, the sample is a tumor sample.

Also provided is a method comprising identifying, selecting, and/or sorting, from a sample comprising CD4+ T cells, CD4+ T cells that: (i) express CXCL13, wherein, optionally, the sample comprises a tumor sample; or (ii) have increased expression of CXCL13 relative to one or more other CD4+ T cells of the sample, wherein, optionally, the sample comprises a tumor sample.

In certain embodiments: (i) the sample comprising CD4+ T cells comprises blood and/or tumor; and/or (ii) the sample comprising CD4+ T cells is from a subject having, or having previously been diagnosed with, a cancer, such as a solid cancer (e.g., melanoma or breast cancer); and/or (iii) the sample comprising CD4+ T cells comprises tumor-infiltrating lymphocytes and/or non-tumor-infiltrating lymphocytes; and/or (iv) the sample comprising CD4+ T cells is from or comprises tumor infiltrated by lymphocytes; and/or (v) the sample comprising CD4+ T cells is from a subject that has previously been administered an anti-PD-1 antibody or antigen-binding fragment thereof, optionally nivolumab or pembrolizumab; and/or (vi) the sample comprising CD4+ T cells has previously been exposed to an anti-PD-1 antibody or antigen-binding fragment thereof, optionally nivolumab or pembrolizumab.

In some embodiments: (i) identifying, selecting, and/or sorting the one or more CD4+ T cells positive for expression of CXCL13 or expressing CXCL13 or having increased expression of CXCL13 further comprises identifying, selecting, and/or sorting one or more of the identified, selected, and/or sorted CD4+ T cells positive for expression of PD-1 or expressing PD-1 or having increased expression of PD-1 relative to one or more other CD4+ T cells of the sample; and/or (ii) identifying, selecting, and/or sorting the one or more CD4+ T cells positive for expression of CD200 or expressing CD200 or having increased expression of CD200 further comprises identifying, selecting, and/or sorting one or more of the of the identified, selected, and/or sorted CD4+ T cells positive for expression of PD-1 or expressing PD-1 or having increased expression of PD-1 relative to one or more other CD4+ T cells of the sample; and/or (iii) the sample comprising CD4+ T cells has an increased frequency of CD4+ T cells positive for expression of PD-1 or expressing PD-1 or having increased expression of PD-1, relative a sample from the subject having an equivalent number of cells as compared to the sample.

Also provided is a method comprising identifying, selecting, and/or sorting, from a sample comprising CD4+ T cells: (i) one or more CD4+ T cells positive for expression of CXCL13 and PD-1; (ii) one or more CD4+ T cells expressing CXCL13 and PD-1; or (iii) one or more CD4+ T cells that have increased expression of CXCL13 and PD-1 relative to one or more other CD4+ T cells of the sample.

Also provided is a method comprising identifying, selecting, and/or sorting, from a sample comprising CD4+ T cells: (i) one or more CD4+ T cells expressing CD200 and PD-1; (ii)

one or more CD4+ T cells that have increased expression of CD200 and PD-1 relative to one or more other CD4+ T cells of the sample; or (iii) one or more CD4+ T cells positive for expression of CD200 and PD-1.

Also provided is a method comprising identifying, selecting, and/or sorting, from a sample comprising CD4+ T cells, one or more CD4+ T cells positive for expression of CD200 or that express CD200 or that have increased expression of CD200 relative to one or more other CD4+ T cells of the sample, wherein the sample is from a subject that has previously been administered an anti-PD-1 antibody or antigen-binding fragment thereof and/or the sample has been exposed to an anti-PD-1 antibody or antigen-binding fragment thereof.

In certain embodiments: (i) the sample comprises blood and/or tumor, wherein, optionally, the sample comprises blood from a subject that has, or has previously been diagnosed with, a cancer, such as a solid cancer (e.g., melanoma or breast cancer), and has further optionally previously been administered an anti-PD-1 antibody or antigen-binding fragment thereof; and/or (ii) the other cells of the sample consist essentially of, or consist of CD4+ T cells; and/or (iii) the sample comprises, consists essentially of, or consists of tumor-infiltrating lymphocytes.

In some embodiments, the one or more identified, selected, and/or sorted CD4+ T cells:
(i) are positive for expression, express, or have increased expression relative to one or more other CD4+ T cells from the sample, and optionally have intermediate or high expression of, (1) one or more memory gene and/or (2) one or more gene or surface marker associated with T follicular helper (TFH) cells; and/or (ii) (1) are negative for TCF7 expression, do not express TCF7, or have reduced TCF7 expression as compared to other CD4+ T cells in the sample; and/or (2) are positive for expression of, express, or have increased expression as compared to one or more other CD4+ T cells from the sample, of one or more coinhibitory marker, one or more inflammatory marker, one or more cytolytic marker, and/or one or more tissue resident memory marker; and/or (iii) are positive for expression of, express, or have increased expression as compared to one or more other CD4+ T cell from the sample, of one or more gene involved in proliferation. In certain further embodiments: (i) the one or more memory gene comprises TCF7, IL7-R, or both; and/or (ii) the one or more gene or surface marker associated with T follicular helper (TFH) cells comprises BCL6, CD200, CXCR5, or any combination thereof; and/or
(iii) the one or more coinhibitory marker comprises TIM-3, LAG-3, or both; and/or (iv) the one or more inflammatory marker comprises CCL3, CCL4, IFNγ, IFN-γ mRNA, or any combination thereof; and/or (v) the one or more cytolytic marker comprises GZMA/K, PRF1 mRNA, or both; and/or (vi) the one or more gene involved in proliferation comprises TYMS, TOP2A, MCM2/4 mRNA, or any combination thereof; and/or (vii) the one or more tissue resident memory marker comprises CD103. In certain further embodiments, the one or more identified, selected, and/or sorted CD4+ T cells: (i) are positive for expression of, express, or have increased expression relative to one or more other CD4+ T cells of the sample, of any one or more (i.e. any one, any two, any three, any four, any five, or all six) of CXCL13, TCF7, IL7R, BCL6, CD200, and CXCR5, and optionally have high expression of any one or more of CXCL13, TCF7, IL7R, BCL6, CD200, and CXCR5; (ii) (a) are positive for expression of, express, or have increased expression relative to one or more other CD4+ T cells of the sample, of CXCL13, and optionally have high expression of CXCL13, (b) are negative for TCF7 expression or do not express TCF7 expression or have reduced TCF7 expression as compared to one or more other CD4+ T cells of the sample, and (c) are positive for expression of, express, or have increased expression relative to one or more other CD4+ T cells from the sample, of TIM-3, LAG-3, IFN-γ mRNA, GZMA/K, PRF1 mRNA, and CD103; or (iii) (a) are positive for expression of, express, or have increased expression relative to one or more other CD4+ T cells of the sample, of CXCL13, and optionally have high expression of CXCL13, and (b) are positive for expression of, express, or have increased expression relative to one or more other CD4+ T cells of the sample, of TYMS, TOP2A, and MCM2/4 mRNA, and, optionally, (c) express, are positive for expression of, or have increased expression relative to one or more other CD4+ T cells of the sample, BTLA and/or IL-21.

In some embodiments, a method comprises identifying, selecting, and/or sorting one or more CD4+ T cells (a) positive for expression of, expressing, or having increased expression relative to one or more other CD4+ T cells of the sample, of CXCL13, CD200, or both, optionally (b) positive for expression of, expressing, or have increased expression of BTLA relative to one or more other CD4+ T cells of the sample, and: (c) (i) positive for expression of, expressing, or having increased expression relative to one or more other CD4+ T cells of the sample, of (1) one or more memory gene and/or (2) one or more gene or surface marker associated with T follicular helper (TFH) cells; and/or (c) (ii) (1) negative for TCF7 expression, not expressing TCF7, or having reduced TCF7 expression, relative to other CD4+ T cells of the sample; and/or (2) are positive for expression of, express, or have increased expression relative to one or more other CD4+ T cells of the sample, of one or more coinhibitory marker, one or more inflammatory marker, one or more cytolytic marker, and/or one or more tissue resident memory marker; and/or (c) (iii) positive for expression of, expressing, or having increased expression relative to one or more other CD4+ T cells of the sample, of one or more gene involved in proliferation. In certain further embodiments: (i) the one or more memory gene comprises TCF7, IL7-R, or both; and/or (ii) the one or more gene or surface marker associated with T follicular helper (TFH) cells comprises BCL6, CD200, CXCR5, or any combination thereof; and/or (iii) the one or more coinhibitory marker comprises TIM-3, LAG-3, or both; and/or (iv) the one or more inflammatory marker comprises CCL3, CCL4, IFNγ, IFN-γ mRNA, or any combination thereof; and/or (v) the one or more cytolytic marker comprises GZMA/K, PRF1 mRNA, or both; and/or (vi) the one or more gene involved in proliferation comprises TYMS, TOP2A, MCM2/4 mRNA, or any combination thereof; and/or (vii) the one or more tissue resident memory marker comprises CD103. In certain further embodiments: (i) the one or more memory gene comprises TCF7, IL7-R, or both; (ii) the one or more gene or surface marker associated with T follicular helper (T_{FH}) cells comprises BCL6, CD200, CXCR5, or any combination thereof; (iii) the one or more coinhibitory marker comprises TIM-3, LAG-3, or both; (iv) the one or more inflammatory marker comprises CCL3, CCL4, IFNγ, IFN-γ mRNA, or any combination thereof; (v) the one or more cytolytic marker comprises GZMA/K, PRF1 mRNA, or both; (vi) the one or more gene involved in proliferation comprises TYMS, TOP2A, MCM2/4 mRNA, or any combination thereof; and (vii) the one or more tissue resident memory marker comprises CD103.

In certain further embodiments, a method comprises identifying, selecting, and/or sorting one or more CD4+ T cells that: (i) have high expression of CXCL13 and/or CD200 and any one or more of TCF7, IL7R, and BCL6; (ii) have high expression of CXCL13, are negative for TCF7 expression or have reduced TCF7 expression as compared to one or more other CD4+ T cells in the sample, and are positive for expression of, express, or have increased expression relative to one or more other CD4+ T cells of the sample, any one or more of TIM-3, LAG-3, IFN-γ mRNA, GZMA/K, PRF1 mRNA, and CD103; or (iii) have high expression of CXCL13 and are positive for expression, express, or have increased expression relative to one or more other CD4+ T cells of the sample, of any one or more of TYMS, TOP2A, and MCM2/4 mRNA, and, optionally, are positive for expression of BTLA, express BTLA, or have increased expression of BTLA relative to one or more other CD4+ T cells of the sample.

In any of the presently disclosed embodiments, a method can further comprise identifying, selecting, and/or sorting from the one or more identified, selected, and/or sorted CD4+ T cells, (a) one or more CD4+ T cells positive for expression of, expressing, or having increased expression relative to one or more other CD4+ T cells of the sample, of TCF7 and/or (b) one or more CD4+ T cells that are negative for expression of TCF7 or that do not express TCF7 or that have reduced expression of TCF7 as compared to one or more other CD4+ T cells from the sample and/or as compared to other of the one or more identified, selected, and/or sorted CD4+ T cells.

In any of the presently disclosed embodiments, a method can further comprise: (1) identifying, selecting, and/or sorting one or more CD4+ T cells positive for expression of CXCL13 or expressing CXCL13 comprises identifying, selecting, and/or sorting one or more CD4+ T cells positive for expression of CD200 or expressing CD200, and optionally identifying, selecting, and/or sorting one or more CD4+ T cells that have high expression of CD200 or that have increased expression of CD200 relative to one or more other CD4+ T cells from the sample and/or as compared to other of the one or more identified, selected, and/or sorted CD4+ T cells; and/or (2) identifying, selecting, and/or sorting one or more CD4+ T cells positive for expression of CXCL13 or expressing CXCL13 further comprises identifying, selecting, and/or sorting one or more CD4+ T cells that express PD-1 or that are positive for expression of PD-1 or that have high expression of PD-1 or that have increased expression of PD-1 relative to one or more other CD4+ T cells from the sample and/or relative to other of the one or more identified, selected, and/or sorted CD4+ T cells.

In any of the presently disclosed embodiments, a method can: (i) further comprise identifying, selecting, and/or sorting, from the one or more identified, selected, and/or sorted CD4+ T cells, one or more CD4+ T cells positive for expression of CXCR6, expressing CXCR6, or having increased expression of CXCR6 relative to one or more other CD4+ T cells in the sample and/or relative to other of the one or more identified, selected, and/or sorted CD4+ T cells; and/or (ii) further comprise identifying, selecting, and/or sorting, from the one or more identified, selected, and/or sorted CD4+ T cells, one or more CD4+ T cells that are negative for expression of CXCR6 or that do not express CXCR6 or that have reduced expression of CXCR6 relative to other CD4+ T cells of the sample and/or as relative to other of the one or more identified, selected, and/or sorted CD4+ T cells; and/or (iii) one or more identified, selected, and/or sorted CD4+ T cells are negative for CD25 expression or do not express CD25 or have reduced expression of CD25 relative to one or more other CD4+ T cells of the sample and/or as compared to other of the one or more identified, selected, and/or sorted CD4+ T cells.

Also provided is a method comprising identifying, selecting, and/or sorting, from a sample comprising CD4+ T cells, one or more CD4+ T cells that: (i) optionally are negative for expression of CD25 or do not express CD25 or have reduced expression of CD25 relative to one or more other CD4+ T cells in the sample; (ii) are positive for expression of PD-1 and CD200 or express PD-1 and CD200 or have increased expression relative to one or more other CD4+ T cells in the sample and optionally have high expression of PD-1 and/or CD200; and (iii) are positive for expression of CXCR6 or express CXCR6 and optionally have high expression of CXCR6 or increased expression of CXCR6 as compared to one or more other CD4+ T cells in the sample, wherein, optionally, (a) the sample comprises blood and/or tumor from a subject; and/or (b) the sample is from a subject having, or having previously been diagnosed with, a cancer, such as a solid cancer (e.g., melanoma or breast cancer).

In any of the presently disclosed embodiments, identifying, selecting, and/or sorting can comprise use of flow cytometry.

In any of the presently disclosed embodiments, a method can further comprise isolating the one or more identified, selected, and/or sorted CD4+ T cells, wherein, optionally, isolating the one or more identified, selected, and/or CD4+ T cells comprises removing from the sample:
(i) the one or more sorted CD4+ T cells; and/or (ii) CD4+ T cells positive for expression of CD25, expressing CD25, or having increased expression of CD25 relative to other CD4+ T cells of the sample; and/or
(iii) CD4+ T cells negative for expression of CD127, do not expressing CD127, or having reduced expression of CD127 relative to other CD4+ T cells of the sample; and/or
(iv) CD4+ T cells negative for expression of, not expressing, or having reduced expression relative to one or more other CD4+ T cells of the sample, of any one or more of CXCL13, PD-1 and CD200.

In any of the presently disclosed embodiments, a method can further comprise sorting the one or more identified, selected, and/or sorted CD4+ T cells away from CD4+ T cells negative for expression of, not expressing, or having reduced expression as compared to one or more other CD4+ T cells of the sample, of any one or more of CXCL13, PD-1, and CD200.

In any of the presently disclosed embodiments, a method can further comprise comprising culturing and/or expanding the one or more one or more identified, selected, sorted, and/or isolated CD4+ T cells.

In any of the presently disclosed embodiments, a method can further comprise administering the one or more of the one or more identified, selected, sorted, and/or isolated CD4+ T cells to a subject having a cancer, optionally a solid cancer (e.g., melanoma or breast cancer), wherein, optionally, the subject having a cancer is the subject from which the sample was sourced.

In any of the presently disclosed embodiments, a method can further comprise sequencing a TRBV gene segment, a TRBD gene segment, a TRBJ gene segment, a TRAV gene segment, a TRAJ gene segment, or any combination thereof, from one or more of the one or more identified, selected, sorted, and/or isolated CD4+ T cells.

In certain further embodiments, a method can further comprise introducing: (1) a polynucleotide that encodes a TCR Vβ from an identified, selected, sorted, and/or isolated CD4+ T cell; and/or (2) a polynucleotide that encodes a TCR Vα from an identified, selected, sorted, and/or isolated CD4+ T cell; and/or (3) a polynucleotide that encodes a TCR Vβ and a TCR Vα, wherein the TCR Vβ comprises CDR1β, CDR2β, and/or CDR3β from ane identified, selected, sorted, and/or isolated CD4+ T cell, and/or wherein the TCR Vα comprises CDR1, CDR2a, and/or CDR3a from an/the identified, selected, sorted, and/or isolated CD4+ T cell, into one or more host cells, optionally comprising one or more T cells. In certain further embodiments, the one or more host T cells comprise CD4+ T cells and/or CD8+ T cells.

Also provided is a CD4+ T cell or a population of CD4+ T cells or a host cell: (i) identified, selected, sorted, isolated, cultured, expanded, and/or activated by the method as described herein; or (ii) made by a method as described herein.

Also provided is a method comprising expanding: (1) one or more CD4+ T cells obtained, selected, or isolated from a sample comprising a plurality of CD4+ T cells, wherein the one or more CD4+ T cells: (i) is/are positive for expression of CXCL13 or expresses CXCL13 or has increased expression of CXCL13 relative to one or more other CD4+ T cells of the plurality; (ii) is/are positive for expression of, expresses, or has increased expression relative to one or more other CD4+ T cells of the plurality, of PD-1 and CXCL13; (iii) is/are positive for expression of CD200, expresses CD200 or has increased expression of CD200 relative to one or more other CD4+ T cells of the plurality; or (iv) is/are positive for expression of PD-1 and CD200, expresses PD-1 and CD200, or has increased expression of PD-1 and/or of CD200 as relative to one or more other CD4+ T cells of the plurality; or (2) one or more CD4+ T cells that: (i) express CXCL13; (ii) express PD-1 and CXCL13; (iii) express CD200; and/or (iv) express PD-1 and CD200, and, optionally, have increased expression of CXCL13, PD-1 and CXCL13, CD200, and/or PD-1 and CD200 as compared to other CD4+ T cells of a sample from which the one or more CD4+ T cells that are expanded was/were derived. In certain embodiments, the CD4+ T cell or the one or more CD4+ T cells: (i) express or has/have increased expression of (1) one or more memory gene and/or (2) one or more gene or surface marker associated with T follicular helper (T_{FH}) cells, as compared to other CD4+ T cells in the sample; (ii) (1) is/are negative for TCF7 expression or has/have reduced TCF7 expression, as compared to other CD4+ T cells in the sample; and/or (2) express one or more coinhibitory marker, one or more inflammatory marker, one or more cytolytic marker, and/or one or more tissue resident memory marker; and/or (iii) express one or more gene involved in proliferation. In certain further embodiments: (i) the one or more memory gene comprises TCF7, IL7-R, or both; and/or (ii) the one or more gene or surface marker associated with T follicular helper (TFH) cells comprises BCL6, CD200, CXCR5, or any combination thereof; and/or (iii) the one or more coinhibitory marker comprises TIM-3, LAG-3, or both; and/or (iv) the one or more inflammatory marker comprises CCL3, CCL4, IFNγ, IFN-γ mRNA, or any combination thereof; and/or (v) the one or more cytolytic marker comprises GZMA/K, PRF1 mRNA, or both; and/or (vi) the one or more gene involved in proliferation comprises TYMS, TOP2A, MCM2/4 mRNA, or any combination thereof; and/or (vii) the one or more tissue resident memory marker comprises CD103.

In some embodiments, the CD4+ T cell or the one or more CD4+ T cells: (i) is/are positive for expression, express, or has/have increased expression of CXCL13 and any one or more of TCF7, IL7R, BCL6, and CD200, relative to one or more other CD4+ T cells from the sample; (ii) is/are positive for expression, express, or has/have increased expression of CXCL13 relative to other CD4+ T cells from the sample, are negative for TCF7 expression or have reduced TCF7 expression, relative to one or more other CD4+ T cells from the sample, and express one or more of TIM-3, LAG-3, IFN-γ mRNA, GZMA/K, PRF1 mRNA, and CD103; or (iii) is/are positive for expression of CXCL13, express CXCL13, or has/have increased expression of CXCL13 relative to one or more other CD4+ T cells from the sample and express one or more of TYMS, TOP2A, and MCM2/4 mRNA, and optionally, express BTLA.

Also provided is a CD4+ T cell expressing: (i) CXCL13; (ii) PD-1 and CXCL13; (iii) CD200; and/or (iv) PD-1 and CD200.

Also provided is a composition comprising a plurality of CD4+ T cells, wherein 5% or more, 6% or more, 7% or more, 8% or more, 9% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, or 100% of the CD4+ T cells in the composition express: (i) CXCL13; (ii) PD-1 and CXCL13; (iii) CD200; and/or (iv) PD-1 and CD200. In some embodiments, the composition further comprises a pharmaceutically acceptable carrier, excipient, or diluent. In some embodiments, the CD4+ T cell:
(i) further expresses (1) one or more memory gene and/or (2) one or more gene or surface marker associated with T follicular helper (TFH) cells; (ii) (1) does not express TCF7 or is negative for TCF7 expression; and/or (2) expresses one or more coinhibitory marker, one or more inflammatory marker, one or more cytolytic marker, and/or one or more tissue resident memory marker; (iii) expresses one or more gene involved in proliferation; (iv) expresses BTLA; (v) expresses CXCR6; and/or (vi) does not express CXCR6 or is negative for expression of CXCR6, and, optionally, does not express CD25 or is negative for CD25 expression.

Also provided is a CD4+ T cell from a sample comprising a plurality of CD4+ T cells, wherein the CD4+ T cell: (i) expresses CXCL13, optionally having increased expression of CXCL13 as compared to one or more other CD4+ T cells of the plurality; (ii) expresses PD-1 and CXCL13, optionally having increased expression of PD-1 and/or of CXCL13 as compared to one or more other CD4+ T cells of the plurality; (iii) expresses CD200, optionally having increased expression of CD200 as compared to one or more other CD4+ T cells of the plurality; and/or (iv) expresses PD-1 and CD200, optionally having increased expression of PD-1 and/or of CXCL13 as compared to one or more other CD4+ T cells of the plurality, wherein, optionally, the CD4+ T cell:
(a) is negative for CD25 expression or has reduced expression of CD25 as compared to one or more other CD4+ T cells of the plurality; and/or
(b) expresses CXCR6, optionally at an increased level as compared to one or more other CD4+ T cells in the plurality; and/or
(c) is negative for expression of CXCR6 or has reduced expression of CXCR6 as compared to one or more other CD4+ T cells of the plurality; and/or
(d) is from a sample from a subject who had previously been administered an anti-PD-1 antibody or antigen-binding fragment thereof.

In some embodiments, the CD4+ T cell: (i) has increased expression of (1) one or more memory gene and/or (2) one or more gene or surface marker associated with T follicular helper (TFH) cells, as compared to other CD4+ T cells in the sample; (ii) (1) is/are negative for TCF7 expression or has/have reduced TCF7 expression, as compared to other CD4+ T cells in the sample; and/or (2) expresses/express one or more coinhibitory marker, one or more inflammatory marker, one or more cytolytic marker, and or one or more tissue resident memory marker; and/or (iii) expresses/express one or more gene involved in proliferation. In some embodiments: (i) the one or more memory gene comprises TCF7, IL-7R, or both; (ii) the one or more gene or surface marker associated with T follicular helper (TFH) cells comprises BCL6, CD200, CXCR5, or any combination thereof; and/or (iii) the one or more coinhibitory marker comprises TIM-3, LAG-3, or both; and/or (iv) the one or more inflammatory marker comprises CCL3, CCL4, IFNγ, IFN-γ mRNA, or any combination thereof; and/or (v) the one or more cytolytic marker comprises GZMA/K, PRF1 mRNA, or both; and/or (vi) the one or more gene involved in proliferation comprises TYMS, TOP2A, MCM2/4 mRNA, or any combination thereof; and/or (vii) the one or more tissue resident memory marker comprises CD103.

In some embodiments, the CD4+ T cell expresses (i) CXCL13, (ii) PD-1 and CXCL13, (iii) CD200; and/or (iv) PD-1 and CD200, and further expresses: (a) TCF7, IL7R, BCL6, CD200, CXCR5, or any combination thereof; (b) TIM-3, LAG-3, IFN-γ mRNA, GZMA/K, PRF1 mRNA, CD103, or any combination thereof; (c) TYMS, TOP1A, MCM2/4 mRNA, or any combination thereof; and/or (d) BTLA.

In some embodiments, the CD4+ T cell: (i) has increased expression of CXCL13 and any one or more of TCF7, IL7R, BCL6, and CD200, as compared to other CD4+ T cells from the sample; (ii) has increased expression of CXCL13 as compared to other CD4+ T cells from the sample, are negative for TCF7 expression or have reduced TCF7 expression, as compared to other CD4+ T cells in the sample, and express one or more of TIM-3, LAG-3, IFN-γ mRNA, GZMA/K, PRF1 mRNA, and CD103; or (iii) has increased expression of CXCL13 as compared to other CD4+ T cells from the sample and express one or more of TYMS, TOP2A, and MCM2/4 mRNA, and optionally, expresses BTLA.

In some embodiments, the CD4+ T cell was obtained from a sample: (i) comprising tumor, tumor infiltrated by lymphocytes, tumor infiltrating lymphocytes, and/or blood; and/or (ii) from a subject having, or having previously been diagnosed with, a cancer, such as a solid cancer (e.g., melanoma or breast cancer); and/or (iii) from a subject who had previously been administered an anti-PD-1 antibody or antigen-binding fragment thereof.

In some embodiments, the CD4+ T cell is specific for tumor antigen or a tumor neoantigen, optionally an antigen or neoantigen from a solid tumor.

Also provided is a composition comprising a plurality of the CD4+ T cell and optionally one or both of: (i) a plurality of tumor antigen-specific or tumor neoantigen-specific CD8+ T cells; and (ii) a pharmaceutically acceptable carrier, excipient, or diluent.

Also provided is a population of CD4+ T cells or a composition comprising CD4+ T cells, wherein 5% or more, 6% or more, 7% or more, 8% or more, 9% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, or 100% of the CD4+ T cells in the population or composition, respectively, are CD4+ T cells as described herein.

Also provided is a cell population comprising a plurality of CD4+ T cells as described herein, wherein, optionally, the population comprises the CD4+ T cells in an amount comprising from about 2-fold to about 20-fold (e.g., about 2-, about 3-, about 4-, about 5-, about 6, about 7, about 8-, about 9-, about 10-, about 11-, about 12-, about 13-, about 14-, about 15-, about 16-, about 17-, about 18-, about 19-, or about 20-fold) higher than the amount of such CD4+ T cells present in a subject sample (e.g. comprising tumor, such as a solid tumor sample, and/or blood) comprising an equivalent number of cells as the population.

Also provided is a method of treating a cancer in a subject, comprising administering to the subject an effective amount of: (i) a CD4+ T cell or CD4+ T cell population as described herein; and/or (ii) a composition as described herein; and/or (iii) a host cell made by a method as described herein, wherein optionally the host cell comprises a T cell, optionally a CD4+ T cell.

In some embodiments, a host cell or CD4+ T cell or composition or CD4+ T cell population of the present disclosure is provided, wherein the host cell comprises a T cell, further optionally a CD4+ T cell, for use in a method of treating cancer, optionally a solid cancer, in a subject.

In some embodiments, a host cell or CD4+ T cell or composition or CD4+ T cell population of the present disclosure is provided, wherein the host cell comprises a T cell, further optionally a CD4+ T cell, for use in the manufacture of a medicament for treating cancer, optionally a solid cancer, in a subject.

In some embodiments, the solid cancer is selected from: melanoma; breast cancer; a cancer of the head or neck; pancreatic cancer; cholangiocarcinoma; hepatocellular cancer; breast cancer including triple-negative breast cancer (TNBC); gastric cancer; non-small-cell lung cancer; prostate cancer; esophageal cancer; mesothelioma; small-cell lung cancer; colorectal cancer; glioblastoma; carcinoma; sarscoma; chondrosarcoma; fibrosarcoma (fibroblastic sarcoma); Dermatofibrosarcoma protuberans (DFSP); osteosarcoma; rhabdomyosarcoma; Ewing's sarcoma; a gastrointestinal stromal tumor; Leiomyosarcoma; angiosarcoma (vascular sarcoma); Kaposi's sarcoma; liposarcoma; pleomorphic sarcoma; synovial sarcoma; a lung carcinoma (e.g., Adenocarcinoma, Squamous Cell Carcinoma (Epidermoid Carcinoma); Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carciroma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma); an ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, or an adenocarcinoma of the prostate.

In some embodiments, the solid cancer comprises a melanoma, a breast cancer, or both.

Also provided is a method for: (1) identifying a subject as being at increased risk of relapse and/or progression of a solid cancer, the method comprising identifying a subject for whom less than 30% of CD4+ T cells present in a tumor sample of the solid cancer express CXCL13, CXCL13 and PD-1, CD200, or CD200 and PD-1, wherein, optionally, expression of CD200 and/or PD-1 is determined using flow cytometry comprising an isotype control antibody or antigen-binding fragment thereof; or (2) identifying a subject as having a positive prognosis (e.g. increased likelihood of longer survival, longer disease-free survival, longer progression-free survival, or disease remission) for and/or as being at a reduced risk for progression or relapse of a solid cancer, the method comprising identifying a subject for whom 30% or more of CD4+ T cells present in a tumor sample of the solid cancer express CXCL13, CXCL13 and PD-1, CD200, or CD200 and PD-1, thereby identifying the subject as having a positive prognosis and/or as being at reduced risk for progression or relapse of a solid cancer as compared to a subject in whom less than 30% of CD4+ T cells that infiltrate a tumor of the solid cancer express CXCL13, CXCL13 and PD-1, CD200, or CD200 and PD-1.

Also provided is a method for treating or for reducing a risk of a subject experiencing relapse and/or progression of a solid cancer, the method comprising administering, to a subject for whom less than 30% of CD4+ T cells present in a tumor sample of the solid cancer express CXCL13, CXCL13 and PD-1, CD200, or CD200 and PD-1, one or more therapies for the solid cancer, wherein, optionally, the one or more therapies comprise surgery, local radiation therapy, systemic radiation therapy, proton therapy, immunotherapy (e.g. comprising a cytokine, an antibody or antigen-binding fragment thereof, a fusion protein, antigen-specific T cells, antigen-specific NK cells, antigen-specific phagocytic cells, or any combination thereof), or any combination thereof.

In some embodiments, the CD4+ T cells that express CXCL13, CXCL13 and PD-1, CD200, or CD200 and PD-1 have increased expression of CXCL13, CXCL13 and PD-1, CD200, or CD200 and PD-1, respectively, as compared to CD4+ T cells of the subject that do not infiltrate a (optionally the) tumor of the solid cancer and/or as compared to one or more other CD4+ T cells present in the tumor sample.

In some embodiments, the CD4+ T cells that express CXCL13, CXCL13 and PD-1, CD200, or CD200 and PD-1 also: (i) express (1) one or more memory gene and/or (2) one or more gene or surface marker associated with T follicular helper (TFH) cells; (ii) (1) are negative for TCF7 expression; and/or (2) express one or more coinhibitory marker, one or more inflammatory marker, one or more cytolytic marker, and/or one or more tissue resident memory marker; and/or (iii) express one or more gene involved in proliferation.

In some embodiments: (i) the one or more memory gene comprises TCF7, IL-7R, or both;
(ii) the one or more gene or surface marker associated with T follicular helper (TFH) cells comprises BCL6, CD200, CXCR5, or any combination thereof; and/or
(iii) the one or more coinhibitory marker comprises TIM-3, LAG-3, or both; and/or
(iv) the one or more inflammatory marker comprises CCL3, CCL4, IFNγ, IFN-γ mRNA, or any combination thereof; and/or (v) the one or more cytolytic marker comprises GZMA/K, PRF1 mRNA, or both; and/or (vi) the one or more gene involved in proliferation comprises TYMS, TOP2A, MCM2/4 mRNA, or any combination thereof; and/or (vii) the one or more tissue resident memory marker comprises CD103.

In some embodiments, the CD4+ T cells that express CXCL13, CXCL13 and PD-1, CD200, or CD200 and PD-1 also express: (a) TCF7, IL7R, BCL6, CD200, CXCR5, or any combination thereof, optionally at a level that is increased as compared to the level(s) expressed by CD4+ T cells that do not infiltrate a (optionally the) tumor of the solid cancer and/or as compared to one or more other CD4+ T cells present in the tumor sample; (b) TIM-3, LAG-3, IFN-γ mRNA, GZMA/K, PRF1 mRNA, CD103, or any combination thereof, optionally at a level that is increased as compared to the level(s) expressed by CD4+ T cells that do not infiltrate a (optionally the) tumor of the solid cancer; and/or as compared to one or more other CD4+ T cells present in the tumor sample; (c) TYMS, TOP1A, MCM2/4 mRNA, or any combination thereof, optionally at a level that is increased as compared to the level(s) expressed by CD4+ T cells that do not infiltrate a tumor of the solid cancer and/or as compared to one or more other CD4+ T cells present in the tumor sample; and/or (d) BTLA, optionally at a level that is increased as compared to the level(s) expressed by CD4+ T cells that do not infiltrate a tumor of the solid cancer and/or as compared to one or more other CD4+ T cells present in the tumor sample.

In some embodiments, the CD4+ T cells that express CXCL13, CXCL13 and PD-1, CD200, or CD200 and PD-1 also express CXCR6.

In some embodiments, the CD4+ T cells that express CXCL13, CXCL13 and PD-1, CD200, or CD200 and PD-1 are negative for expression of CXCR6 or have reduced expression of CXCR6 as compared to one or more other CD4+ T cells from the subject, optionally that infiltrate a tumor of the solid cancer.

Also provided is a method for identifying a population of CD4+ T cells from a sample, the method comprising: (1) identifying CD4+ T cells that: (i) express PD-1 and CD200, optionally at increased levels as compared to other CD4+ T cells in the sample, and, optionally, do not express or have reduced expression of CD25 as compared to one or more other CD4+ T cells from the sample; and (ii) (a) express CXCR6, optionally at an increased level as compared to other CD4+ T cells from the sample, wherein the identified population comprises a T effector cell phenotype and has reduced proliferative capacity; or (b) do not express CXCR6 or have reduced expression of CXCR6 as compared to one or more other CD4+ T cells from the sample, wherein the identified population comprises a T follicular helper cell phenotype and/or comprises stem and/or progenitor CD4+ T cells with proliferative capacity; or (2) identifying CD4+ T cells that: (i) express PD-1 and CD200, optionally at increased levels as compared to other CD4+ T cells from the sample, and, optionally, do not express or have reduced expression of CD25 as compared to one or more other CD4+ T cells from the sample; and (ii) (a) express TCF7, optionally at an increased level as compared to one or more other CD4+ T cells from the sample, wherein the identified population comprises a T follicular helper cell phenotype and/or comprises stem and/or progenitor CD4+ T cells with proliferative capacity; or (b) do not express TCF7 or have reduced expression of TCF7 as compared to one or more other CD4+ T cells from sample, wherein the identified population comprises a T effector cell phenotype and has reduced proliferative capacity; or (3) identifying CD4+ T cells that: (i) express CXCL13 and optionally PD-1, optionally at increased levels as compared to one or more other CD4+ T cells from the sample, and, optionally, do not express or have reduced expression of CD25 as compared to one or more other CD4+ T cells from the sample; and (ii) (a) express CXCR6, optionally at an increased level as compared to one or more other CD4+ T cells from the sample, wherein the identified population comprises a T effector cell phenotype and has reduced proliferative capacity; or (b) do not express CXCR6 or have reduced expression of CXCR6 as compared to one or more other CD4+ T cells from the sample, wherein the identified population comprises a T follicular helper cell phenotype and/or comprises stem and/or progenitor CD4+ T cells with proliferative capacity; or (4) identifying CD4+ T cells that: (i) express CXCL13 and optionally PD-1, optionally at increased levels as compared to one or more other CD4+ T cells from the sample, and, optionally, do not express or have reduced expression of CD25 as compared to one or more other CD4+ T cells from the sample; and (ii) (a) express CXCR6, optionally at an increased level as compared to one or more other CD4+ T cells from the sample, wherein the identified population comprises a T effector cell phenotype and has reduced proliferative capacity; or (b) do not express CXCR6 or have reduced expression of CXCR6 as compared to one or more other CD4+ T cells from the sample, wherein the identified population comprises a T follicular helper cell phenotype and/or comprises stem and/or progenitor CD4+ T cells with proliferative capacity.

Also provided is a method for identifying a CD4+ T cell, such as for use in adoptive cell therapy, or for identifying a CD4+ T cell having and/or capable of contributing to an antitumor effect, the method comprising identifying, from a sample comprising CD4+ T cells, one or more CD4+ T cells that: (i) express CXCL13; (ii) have increased expression of CXCL13 as compared to one or more other CD4+ T cells from the sample; (iii) express CD200; and/or (iv) have increased expression of CD200 as compared to one or more other CD4+ T cells from the sample.

In some embodiments: (i) the identified CD4+ T cells express PD-1 and/or have increased expression of PD-1 as compared to one or more other CD4+ T cells from the sample; and/or (ii) the method further comprises identifying, from the one or more identified CD4+ T cells, CD4+ T cells that are negative for expression of CXCR6 or that have reduced expression of CXCR6 as compared to one or more other of the one or more identified CD4+ T cells; and/or (iii) the method further comprises identifying, from the one or more identified CD4+ T cells, CD4+ T cells that express CXCR6 or that have increased expression of CXCR6 as compared to one or more other of the one or more identified CD4+ T cells; and/or (iv) the method further comprises identifying, from the one or more identified CD4+ T cells, CD4+ T cells that are negative for expression of TCF7 or that have reduced expression of TCF7 as compared to one or more other of the one or more identified CD4+ T cells; and/or (v) the method further comprises identifying, from the one or more identified CD4+ T cells, CD4+ T cells that express TCF7 or that have increased expression of TCF7 as compared to one or more other of the one or more identified CD4+ T cells; and/or (vi) the method further comprises sorting the one or more identified CD4+ T cells away from other cells; and/or (vii) the method further comprises expanding the one or more identified or sorted CD4+ T cells; and/or (viii) the sample comprises tumor infiltrated by lymphocytes and/or tumor infiltrating lymphocytes; and/or (ix) the method further comprises exposing the identified, sorted, and/or expanded CD4+ T cells to: (1) one or more peptides that comprise a tumor antigen or tumor neoantigen, optionally wherein the tumor antigen or tumor neoantigen is present in the subject and/or against which the one or more identified, selected, sorted, and/or isolated CD4+ T cells are reactive; and/or (2) antigen-presenting cells that present a tumor antigen or tumor neoantigen, optionally against which the one or more identified, selected, sorted, and/or isolated CD4+ T cells are known to be reactive; and/or (3) one or more activating cytokine; and/or (4) one or more agent that binds to a stimulatory or costimulatory protein expressed on the cell surface of the one or more identified, sorted, and/or expanded CD4+ T cells, wherein binding by the one or more agent to the stimulatory or costimulatory protein stimulates the one or more identified, selected, sorted, and/or isolated CD4+ T cells, wherein, optionally, the one or more agent that binds to a stimulatory or costimulatory protein comprises an antibody, or an antigen-binding fragment thereof, that binds to CD3, CD28, CD27, 4-1BB, OX40, ICOS, GITR, or any combination thereof.

Also provided is a method for identifying a T cell receptor, or one or more variable domains thereof, or one or more complementarity determining regions thereof, for use in cellular immunotherapy, the method comprising sequencing a TRAV gene segment, a TRAJ gene segment, a TRBV gene segment, a TRBD gene segment, and a TRBJ gene segment from one or more CD4+ T cells obtained from a sample, wherein the one or more CD4+ T cells: (i) express CXCL13; (ii) have increased expression of CXCL13 as compared to one or more other CD4+ T cells from the sample; (iii) express CD200; and/or
(iv) have increased expression of CD200 as compared to one or more other CD4+ T cells from the sample. In some embodiments, the one or more CD4+ T cells: (i) express PD-1 and/or have increased expression of PD-1 as compared to one or more other CD4+ T cells from the sample; and/or (ii) (a) are negative for expression of CXCR6 or have reduced expression of CXCR6 as compared to one or more other CD4+ T cells from the sample or (b) express CXCR6 or have increased expression of CXCR6 as compared to one or more other CD4+ T cells from the sample; and/or (iii) (a) express TCF7 or have increased expression of TCF7 as compared to one or more other CD4+ T cells from the sample, or (b) are negative for expression of TCF7 or have reduced expression of TCF7 as compared to one or more other CD4+ T cells from the sample.

In some embodiments, the sample comprises tumor infiltrated by lymphocytes and/or tumor infiltrating lymphocytes.

Also provided is a population of CD4+ T cells or a composition comprising CD4+ T cells, wherein 5% or more, 6% or more, 7% or more, 8% or more, 9% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, or 100% of the CD4+ T cells in the population or composition are: (i) CXCL13+; (ii) CXCL13+PD-1+; (iii) CD200+; (iv) CD200+PD-1+; (v) CD200+PD-1+ CXCR6-; (vi) CD200+PD-1+ CXCR6+; (vii) TCF7+; (vii) TCF7-; or (viii) any combination of (i)-(viii). In some embodiments, the CD4+ T cells were obtained from a sample and have increased expression of CXCL13, PD-1, and/or CD200 as compared to one or more other CD4+ T cells from the sample.

Also provided is a kit for sorting or for identifying CD4+ T cells, comprising: (i) a reagent for detecting expression of CXCL13; and/or (ii) a reagent for detecting expression of CD200; and, optionally, (iii) instructions for using the reagent of (i) and/or the reagent of (ii) to detect expression of CXCL13 or CD200, respectively, on a cell of interest, such as a T cell, such as a CD4+ T cell. In some embodiments, the kit further comprises:
(iv) a reagent for detecting expression of PD-1 or for detecting an anti-PD-1 antibody or antigen-binding fragment thereof.

In some embodiments, the kit further comprises: (v) a reagent for detecting expression of CXCR6; and/or (vi) a reagent for detecting expression of TCF7.

A CD4+ T cell or cells of the present disclosure can be reactive to a tumor antigen or tumor neoantigen. Reactivity to a tumor antigen or tumor neoantigen can be determined using any of a number of known techniques, such as, for example, exposing the CD4+ T cells to a peptide tumor antigen or neoantigen and assaying T cell binding, activation or induction, as well as by determining T cell responses that are antigen-specific, such as determination of T cell proliferation, T cell cytokine release, antigen-specific T cell stimulation, MHC restricted T cell stimulation, CTL activity (e.g., by detecting 51Cr or Europium release from pre-loaded target cells), changes in T cell phenotypic marker expression, and other measures of T-cell functions. Procedures for performing these and similar assays are may be found, for example, in Lefkovits (Immunology Methods Manual: The Comprehensive Sourcebook of Techniques, 1998). See, also, Current Protocols in Immunology; Weir, Handbook of Experimental Immunology, Blackwell Scientific, Boston, MA (1986); Mishell and Shigii (eds.) Selected Methods in Cellular Immunology, Freeman Publishing, San Francisco, CA (1979); Green and Reed, Science 281:1309 (1998) and references cited therein.

Levels of cytokines may be determined according to methods described herein and practiced in the art, including for example, ELISA, ELISPOT, intracellular cytokine staining, and flow cytometry and combinations thereof (e.g., intracellular cytokine staining and flow cytometry). Immune cell proliferation and clonal expansion resulting from an antigen-specific elicitation or stimulation of an immune response may be determined by isolating lymphocytes, such as circulating lymphocytes in samples of peripheral blood cells or cells from lymph nodes or cells from a sample or cultured cells, stimulating the cells with antigen, and measuring cytokine production, cell proliferation and/or cell viability, such as by incorporation of tritiated thymidine or non-radioactive assays, such as MTT assays and the like. The effect of an immunogen described herein on the balance between a Th1 immune response and a Th2 immune response may be examined, for example, by determining levels of Th1 cytokines, such as IFN-γ, IL-12, IL-2, and TNF-β, and Type 2 cytokines, such as IL-4, IL-5, IL-9, IL 10, and IL-13.

A “binding protein” refers herein to a protein or polypeptide that possesses the ability to specifically and non-covalently associate, unite, or combine with a target (e.g., a proliferative disease- or hyperproliferative disease-associated antigen). In certain embodiments, a binding protein of the present disclosure comprises a binding domain that specifically binds to an antigen that is expressed by a diseased cell or is otherwise associated with a disease or disorder. A “binding domain” (also referred to as a “binding region” or “binding moiety”), as used herein, refers to a molecule or portion thereof (e.g., peptide, oligopeptide, polypeptide, protein (e.g., a binding protein)) that possesses the ability to specifically and non-covalently associate, unite, or combine with a target (e.g., a proliferative or hyperproliferative disease-associated antigen). A binding domain includes any naturally occurring, synthetic, semi-synthetic, or recombinantly produced binding partner for a biological molecule, a molecular complex (i.e., complex comprising two or more biological molecules), or other target of interest. Exemplary binding domains include single chain immunoglobulin variable regions (e.g., scTCR, scFv, Fab, TCR variable regions (scTv)), Fabs, receptor ectodomains, ligands (e.g., cytokines, chemokines), or synthetic polypeptides selected for their specific ability to bind to a biological molecule, a molecular complex or other target of interest.

“Antigen” or “Ag” as used herein refers to an immunogenic molecule that provokes an immune response. This immune response may involve antibody production, activation of specific immunologically-competent cells (e.g., T cells), or both. An antigen (immunogenic molecule) may be, for example, a peptide, glycopeptide, polypeptide, glycopolypeptide, polynucleotide, polysaccharide, lipid or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can be produced by cells that have been modified or genetically engineered to express an antigen.

The term “epitope” or “antigenic epitope” includes any molecule, structure, amino acid sequence or protein determinant that is recognized and specifically bound by a cognate binding molecule, such as an immunoglobulin, T cell receptor (TCR), chimeric antigen receptor, or other binding molecule, domain or protein. Epitopic determinants generally contain chemically active surface groupings of molecules, such as amino acids or sugar side chains, and can have specific three dimensional structural characteristics, as well as specific charge characteristics.

As used herein, a “neoantigen” refers to a host cellular product containing a structural change, alteration or mutation that creates a new antigen or antigenic epitope that has not previously been observed in the subject's genome (i.e., in a sample of healthy tissue from the subject) or been “seen” or recognized by the host's immune system. Neoantigens may originate, for example, from coding polynucleotides having alterations (substitution, addition, deletion) that result in an altered or mutated product, or from the insertion of an exogenous nucleic acid molecule or protein into a cell, or from exposure to environmental factors (e.g., chemical, radiological) resulting in a genetic change. Neoantigens may arise separately from a tumor antigen, or may arise from or be associated with a tumor antigen. “Tumor neoantigen” (or “tumor-specific neoantigen”) refers to a protein comprising a neoantigenic determinant associated with, arising from, or arising within a tumor cell or plurality of cells within a tumor. Tumor neoantigenic determinants are found on, for example, antigenic tumor proteins or peptides that contain one or more somatic mutations encoded by the DNA of tumor cells, as well as proteins or peptides from viral open reading frames associated with virus-associated tumors (e.g., cervical cancers, some head and neck cancers). For example, tumor neoantigens may arise within or from any of the exemplary tumor or other antigens, as well as from “driver” cancer antigens (e.g., G12D neoantigen from KRAS described in Tran et al., N. Eng. J. Med. 375:2255-2262 (2016)), as well as in mutated B-Raf, SF31, MYD88, DDX3X, MAPK1, GNB1, and others).

The terms “complementarity determining region,” and “CDR,” are synonymous with “hypervariable region” or “HVR,” and are known in the art to refer to sequences of amino acids within TCR or antibody variable regions, which confer antigen specificity and/or binding affinity and are separated from one another by framework sequences or regions. In general, there are three CDRs in each variable region (e.g., in the case of a TCR, αCDR1, αCDR2, αCDR3, βCDR1, βCDR2, and βCDR3; in the case of an antibody, HCDR1, HCDR2, HCDR3, LCDR1, LCDR2, and LCDR3). In the case of TCRs, CDR3 is generally the main CDR responsible for specifically binding to a processed antigen or an antigenic epitope. CDR1 and CDR2 mainly interact with the MHC.

CDR1 and CDR2 are encoded within the variable gene segment of a TCR variable region-coding sequence, whereas CDR3 is encoded by the region spanning the variable and joining segments for Vα, or the region spanning variable, diversity, and joining segments for VB. Thus, if the identity of the variable gene segment of a Vα or Vβ is known, the sequences of their corresponding CDR1 and CDR2 can be deduced; e.g., according to a numbering scheme as described herein. Compared with CDR1 and CDR2, CDR3, and in particular CDR3β, is typically significantly more diverse due to the addition and loss of nucleotides during the recombination process.

TCR variable domain sequences can be aligned to a numbering scheme (e.g., Kabat, Chothia, EU, IMGT, Enhanced Chothia, and Aho), allowing equivalent residue positions to be annotated and for different molecules to be compared using, for example, ANARCI software tool (2016, Bioinformatics 15:298-300). A numbering scheme provides a standardized delineation of framework regions and CDRs in the TCR variable domains. In certain embodiments, a CDR of the present disclosure is identified according to the IMGT numbering scheme (Lefranc et al., Dev. Comp. Immunol. 27:55, 2003; imgt.org/IMGTindex/V-QUEST.php). In some embodiments, a CDR3 is defined in accordance with the IMGT junction definition. In some embodiments, a CDR3 is defined in accordance with the IMGT definition.

As used herein, a “variant” of a CDR refers to a functional variant of a CDR sequence (i.e., capable of binding the target antigen with an avidity or affinity that is similar to the unmodified CDR, such as within about 30%, about 25%, about 20%, about 15%, about 10%, about 5%, about 1% of the avidity or affinity of the unmodified CDR, or has the same or an improved avidity or affinity as compared to the unmodified CDR, such as an avidity or affinity that is increased by about 1%, about 5%, about 10%, about 15%, about 20%, about 25%, about 30%, or more). An exemplary variant CDR may have up to 1-3 amino acid substitutions, deletions, or combinations thereof, provided it is functional (binding) variant CDR.

As used herein, “specifically binds” or “specific for” refers to an association or union of a binding protein (e.g., a T cell receptor or a chimeric antigen receptor) or a binding domain (or binding protein thereof) to a target molecule (e.g., an antigen that is associated with a proliferative disease such as a cancer) with an affinity or Kₐ (i.e., an equilibrium association constant of a particular binding interaction with units of 1/M) equal to or greater than 10⁵ M⁻¹ (which equals the ratio of the on-rate [Kₒₙ] to the off rate [K_{off}] for this association reaction), while not significantly associating or uniting with any other molecules or components in a sample. Binding proteins or binding domains (or binding proteins thereof) may be classified as “high-affinity” binding proteins or binding domains (or binding proteins thereof) or as “low-affinity” binding proteins or binding domains (or binding proteins thereof). “High-affinity” binding proteins or binding domains refer to those binding proteins or binding domains having a Kₐ of at least 10⁷M⁻¹, at least 10⁸ M⁻¹, at least 10⁹ M1, at least 10¹⁰ M⁻¹, at least 10¹¹ M⁻¹, at least 10¹² M⁻¹, or at least 10¹³ M⁻¹. “Low-affinity” binding proteins or binding domains refer to those binding proteins or binding domains having a Kₐ of up to 10⁷ M⁻¹, up to 10⁶ M⁻¹, up to 10⁵ M⁻¹. Alternatively, affinity may be defined as an equilibrium dissociation constant (Kd) of a particular binding interaction with units of M (e.g., 10⁻⁵ M to 10⁻¹³ M).

In certain embodiments, a receptor or binding domain may have “enhanced affinity,” which refers to selected or engineered receptors or binding domains with stronger binding to a target antigen than a wild type (or parent) binding domain. For example, enhanced affinity may be due to a Kₐ (equilibrium association constant) for the target antigen that is higher than the wild type binding domain, due to a Kₐ (dissociation constant) for the target antigen that is less than that of the wild type binding domain, due to an off-rate (k_{off}) for the target antigen that is less than that of the wild type binding domain, or a combination thereof. In certain embodiments, binding proteins may be codon optimized to enhance expression in a particular host cell, such as T cells (e.g., Scholten et al., Clin. Immunol. 119:135, 2006).

A variety of assays are known for identifying binding domains of the present disclosure that specifically bind a particular target, as well as determining binding domain or binding protein affinities, such as Western blot, ELISA, analytical ultracentrifugation, spectroscopy and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent). Assays for assessing affinity or apparent affinity or relative affinity are also known. In certain examples, apparent affinity for a binding protein is measured by assessing binding to various concentrations of tetramers, for example, by flow cytometry using labeled tetramers. In some examples, apparent K_{D} of a binding protein is measured using 2-fold dilutions of labeled tetramers at a range of concentrations, followed by determination of binding curves by non-linear regression, apparent K_{D} being determined as the concentration of ligand that yielded half-maximal binding.

The term “functional avidity” refers to a biological measure or activation threshold of an in vitro immune cell (e.g., T cell, NK cell, NK-T cell) response to a given concentration of a ligand, wherein the biological measures can include cytokine production (e.g., IFNγ production, IL-2 production, etc.), cytotoxic activity, and proliferation. For example, T cells that biologically (immunologically) respond in vitro to a low antigen dose by producing cytokines, being cytotoxic, or proliferating are considered to have high functional avidity, while T cells having lower functional avidity require higher amounts of antigen before an immune response, similar to the high-avidity T cells, is elicited. It will be understood that functional avidity is different from affinity and avidity. Affinity refers to the strength of any given bond between a binding protein and its antigen/ligand. Some binding proteins are multivalent and bind to multiple antigens-in this case, the strength of the overall connection is the avidity.

Numerous correlations exist between the functional avidity and the effectiveness of an immune response. Some ex vivo studies have shown that distinct T cell functions (e.g., proliferation, cytokines production, etc.) can be triggered at different thresholds (see, e.g., Betts et al., J. Immunol. 172:6407, 2004; Langenkamp et al., Eur. J. Immunol. 32:2046, 2002). Factors that affect functional avidity include (a) the affinity of a TCR for the pMHC-complex, that is, the strength of the interaction between the TCR and pMHC (Cawthon et al., J. Immunol. 167:2577, 2001), (b) expression levels of the TCR and the CD4 or CD8 co receptors, and (c) the distribution and composition of signaling molecules (Viola and Lanzavecchia, Science 273:104, 1996), as well as expression levels of molecules that attenuate T cell function and TCR signaling.

The concentration of antigen needed to induce a half-maximum response between the baseline and maximum response after a specified exposure time is referred to as the “half maximal effective concentration” or “EC50”. The EC50 value is generally presented as a molar (moles/liter) amount, but it is often converted into a logarithmic value as follows-log 10 (EC50). For example, if the EC50 equals 1 μM (10⁻⁶ M), the log 10 (EC50) value is-6. Another value used is pEC50, which is defined as the negative logarithm of the EC50 (− log 10 (EC50)). In the above example, the EC50 equaling 1 μM has a pEC50 value of 6. In certain embodiments, the functional avidity of the binding proteins of this disclosure will be a measure of its ability to promote IFNγ production by T cells, which can be measured using assays described herein. “High functional avidity” TCRs or binding domains thereof refer to those TCRs or binding domains thereof having a EC50 of at least 10⁻⁴ M, at least about 10⁻⁵ M, or at least about 10⁻⁶ M.

In certain embodiments, the binding domain is a scFv comprising heavy chain and light chain variable regions connected by short linker peptide. Any scFv of the present disclosure may be engineered so that the C-terminal end of V_{L} domain is linked by a short peptide sequence to the N-terminal end of the V_{H} domain, or vice versa (i.e., (N) V_{L} (C)-linker-(N) V_{H} (C) or (N) V_{H} (C)-linker-(N) V_{L} (C)). It will be appreciated that a scTCR of the present disclosure may also be so arranged (i.e., (N) Vβ (C)-linker-(N) Vα (C) or (N) Vα (C)-linker-(N) Vβ (C)).

In some embodiments, the binding protein specifically binds to a tumor-associated antigen selected from a: CD19; CD20; BCMA; CD22; CD3; CEACAM6; c-Met; EGFR; EGFRvIII; ErbB2; ErbB3; ErbB4; EphA2; IGFIR; GD2; O-acetyl GD2; O-acetyl GD3; GHRHR; GHR; FLT1; KDR; FLT4; CD44v6; CD151; CA125; CEA; CTLA-4; GITR; BTLA; TGFBR2; TGFBR1; IL6R; gp130; Lewis A; Lewis Y; TNFR1; TNFR2; PD1; PD-L1; PD-L2; HVEM; MAGE-A (e.g., including MAGE-A1, MAGE-A3, and MAGE-A4); mesothelin; NY-ESO-1; PSMA; RANK; ROR1; TNFRSF4; CD40; CD137; TWEAK-R; HLA; tumor- or pathogen-associated peptide bound to HLA; hTERT peptide bound to HLA; tyrosinase peptide bound to HLA; WT-1 peptide bound to HLA; LTBR; LIFRB; LRP5; MUC1; OSMRB; TCRα; TCRβ; CD25; CD28; CD30; CD33; CD52; CD56; CD79a; CD79b; CD80; CD81; CD86; CD123; CD171; CD276; B7H4; TLR7; TLR9; PTCH1; WT-1; HA1-H; Robo1; α-fetoprotein (AFP); Frizzled; OX40; PRAME; and/or SSX-2 antigen.

In some embodiments, the tumor associated antigen is CD19. In particular embodiments, the binding protein comprises a binding domain derived from an anti-CD19 antibody, such as, for example, FMC-63 antibody, MOR208, blinatumomab, MEDI-551, Meck patent anti-CD19 antibody, Xmab5871, or MDX-1342.

In some embodiments, the antigen-specific receptor binding domain is derived from FMC-63 antibody, MOR208, blinatumomab, MEDI-551, Merck patent anti-CD19 antibody, Xmab5871, or MDX-1342 has a VH, or (i.e., and/or) a VL having at least about 80%, 85%, 90%, 95%, 96%, 96%, 98%, 99%, or more amino acid sequence identity to that of the antibody variable regions or scFv thereof from FMC-63 antibody, MOR208, blinatumomab, MEDI-551, Merck patent anti-CD19 antibody, Xmab5871, or MDX-1342, or has CDRs or functional CDR variants according to any one of these antibodies.

In certain embodiments, the binding protein comprises a transmembrane component or transmembrane domain disposed between an extracellular component comprising the binding domain and an intracellular component, which can comprise an effector domain. As used herein, an “effector domain” is an intracellular portion, component, or domain of a binding protein or receptor that is capable of directly or indirectly promoting an immunological response in a cell (e.g., immune system cell, such as a T cell) when receiving an appropriate signal. In certain embodiments, an effector domain is from a protein or portion thereof or protein complex that receives a signal when bound, or when the protein or portion thereof or protein complex binds directly to a target molecule and triggers a signal from the effector domain.

An effector domain may directly promote a cellular immune response when it contains one or more signaling domains or motifs, such as an Intracellular Tyrosine-based Activation Motif (ITAM), as found in costimulatory molecules. Without wishing to be bound by theory, it is believed the ITAMs are important for T cell activation following ligand engagement by a T cell receptor or by a binding protein comprising a T cell effector domain. In certain embodiments, the intracellular component comprises an ITAM. Exemplary effector domains include those from CD27, CD28, 4-1BB (CD137), OX40 (CD134), CD3ε, CD3δ, CD3ξ, CD25, CD27, CD28, CD79A, CD79B, CARD11, DAP10, FcRα, FcRβ, FcRγ, Fyn, HVEM, ICOS, Lck, LAG3, LAT, LRP, NKG2D, NOTCH1, NOTCH2, NOTCH3, NOTCH4, Wnt, ROR2, Ryk, SLAMF1, Slp76, pTα, TCRα, TCRβ, TRIM, Zap70, PTCH2, or any combination thereof. In certain embodiments, an effector domain comprises a lymphocyte receptor signaling domain (e.g., CD3%).

In further embodiments, the intracellular component of the binding protein comprises a costimulatory domain or portion thereof selected from CD27, CD28, 4-1BB (CD137), OX40 (CD134), ICOS (CD278), CD27, CD2, CD5, ICAM-1 (CD54), LFA-1 (CD11a/CD18), GITR, CD30, CD40, BAFF-R, HVEM, LIGHT, NKG2C, SLAMF7, NKp80, CD160, B7-H3 CD94, DAP12, a ligand that specifically binds with CD83, or a functional variant thereof, or any combination thereof. In certain embodiments, the intracellular component comprises a CD28 costimulatory domain or portion thereof (which may optionally include a LL→GG mutation at positions 186-187 of the native CD28 protein; see Nguyen et al., Blood 102:4320, 2003)), a 4-1BB costimulatory domain or portion thereof, or both.

In certain embodiments, an effector domain comprises CD35 or a functional portion thereof. In further embodiments, an effector domain comprises a portion or a domain from CD27. In some embodiments, an effector domain comprises a portion or a domain from CD28. In some embodiments, an effector domain comprises a portion or a domain from 4-1BB. In some embodiments, an effector domain comprises a portion or a domain from OX40. In some embodiments, an effector domain comprises a portion or a domain from 4-1BB and a portion or a domain from CD35.

In some embodiments, an extracellular component and an intracellular component of a binding protein of the present disclosure are connected by a transmembrane component or domain. A “transmembrane component” or “transmembrane domain”, as used herein, is a portion of a transmembrane protein that can insert into or span a cell membrane. Transmembrane components or domains have a three-dimensional structure that is thermodynamically stable in a cell membrane and generally range in length from about 15 amino acids to about 30 amino acids. The structure of a transmembrane component or domain may comprise an alpha helix, a beta barrel, a beta sheet, a beta helix, or any combination thereof. In certain embodiments, a transmembrane component or domain comprises or is derived from a known transmembrane protein (i.e., a CD4 transmembrane domain, a CD8 transmembrane domain, a CD27 transmembrane domain, a CD28 transmembrane domain, or any combination thereof). In particular embodiments, a transmembrane component of a binding protein is derived from CD28 and an intracellular component effector domain comprises a 4-1BB signaling domain and a CD3 domain.

In certain embodiments, the extracellular component of the binding protein further comprises a linker (also referred to herein as a “spacer”) disposed between the binding domain and the transmembrane domain. As used herein when referring to a component of a binding protein that connects the binding and transmembrane domains, a “linker” may be an amino acid sequence having from about two amino acids to about 500 amino acids, which can provide flexibility and room for conformational movement between two regions, domains, motifs, fragments, or modules connected by the linker. For example, a linker of the present disclosure can position the binding domain away from the surface of a host cell expressing the binding protein to enable proper contact between the host cell and a target cell, antigen binding, and activation (Patel et al., Gene Therapy 6:412-419, 1999). Linker length may be varied to maximize antigen recognition based on the selected target molecule, selected binding epitope, or antigen binding domain seize and affinity (see, e.g., Guest et al., J. Immunother. 28:203-11, 2005; PCT Publication No. WO 2014/031687). Exemplary linkers include those having a glycine-serine amino acid chain having from one to about ten repeats of GlyxSery, wherein x and y are each independently an integer from 0 to 10, provided that x and y are not both 0 (e.g., (Gly4Ser) 2, (Gly3Ser) 2, Gly2Ser, or a combination thereof, such as ((Gly3Ser) 2Gly2Ser).

Linkers of the present disclosure also include immunoglobulin constant regions (i.e., CH1, CH2, CH3, or CL, of any isotype) and portions thereof. In certain embodiments, a linker comprises a CH3 domain, a CH2 domain, or both. In certain embodiments, a linker comprises a CH2 domain and a CH3 domain. In further embodiments, the CH2 domain and the CH3 domain are each a same isotype. In particular embodiments, the CH2 domain and the CH3 domain are an IgG4 or IgG1 isotype. In other embodiments, the CH2 domain and the CH3 domain are each a different isotype. In specific embodiments, the CH2 comprises a N297Q mutation. Without wishing to be bound by theory, it is believed that CH2 domains with N297Q mutation do not bind FcγR (see, e.g., Sazinsky et al., PNAS 105 (51): 20167 (2008)). In certain embodiments, the linker comprises a human immunoglobulin constant region or portion thereof.

In any of the embodiments described herein, a linker may comprise a hinge region or portion thereof. Hinge regions are flexible amino acid polymers of variable length and sequence (typically rich in proline and cysteine amino acids) and connect larger and less-flexible regions of immunoglobulin proteins. For example, hinge regions connect the heavy chain constant and Fab regions of antibodies and connect the constant and transmembrane regions of TCRs.

Linkers comprising modified immunoglobulin constant or hinge regions, or portions, thereof, are also contemplated, wherein the modification (e.g., substitution, insertion, deletion) does not substantially affect one or more functional characteristic of interest (e.g. length, flexibility, solubility) of the linker. In some embodiments, the linker comprises a constant region, hinge region, modified constant region, or modified hinge region, that is, or is derived from, an IgG isotype, such as, for example, an IgG1, IgG2, IgG3, or IgG4 isotype.

In particular embodiments, the binding domain of the encoded binding protein is derived from FMC-63 antibody, MOR208, blinatumomab, MEDI-551, Merck patent anti-CD19 antibody, Xmab5871, or MDX-1342; and/or the hinge region is derived from IgG4; and/or the transmembrane component is derived from CD28; and/or the intracellular component comprises a 4-1BB signaling domain and a CD3% domain.

In certain embodiments, one or more of the extracellular component, the binding domain, the linker, the transmembrane domain, the intracellular component, or the costimulatory domain comprises a junction amino acid. “Junction amino acids” or “junction amino acid residues” refer to one or more (e.g., about 2-20) amino acid residues between two adjacent domains, motifs, regions, modules, or fragments of a protein, such as between a binding domain and an adjacent linker, between a transmembrane domain and an adjacent extracellular or intracellular domain, or on one or both ends of a linker that links two domains, motifs, regions, modules, or fragments (e.g., between a linker and an adjacent binding domain or between a linker and an adjacent hinge). Junction amino acids may result from the construct design of a binding protein (e.g., amino acid residues resulting from the use of a restriction enzyme site or self-cleaving peptide sequences during the construction of a polynucleotide encoding a binding protein). For example, a transmembrane domain of a binding protein may have one or more junction amino acids at the amino-terminal end, carboxy-terminal end, or both.

In some embodiments, a binding protein of the present disclosure may further comprise a protein tag (also called a peptide tag or tag peptide herein). Protein tags are unique peptide sequences that are affixed or genetically fused to, or are a part of, a protein of interest and can be recognized or bound by, for example, a heterologous or non-endogenous cognate binding molecule or a substrate (e.g., receptor, ligand, antibody, carbohydrate, or metal matrix). Protein tags are useful for detecting, identifying, isolating, tracking, purifying, enriching for, targeting, or biologically or chemically modifying tagged proteins of interest, particularly when a tagged protein is part of a heterogenous population of cells (e.g., a biological sample like peripheral blood). In the provided binding proteins, the ability of the tag(s) to be specifically bound by the cognate binding molecules is distinct from, or in addition to, the ability of the binding domain(s) to specifically bind the hyperproliferative disease-associated antigen. In certain embodiments, the protein tag is a Myc tag, His tag, Flag tag, Xpress tag, Avi tag, Calmodulin tag, Polyglutamate tag, HA tag, Nus tag, S tag, X tag, SBP tag, Softag, V5 tag, CBP, GST, MBP, GFP, Thioredoxin tag, Strep-Tag (e.g., Strep-Tag® or Strep-Tag II®), or any combination thereof.

In any of the embodiments described herein, a binding protein can be or can comprise a TCR or a CAR, or both. “T cell receptor” (TCR) refers to an immunoglobulin superfamily member (having a variable binding domain, a constant domain, a transmembrane region, and a short cytoplasmic tail; see, e.g., Janeway et al., Immunobiology: The Immune System in Health and Disease, 3^{rd} Ed., Current Biology Publications, p. 4:33, 1997) capable of specifically binding to an antigen peptide bound to a MHC receptor. A TCR can be found on the surface of a cell or in soluble form and generally is comprised of a heterodimer having α and β chains (also known as TCRα and TCRβ, respectively), or γ and δ chains (also known as TCRγ and TCRδ, respectively). Like other immunoglobulins (e.g., antibodies), the extracellular portion of TCR chains (e.g., α-chain, β-chain) contain two immunoglobulin domains, a variable domain (e.g., α-chain variable domain or V_{α}, β-chain variable domain or VB; typically amino acids 1 to 116 based on Kabat numbering (Kabat et al., “Sequences of Proteins of Immunological Interest, US Dept. Health and Human Services, Public Health Service National Institutes of Health, 1991, 5th ed.) at the N-terminus, and one constant domain (e.g., α-chain constant domain or C_{α}, typically amino acids 117 to 259 based on Kabat, β-chain constant domain or C_{β}, typically amino acids 117 to 295 based on Kabat) adjacent to the cell membrane. Also, like antibodies, the variable domains contain complementary determining regions (CDRs) separated by framework regions (FRs) (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In certain embodiments, a TCR is found on the surface of T cells (or T lymphocytes) and associates with the CD3 complex. The source of a TCR as used in the present disclosure may be from various animal species, such as a human, mouse, rat, rabbit or other mammal. Methods for producing engineered TCRs are described in, for example, Bowerman et al., Mol. Immunol., 46 (15): 3000 (2009), the techniques of which are herein incorporated by reference.

The term “variable region” or “variable domain” refers to the domain of an immunoglobulin superfamily binding protein (e.g., a TCR α-chain or β-chain (or γ chain and 8 chain for γδ TCRs)) that is involved in binding of the immunoglobulin superfamily binding protein (e.g., TCR) to antigen. The variable domains of the α-chain and β-chain (Vα and VB, respectively) of a native TCR generally have similar structures, with each domain comprising four generally conserved framework regions (FRs) and three CDRs. The Vα domain is encoded by two separate DNA segments, the variable gene segment, and the joining gene segment (V-J); the Vβ domain is encoded by three separate DNA segments, the variable gene segment, the diversity gene segment, and the joining gene segment (V-D-J). A single Vα or Vβ domain may be sufficient to confer antigen-binding specificity. Furthermore, TCRs that bind a particular antigen may be isolated using a Vα or Vβ domain from a TCR that binds the antigen to screen a library of complementary Vα or Vβ domains, respectively.

CDR1 and CDR2 are encoded within the variable gene segment of a TCR variable region-coding sequence, whereas CDR3 is encoded by the region spanning the variable and joining segments for Vα, or the region spanning variable, diversity, and joining segments for Vβ. Thus, if the identity of the variable gene segment of a Vα or Vβ is known, the sequences of their corresponding CDR1 and CDR2 can be deduced; e.g., according to a numbering scheme as described herein. Compared with CDR1 and CDR2, CDR3, and in particular CDR3B, is typically significantly more diverse due to the addition and loss of nucleotides during the recombination process.

The source of a TCR as used in the present disclosure may be from any of a variety of animal species, such as a human, mouse, rat, rabbit, or other mammal. TCR constant domain sequences may be from, for example, human, mouse, marsupial (e.g. opossum, bandicoot, wallaby), shark, or non-human primate. In certain preferred embodiments, TCR constant domain sequences are human or comprise engineered variants of human sequences. TCR constant domains may be engineered to improve pairing, expression, stability, or any combination of these. See, e.g., Cohen et al., Cancer Res, 2007; Kuball et al., Blood 2007; and Haga-Freidman et al., Journal of Immunology 2009. Examples of engineering in TCR Cα and Cβ include mutation of a native amino acid to a cysteine so that a disulfide bond forms between the introduced cysteine of one TCR constant domain and a native cysteine of the other TCR constant domain. Such mutations can include T48C in Cα, T57C in Cβ, or both. Mutations to improve stability can include a mutation in the Cα transmembrane domain from the sequence LSVIGF to the sequence LLVIVL (“L-V-L” mutation; see Haga-Friedman et al., J Immunol 188:5538-5546 (2012), the TCR mutations and mutant TCR constant domain sequences of which are incorporated herein by reference).

“CD8 co-receptor” or “CD8” refers to the cell surface glycoprotein CD8, either as an alpha-alpha homodimer or an alpha-beta heterodimer. The CD8 co-receptor assists in the function of cytotoxic T cells (CD8+) and functions through signaling via its cytoplasmic tyrosine phosphorylation pathway (Gao and Jakobsen, Immunol. Today 21:630-636, 2000; Cole and Gao, Cell. Mol. Immunol. 1:81-88, 2004). There are five (5) human CD8 beta chain isoforms (see UniProtKB identifier P10966) and a single human CD8 alpha chain isoform (see UniProtKB identifier P01732).

“CD4” is known to refer to an immunoglobulin co-receptor glycoprotein that assists the TCR in communicating with antigen-presenting cells (see, Campbell & Reece, Biology 909 (Benjamin Cummings, Sixth Ed., 2002)). CD4 is found on the surface of immune cells such as T helper cells, monocytes, macrophages, and dendritic cells, and includes four immunoglobulin domains (D1 to D4) that are expressed at the cell surface. During antigen presentation, CD4 is recruited, along with the TCR complex, to bind to different regions of the MHCII molecule (CD4 binds MHCII β2, while the TCR complex binds MHCII α1/β1). Without wishing to be bound by theory, it is believed that close proximity to the TCR complex allows CD4-associated kinase molecules to phosphorylate the immunoreceptor tyrosine activation motifs (ITAMs) present on the cytoplasmic domains of CD3. This activity is thought to amplify the signal generated by the activated TCR in order to produce or recruit various types of immune system cells, including T helper cells, and immune responses.

In certain embodiments, a TCR is found on the surface of T cells (or T lymphocytes) and associates with a CD3 complex.

“CD3” is a multi-protein complex of six chains (see, Abbas and Lichtman, 2003; Janeway et al., p. 172 and 178, 1999) that is associated with antigen signaling in T cells. In mammals, the complex comprises a CD3γ chain, a CD3δ chain, two CD3ε chains, and a homodimer of CD3ξ chains. The CD3γ, CD3β, and CD3ε chains are related cell surface proteins of the immunoglobulin superfamily containing a single immunoglobulin domain. The transmembrane regions of the CD3γ, CD3β, and CD3ε chains are negatively charged, which is believed to allow these chains to associate with positively charged regions of T cell receptor chains. The intracellular tails of the CD3γ, CD3β, and CD3ε chains each contain a single conserved motif known as an immunoreceptor tyrosine based activation motif or ITAM, whereas each CD3ξ chain has three. Without wishing to be bound by theory, it is believed that the ITAMs are important for the signaling capacity of a TCR complex. CD3 as used in the present disclosure may be from various animal species, including human, mouse, rat, or other mammals.

As used herein, “TCR complex” refers to a complex formed by the association of CD3 with TCR. For example, a TCR complex can be composed of a CD3γ chain, a CD3β chain, two CD3ε chains, a homodimer of CD3ξ chains, a TCRα chain, and a TCRβ chain. Alternatively, a TCR complex can be composed of a CD3γ chain, a CD3β chain, two CD3ε chains, a homodimer of CD3ξ chains, a TCRγ chain, and a TCRβ chain.

A “component of a TCR complex”, as used herein, refers to a TCR chain (i.e., TCRα, TCRβ, TCRγ or TCRδ), a CD3 chain (i.e., CD3γ, CD3δ, CD3ε or CD3ξ), or a complex formed by two or more TCR chains or CD3 chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains).

“Major histocompatibility complex molecules” (MHC molecules) refer to glycoproteins that deliver peptide antigens to a cell surface. MHC class I molecules are heterodimers consisting of a membrane spanning a chain (with three a domains) and a non-covalently associated β2 microglobulin. MHC class II molecules are composed of two transmembrane glycoproteins, α and β, both of which span the membrane. Each chain has two domains. MHC class I molecules deliver peptides originating in the cytosol to the cell surface, where a peptide: MHC complex is recognized by CD8⁺ T cells. MHC class II molecules deliver peptides originating in the vesicular system to the cell surface, where they are recognized by CD4⁺ T cells. An MHC molecule may be from various animal species, including human, mouse, rat, cat, dog, goat, horse, or other mammals.

“Chimeric antigen receptor” (CAR) refers to a binding protein of the present disclosure engineered to contain two or more naturally-occurring amino acid sequences linked together in a way that does not occur naturally or does not occur naturally in a host cell, which binding protein can function as a receptor when present on a surface of a cell. CARs of the present disclosure include an extracellular portion comprising an antigen-binding domain (e.g., obtained or derived from an immunoglobulin or immunoglobulin-like molecule, such as an scFv derived from an antibody or a scTCR derived from a TCR specific for a cancer antigen, or an antigen-binding domain derived or obtained from a killer immunoreceptor from an NK cell) linked to a transmembrane domain and one or more intracellular signaling domains (optionally containing co-stimulatory domain(s)) (see, e.g., Sadelain et al., Cancer Discov., 3 (4): 388 (2013); see also Harris and Kranz, Trends Pharmacol. Sci., 37 (3): 220 (2016); Stone et al., Cancer Immunol. Immunother., 63 (11): 1163 (2014)).

Methods of making binding proteins, including CARs, are known in the art and are described, for example, in U.S. Pat. Nos. 6,410,319; 7,446,191; U.S. Patent Publication No. 2010/065818; U.S. Pat. No. 8,822,647; PCT Publication No. WO 2014/031687; U.S. Pat. No. 7,514,537; and Brentjens et al., 2007, Clin. Cancer Res. 13:5426, the techniques of which are herein incorporated by reference.

In certain embodiments, the antigen-binding fragment of the TCR comprises a single chain TCR (scTCR), which comprises both the TCR Vα and TCR Vβ domains, but only a single TCR constant domain (Cα or Cβ). In certain embodiments, the antigen-binding fragment of the TCR or CAR is chimeric (e.g., comprises amino acid residues or motifs from more than one donor or species), humanized (e.g., comprises residues from a non-human organism that are altered or substituted so as to reduce the risk of immunogenicity in a human), or human.

In some embodiments, a binding protein comprises a scTv or a soluble TCR.

In certain embodiments, a binding protein comprises a TCR-CAR, which generally comprises at least a soluble antigen-binding portion of a TCR fused to a CAR intracellular signaling domain(s) (see, e.g., Walseng et al., Scientific Reports 7:10713 (2017), the TCR-CAR constructs of which are hereby incorporated by reference in their entirety).

Methods useful for isolating and purifying recombinantly produced soluble binding proteins, by way of example, may include obtaining supernatants from suitable host cell/vector systems that secrete the recombinant soluble binding protein into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/recombinant soluble binding protein described herein include batch cell culture, which is monitored and controlled to maintain appropriate culture conditions. Purification of the soluble binding protein may be performed according to methods described herein and known in the art and that comport with laws and guidelines of domestic and foreign regulatory agencies.

Binding proteins as described herein may be functionally characterized according to any of a large number of art-accepted methodologies.

In another aspect, nucleic acid molecules are provided that encode any one or more of the binding proteins (e.g. TCR, scTCR, TCR-CAR) described herein. A polynucleotide encoding a desired binding protein can be obtained or produced using recombinant methods known in the art using standard techniques, such as screening libraries from cells expressing a desired sequence or a portion thereof, by deriving a sequence from a vector known to include the same, or by isolating a sequence or a portion thereof directly from cells or tissues containing the same. Alternatively, a sequence of interest can be produced synthetically. Such nucleic acid molecules can be inserted into an appropriate vector (e.g., viral vector or non-viral plasmid vector) for introduction into a host cell of interest (e.g., an immune cell, such as a T cell).

Markers may be used to identify or monitor expression of a heterologous polynucleotide by a host cell transduced with the same, or to detect cells expressing a binding protein of interest. In certain embodiments, the polynucleotide encoding the marker is located 3′ to the polynucleotide encoding the intracellular component of the binding protein, or is located 5′ to the polynucleotide encoding the extracellular component. Exemplary markers include green fluorescent protein, an extracellular domain of human CD2, a truncated human EGFR (huEGFRt; see Wang et al., Blood 118:1255 (2011)), a truncated human CD19 (huCD19t), a truncated human CD34 (huCD34t), or a truncated human NGFR (huNGFRt). In certain embodiments, the encoded marker comprises EGFRt, CD19t, CD34t, or NGFRt.

In any of the embodiments described herein, a binding-protein-encoding polynucleotide can further comprise a polynucleotide that encodes a self-cleaving polypeptide, wherein the polynucleotide encoding the self-cleaving polypeptide is located between the polynucleotide encoding the intracellular component and the polynucleotide encoding the marker. When the polynucleotide is expressed by a host cell comprising the same, the binding protein and the marker are expressed as separate molecules at the host cell surface.

In certain embodiments, a self-cleaving polypeptide comprises a 2A peptide from porcine teschovirus-1 (P2A), Thosea asigna virus (T2A), equine rhinitis A virus (E2A), or foot-and-mouth disease virus (F2A)). Further exemplary nucleic acid and amino acid sequences of 2A peptides are set forth in, for example, Kim et al. (PLOS One 6: e18556, 2011, which 2A nucleic acid and amino acid sequences are incorporated herein by reference in their entirety).

In any of the embodiments described herein, a polynucleotide of the present disclosure may be codon optimized for a host cell containing the polynucleotide (see, e.g, Scholten et al., Clin. Immunol. 119:135-145 (2006)).

In further aspects, expression constructs are provided, wherein the expression constructs comprise a polynucleotide of the present disclosure operably linked to an expression control sequence (e.g., a promoter). In certain embodiments, the expression construct is comprised in a vector. An exemplary vector may comprise a polynucleotide capable of transporting another polynucleotide to which it has been linked, or which is capable of replication in a host organism. Some examples of vectors include plasmids, viral vectors, cosmids, and others. Some vectors may be capable of autonomous replication in a host cell into which they are introduced (e.g. bacterial vectors having a bacterial origin of replication and episomal mammalian vectors), whereas other vectors may be integrated into the genome of a host cell or promote integration of the polynucleotide insert upon introduction into the host cell and thereby replicate along with the host genome (e.g., lentiviral vector, retroviral vector). Additionally, some vectors are capable of directing the expression of genes to which they are operatively linked (these vectors may be referred to as “expression vectors”). According to related embodiments, it is further understood that, if one or more agents (e.g., polynucleotides encoding fusion proteins as described herein) are co-administered to a subject, that each agent may reside in separate or the same vectors, and multiple vectors (each containing a different agent or the same agent) may be introduced to a cell or cell population or administered to a subject.

In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the vector comprises a plasmid vector or a viral vector (e.g., a vector selected from lentiviral vector or a γ-retroviral vector). In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing CAR transgenes are known in the art and have been previous described, for example, in: U.S. Pat. No. 8,119,772; Walchli et al., PLOS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

Other vectors developed for gene therapy can also be used with the compositions and methods of this disclosure. Such vectors include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

Construction of an expression vector for producing a binding protein of this disclosure can be generated to obtain efficient transcription and translation. For example, a polynucleotide contained in a recombinant expression construct includes at least one appropriate expression control sequence (also called a regulatory sequence), such as a leader sequence and particularly a promoter operably (i.e., operatively) linked to the nucleotide sequence encoding the immunogen.

In certain embodiments, polynucleotides of the present disclosure are used to transfect/transduce a host cell (e.g., a T cell) for use in adoptive transfer therapy (e.g., targeting a cancer antigen). Methods for transfecting/transducing T cells with desired nucleic acids have been described (e.g., U.S. Patent Application Pub. No. US 2004/0087025) as have adoptive transfer procedures using T cells of desired target-specificity (e.g., Schmitt et al., Hum. Gen. 20:1240, 2009; Dossett et al., Mol. Ther. 17:742, 2009; Till et al., Blood 112:2261, 2008; Wang et al., Hum. Gene Ther. 18:712, 2007; Kuball et al., Blood 109:2331, 2007; US 2011/0243972; US 2011/0189141; Leen et al., Ann. Rev. Immunol. 25:243, 2007), such that adaptation of these methodologies to the presently disclosed embodiments is contemplated, based on the teachings herein, including those directed to binding proteins of the present disclosure.

Accordingly, in another aspect, host cells are provided that comprise a polynucleotide of the present disclosure and express the encoded binding protein, wherein the encoded binding protein locates to the cell surface of the host cell when expressed. In certain embodiments, the host cell is a hematopoietic progenitor cell or a human immune system cell. In further embodiments, the immune system cell is a CD8+ T cell, a CD4+ T cell, a CD4-CD8-double negative T cell, a γδ T cell, a natural killer cell (e.g., NK cell or NK-T cell), a dendritic cell, or any combination thereof. In certain embodiments, the immune system cell is a CD8+ T cell. In certain embodiments, the T cell is a naïve T cell, a central memory T cell, a stem cell memory T cell, an effector memory T cell, or any combination thereof.

A host cell may include any individual cell or cell culture which may receive a vector or the incorporation of nucleic acids or express proteins. The term also encompasses progeny of the host cell, whether genetically or phenotypically the same or different. It will be appreciated that a polynucleotide encoding a binding protein of this disclosure is “heterologous” with regard to progeny of a host cell of the present disclosure, as well as to the host cell. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

In any of the foregoing embodiments, an immune host cell may be modified to reduce or eliminate expression of one or more endogenous genes that encode a polypeptide involved in immune signaling or other related activities. Exemplary gene knockouts include those that encode PD-1, LAG-3, CTLA4, TIM3, an HLA molecule, a TCR molecule, FAS, or the like. Without wishing to be bound by theory, certain endogenously expressed immune cell proteins may be recognized as foreign by an allogeneic host receiving the modified immune cells, which may result in elimination of the modified immune cells (e.g., an HLA allele), or may downregulate the immune activity of the modified immune cells (e.g., PD-1, LAG-3, CTLA4, FAS), or may interfere with the binding activity of a heterologously expressed binding protein of the present disclosure.

Accordingly, decreasing or eliminating expression or activity of such endogenous genes or proteins can improve the activity, tolerance, or persistence of the immune cells in an autologous or allogeneic host setting, and may allow for universal administration of the cells (e.g., to any recipient regardless of HLA type).

Moreover, an immune cell (e.g. a CD4+ T cell) can include a heterologous polynucleotide encoding a TCR or an antigen-binding fragment thereof, optionally from a CD4+ T cell identified according to the presently disclosed methods. The heterologous polynucleotide can be introduced into the immune cell by any appropriate method, including using gene editing techniques, viral transduction, electroporation, or the like.

In certain embodiments, an immune cell is a donor cell (e.g., allogeneic) or an autologous cell. In certain embodiments, an immune cell of this disclosure comprises a chromosomal gene knockout of one or more of a gene that encodes PD-1, LAG-3, CTLA4, TIM3, TIGIT, an HLA component (e.g., a gene that encodes an α1 macroglobulin, an α2 macroglobulin, an α3 macroglobulin, a β1 microglobulin, or a β2 microglobulin), FAS, or a TCR component (e.g., a gene that encodes a TCR variable region or a TCR constant region) (see, e.g., Torikai et al., Nature Sci. Rep. 6:21757 (2016); Torikai et al., Blood 119 (24): 5697 (2012); and Torikai et al., Blood 122 (8): 1341 (2013), the gene-editing techniques, compositions, and adoptive cell therapies of which are herein incorporated by reference in their entirety).

As used herein, the term “chromosomal gene knockout” refers to a genetic alteration or introduced inhibitory agent in a host cell that prevents (e.g., reduces, delays, suppresses, or abrogates) production, by the host cell, of a functionally active endogenous polypeptide product. Alterations resulting in a chromosomal gene knockout can include, for example, introduced nonsense mutations (including the formation of premature stop codons), missense mutations, gene deletion, and strand breaks, as well as the heterologous expression of inhibitory nucleic acid molecules that inhibit endogenous gene expression in the host cell.

In certain embodiments, a chromosomal gene knock-out or gene knock-in (e.g., knocking-in a binding protein-encoding polynucleotide of the present disclosure) is made by chromosomal editing of a host cell. Chromosomal editing can be performed using, for example, endonucleases. As used herein “endonuclease” refers to an enzyme capable of catalyzing cleavage of a phosphodiester bond within a polynucleotide chain. In certain embodiments, an endonuclease is capable of cleaving a targeted gene thereby inactivating or “knocking out” the targeted gene. An endonuclease may be a naturally occurring, recombinant, genetically modified, or fusion endonuclease. The nucleic acid strand breaks caused by the endonuclease are commonly repaired through the distinct mechanisms of homologous recombination or non-homologous end joining (NHEJ). During homologous recombination, a donor nucleic acid molecule may be used for a donor gene “knock-in”, for target gene “knock-out”, and optionally to inactivate a target gene through a donor gene knock in or target gene knock out event. NHEJ is an error-prone repair process that often results in changes to the DNA sequence at the site of the cleavage, e.g., a substitution, deletion, or addition of at least one nucleotide. NHEJ may be used to “knock-out” a target gene. Examples of endonucleases include zinc finger nucleases, TALE-nucleases, CRISPR-Cas nucleases, meganucleases, and megaTALs.

As used herein, a “zinc finger nuclease” (ZFN) refers to a fusion protein comprising a zinc finger DNA-binding domain fused to a non-specific DNA cleavage domain, such as a Fokl endonuclease. Each zinc finger motif of about 30 amino acids binds to about 3 base pairs of DNA, and amino acids at certain residues can be changed to alter triplet sequence specificity (see, e.g., Desjarlais et al., Proc. Natl. Acad. Sci. 90:2256-2260, 1993; Wolfe et al., J. Mol. Biol. 285:1917-1934, 1999). Multiple zinc finger motifs can be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair. Alternatively, a DSB generated by a ZFN can result in knock out of target gene via repair by non-homologous end joining (NHEJ), which is an error-prone cellular repair pathway that results in the insertion or deletion of nucleotides at the cleavage site. In certain embodiments, a gene knockout comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a FokI endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the Repeat Variable Diresidue (RVD), correlate with specific nucleotide recognition. The natural (canonical) code for DNA recognition of these TALEs has been determined such that an HD (histine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also known (see, e.g., U.S. Patent Publication No. US 2011/0301073, which atypical RVDs are incorporated by reference herein in their entirety). TALENs can be used to direct site-specific double-strand breaks (DSB) in the genome of T cells. Non-homologous end joining (NHEJ) ligates DNA from both sides of a double-strand break in which there is little or no sequence overlap for annealing, thereby introducing errors that knock out gene expression. Alternatively, homology directed repair can introduce a transgene at the site of DSB providing homologous flanking sequences are present in the transgene. In certain embodiments, a gene knockout comprises an insertion, a deletion, a mutation or a combination thereof, and made using a TALEN molecule.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA: tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences can be introduced at the site of DSB via homology directed repair. The crRNA and tracrRNA can be engineered into a single guide RNA (sgRNA or gRNA) (see, e.g., Jinek et al., Science 337:816-21, 2012). Further, the region of the guide RNA complementary to the target site can be altered or programed to target a desired sequence (Xie et al., PLOS One 9: e100448, 2014; U.S. Pat. Appl. Pub. No. US 2014/0068797, U.S. Pat. Appl. Pub. No. US 2014/0186843; U.S. Pat. No. 8,697,359, and PCT Publication No. WO 2015/071474; each of which is incorporated by reference). In certain embodiments, a gene knockout comprises an insertion, a deletion, a mutation or a combination thereof, and made using a CRISPR/Cas nuclease system.

Exemplary gRNA sequences and methods of using the same to knock out endogenous genes that encode immune cell proteins include those described in Ren et al., Clin. Cancer Res. 23 (9): 2255-2266 (2017), the gRNAs, CAS9 DNAs, vectors, and gene knockout techniques of which are hereby incorporated by reference in their entirety.

As used herein, a “meganuclease,” also referred to as a “homing endonuclease,” refers to an endodeoxyribonuclease characterized by a large recognition site (double stranded DNA sequences of about 12 to about 40 base pairs). Meganucleases can be divided into five families based on sequence and structure motifs: LAGLIDADG, GIY-YIG, HNH, His-Cys box and PD-(D/E) XK. Exemplary meganucleases include I-Scel, I-Ceul, PI-PspI, PI-Sce, I-SceIV, I-CsmI, I-PanI, I-SceII, I-PpoI, I-SceIII, I-Crel, I-TevI, I-TevII and I-TevIII, whose recognition sequences are known (see, e.g., U.S. Pat. Nos. 5,420,032 and 6,833,252; Belfort et al., Nucleic Acids Res. 25:3379-3388, 1997; Dujon et al., Gene 82:115-118, 1989; Perler et al., Nucleic Acids Res. 22:1125-1127, 1994; Jasin, Trends Genet. 12:224-228, 1996; Gimble et al., J. Mol. Biol. 263:163-180, 1996; Argast et al., J. Mol. Biol. 280:345-353, 1998).

In certain embodiments, naturally-occurring meganucleases may be used to promote site-specific genome modification of a target selected from PD-1, LAG3, TIM3, CTLA4, TIGIT, an HLA-encoding gene, FAS, or a TCR component-encoding gene. In other embodiments, an engineered meganuclease having a novel binding specificity for a target gene is used for site-specific genome modification (see, e.g., Porteus et al., Nat. Biotechnol. 23:967-73, 2005; Sussman et al., J. Mol. Biol. 342:31-41, 2004; Epinat et al., Nucleic Acids Res. 31:2952-62, 2003; Chevalier et al., Molec. Cell 10:895-905, 2002; Ashworth et al., Nature 441:656-659, 2006; Paques et al., Curr. Gene Ther. 7:49-66, 2007; U.S. Patent Publication Nos. US 2007/0117128; US 2006/0206949; US 2006/0153826; US 2006/0078552; and US 2004/0002092). In further embodiments, a chromosomal gene knockout is generated using a homing endonuclease that has been modified with modular DNA binding domains of TALENs to make a fusion protein known as a megaTAL. MegaTALs can be utilized to not only knock-out one or more target genes, but to also introduce (knock in) heterologous or exogenous polynucleotides when used in combination with an exogenous donor template encoding a polypeptide of interest.

In certain embodiments, a chromosomal gene knockout comprises an inhibitory nucleic acid molecule that is introduced into a host cell (e.g., an immune cell) comprising a heterologous polynucleotide encoding an antigen-specific receptor that specifically binds to a tumor associated antigen, wherein the inhibitory nucleic acid molecule encodes a target-specific inhibitor and wherein the encoded target-specific inhibitor inhibits endogenous gene expression (e.g., of PD-1, TIM3, LAG3, CTLA4, TIGIT, an HLA component, FAS, or a TCR component, or any combination thereof) in the host immune cell.

A chromosomal gene knockout can be confirmed directly by DNA sequencing of the host immune cell following use of the knockout procedure or agent. Chromosomal gene knockouts can also be inferred from the absence of gene expression (e.g., the absence of an mRNA or polypeptide product encoded by the gene) following the knockout.

In another aspect, compositions are provided herein that comprise an immune cell of the present disclosure and a pharmaceutically acceptable carrier, diluent, or excipient.

In certain embodiments, an antigen is a cancer antigen that comprises a: CD19; CD20; BCMA; CD22; CD3; CEACAM6; c-Met; EGFR; EGFRvIII; ErbB2; ErbB3; ErbB4; EphA2; IGFIR; GD2; O-acetyl GD2; O-acetyl GD3; GHRHR; GHR; FLT1; KDR; FLT4; CD44v6; CD151; CA125; CEA; CTLA-4; GITR; BTLA; TGFBR2; TGFBR1; IL6R; gp130; Lewis A; Lewis Y; TNFR1; TNFR2; PD1; PD-L1; PD-L2; HVEM; MAGE-A (e.g., including MAGE-A1, MAGE-A3, and MAGE-A4); mesothelin; NY-ESO-1; PSMA; RANK; ROR1; TNFRSF4; CD40; CD137; TWEAK-R; HLA; tumor- or pathogen-associated peptide bound to HLA; hTERT peptide bound to HLA; tyrosinase peptide bound to HLA; WT-1 peptide bound to HLA; LTR; LIFRB; LRP5; MUC1; OSMRB; TCRα; TCRβ; CD25; CD28; CD30; CD33; CD52; CD56; CD79a; CD79b; CD80; CD81; CD86; CD123; CD171; CD276; B7H4; TLR7; TLR9; PTCH1; WT-1; HA1-H; Robo1; α-fetoprotein (AFP); Frizzled; OX40; PRAME; and/or SSX-2 antigen.

An effective amount of a therapeutic or pharmaceutical composition refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutic amount” may be used in reference to treatment, whereas “prophylactically effective amount” may be used to describe administrating an effective amount to a subject that is susceptible or at risk of developing a disease or disease-state (e.g., recurrence) as a preventative course.

A “therapeutically effective amount” or “effective amount” of a host cell or other agent of this disclosure refers to an amount of host cells or agents sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient or a cell expressing a single active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially or simultaneously. A combination may also be a cell expressing more than one active ingredient, or a immune cell, a chemotherapeutic agent, or another relevant therapeutic.

The number of cells will depend upon the ultimate use for which the composition is intended as well the type of cells included therein. For therapeutic uses provided herein, cells are generally in a volume of a liter or less, 500 mls or less, 250 mls or less, or 100 mls or less. In embodiments, the density of the desired cells is typically greater than 10⁴ cells/ml and generally is greater than 10⁷ cells/ml, generally 10⁸ cells/ml or greater. The cells may be administered as a single infusion or in multiple infusions over a range of time. A clinically relevant number of immune cells can be apportioned into multiple infusions that cumulatively equal or exceed 10⁴, 10⁵, 10⁶, 10⁷, 10⁸, 10⁹, 10¹⁰, or 10¹¹ cells.

Also contemplated are pharmaceutical compositions that comprise cells as disclosed herein and a pharmaceutically acceptable carrier, diluents, or excipient. The term “pharmaceutically acceptable excipient or carrier” or “physiologically acceptable excipient or carrier” refer to biologically compatible vehicles, e.g., physiological saline, which are described in greater detail herein, that are suitable for administration to a human or other non-human mammalian subject and generally recognized as safe or not causing a serious adverse event. Suitable excipients include water, saline, dextrose, glycerol, or the like and combinations thereof. In embodiments, compositions comprising binding proteins or host cells as disclosed herein further comprise a suitable infusion media. Suitable infusion media can be any isotonic medium formulation, typically normal saline, Normosol R (Abbott) or Plasma-Lyte A (Baxter), 5% dextrose in water, Ringer's lactate can be utilized. An infusion medium can be supplemented with human serum albumin or other human serum components.

### Therapies for Cancer and Other Diseases and Disorders

Also provided are methods for treating a cancer, and uses of presently disclosed compositions for treating a cancer or in the preparation of a medicament for treating a cancer.

In some embodiments, a composition (e.g., modified immune cell composition) of the present disclosure is administered to a subject who previously received lymphodepletion chemotherapy. As used herein, the term “chemotherapeutic agent” (which may also be called a “chemotherapeutic” or a “chemotherapy” herein) refers to a chemical agent, drug, or other therapeutic modality that targets diseased cells (e.g., cancer cells) for inhibition or death. Chemotherapeutic agents of the present disclosure encompass different structures, forms, and systems of delivery, and are to be understood in terms of their functionality for inhibiting or killing diseased cells.

Lymphocytes may be depleted using irradiation or chemotherapy to kill lymphocytes, reduce tumor burden, or facilitate survival of subsequently transferred modified immune cells of the present disclosure. In some embodiments, lymphodepletion chemotherapy comprises an alkylating agent, e.g., cyclophosphamide. In further embodiments, the subject has received cyclophosphamide administered at about 100, 125, 150, 175, 200, 225, 250, 275, 300, 350, 375, 400, 425, 450, 475, or about 500 mg/m². In some embodiments, the subject has received cyclophosphamide at about 300 mg/m². In any of the herein disclosed embodiments, lymphodepletion can comprise a platin (e.g., oxaliplatin), fludarabine (optionally administered at about 10, 15, 20, 25, 30, 35, 40, 45, or 50 mg/m²), or both, or in combination with an alkylating agent such as cyclophosphamide.

In some embodiments, the subject has received cyclophosphamide and fludarabine. In some embodiments, the subject has received oxaliplatin and cyclophosphamide. In some embodiments, the subject has received cyclophosphamide, fludarabine, and oxaliplatin, which may be in any combination or combinations; e.g., in one combination comprising fludarabine and cyclophosphamide and another combination comprising oxaliplatin and cyclophosphamide.

In particular embodiments, the subject has received lymphodepletion chemotherapy comprising cyclophosphamide at about 300 mg/m² and fludarabine at about 30 mg/m².

Other chemotherapeutic agents are described herein and may be used in any combination with a composition of this disclosure, with or without a lymphodepletion chemotherapy, or as a secondary therapy.

In certain aspects, a composition of the present disclosure is used with an with an inhibitor of an immune suppression component or an agonist of a stimulatory immune checkpoint molecule, as described herein, to enhance an antitumor response by the immune system and to, ultimately, treat a tumor or associated cancer.

As used herein, the term “immune suppression component” or “immunosuppression component” refers to one or more cells, proteins, molecules, compounds or complexes providing inhibitory signals to assist in controlling or suppressing an immune response. For example, immune suppression components include those molecules that partially or totally block immune stimulation; decrease, prevent or delay immune activation; or increase, activate, or up regulate immune suppression. Exemplary immunosuppression component targets are described in further detail herein and include PD-1, PD-L1, CTLA4, Tim-3, LAG-3, TIGIT, or any combination thereof.

An inhibitor of an immune suppression component may be a compound, an antibody, an antibody fragment or fusion polypeptide (e.g., Fc fusion, such as CTLA4-Fc or LAG3-Fc), an antisense molecule, a ribozyme or RNAi molecule, or a low molecular weight organic molecule. In any of the embodiments disclosed herein, a method may comprise administering a modified immune cell with one or more inhibitor of any one of the following immune suppression components, singly or in any combination.

### Agonists of Stimulatory Immune Checkpoint Molecules

In certain embodiments, a cell composition is used in combination (e.g., concurrently, sequentially, or simultaneously) with an agent that increases the activity (i.e., is an agonist) of a stimulatory immune checkpoint molecule. For example, a cell composition can be used in combination with a CD137 (4-1BB) agonist (such as, for example, urelumab), a CD134 (OX-40) agonist (such as, for example, MEDI6469, MEDI6383, or MEDI0562), lenalidomide, pomalidomide, a CD27 agonist (such as, for example, CDX-1127), a CD28 agonist (such as, for example, TGN1412, CD80, or CD86), a CD40 agonist (such as, for example, CP-870,893, rhuCD40L, or SGN-40), a CD122 agonist (such as, for example, IL-2), an agonist of GITR (such as, for example, humanized monoclonal antibodies described in PCT Patent Publication No. WO 2016/054638), or an agonist of ICOS (CD278) (such as, for example, GSK3359609, mAb 88.2, JTX-2011, Icos 145-1, or Icos 314-8), or any combination thereof. In any of the embodiments disclosed herein, a method may comprise administering a cell composition with one or more agonist of a stimulatory immune checkpoint molecule, including any of the foregoing, singly or in any combination.

Subjects that can be treated by the present invention are, in general, human and other primate subjects, such as monkeys and apes for veterinary medicine purposes. In any of the aforementioned embodiments, the subject may be a human subject. The subjects can be male or female and can be any suitable age, including infant, juvenile, adolescent, adult, and geriatric subjects. Cell compositions according to the present disclosure may be administered in a manner appropriate to the disease, condition, or disorder to be treated as determined by persons skilled in the medical art. In any of the above embodiments, a cell composition as described herein is administered intravenously, intraperitoneally, intratumorally, into the bone marrow, into a lymph node, or into the cerebrospinal fluid. An appropriate dose, suitable duration, and frequency of administration of the compositions will be determined by such factors as a condition of the patient; size, type, and severity of the disease, condition, or disorder; the undesired type or level or activity of the immunotherapy cells, the particular form of the active ingredient; and the method of administration.

Certain methods of treatment or prevention contemplated herein include administering a cell composition (comprising CD4+ T cells according to the present disclosure, which may be autologous, allogeneic or syngeneic, or any combination thereof, to a subject). In some embodiments, a cell composition comprises polyclonal CD4+ T cells, monoclonal CD4+ T cells, neoantigen-specific CD4+ T cells, tumor antigen-specific CD4+ T cells, or any combination thereof.

Certain methods of treatment or prevention contemplated herein include administering a cell composition (comprising modified immune cells that may be autologous, allogeneic or syngeneic, or any combination thereof) wherein cells in the composition contain a desired polynucleotide as described herein that is stably integrated into the chromosome of the cell. For example, such a cell composition may be generated ex vivo using autologous, allogeneic or syngeneic immune system cells (e.g., T cells, antigen-presenting cells, natural killer cells) in order to administer a desired, binding protein-expressing T-cell composition to a subject as an adoptive immunotherapy. In certain embodiments, the host cell is a hematopoietic progenitor cell or a human immune cell. In certain embodiments, the immune system cell is a CD4+ T cell, a CD8+ T cell, a CD4-CD8-double negative T cell, a γδ T cell, a natural killer cell, a dendritic cell, or any combination thereof. In certain embodiments, the immune system cell is a naïve T cell, a central memory T cell, an effector memory T cell, a stem cell memory T cell, or any combination thereof. Methods of generating immune cell compositions are described further herein.

In any of the presently disclosed treatment methods, the encoded binding protein of the modified immune cells in the dose can comprise a chimeric antigen receptor (CAR), a T cell receptor (TCR), a single chain TCR (scTCR), or any combination thereof. In certain embodiments, the antigen comprises a cancer antigen (such as a neoantigen or a tumor-associated antigen or a cancer testis antigen), a viral antigen, or an autoimmune antigen. In some embodiments, the antigen is a cancer antigen that comprises a: CD19; CD20; BCMA; CD22; CD3; CEACAM6; c-Met; EGFR; EGFRvIII; ErbB2; ErbB3; ErbB4; EphA2; IGFIR; GD2; O-acetyl GD2; O-acetyl GD3; GHRHR; GHR; FLT1; KDR; FLT4; CD44v6; CD151; CA125; CEA; CTLA-4; GITR; BTLA; TGFBR2; TGFBR1; IL6R; gp130; Lewis A; Lewis Y; TNFR1; TNFR2; PD1; PD-L1; PD-L2; HVEM; MAGE-A (e.g., including MAGE-A1, MAGE-A3, and MAGE-A4); mesothelin; NY-ESO-1; PSMA; RANK; ROR1; TNFRSF4; CD40; CD137; TWEAK-R; HLA; tumor- or pathogen-associated peptide bound to HLA; hTERT peptide bound to HLA; tyrosinase peptide bound to HLA; WT-1 peptide bound to HLA; LTBR; LIFRB; LRP5; MUC1; OSMRB; TCRα; TCRβ; CD25; CD28; CD30; CD33; CD52; CD56; CD79a; CD79b; CD80; CD81; CD86; CD123; CD171; CD276; B7H4; TLR7; TLR9; PTCH1; WT-1; HA1-H; Robo1; α-fetoprotein (AFP); Frizzled; OX40; PRAME; and/or SSX-2 antigen.

In certain embodiments, a disease comprises a proliferative disease, such as a hyperproliferative disease. Exemplary proliferative disorders include tumors, cancers, neoplastic tissue, carcinoma, sarcoma, malignant cells, pre-malignant cells, as well as non-neoplastic or non-malignant hyperproliferative disorders (e.g., adenoma, fibroma, lipoma, leiomyoma, hemangioma, fibrosis, restenosis, as well as autoimmune diseases such as rheumatoid arthritis, osteoarthritis, psoriasis, inflammatory bowel disease, or the like).

Furthermore, “cancer” may refer to any accelerated or dysregulated proliferation of cells, including solid tumors, ascites tumors, blood or lymph or other malignancies; connective tissue malignancies; metastatic disease; minimal residual disease following transplantation of organs or stem cells; multi-drug resistant cancers, primary or secondary malignancies, angiogenesis related to malignancy, or other forms of cancer. Exemplary cancers treatable according to presently disclosed methods and compositions include both those characterized by solid tumors (e.g., triple negative breast cancer (TNBC), non small-cell lung cancer (NSCLC)) and hematological malignancies (e.g., ALL, CLL, and MCL). In some embodiments, a cancer comprises a melanoma, a breast cancer, or both.

In general, cancers treatable by presently disclosed methods and compositions include carcinomas, sarcomas, gliomas, lymphomas, leukemias, myelomas, cancers of the head or neck, melanoma, pancreatic cancer, cholangiocarcinoma, hepatocellular cancer, breast cancer, gastric cancer, non-small-cell lung cancer, prostate cancer, esophageal cancer, mesothelioma, small-cell lung cancer, colorectal cancer, glioblastoma, Askin's tumor, sarcoma botryoides, chondrosarcoma, Ewing's sarcoma, PNET, malignant hemangioendothelioma, malignant schwannoma, osteosarcoma, alveolar soft part sarcoma, angiosarcoma, cystosarcoma phyllodes, dermatofibrosarcoma protuberans (DFSP), desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, undifferentiated pleomorphic sarcoma, malignant peripheral nerve sheath tumor (MPNST), neurofibrosarcoma, rhabdomyosarcoma, synovial sarcoma, undifferentiated pleomorphic sarcoma, squamous cell carcinoma, basal cell carcinoma, adenocarcinoma, linitis plastic, vipoma, cholangiocarcinoma, hepatocellular carcinoma, adenoid cystic carcinoma, renal cell carcinoma, Grawitz tumor, ependymoma, astrocytoma, oligodendroglioma, brainstem glioma, optice nerve glioma, a mixed glioma, Hodgkin's lymphoma, a B-cell lymphoma, non-Hodgkin's lymphoma (NHL), Burkitt's lymphoma, small lymphocytic lymphoma (SLL), diffuse large B-cell lymphoma, follicular lymphoma, immunoblastic large cell lymphoma, precursor B-lymphoblastic lymphoma, and mantle cell lymphoma, Waldenström's macroglobulinemia, CD37+ dendritic cell lymphoma, lymphoplasmacytic lymphoma, splenic marginal zone lymphoma, extra-nodal marginal zone B-cell lymphoma of mucosa-associated (MALT) lymphoid tissue, nodal marginal zone B-cell lymphoma, mediastinal (thymic) large B-cell lymphoma, intravascular large B-cell lymphoma, primary effusion lymphoma, adult T-cell lymphoma, extranodal NK/T-cell lymphoma, nasal type, enteropathy-associated T-cell lymphoma, hepatosplenic T-cell lymphoma, blastic NK cell lymphoma, Sezary syndrome, angioimmunoblastic T cell lymphoma, anaplastic large cell lymphoma, chondrosarcoma; fibrosarcoma (fibroblastic sarcoma); Dermatofibrosarcoma protuberans (DFSP); osteosarcoma; rhabdomyosarcoma; Ewing's sarcoma; a gastrointestinal stromal tumor; Leiomyosarcoma; angiosarcoma (vascular sarcoma); Kaposi's sarcoma; liposarcoma; pleomorphic sarcoma; or synovial sarcoma; lung carcinoma (e.g., Adenocarcinoma, Squamous Cell Carcinoma (Epidermoid Carcinoma); Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carciroma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma); ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, or an adenocarcinoma of the prostate.

As used herein, administration of a composition or therapy refers to delivering the same to a subject, regardless of the route or mode of delivery. Administration may be effected continuously or intermittently, and parenterally. Administration may be for treating a subject already confirmed as having a recognized condition, disease or disease state, or for treating a subject susceptible to or at risk of developing such a condition, disease or disease state. Co-administration with an adjunctive therapy may include simultaneous and/or sequential delivery of multiple agents in any order and on any dosing schedule (e.g., binding protein-expressing recombinant (i.e., engineered) host cells with one or more cytokines; immunosuppressive therapy such as calcineurin inhibitors, corticosteroids, microtubule inhibitors, low dose of a mycophenolic acid prodrug, or any combination thereof).

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's condition, the undesired type or level or activity of the tagged immunotherapy cells, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease associated with disease or disorder. Prophylactic benefit of the compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

In certain embodiments, a plurality of doses of a cell composition as described herein are administered to the subject, which may, in some embodiments, be administered at intervals between administrations of about two to about four weeks. In some embodiments, a first dose is provided, and a second dose is provided from about 21 to about 28 days thereafter.

The cell compositions administered in first and second or subsequent doses may be the same or different in terms of, for example, the concentration of cells, the type of cells (e.g., CD4+ or CD8+), or both. In certain embodiments, a second dose comprises a composition comprising CD4+ T cells and CD8+ T cells in about a 1:1 ratio; e.g., 50% CD4+ T cells and 50% CD8+ T cells, +/−20%, or +/−15%, or +/−10%, or +/−5%, or +/−4%, or +/−3%, or +/−2%, or +/−1%. In some embodiments comprising administration of a modified immune cell of this disclosure, a second dose comprises In some embodiments, a dose comprises (a) about 1×10⁵ cells/kg; (b) about 2×10⁵ cells/kg; (c) about 3.3×10⁵ cells/kg; (d) about 1×10⁶ cells/kg; (e) about 2×10⁶ cells/kg; (f) about 3.3×10⁶ cells/kg; (g) about 1×10⁷ cells/kg; or (h) about 2×10⁷ cells/kg. In some embodiments comprising a cell composition of this disclosure, a second dose comprises a same dose of a cell composition as compared to the first dose. In other embodiments, a second dose comprises a reduced amount of immune cells as compared to the first dose. In still other embodiments, a second dose comprises an increased amount of immune cells as compared to the first dose. In particular embodiments, a first dose comprises about 1×10⁶ or about 2×10⁶ of a T cell/kg and the second dose comprises about 3.3×10⁶ of the T cell/kg. In other embodiments, a first dose comprises about 3.3×10⁶ of a T cell/kg and the second dose comprises about 1.0×10⁷ or about 2×10⁷ of the T cell/kg. In other embodiments, a first dose comprises about 1×10⁶ of a T cell/kg and the second dose comprises about 1.0×10⁷ or about 2×10⁶ of the T cell/kg. It will be appreciated that each of a first and second dose of a cell composition of this disclosure can independently comprise any of the doses enumerated herein, or any dose therebetween.

In any of the embodiments disclosed herein, a dose (e.g., either or both of a first dose and a second dose, or any subsequent dose) may be administered to the subject over a period from about 1 minute to about 1 hour, or from about 5 minutes to about 50 minutes, or from about 10 minutes to about 40 minutes, or from about 20 minutes to about 30 minutes. In further embodiments, a dose of a cell composition is administered to the subject over a period from about 20 minutes to about 30 minutes.

In any of the presently disclosed embodiments, a dose comprising an immune cell composition may be administered to the subject intravenously, intratumorally, intrathecally, or into bone marrow.

If the subject composition (e.g., any composition described herein) is administered parenterally, the composition may also include sterile aqueous or oleaginous solution or suspension. Suitable non-toxic parenterally acceptable diluents or solvents include water, Ringer's solution, isotonic salt solution, 1,3-butanediol, ethanol, propylene glycol or polythethylene glycols in mixtures with water. Aqueous solutions or suspensions may further comprise one or more buffering agents, such as sodium acetate, sodium citrate, sodium borate or sodium tartrate. Material used in preparing any dosage unit formulation should be pharmaceutically acceptable and substantially non-toxic in the amounts employed. In addition, the active compounds may be incorporated into sustained-release preparation and formulations. Dosage unit form, as used herein, refers to physically discrete units suited as unitary dosages for the subject to be treated; each unit may contain a predetermined quantity of recombinant cells or active compound calculated to produce the desired therapeutic effect in association with an appropriate pharmaceutical carrier.

In general, an appropriate dosage and treatment regimen provides the active molecules or cells in an amount sufficient to provide therapeutic or prophylactic benefit. Such a response can be monitored by establishing an improved clinical outcome (e.g., stable disease, more frequent remissions, complete or partial, or longer disease-free survival) in treated subjects as compared to non-treated subjects. Increases in preexisting immune responses to a tumor protein generally correlate with an improved clinical outcome. Such immune responses may generally be evaluated using imaging techniques for solid tumors or standard proliferation, cytotoxicity or cytokine assays, which are routine in the art and may be performed using samples obtained from a subject before and after treatment.

The level of a CTL immune response may be determined by any one of numerous immunological methods described herein and practiced in the art. The level of a CTL immune response may be determined prior to and following administration of any one of the herein described binding proteins expressed by, for example, a T cell. Cytotoxicity assays for determining CTL activity may be performed using any one of several techniques and methods routinely practiced in the art (see, e.g., Henkart et al., “Cytotoxic T-Lymphocytes” in Fundamental Immunology, Paul (ed.) (2003 Lippincott Williams & Wilkins, Philadelphia, PA), pages 1127-50, and references cited therein).

Antigen-specific T cell responses are typically determined by comparisons of observed T cell responses according to any of the herein described T cell functional parameters (e.g., proliferation, cytokine release, CTL activity, altered cell surface marker phenotype, etc.) that may be made between T cells that are exposed to a cognate antigen in an appropriate context (e.g., the antigen used to prime or activate the T cells, when presented by immunocompatible antigen-presenting cells) and T cells from the same source population that are exposed instead to a structurally distinct or irrelevant control antigen. A response to the cognate antigen that is greater, with statistical significance, than the response to the control antigen signifies antigen-specificity. Persistence, spread, antitumor activity, and phenotype of modified immune cells can be determined using markers and assays known in the art and including those described herein (e.g., by flow cytometry gating for a surface-expressed transduction marker that is co-expressed with the binding protein (e.g., EGFRt or the like), for a T cell activation or exhaustion marker (e.g., TIM-3, LAG-3, PD-1, TIGIT, CD137), by radiation imaging or histology to determine tumor burden, mass, volume, or spread, or the like).

A biological sample may be obtained from a subject for determining the presence and level of an immune response to a binding protein or cell as described herein. A “biological sample” as used herein may be a blood sample (from which serum or plasma may be prepared), biopsy specimen, body fluids (e.g., lung lavage, ascites, mucosal washings, synovial fluid), bone marrow, lymph nodes, tissue explant, tumor, organ culture, or any other tissue or cell preparation from the subject or a biological source. Biological samples may also be obtained from the subject prior to receiving any immunogenic composition, which biological sample is useful as a control for establishing baseline (i.e., pre-immunization) data.

The pharmaceutical compositions described herein may be presented in unit-dose or multi-dose containers, such as infusion bags, sealed ampoules or vials. Such containers may be frozen to preserve the stability of the formulation until use for, e.g., therapy or analysis. The development of suitable dosing and treatment regimens for using the particular compositions described herein in a variety of treatment regimens, including e.g., parenteral or intravenous administration or formulation.

Lymphodepletion chemotherapy agents are used as pre-conditioning agents in some adoptive cell therapies and, in some cases, are administered to a subject prior to the subject receiving a cell therapy. In some embodiments of the present disclosure, a subject receives and completes a lymphodepletion chemotherapy treatment (such as any of the lymphodepletion chemotherapies disclosed herein, including cyclophosphamide, fludarabine, oxaliplatin, or any combination or combinations thereof) at least about 12 hours, at least about 24 hours, at least about 36 hours, at least about 48 hours, at least about 60 hours, at least about 72 hours, at least about 84 hours, at least about 96 hours, or more, prior to receiving a dose of a cell composition of this disclosure. In further embodiments, a subject administered a dose of a cell composition of this disclosure had previously been administered lymphodepleting chemotherapy about 36 to about 96 hours prior to being administered the dose. It will be understood that in embodiments wherein a subject receives a first dose of a cell composition and a subsequent second dose of the cell composition, as described herein, a lymphodepletion chemotherapy may be administered before, concurrent with, simultaneous with, or after either or both of the first and second dose of the cell composition.

In still further embodiments, the subject being treated receives, is receiving, or further receives immunosuppressive therapy, such as calcineurin inhibitors, corticosteroids, microtubule inhibitors, low dose of a mycophenolic acid prodrug, or any combination thereof. In yet further embodiments, the subject being treated has received a non-myeloablative or a myeloablative hematopoietic cell transplant, wherein the treatment may be administered at least two to at least three months after the non-myeloablative hematopoietic cell transplant.

Methods according to this disclosure may include administering one or more additional agents to treat the disease or disorder in a combination therapy, or comprises administering one or more agents to treat the disease or disorder. For example, in certain embodiments, a combination therapy comprises administering a cell composition with (concurrently, simultaneously, or sequentially) an immune checkpoint inhibitor. In some embodiments, a therapy or combination therapy comprises administering a an agonist of a stimulatory immune checkpoint agent. In further embodiments, a combination therapy comprises administering a cell composition with a secondary therapy, such as chemotherapeutic agent, a radiation therapy, a surgery, an antibody, or any combination thereof.

As used herein, the term “immune suppression agent” or “immunosuppression agent” refers to one or more cells, proteins, molecules, compounds or complexes providing inhibitory signals to assist in controlling or suppressing an immune response. For example, immune suppression agents include those molecules that partially or totally block immune stimulation; decrease, prevent or delay immune activation; or increase, activate, or up regulate immune suppression. Exemplary immunosuppression agents to target (e.g., with an immune checkpoint inhibitor) include PD-1, PD-L1, PD-L2, CTLA4, TIGIT, LAG3, Tim-3, or any combination thereof.

An immune suppression agent inhibitor (also referred to as an immune checkpoint inhibitor) may be a compound, an antibody, an antibody fragment or fusion polypeptide (e.g., Fc fusion, such as CTLA4-Fc or LAG3-Fc), an antisense molecule, a ribozyme or RNAi molecule, or a low molecular weight organic molecule. In any of the embodiments disclosed herein, a method may comprise administering a cell composition with one or more inhibitor of any one of the following immune suppression components, singly or in any combination.

In certain embodiments, a therapy or combination therapy comprises a PD-1 inhibitor, for example a PD-1-specific antibody or binding fragment thereof, such as pidilizumab, nivolumab (Keytruda, formerly MDX-1106), pembrolizumab (Opdivo, formerly MK-3475), MEDI0680 (formerly AMP-514), AMP-224, BMS-936558 or any combination thereof. In further embodiments, a cell composition of the present disclosure (or an engineered host cell expressing the same) is used in combination with a PD-L1 specific antibody or binding fragment thereof, such as BMS-936559, durvalumab (MEDI4736), atezolizumab (RG7446), avelumab (MSB0010718C), MPDL3280A, or any combination thereof.

In certain embodiments, a combination therapy comprises a cell composition of the present disclosure and a secondary therapy comprising one or more of: an antibody or antigen binding-fragment thereof that is specific for a cancer antigen, a radiation treatment, a surgery, a chemotherapeutic agent, a cytokine, RNAi, or any combination thereof.

In certain embodiments, a therapeutic method or combination therapy method comprises or further comprises administering a radiation treatment or a surgery. Radiation therapy is well-known in the art and includes X-ray therapies, such as gamma-irradiation, radiopharmaceutical therapies, and proton therapy or proton radiotherapy. Surgeries and surgical techniques appropriate to treating a given cancer or non-inflamed solid tumor in a subject are well-known to those of ordinary skill in the art. In certain

In certain embodiments, a combination therapy method comprises administering a cell composition and further administering a chemotherapeutic agent. A chemotherapeutic agent includes, but is not limited to, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor. Illustrative chemotherapeutic agents include, without limitation, the following groups: anti-metabolites/anticancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, temozolamide, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors.

Cytokines can be used to manipulate host immune response towards anticancer activity. See, e.g., Floros & Tarhini, Semin. Oncol. 42 (4): 539-548, 2015. Cytokines useful for promoting immune anticancer or antitumor response include, for example, IFN-α, IL-2, IL-3, IL-4, IL-10, IL-12, IL-13, IL-15, IL-16, IL-17, IL-18, IL-21, IL-24, and GM-CSF, singly or in any combination with a cell composition of this disclosure.

Another cancer therapy approach involves reducing expression of oncogenes and other genes needed for growth, maintenance, proliferation, and immune evasion by cancer cells. RNA interference, and in particular the use of microRNAs (miRNAs) small inhibitory RNAs (siRNAs) provides an approach for knocking down expression of cancer genes. See, e.g., Larsson et al., Cancer Treat. Rev. 16 (55): 128-135, 2017. Techniques for making and using RNA for cancer therapy are known to those having ordinary skill in the art.

In any of the embodiments disclosed herein, any of the therapeutic agents (e.g., a cell composition, an inhibitor of an immune suppression component, an agonist of a stimulatory immune checkpoint molecule, an antitumor lymphocyte, a chemotherapeutic agent, a radiation therapy, a surgery, a cytokine, or an inhibitory RNA) may be administered once or more than once to the subject over the course of a treatment, and, in combinations, may be administered to the subject in any order (e.g., simultaneously, concurrently, or in any sequence) or any combination. An appropriate dose, suitable duration, and frequency of administration of the compositions will be determined by such factors as a condition of the patient; size, type, spread, growth, and severity of the tumor or cancer; particular form of the active ingredient; and the method of administration.

In certain embodiments, a plurality of doses of a cell composition as described herein is administered to the subject, which may be administered at intervals between administrations of about two to about four weeks. In further embodiments, a cytokine (e.g., IL-2, IL-15, IL-21) is administered sequentially, provided that the subject was administered the recombinant host cell at least three or four times before cytokine administration. In certain embodiments, the cytokine is administered concurrently with the cell composition. In certain embodiments, the cytokine is administered subcutaneously.

In still further embodiments, the subject being treated is further receiving immunosuppressive therapy, such as calcineurin inhibitors, corticosteroids, microtubule inhibitors, low dose of a mycophenolic acid prodrug, or any combination thereof. In yet further embodiments, the subject being treated has received a non-myeloablative or a myeloablative hematopoietic cell transplant, wherein the treatment may be administered at least two to at least three months after the non-myeloablative hematopoietic cell transplant.

The present disclosure includes the following non-limiting numbered Embodiments:

Embodiment 1. A method comprising identifying, selecting, and/or sorting, from a sample comprising CD4+ T cells, one or more CD4+ T cells positive for expression of CD200.

Embodiment 2. A method comprising identifying, selecting, and/or sorting, from a tumor sample comprising CD4+ T cells, one or more CD4+ T cells positive for expression of CD200.

Embodiment 3. A method comprising identifying, selecting, and/or sorting, from a sample comprising CD4+ T cells, CD4+ T cells that express CD200, wherein, optionally, the sample comprises a tumor sample.

Embodiment 4. A method comprising identifying, selecting, and/or sorting, from a sample comprising CD4+ T cells, CD4+ T cells that have increased expression of CD200 relative to one or more other CD4+ T cells of the sample, wherein, optionally, the sample comprises a tumor sample.

Embodiment 5. A method comprising identifying, selecting, and/or sorting, from a sample comprising CD4+ T cells, one or more CD4+ T cells positive for expression of CXCL13.

Embodiment 6. A method comprising identifying, selecting, and/or sorting, from a tumor sample comprising CD4+ T cells, one or more CD4+ T cells positive for expression of CXCL13.

Embodiment 7. A method comprising identifying, selecting, and/or sorting, from a sample comprising CD4+ T cells, CD4+ T cells that express CXCL13, wherein, optionally, the sample comprises a tumor sample.

Embodiment 8. A method comprising identifying, selecting, and/or sorting, from a sample comprising CD4+ T cells, CD4+ T cells that have increased expression of CXCL13 relative to one or more other CD4+ T cells of the sample, wherein, optionally, the sample comprises a tumor sample.

Embodiment 9. The method of any one of Embodiments 1-8, wherein:
(i) the sample comprising CD4+ T cells comprises blood and/or tumor; and/or
(ii) the sample comprising CD4+ T cells is from a subject having, or having previously been diagnosed with, a cancer, such as a solid cancer (e.g., melanoma or breast cancer); and/or
(iii) the sample comprising CD4+ T cells comprises tumor-infiltrating lymphocytes and/or non-tumor-infiltrating lymphocytes; and/or
(iv) the sample comprising CD4+ T cells is from or comprises tumor infiltrated by lymphocytes; and/or
(v) the sample comprising CD4+ T cells is from a subject that has previously been administered an anti-PD-1 antibody or antigen-binding fragment thereof, optionally nivolumab or pembrolizumab; and/or
(vi) the sample comprising CD4+ T cells has previously been exposed to an anti-PD-1 antibody or antigen-binding fragment thereof, optionally nivolumab or pembrolizumab.

Embodiment 10. The method of any one of Embodiments 1-9, wherein:
(i) identifying, selecting, and/or sorting the one or more CD4+ T cells positive for expression of CXCL13 or expressing CXCL13 or having increased expression of CXCL13 further comprises identifying, selecting, and/or sorting one or more of the identified, selected, and/or sorted CD4+ T cells positive for expression of PD-1 or expressing PD-1 or having increased expression of PD-1 relative to one or more other CD4+ T cells of the sample; and/or
(ii) identifying, selecting, and/or sorting the one or more CD4+ T cells positive for expression of CD200 or expressing CD200 or having increased expression of CD200 further comprises identifying, selecting, and/or sorting one or more of the of the identified, selected, and/or sorted CD4+ T cells positive for expression of PD-1 or expressing PD-1 or having increased expression of PD-1 relative to one or more other CD4+ T cells of the sample; and/or
(iii) the sample comprising CD4+ T cells has an increased frequency of CD4+ T cells positive for expression of PD-1 or expressing PD-1 or having increased expression of PD-1, relative a sample from the subject having an equivalent number of cells as compared to the sample.

Embodiment 11. A method comprising identifying, selecting, and/or sorting, from a sample comprising CD4+ T cells, one or more CD4+ T cells positive for expression of CXCL13 and PD-1.

Embodiment 12. A method comprising identifying, selecting, and/or sorting, from a sample comprising CD4+ T cells, one or more CD4+ T cells expressing CXCL13 and PD-1.

Embodiment 13. A method comprising identifying, selecting, and/or sorting, from a sample comprising CD4+ T cells, one or more CD4+ T cells that have increased expression of CXCL13 and PD-1 relative to one or more other CD4+ T cells of the sample.

Embodiment 14. A method comprising identifying, selecting, and/or sorting, from a sample comprising CD4+ T cells, one or more CD4+ T cells expressing CD200 and PD-1.

Embodiment 15. A method comprising identifying, selecting, and/or sorting, from a sample comprising CD4+ T cells, one or more CD4+ T cells that have increased expression of CD200 and PD-1 relative to one or more other CD4+ T cells of the sample.

Embodiment 16. A method comprising identifying, selecting, and/or sorting, from a sample comprising CD4+ T cells, one or more CD4+ T cells positive for expression of CD200 and PD-1.

Embodiment 17. A method comprising identifying, selecting, and/or sorting, from a sample comprising CD4+ T cells, one or more CD4+ T cells positive for expression of CD200 or that express CD200 or that have increased expression of CD200 relative to one or more other CD4+ T cells of the sample, wherein the sample is from a subject that has previously been administered an anti-PD-1 antibody or antigen-binding fragment thereof and/or the sample has been exposed to an anti-PD-1 antibody or antigen-binding fragment thereof.

Embodiment 18. The method of any one of Embodiments 1-17, wherein:
the sample comprises blood and/or tumor, wherein, optionally, the sample comprises blood from a subject that has, or has previously been diagnosed with, a cancer, such as a solid cancer (e.g., melanoma or breast cancer), and has further optionally previously been administered an anti-PD-1 antibody or antigen-binding fragment thereof.

Embodiment 19. The method of any one of Embodiments 1-18, wherein, optionally, other cells of the sample consist essentially of, or consist of CD4+ T cells.

Embodiment 20. The method of any one of Embodiments 1-19, wherein the sample comprises, consists essentially of, or consists of tumor-infiltrating lymphocytes.

Embodiment 21. The method of any one of Embodiments 1-20, wherein the one or more identified, selected, and/or sorted CD4+ T cells:
(i) are positive for expression, express, or have increased expression relative to one or more other CD4+ T cells from the sample, and optionally have intermediate or high expression of, (1) one or more memory gene and/or (2) one or more gene or surface marker associated with T follicular helper (TFH) cells; and/or
(ii) (1) are negative for TCF7 expression, do not express TCF7, or have reduced TCF7 expression as compared to other CD4+ T cells in the sample; and/or (2) are positive for expression of, express, or have increased expression as compared to one or more other CD4+ T cells from the sample, of one or more coinhibitory marker, one or more inflammatory marker, one or more cytolytic marker, and/or one or more tissue resident memory marker; and/or
(iii) are positive for expression of, express, or have increased expression as compared to one or more other CD4+ T cell from the sample, of one or more gene involved in proliferation.

Embodiment 21A. The method of Embodiment 21, wherein:
(i) the one or more memory gene comprises TCF7, IL7-R, or both; and/or
(ii) the one or more gene or surface marker associated with T follicular helper (TFH) cells comprises BCL6, CD200, CXCR5, or any combination thereof; and/or
(iii) the one or more coinhibitory marker comprises TIM-3, LAG-3, or both; and/or
(iv) the one or more inflammatory marker comprises CCL3, CCL4, IFNγ, IFN-γ mRNA, or any combination thereof; and/or
(v) the one or more cytolytic marker comprises GZMA/K, PRF1 mRNA, or both; and/or
(vi) the one or more gene involved in proliferation comprises TYMS, TOP2A, MCM2/4 mRNA, or any combination thereof; and/or
(vii) the one or more tissue resident memory marker comprises CD103.

Embodiment 22. The method of Embodiment 21 or Embodiment 21A, wherein the one or more identified, selected, and/or sorted CD4+ T cells:
(i) are positive for expression of, express, or have increased expression relative to one or more other CD4+ T cells of the sample, of any one or more (i.e. any one, any two, any three, any four, any five, or all six) of CXCL13, TCF7, IL7R, BCL6, CD200, and CXCR5, and optionally have high expression of any one or more of CXCL13, TCF7, IL7R, BCL6, CD200, and CXCR5;
(ii) (a) are positive for expression of, express, or have increased expression relative to one or more other CD4+ T cells of the sample, of CXCL13, and optionally have high expression of CXCL13, (b) are negative for TCF7 expression or do not express TCF7 expression or have reduced TCF7 expression as compared to one or more other CD4+ T cells of the sample, and (c) are positive for expression of, express, or have increased expression relative to one or more other CD4+ T cells from the sample, of TIM-3, LAG-3, IFN-γ mRNA, GZMA/K, PRF1 mRNA, and CD103; or
(iii) (a) are positive for expression of, express, or have increased expression relative to one or more other CD4+ T cells of the sample, of CXCL13, and optionally have high expression of CXCL13, and (b) are positive for expression of, express, or have increased expression relative to one or more other CD4+ T cells of the sample, of TYMS, TOP2A, and MCM2/4 mRNA,
and, optionally, (c) express, are positive for expression of, or have increased expression relative to one or more other CD4+ T cells of the sample of, BTLA and/or IL-21.

Embodiment 23. The method of any one of Embodiments 1-22, comprising identifying, selecting, and/or sorting one or more CD4+ T cells (a) positive for expression of, expressing, or having increased expression relative to one or more other CD4+ T cells of the sample, of CXCL13, CD200, or both, optionally (b) positive for expression of, expressing, or have increased expression of BTLA relative to one or more other CD4+ T cells of the sample, and:
(c) (i) positive for expression of, expressing, or having increased expression relative to one or more other CD4+ T cells of the sample, of (1) one or more memory gene and/or (2) one or more gene or surface marker associated with T follicular helper (TFH) cells; and/or
(c) (ii) (1) negative for TCF7 expression, not expressing TCF7, or having reduced TCF7 expression, relative to other CD4+ T cells of the sample; and/or (2) are positive for expression of, express, or have increased expression relative to one or more other CD4+ T cells of the sample, of one or more coinhibitory marker, one or more inflammatory marker, one or more cytolytic marker, and/or one or more tissue resident memory marker; and/or
(c) (iii) positive for expression of, expressing, or having increased expression relative to one or more other CD4+ T cells of the sample, of one or more gene involved in proliferation.

Embodiment 24. The method of Embodiment 23, wherein:
(i) the one or more memory gene comprises TCF7, IL7-R, or both; and/or
(ii) the one or more gene or surface marker associated with T follicular helper (T_{FH}) cells comprises BCL6, CD200, CXCR5, or any combination thereof; and/or
(iii) the one or more coinhibitory marker comprises TIM-3, LAG-3, or both; and/or
(iv) the one or more inflammatory marker comprises CCL3, CCL4, IFN-γ, IFN-γ mRNA, or any combination thereof; and/or
(v) the one or more cytolytic marker comprises GZMA/K, PRF1 mRNA, or both; and/or
(vi) the one or more gene involved in proliferation comprises TYMS, TOP2A, MCM2/4 mRNA, or any combination thereof; and/or
(vii) the one or more tissue resident memory marker comprises CD103.

Embodiment 25. The method of Embodiment 24, wherein:
(i) the one or more memory gene comprises TCF7, IL7-R, or both;
(ii) the one or more gene or surface marker associated with T follicular helper (TFH) cells comprises BCL6, CD200, CXCR5, or any combination thereof;
(iii) the one or more coinhibitory marker comprises TIM-3, LAG-3, or both;
(iv) the one or more inflammatory marker comprises CCL3, CCL4, IFN-γ, IFN-γ mRNA, or any combination thereof;
(v) the one or more cytolytic marker comprises GZMA/K, PRF1 mRNA, or both;
(vi) the one or more gene involved in proliferation comprises TYMS, TOP2A, MCM2/4 mRNA, or any combination thereof; and
(vii) the one or more tissue resident memory marker comprises CD103.

Embodiment 26. The method of Embodiment 24 or 25, comprising identifying, selecting, and/or sorting one or more CD4+ T cells that:
(i) have high expression of CXCL13 and/or CD200 and any one or more of TCF7, IL7R, and BCL6;
(ii) have high expression of CXCL13, are negative for TCF7 expression or have reduced TCF7 expression as compared to one or more other CD4+ T cells in the sample, and are positive for expression of, express, or have increased expression relative to one or more other CD4+ T cells of the sample, any one or more of TIM-3, LAG-3, IFN-γ mRNA, GZMA/K, PRF1 mRNA, and CD103; or
(iii) have high expression of CXCL13 and are positive for expression, express, or have increased expression relative to one or more other CD4+ T cells of the sample, of any one or more of TYMS, TOP2A, and MCM2/4 mRNA, and,
optionally, are positive for expression of BTLA, express BTLA, or have increased expression of BTLA relative to one or more other CD4+ T cells of the sample.

Embodiment 27. The method of any one of Embodiments 1-26, further comprising identifying, selecting, and/or sorting from the one or more identified, selected, and/or sorted CD4+ T cells, (a) one or more CD4+ T cells positive for expression of, expressing, or having increased expression relative to one or more other CD4+ T cells of the sample, of TCF7 and/or (b) one or more CD4+ T cells that are negative for expression of TCF7 or that do not express TCF7 or that have reduced expression of TCF7 as compared to one or more other CD4+ T cells from the sample and/or as compared to other of the one or more identified, selected, and/or sorted CD4+ T cells.

Embodiment 28. The method of any one of Embodiments 1-27, wherein identifying, selecting, and/or sorting one or more CD4+ T cells positive for expression of CXCL13 or expressing CXCL13 comprises identifying, selecting, and/or sorting one or more CD4+ T cells positive for expression of CD200 or expressing CD200, and optionally identifying, selecting, and/or sorting one or more CD4+ T cells that have high expression of CD200 or that have increased expression of CD200 relative to one or more other CD4+ T cells from the sample and/or as compared to other of the one or more identified, selected, and/or sorted CD4+ T cells.

Embodiment 29. The method of any one of Embodiments 1-28, wherein identifying, selecting, and/or sorting one or more CD4+ T cells positive for expression of CXCL13 or expressing CXCL13 further comprises identifying, selecting, and/or sorting one or more CD4+ T cells that express PD-1 or that are positive for expression of PD-1 or that have high expression of PD-1 or that have increased expression of PD-1 relative to one or more other CD4+ T cells from the sample and/or relative to other of the one or more identified, selected, and/or sorted CD4+ T cells.

Embodiment 30. The method of any one of Embodiments 1-29:
(i) further comprising identifying, selecting, and/or sorting, from the one or more identified, selected, and/or sorted CD4+ T cells, one or more CD4+ T cells positive for expression of CXCR6, expressing CXCR6, or having increased expression of CXCR6 relative to one or more other CD4+ T cells in the sample and/or relative to other of the one or more identified, selected, and/or sorted CD4+ T cells; and/or
(ii) further comprising identifying, selecting, and/or sorting, from the one or more identified, selected, and/or sorted CD4+ T cells, one or more CD4+ T cells that are negative for expression of CXCR6 or that do not express CXCR6 or that have reduced expression of CXCR6 relative to other CD4+ T cells of the sample and/or as relative to other of the one or more identified, selected, and/or sorted CD4+ T cells; and/or
(iii) wherein the one or more identified, selected, and/or sorted CD4+ T cells are negative for CD25 expression or do not express CD25 or have reduced expression of CD25 relative to one or more other CD4+ T cells of the sample and/or as compared to other of the one or more identified, selected, and/or sorted CD4+ T cells.

Embodiment 31. A method comprising identifying, selecting, and/or sorting, from a sample comprising CD4+ T cells, one or more CD4+ T cells that:
(i) optionally are negative for expression of CD25 or do not express CD25 or have reduced expression of CD25 relative to one or more other CD4+ T cells in the sample;
(ii) are positive for expression of PD-1 and CD200 or express PD-1 and CD200 or have increased expression relative to one or more other CD4+ T cells in the sample and optionally have high expression of PD-1 and/or CD200; and
iii) are positive for expression of CXCR6 or express CXCR6 and optionally have high expression of CXCR6 or increased expression of CXCR6 as compared to one or more other CD4+ T cells in the sample.

Embodiment 32. The method of Embodiment 31, wherein:
(i) the sample comprises blood and/or tumor from a subject; and/or
(ii) the sample is from a subject having, or having previously been diagnosed with, a cancer, such as a solid cancer (e.g., melanoma or breast cancer).

Embodiment 33. The method of Embodiment 31 or 32, wherein the sample comprises tumor infiltrated by lymphocytes, and optionally wherein the other CD4+ T cells in the sample comprise, consist essentially of, or consist of CD4+ T cells that did not infiltrate the tumor.

Embodiment 34. The method of any one of Embodiments 1-33, wherein identifying, selecting, and/or sorting comprises use of flow cytometry.

Embodiment 35. The method of any one of Embodiments 1-34, wherein the sample is from a subject that has previously been administered an anti-PD-1 antibody or antigen-binding fragment thereof, and/or the sample has been exposed to an anti-PD-1 antibody or antigen-binding fragment thereof, wherein the method comprises using an antibody or antigen-binding fragment thereof that binds to the anti-PD-1 antibody or antigen-binding fragment thereof, wherein, optionally, the anti-PD-1 antibody or antigen-binding fragment of comprises nivolumab or pembrolizumab and the method comprises using an anti-IgG4 antibody or antigen-binding fragment thereof.

Embodiment 36. The method of any one of Embodiments 1-35, further comprising isolating the one or more identified, selected, and/or sorted CD4+ T cells, wherein, optionally, isolating the one or more identified, selected, and/or CD4+ T cells comprises removing from the sample: (i) the one or more sorted CD4+ T cells; and/or
(ii) CD4+ T cells positive for expression of CD25, expressing CD25, or having increased expression of CD25 relative to other CD4+ T cells of the sample; and/or (iii) CD4+ T cells negative for expression of CD127, do not expressing CD127, or having reduced expression of CD127 relative to other CD4+ T cells of the sample; and/or (iii) CD4+ T cells negative for expression of, not expressing, or having reduced expression relative to one or more other CD4+ T cells of the sample, of any one or more of CXCL13, PD-1 and CD200.

Embodiment 37. The method of any one of Embodiments 1-36, comprising sorting the one or more identified, selected, and/or sorted CD4+ T cells away from CD4+ T cells negative for expression of, not expressing, or having reduced expression as compared to one or more other CD4+ T cells of the sample, of any one or more of CXCL13, PD-1, and CD200.

Embodiment 38. The method of any one of Embodiments 1-37, further comprising culturing the one or more one or more identified, selected, sorted, and/or isolated CD4+ T cells.

Embodiment 39. The method of any one of Embodiments 1-38, further comprising expanding the one or more identified, selected, sorted, and/or isolated CD4+ T cells.

Embodiment 40. The method of any one of Embodiments 1-39, further comprising exposing the one or more identified, selected, sorted, and/or isolated CD4+ T cells to: (i) one or more peptides that comprise a tumor antigen or tumor neoantigen, optionally wherein the tumor antigen or tumor neoantigen is present in the subject or sample and/or against which the one or more identified, selected, sorted, and/or isolated CD4+ T cells are reactive; and/or (ii)

antigen-presenting cells that present a tumor antigen or tumor neoantigen against which the one or more identified, selected, sorted, and/or isolated CD4+ T cells are reactive; and/or (iii) one or more activating cytokine; and/or (iv) one or more agent that binds to a stimulatory or costimulatory protein expressed on the cell surface of the one or more identified, selected, sorted, and/or isolated CD4+ T cells, wherein binding by the one or more agent to the stimulatory or costimulatory protein stimulates the one or more identified, selected, sorted, and/or isolated CD4+ T cells, wherein, optionally, the one or more agent that binds to a stimulatory or costimulatory protein comprises an antibody, or an antigen-binding fragment thereof, that binds to CD3, CD28, CD27, 4-1BB, OX40, ICOS, GITR, or any combination thereof.

Embodiment 41. The method of any one of Embodiments 1-40, further comprising administering the one or more of the one or more identified, selected, sorted, and/or isolated CD4+ T cells to a subject having a cancer, optionally a solid cancer (e.g., melanoma or breast cancer).

Embodiment 42. The method of Embodiment 41, wherein the subject having a cancer is the subject from which the sample was sourced.

Embodiment 43. The method of any one of Embodiments 1-42, further comprising sequencing a TRBV gene segment, a TRBD gene segment, a TRBJ gene segment, a TRAV gene segment, a TRAJ gene segment, or any combination thereof, from one or more of the one or more identified, selected, sorted, and/or isolated CD4+ T cells.

Embodiment 44. The method of Embodiment 43, comprising sequencing a TRBV gene segment, a TRBD gene segment, a TRBJ gene segment, a TRAV gene segment, and a TRAJ gene segment from one or more of the one or more identified, selected, sorted, and/or isolated CD4+ T cells, and optionally further sequencing a TRBC gene segment and/or a TRAC gene segment from one or more of the one or more identified, selected, sorted, and/or isolated CD4+ T cells.

Embodiment 45. The method of Embodiment 43 or 44, further comprising introducing: (1) a polynucleotide that encodes a TCR Vβ from an identified, selected, sorted, and/or isolated CD4+ T cell; and/or (2) a polynucleotide that encodes a TCR Vα from an identified, selected, sorted, and/or isolated CD4+ T cell; and/or (3) a polynucleotide that encodes a TCR Vβ and a TCR Vα, wherein the TCR Vβ comprises CDR1β, CDR2β, and/or CDR3B from ane identified, selected, sorted, and/or isolated CD4+ T cell, and/or wherein the TCR Vα comprises CDR1, CDR2a, and/or CDR3a from an/the identified, selected, sorted, and/or isolated CD4+ T cell, into one or more host cells, optionally comprising one or more T cells.

Embodiment 46. The method of Embodiment 45, comprising introducing: (1) a polynucleotide that encodes a TCRβ chain from an identified, selected, sorted, and/or isolated CD4+ T cell; and/or (2) a polynucleotide that encodes a TCRα chain from an identified, selected, sorted, and/or isolated CD4+ T cell, into one or more host cells, optionally comprising one or more T cells.

Embodiment 47. The method of Embodiment 45 or 46, wherein the one or more host T cells comprise CD4+ T cells and/or CD8+ T cells.

Embodiment 48. A CD4+ T cell or a population of CD4+ T cells or a host cell:
(i) identified, selected, sorted, isolated, cultured, expanded, and/or activated by the method of any one of Embodiments 1-41; or (ii) made by the method of any one of Embodiments 42-47.

Embodiment 49. A method comprising sequencing: a TRBV gene segment; a TRBD gene segment; a TRBJ gene segment; a TRAV gene segment; a TRAJ gene segment; or any combination thereof, from a CD4+ T cell selected or obtained from a sample comprising a plurality of CD4+ T cells, wherein the CD4+ T cell: (i) is positive for expression of CXCL13 or expresses CXCL13 or has increased expression of CXCL13 relative to one or more other CD4+ T cells of the plurality; (ii) is positive for expression of, expresses, or has increased expression relative to one or more other CD4+ T cells of the plurality, of PD-1 and CXCL13; (iii) is positive for expression of CD200, expresses CD200 or has increased expression of CD200 relative to one or more other CD4+ T cells of the plurality; or (iv) is positive for expression of PD-1 and CD200, expresses PD-1 and CD200, or has increased expression of PD-1 and/or of CD200 as relative to one or more other CD4+ T cells of the plurality.

Embodiment 50. The method of Embodiment 49, further comprising introducing, into one or more host cells: (1) a polynucleotide that encodes a TCR Vβ domain from the CD4+ T cell; and/or (2) a polynucleotide that encodes a TCR Vα domain from the CD4+ T cell; and/or (3) a polynucleotide that encodes a TCR Vβ and a TCR Vα, wherein the TCR Vβ comprises CDR1β, CDR2β, and/or CDR3B from the CD4+ T cell, and/or wherein the TCR Vα comprises CDR1, CDR2a, and/or CDR3a from the CD4+ T cell, wherein, optionally, the one or more host cells comprise T cells and/or the polynucleotide encodes a TCR.

Embodiment 51. The method of Embodiment 49 or 50, wherein the CD4+ T cell was selected or obtained from a sample comprising (1) tumor infiltrated by lymphocytes, (2) tumor-infiltrating lymphocytes, or (3) blood.

Embodiment 52. The method of any one of Embodiments 49-51, wherein the CD4+ T cell was selected or obtained from a sample from a subject having, or having previously been diagnosed with, a cancer, such as a solid cancer.

Embodiment 53. The method of any one of Embodiments 49-52, wherein the CD4+ T cell is specific for one or more tumor antigen or tumor neoantigen.

Embodiment 54. The method of any one of Embodiments 49-53, wherein the CD4+ T cell is a tumor infiltrating lymphocyte and wherein the other CD4+ T cells of the plurality comprise tumor infiltrating lymphocytes and/or non-tumor infiltrating lymphocytes.

Embodiment 55. A method comprising expanding one or more CD4+ T cells obtained, selected, or isolated from a sample comprising a plurality of CD4+ T cells, wherein the one or more CD4+ T cells: (i) is/are positive for expression of CXCL13 or expresses CXCL13 or has increased expression of CXCL13 relative to one or more other CD4+ T cells of the plurality; (ii) is/are positive for expression of, expresses, or has increased expression relative to one or more other CD4+ T cells of the plurality, of PD-1 and CXCL13; (iii) is/are positive for expression of CD200, expresses CD200 or has increased expression of CD200 relative to one or more other CD4+ T cells of the plurality; or (iv) is/are positive for expression of PD-1 and CD200, expresses PD-1 and CD200, or has increased expression of PD-1 and/or of CD200 as relative to one or more other CD4+ T cells of the plurality.

Embodiment 56. A method comprising expanding one or more CD4+ T cells that:
(i) express CXCL13; (ii) express PD-1 and CXCL13; (iii) express CD200; and/or (iv) express PD-1 and CD200, and, optionally, have increased expression of CXCL13, PD-1 and CXCL13, CD200, and/or PD-1 and CD200 as compared to other CD4+ T cells of a sample from which the one or more CD4+ T cells that are expanded was/were derived.

Embodiment 57. The method of any one of Embodiments 49-56, wherein the CD4+ T cell or the one or more CD4+ T cells: (i) express or has/have increased expression of (1) one or more memory gene and/or (2) one or more gene or surface marker associated with T follicular helper (T_{FH}) cells, as compared to other CD4+ T cells in the sample; (ii) (1) is/are negative for TCF7 expression or has/have reduced TCF7 expression, as compared to other CD4+ T cells in the sample; and/or (2) express one or more coinhibitory marker, one or more inflammatory marker, one or more cytolytic marker, and/or one or more tissue resident memory marker; and/or (iii) express one or more gene involved in proliferation.

Embodiment 58. The method of Embodiment 57, wherein: (i) the one or more memory gene comprises TCF7, IL7-R, or both; and/or (ii) the one or more gene or surface marker associated with T follicular helper (T_{FH}) cells comprises BCL6, CD200, CXCR5, or any combination thereof; and/or (iii) the one or more coinhibitory marker comprises TIM-3, LAG-3, or both; and/or (iv) the one or more inflammatory marker comprises CCL3, CCL4, IFNγ, IFN-γ mRNA, or any combination thereof; and/or (v) the one or more cytolytic marker comprises GZMA/K, PRF1 mRNA, or both; and/or (vi) the one or more gene involved in proliferation comprises TYMS, TOP2A, MCM2/4 mRNA, or any combination thereof; and/or (vii) the one or more tissue resident memory marker comprises CD103.

Embodiment 59. The method of any one of Embodiments 49-58, wherein the CD4+ T cell or the one or more CD4+ T cells: (i) is/are positive for expression, express, or has/have increased expression of CXCL13 and any one or more of TCF7, IL7R, BCL6, and CD200, relative to one or more other CD4+ T cells from the sample; (ii) is/are positive for expression, express, or has/have increased expression of CXCL 13 relative to other CD4+ T cells from the sample, are negative for TCF7 expression or have reduced TCF7 expression, relative to one or more other CD4+ T cells from the sample, and express one or more of TIM-3, LAG-3, IFN-γ mRNA, GZMA/K, PRF1 mRNA, and CD103; or (iii) is/are positive for expression of CXCL13, express CXCL13, or has/have increased expression of CXCL13 relative to one or more other CD4+ T cells from the sample and express one or more of TYMS, TOP2A, and MCM2/4 mRNA, and optionally, express BTLA.

Embodiment 60. A CD4+ T cell expressing: (i) CXCL13; (ii) PD-1 and CXCL13;
(iii) CD200; and/or (iv) PD-1 and CD200.

Embodiment 61. A composition comprising a plurality of CD4+ T cells, wherein 5% or more, 6% or more, 7% or more, 8% or more, 9% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, or 100% of the CD4+ T cells in the composition are according to Embodiment 60.

Embodiment 62. The composition of Embodiment 61, further comprising a pharmaceutically acceptable carrier, excipient, or diluent.

Embodiment 63. The composition of Embodiment 61 or 62, wherein the CD4+ T cell: (i) further expresses (1) one or more memory gene and/or (2) one or more gene or surface marker associated with T follicular helper (T_{FH}) cells; (ii) (1) does not express TCF7 or is negative for TCF7 expression; and/or (2) expresses one or more coinhibitory marker, one or more inflammatory marker, one or more cytolytic marker, and/or one or more tissue resident memory marker; (iii) expresses one or more gene involved in proliferation; (iv) expresses BTLA; (v) expresses CXCR6; and/or (vi) does not express CXCR6 or is negative for expression of CXCR6, and, optionally, does not express CD25 or is negative for CD25 expression.

Embodiment 64. A CD4+ T cell from a sample comprising a plurality of CD4+ T cells, wherein the CD4+ T cell: (i) expresses CXCL13, optionally having increased expression of CXCL13 as compared to one or more other CD4+ T cells of the plurality; (ii) expresses PD-1 and CXCL13, optionally having increased expression of PD-1 and/or of CXCL13 as compared to one or more other CD4+ T cells of the plurality; (iii) expresses CD200, optionally having increased expression of CD200 as compared to one or more other CD4+ T cells of the plurality; and/or (iv) expresses PD-1 and CD200, optionally having increased expression of PD-1 and/or of CXCL13 as compared to one or more other CD4+ T cells of the plurality, wherein, optionally, the CD4+ T cell:
(e) is negative for CD25 expression or has reduced expression of CD25 as compared to one or more other CD4+ T cells of the plurality; and/or
(f) expresses CXCR6, optionally at an increased level as compared to one or more other CD4+ T cells in the plurality; and/or
(g) is negative for expression of CXCR6 or has reduced expression of CXCR6 as compared to one or more other CD4+ T cells of the plurality; and/or
(h) is from a sample from a subject who had previously been administered an anti-PD-1 antibody or antigen-binding fragment thereof.

Embodiment 65. The CD4+ T cell of Embodiment 64, wherein the CD4+ T cell:
(i) has increased expression of (1) one or more memory gene and/or (2) one or more gene or surface marker associated with T follicular helper (TFH) cells, as compared to other CD4+ T cells in the sample;
(ii) (1) is/are negative for TCF7 expression or has/have reduced TCF7 expression, as compared to other CD4+ T cells in the sample; and/or (2) expresses/express one or more coinhibitory marker, one or more inflammatory marker, one or more cytolytic marker, and/or one or more tissue resident memory marker; and/or
(iii) expresses/express one or more gene involved in proliferation.

Embodiment 66. The CD4+ T cell of Embodiment 64 or 65, wherein:
(i) the one or more memory gene comprises TCF7, IL-7R, or both;
(ii) the one or more gene or surface marker associated with T follicular helper (TFH) cells comprises BCL6, CD200, CXCR5, or any combination thereof; and/or
(iii) the one or more coinhibitory marker comprises TIM-3, LAG-3, or both; and/or
(iv) the one or more inflammatory marker comprises CCL3, CCL4, IFNγ, IFN-γ mRNA, or any combination thereof; and/or
(v) the one or more cytolytic marker comprises GZMA/K, PRF1 mRNA, or both; and/or
(vi) the one or more gene involved in proliferation comprises TYMS, TOP2A, MCM2/4 mRNA, or any combination thereof; and/or
(vii) the one or more tissue resident memory marker comprises CD103.

Embodiment 67. The CD4+ T cell of Embodiment 66, wherein the CD4+ T cell expresses (i) CXCL13; (ii) PD-1 and CXCL13; (iii) CD200; and/or (iv) PD-1 and CD200, and further expresses:
(a) TCF7, IL7R, BCL6, CD200, CXCR5, or any combination thereof;
(b) TIM-3, LAG-3, IFN-γ mRNA, GZMA/K, PRF1 mRNA, CD103, or any combination thereof;
(c) TYMS, TOP1A, MCM2/4 mRNA, or any combination thereof; and/or BTLA. (d)

Embodiment 68. The CD4+ T cell of Embodiment 66 or 67, wherein the CD4+ T cell:
(i) has increased expression of CXCL13 and any one or more of TCF7, IL7R, BCL6, and CD200, as compared to other CD4+ T cells from the sample;
(ii) has increased expression of CXCL13 as compared to other CD4+ T cells from the sample, are negative for TCF7 expression or have reduced TCF7 expression, as compared to other CD4+ T cells in the sample, and express one or more of TIM-3, LAG-3, IFN-γ mRNA, GZMA/K, PRF1 mRNA, and CD103; or
(iii) has increased expression of CXCL13 as compared to other CD4+ T cells from the sample and express one or more of TYMS, TOP2A, and MCM2/4 mRNA, and optionally, expresses BTLA.

Embodiment 69. The CD4+ T cell of any one of Embodiments 61-68, which was obtained from a sample: (i) comprising tumor, tumor infiltrated by lymphocytes, tumor infiltrating lymphocytes, and/or blood; and/or (ii) from a subject having, or having previously been diagnosed with, a cancer, such as a solid cancer (e.g., melanoma or breast cancer); and/or (iii) from a subject who had previously been administered an anti-PD-1 antibody or antigen-binding fragment thereof.

Embodiment 70. The CD4+ T cell of any one of Embodiments 48, 60, and 64-69, which is specific for tumor antigen or a tumor neoantigen, optionally an antigen or neoantigen from a solid tumor.

Embodiment 71. A composition comprising a plurality of the CD4+ T cell of any one of Embodiments 48, 60, and 64-70, and optionally one or both of: (i) a plurality of tumor antigen-specific or tumor neoantigen-specific CD8+ T cells; and (ii) a pharmaceutically acceptable carrier, excipient, or diluent.

Embodiment 72. A population of CD4+ T cells or a composition comprising CD4+ T cells, wherein 5% or more, 6% or more, 7% or more, 8% or more, 9% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, or 100% of the CD4+ T cells in the population or composition, respectively, are CD4+ T cells according to any one of Embodiments 48, 60, and 64-70.

Embodiment 73. A cell population comprising a plurality of CD4+ T cells according to any one of Embodiments 48, 60, and 64-70, wherein, optionally, the population comprises the CD4+ T cells in an amount comprising from about 2-fold to about 20-fold (e.g., about 2-, about 3-, about 4-, about 5-, about 6, about 7, about 8-, about 9-, about 10-, about 11-, about 12-, about 13-, about 14-, about 15-, about 16-, about 17-, about 18-, about 19-, or about 20-fold) higher than the amount of such CD4+ T cells present in a subject sample (e.g. comprising tumor, such as a solid tumor sample, and/or blood) comprising an equivalent number of cells as the population.

Embodiment 74. A method of treating a cancer in a subject, comprising administering to the subject an effective amount of: (i) the CD4+ T cell or CD4+ T cell population of any one of Embodiments 48, 60, 64-70, and 72 or 73, respectively; and/or (ii) the composition of Embodiment 71 or 72; and/or (iii) a host cell made by the method of Embodiment 45 or 46 or 50-54, wherein optionally the host cell comprises a T cell, optionally a CD4+ T cell.

Embodiment 75. The CD4+ T cell or CD4+ T cell population of any one of Embodiments 48, 60, 64-70, and 72 or 73, respectively, the composition of Embodiment 71 or 72, and/or a host cell made by the method of any one of Embodiments 45, 46, and 50-54, wherein, optionally, the host cell comprises a T cell, further optionally a CD4+ T cell, for use in a method of treating cancer, optionally a solid cancer, in a subject.

Embodiment 76. The CD4+ T cell or CD4+ T cell population of any one of Embodiments 48, 60, 64-70, and 72 or 73, respectively, the composition of Embodiment 71 or 72, and/or a host cell made by the method of any one of Embodiments 45, 46, and 50-54, wherein, optionally, the host cell comprises a T cell, further optionally a CD4+ T cell, for use in the manufacture of a medicament for treating cancer, optionally a solid cancer, in a subject.

Embodiment 77. The method of any one of Embodiments 9-48, 52-54, and 75, the use of Embodiment 76, the CD4+ T cell or CD4+ T cell population of any one of Embodiments 48, 64-70, 72, and 73, and/or the composition of Embodiment 71 or 72, wherein the solid cancer is selected from: melanoma; breast cancer; a cancer of the head or neck; pancreatic cancer; cholangiocarcinoma; hepatocellular cancer; breast cancer including triple-negative breast cancer (TNBC); gastric cancer; non-small-cell lung cancer; prostate cancer; esophageal cancer; mesothelioma; small-cell lung cancer; colorectal cancer; glioblastoma; carcinoma; sarscoma; chondrosarcoma; fibrosarcoma (fibroblastic sarcoma); Dermatofibrosarcoma protuberans (DFSP); osteosarcoma; rhabdomyosarcoma; Ewing's sarcoma; a gastrointestinal stromal tumor; Leiomyosarcoma; angiosarcoma (vascular sarcoma); Kaposi's sarcoma; liposarcoma; pleomorphic sarcoma; synovial sarcoma; a lung carcinoma (e.g., Adenocarcinoma, Squamous Cell Carcinoma (Epidermoid Carcinoma); Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carciroma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma); an ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, or an adenocarcinoma of the prostate.

Embodiment 78. The method of Embodiment 77, the use of Embodiment 77, the CD4+ T cell or CD4+ T cell population of Embodiment 77, and/or the composition of Embodiment 77, wherein the solid cancer comprises a melanoma, a breast cancer, or both.

Embodiment 79. A method for identifying a subject as being at increased risk of relapse and/or progression of a solid cancer, the method comprising identifying a subject for whom less than 30% of CD4+ T cells present in a tumor sample of the solid cancer express CXCL13, CXCL13 and PD-1, CD200, or CD200 and PD-1, wherein, optionally, expression of CD200 and/or PD-1 is determined using flow cytometry comprising an isotype control antibody or antigen-binding fragment thereof.

Embodiment 80. A method for reducing a risk of a subject experiencing relapse and/or progression of a solid cancer, the method comprising administering, to a subject for whom less than 30% of CD4+ T cells present in a tumor sample of the solid cancer express CXCL13, CXCL13 and PD-1, CD200, or CD200 and PD-1, one or more therapies for the solid cancer, wherein, optionally, the one or more therapies comprise surgery, local radiation therapy, systemic radiation therapy, proton therapy, immunotherapy (e.g. comprising a cytokine, an antibody or antigen-binding fragment thereof, a fusion protein, antigen-specific T cells, antigen-specific NK cells, antigen-specific phagocytic cells, or any combination thereof), or any combination thereof.

Embodiment 81. A method for treating a solid cancer, the method comprising administering, to a subject for whom less than 30% of CD4+ T cells present in a tumor sample of the solid cancer express CXCL13, CXCL13 and PD-1, CD200, or CD200 and PD-1, one or more therapies for the solid cancer wherein, optionally, the one or more therapies comprise surgery, local radiation therapy, systemic radiation therapy, proton therapy, immunotherapy (e.g. comprising a cytokine, an antibody or antigen-binding fragment thereof, a fusion protein, antigen-specific T cells, antigen-specific NK cells, antigen-specific phagocytic cells, or any combination thereof), or any combination thereof.

Embodiment 82. A method for identifying a subject as having a positive prognosis (e.g. increased likelihood of longer survival, longer disease-free survival, longer progression-free survival, or disease remission) for and/or as being at a reduced risk for progression or relapse of a solid cancer, the method comprising identifying a subject for whom 30% or more of CD4+ T cells present in a tumor sample of the solid cancer express CXCL13, CXCL13 and PD-1, CD200, or CD200 and PD-1, thereby identifying the subject as having a positive prognosis and/or as being at reduced risk for progression or relapse of a solid cancer as compared to a subject in whom less than 30% of CD4+ T cells that infiltrate a tumor of the solid cancer express CXCL13, CXCL13 and PD-1, CD200, or CD200 and PD-1.

Embodiment 83. A method for identifying a subject with a solid cancer as needing a reduced a number of courses of therapy, a reduced number of types of therapy, a reduced duration of therapy, and/or a reduced exposure to a toxicity associated with therapy for the solid cancer in the subject, the method comprising determining that 30% or more of CD4+ T cells present in a tumor sample of the solid cancer express CXCL13, CXCL13 and PD-1, CD200, or CD200 and PD-1, thereby identifying the subject as needing a reduced number, number of type, and/or duration of therapy for the solid cancer, as compared to the subject prior to the determining and/or as compared to a subject in which less than 30% of CD4+ T cells that infiltrate a tumor of the solid cancer express CXCL13, CXCL13 and PD-1, CD200, or CD200 and PD-1.

Embodiment 84. A method for determining a prognosis of a solid cancer in a subject, the method comprising determining whether: (i) 30% or more of CD4+ T cells present in a tumor sample of the solid cancer express CXCL13, CXCL13 and PD-1, CD200, or CD200 and PD-1, thereby identifying the subject as having a better prognosis; or (ii) less than 30% of CD4+ T cells present in a tumor sample of the solid cancer express CXCL13, CXCL13 and PD-1, CD200, or CD200 and PD-1, thereby identifying the subject has having a worse prognosis.

Embodiment 85. The method of any one of Embodiments 79-84, wherein the CD4+ T cells that express CXCL13, CXCL13 and PD-1, CD200, or CD200 and PD-1 have increased expression of CXCL13, CXCL13 and PD-1, CD200, or CD200 and PD-1, respectively, as compared to CD4+ T cells of the subject that do not infiltrate a (optionally the) tumor of the solid cancer and/or as compared to one or more other CD4+ T cells present in the tumor sample.

Embodiment 86. The method of any one of Embodiments 79-85, wherein the CD4+ T cells that Embodiment express CXCL13, CXCL13 and PD-1, CD200, or CD200 and PD-1 also: (i) express (1) one or more memory gene and/or (2) one or more gene or surface marker associated with T follicular helper (TFH) cells; (ii) (1) are negative for TCF7 expression; and/or (2) express one or more coinhibitory marker, one or more inflammatory marker, one or more cytolytic marker, and/or one or more tissue resident memory marker; and/or (iii) express one or more gene involved in proliferation.

Embodiment 87. The method of Embodiment 86, wherein: (i) the one or more memory gene comprises TCF7, IL-7R, or both; (ii) the one or more gene or surface marker associated with T follicular helper (TFH) cells comprises BCL6, CD200, CXCR5, or any combination thereof; and/or (iii) the one or more coinhibitory marker comprises TIM-3, LAG-3, or both; and/or (iv) the one or more inflammatory marker comprises CCL3, CCL4, IFNγ, IFN-γ mRNA, or any combination thereof; and/or (v) the one or more cytolytic marker comprises GZMA/K, PRF1 mRNA, or both; and/or (vi) the one or more gene involved in proliferation comprises TYMS, TOP2A, MCM2/4 mRNA, or any combination thereof; and/or (vii) the one or more tissue resident memory marker comprises CD103.

Embodiment 88. The method of Embodiment 87, wherein the CD4+ T cells that express CXCL13, CXCL13 and PD-1, CD200, or CD200 and PD-1 also express: (a) TCF7, IL7R, BCL6, CD200, CXCR5, or any combination thereof, optionally at a level that is increased as compared to the level(s) expressed by CD4+ T cells that do not infiltrate a (optionally the) tumor of the solid cancer and/or as compared to one or more other CD4+ T cells present in the tumor sample; (b) TIM-3, LAG-3, IFN-γ mRNA, GZMA/K, PRF1 mRNA, CD103, or any combination thereof, optionally at a level that is increased as compared to the level(s) expressed by CD4+ T cells that do not infiltrate a (optionally the) tumor of the solid cancer; and/or as compared to one or more other CD4+ T cells present in the tumor sample; (c) TYMS, TOP1A, MCM2/4 mRNA, or any combination thereof, optionally at a level that is increased as compared to the level(s) expressed by CD4+ T cells that do not infiltrate a tumor of the solid cancer and/or as compared to one or more other CD4+ T cells present in the tumor sample; and/or (d) BTLA, optionally at a level that is increased as compared to the level(s) expressed by CD4+ T cells that do not infiltrate a tumor of the solid cancer and/or as compared to one or more other CD4+ T cells present in the tumor sample.

Embodiment 89. The method of any one of Embodiments 79-88, wherein the CD4+ T cells that express CXCL13, CXCL13 and PD-1, CD200, or CD200 and PD-1 also express CXCR6.

Embodiment 90. The method of any one of Embodiments 79-89, wherein the CD4+ T cells that express CXCL13, CXCL13 and PD-1, CD200, or CD200 and PD-1 are negative for expression of CXCR6 or have reduced expression of CXCR6 as compared to one or more other CD4+ T cells from the subject, optionally that infiltrate a tumor of the solid cancer.

Embodiment 91. A host cell made by the method of any one of Embodiments 50-54, wherein, optionally, the host cell comprises a T cell, further optionally a CD4+ T cell.

Embodiment 92. A method for identifying a population of CD4+ T cells from a sample, the method comprising identifying CD4+ T cells that: (i) express PD-1 and CD200, optionally at increased levels as compared to other CD4+ T cells in the sample, and, optionally, do not express or have reduced expression of CD25 as compared to one or more other CD4+ T cells from the sample; and (ii) (a) express CXCR6, optionally at an increased level as compared to other CD4+ T cells from the sample, wherein the identified population comprises a T effector cell phenotype and has reduced proliferative capacity; or (b) do not express CXCR6 or have reduced expression of CXCR6 as compared to one or more other CD4+ T cells from the sample, wherein the identified population comprises a T follicular helper cell phenotype and/or comprises stem and/or progenitor CD4+ T cells with proliferative capacity.

Embodiment 93. A method for identifying a population of CD4+ T cells from a sample, the method comprising identifying CD4+ T cells that: (i) express PD-1 and CD200, optionally at increased levels as compared to other CD4+ T cells from the sample, and, optionally, do not express or have reduced expression of CD25 as compared to one or more other CD4+ T cells from the sample; and (ii) (a) express TCF7, optionally at an increased level as compared to one or more other CD4+ T cells from the sample, wherein the identified population comprises a T follicular helper cell phenotype and/or comprises stem and/or progenitor CD4+ T cells with proliferative capacity; or (b) do not express TCF7 or have reduced expression of TCF7 as compared to one or more other CD4+ T cells from sample, wherein the identified population comprises a T effector cell phenotype and has reduced proliferative capacity.

Embodiment 94. A method for identifying a population of CD4+ T cells from a sample, the method comprising identifying CD4+ T cells that: (i) express CXCL13 and optionally PD-1, optionally at increased levels as compared to one or more other CD4+ T cells from the sample, and, optionally, do not express or have reduced expression of CD25 as compared to one or more other CD4+ T cells from the sample; and (ii) (a) express CXCR6, optionally at an increased level as compared to one or more other CD4+ T cells from the sample, wherein the identified population comprises a T effector cell phenotype and has reduced proliferative capacity; or (b) do not express CXCR6 or have reduced expression of CXCR6 as compared to one or more other CD4+ T cells from the sample, wherein the identified population comprises a T follicular helper cell phenotype and/or comprises stem and/or progenitor CD4+ T cells with proliferative capacity.

Embodiment 95. A method for identifying a population of CD4+ T cells from a sample, the method comprising identifying CD4+ T cells that: (i) express CXCL13 and optionally PD-1, optionally at increased levels as compared to one or more other CD4+ T cells from the sample, and, optionally, do not express or have reduced expression of CD25 as compared to one or more other CD4+ T cells from the sample; and (ii) (a) express CXCR6, optionally at an increased level as compared to one or more other CD4+ T cells from the sample, wherein the identified population comprises a T effector cell phenotype and has reduced proliferative capacity; or (b) do not express CXCR6 or have reduced expression of CXCR6 as compared to one or more other CD4+ T cells from the sample, wherein the identified population comprises a T follicular helper cell phenotype and/or comprises stem and/or progenitor CD4+ T cells with proliferative capacity.

Embodiment 96. A method for identifying a CD4+ T cell, such as for use in adoptive cell therapy, the method comprising identifying, from a sample comprising CD4+ T cells, one or more CD4+ T cells that: (i) express CXCL13; (ii) have increased expression of CXCL13 as compared to one or more other CD4+ T cells from the sample; (iii) express CD200; and/or (iv) have increased expression of CD200 as compared to one or more other CD4+ T cells from the sample.

Embodiment 97. A method for identifying a CD4+ T cell having and/or capable of contributing to an antitumor effect, the method comprising identifying, from a sample comprising CD4+ T cells, one or more CD4+ T cells that: (i) express CXCL13;
(ii) have increased expression of CXCL13 as compared to one or more other CD4+ T cells from the sample; (iii) express CD200; and/or (iv) have increased expression of CD200 as compared to one or more other CD4+ T cells from the sample.

Embodiment 98. The method of Embodiment 96 or 97, wherein the identified CD4+ T cells express PD-1 and/or have increased expression of PD-1 as compared to one or more other CD4+ T cells from the sample.

Embodiment 99. The method of any one of Embodiments 96-98, further comprising identifying, from the one or more identified CD4+ T cells, CD4+ T cells that are negative for expression of CXCR6 or that have reduced expression of CXCR6 as compared to one or more other of the one or more identified CD4+ T cells.

Embodiment 100. The method of any one of Embodiments 96-99, further comprising identifying, from the one or more identified CD4+ T cells, CD4+ T cells that express CXCR6 or that have increased expression of CXCR6 as compared to one or more other of the one or more identified CD4+ T cells.

Embodiment 101. The method of any one of Embodiments 96-100, further comprising identifying, from the one or more identified CD4+ T cells, CD4+ T cells that are negative for expression of TCF7 or that have reduced expression of TCF7 as compared to one or more other of the one or more identified CD4+ T cells.

Embodiment 102. The method of any one of Embodiments 96-101, further comprising identifying, from the one or more identified CD4+ T cells, CD4+ T cells that express TCF7 or that have increased expression of TCF7 as compared to one or more other of the one or more identified CD4+ T cells.

Embodiment 103. The method of any one of Embodiments 96-102, further comprising sorting the one or more identified CD4+ T cells away from other cells.

Embodiment 104. The method of any one of Embodiments 96-103, further comprising expanding the one or more identified or sorted CD4+ T cells.

Embodiment 105. The method of any one of Embodiments 96-104, wherein the sample comprises tumor infiltrated by lymphocytes and/or tumor infiltrating lymphocytes.

Embodiment 106. The method of any one of Embodiments 96-105, further comprising exposing the identified, sorted, and/or expanded CD4+ T cells to: (i) one or more peptides that comprise a tumor antigen or tumor neoantigen, optionally wherein the tumor antigen or tumor neoantigen is present in the subject and/or against which the one or more identified, selected, sorted, and/or isolated CD4+ T cells are reactive; and/or (ii) antigen-presenting cells that present a tumor antigen or tumor neoantigen, optionally against which the one or more identified, selected, sorted, and/or isolated CD4+ T cells are known to be reactive; and/or (iii) one or more activating cytokine; and/or (iv) one or more agent that binds to a stimulatory or costimulatory protein expressed on the cell surface of the one or more identified, sorted, and/or expanded CD4+ T cells, wherein binding by the one or more agent to the stimulatory or costimulatory protein stimulates the one or more identified, selected, sorted, and/or isolated CD4+ T cells, wherein, optionally, the one or more agent that binds to a stimulatory or costimulatory protein comprises an antibody, or an antigen-binding fragment thereof, that binds to CD3, CD28, CD27, 4-1BB, OX40, ICOS, GITR, or any combination thereof.

Embodiment 107. A method for identifying a T cell receptor, or one or more variable domains thereof, or one or more complementarity determining regions thereof, for use in cellular immunotherapy, the method comprising sequencing a TRAV gene segment, a TRAJ gene segment, a TRBV gene segment, a TRBD gene segment, and a TRBJ gene segment from one or more CD4+ T cells obtained from a sample, wherein the one or more CD4+ T cells: (i) express CXCL13; (ii) have increased expression of CXCL13 as compared to one or more other CD4+ T cells from the sample; (iii) express CD200; and/or
(iv) have increased expression of CD200 as compared to one or more other CD4+ T cells from the sample.

Embodiment 108. The method of Embodiment 107, wherein the one or more CD4+ T cells: (i) express PD-1 and/or have increased expression of PD-1 as compared to one or more other CD4+ T cells from the sample; and/or (ii) (a) are negative for expression of CXCR6 or have reduced expression of CXCR6 as compared to one or more other CD4+ T cells from the sample or (b) express CXCR6 or have increased expression of CXCR6 as compared to one or more other CD4+ T cells from the sample; and/or (iii) (a) express TCF7 or have increased expression of TCF7 as compared to one or more other CD4+ T cells from the sample, or (b) are negative for expression of TCF7 or have reduced expression of TCF7 as compared to one or more other CD4+ T cells from the sample.

Embodiment 109. The method of Embodiment 107 or 108, wherein the sample comprises tumor infiltrated by lymphocytes and/or tumor infiltrating lymphocytes.

Embodiment 110. The method of any one of Embodiments 107-109, further comprising introducing one or more polynucleotide encoding: the T cell receptor, or one or more variable domains thereof, or one or more complementarity determining regions thereof, into a host cell, optionally an immune cell, further optionally a T cell such as a CD4+ T cell.

Embodiment 111. The method of any one of Embodiments 107-110, wherein the one or more CD4+ T cells are specific for a tumor antigen or a tumor neoantigen.

Embodiment 112. A method for enriching for a CD4+ T cell having antitumor activity and/or capable of contributing to antitumor activity and/or expressing a T cell receptor specific for a tumor antigen or tumor neoantigen, the method comprising
(1) identifying, from a sample comprising CD4+ T cells, one or more CD4+ T cells that: (i) express CXCL13; (ii) have increased expression of CXCL13 as compared to one or more (i) other CD4+ T cells from the sample; (iii) express CD200; and/or (iv) have increased expression of CD200 as compared to one or more other CD4+ T cells from the sample; and (2) enriching for the identified CD4+ T cells.

Embodiment 113. The method of Embodiment 112, wherein the identified CD4+ T cells express PD-1 and/or have increased expression of PD-1 as compared to one or more other CD4+ T cells from the sample.

Embodiment 114. The method of Embodiment 112 or 113, further comprising identifying, from the one or more identified CD4+ T cells: (i) (a) CD4+ T cells that do not express CXCR6 or that have reduced expression of CXCR6 as compared to one or more other of the one or more identified CD4+ T cells and/or (b) CD4+ T cells that express CXCR6 or that have increased expression of CXCR6 as compared to one or more other of the one or more the identified CD4+ T cells; and/or (ii) (a) CD4+ T cells that are negative for expression of TCF7 or that have reduced expression of TCF7 as compared to one or more other of the one or more identified CD4+ T cells and/or (b) CD4+ T cells that express TCF7 or that have increased expression of TCF7 as compared to one or more other of the one or more identified CD4+ T cells.

Embodiment 115. The method of any one of Embodiments 112-114, wherein the sample comprises tumor infiltrated by lymphocytes and/or tumor infiltrating lymphocytes.

Embodiment 116. A population of CD4+ T cells or a composition comprising CD4+ T cells, wherein 5% or more, 6% or more, 7% or more, 8% or more, 9% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, or 100% of the CD4+ T cells in the population or composition are: (i) CXCL13+; (ii) CXCL13+PD-1+; (iii) CD200+; (iv) CD200+PD-1+; (v) CD200+PD-1+ CXCR6-; (vi) CD200+PD-1+ CXCR6+; (vii) TCF7+; (vii) TCF7-; or (viii) any combination of (i)-(viii).

Embodiment 117. The population or composition of Embodiment 116, wherein the CD4+ T cells were obtained from a sample and have increased expression of CXCL13, PD-1, and/or CD200 as compared to one or more other CD4+ T cells from the sample.

Embodiment 118. The method, CD4+ T cell, population of CD4+ T cells, host cell, composition, or population of any one of the preceding Embodiments, wherein the CD4+ T cell, the one or more CD4+ T cells, the one or more sorted, selected, and/or identified T cells, or the host cell, respectively, produces more interferon-gamma (IFN-γ) when in the presence of a tumor antigen peptide or tumor neoantigen peptide as compared to when in the presence of a wild-type peptide.

Embodiment 119. A kit for sorting or for identifying CD4+ T cells, comprising: (i) a reagent for detecting expression of CXCL13; and/or (ii) a reagent for detecting expression of CD200; and, optionally, (iii) instructions for using the reagent of (i) and/or the reagent of (ii) to detect expression of CXCL13 or CD200, respectively, on a cell of interest, such as a T cell, such as a CD4+ T cell.

Embodiment 120. The kit of Embodiment 119, further comprising: (iii) a reagent for detecting expression of PD-1 or for detecting an anti-PD-1 antibody or antigen-binding fragment thereof.

Embodiment 121. The kit of Embodiment 119 or 120, further comprising: (iv) a reagent for detecting expression of CXCR6; and/or (v) a reagent for detecting expression of TCF7.

Embodiment 122. The kit of any one of Embodiments 119-121, wherein the reagent of (i), (ii), (iii), (iv), and/or (v) comprises an antibody or antigen-binding fragment that binds specifically to CXCL13, CD200, PD-1, CXCR6, or TCF7, respectively.

Embodiment 123. The kit of any one of Embodiments 119-122, wherein the reagent of (i), (ii), (iii), (iv), and/or (v) comprises a labeled nucleic acid probe specific mRNA of CXCL13, CD200, PD-1, CXCR6, or TCF7, respectively.

### EXAMPLES

### Example 1

Identification of Neoantigen-Specific Cd4+ T Cell Tcrvβ Clonotypes from Melanoma Tumors

The specificity of CD4+ T cells for tumor antigens in patients with melanoma was identified and then single cell sequencing and matching of TCR clonotypes were used to characterize the transcriptional signatures of tumor infiltrating populations. These data show that the frequency of tumor infiltrating CD4+ T cells expressing a gene expression signature of tumor-reactive T cells correlate with differences in frequency and functional states of CD8+ T cells, myeloid cells and B cells in the tumor microenvironment.

Briefly, matching of information obtained by scRNA sequencing data to the antigen specificity of individual T cells infiltrating tumors requires identification of clonal TCRαβ (alpha/beta) sequences and their antigen specificity. To this end, whole exome sequencing on tumor and normal cells and RNA sequencing on tumor cells from 4 patients was performed to identify and rank prevalent nonsynonymous mutations that could serve as neoantigens. The 30 to 45 highest-expressed mutations in each tumor were selected to screen for recognition by the patients' CD4+ T cells. For each candidate neoantigen, 20-mer peptides with the variant amino acid at either position 7 or 13 were synthesized for screening (not shown).

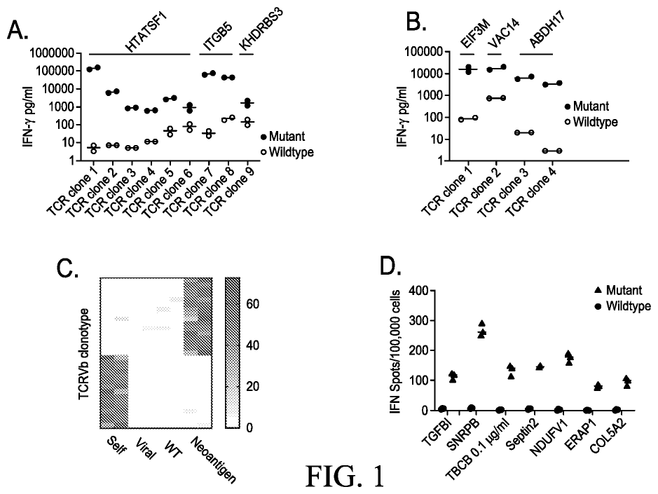

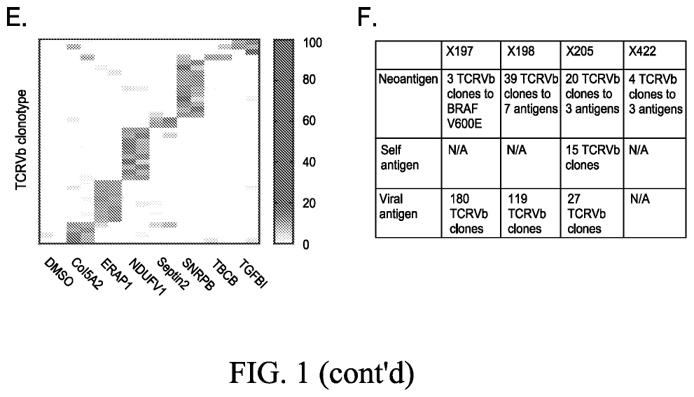

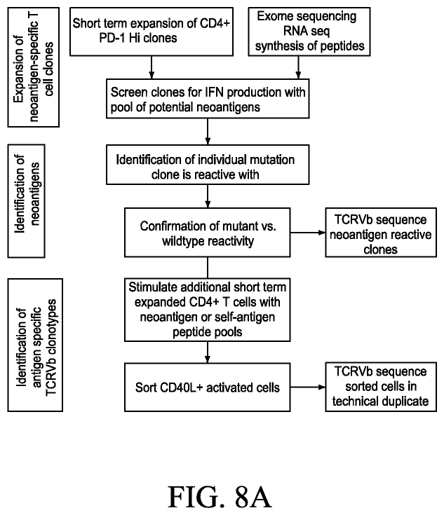

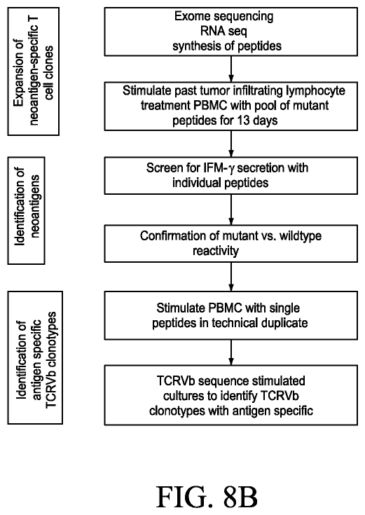

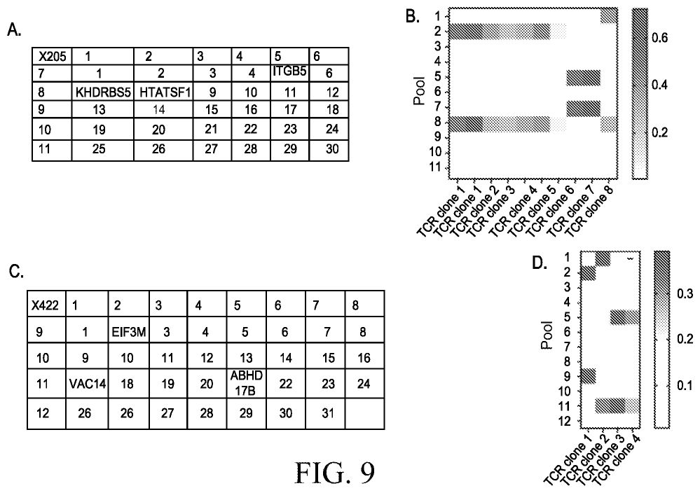

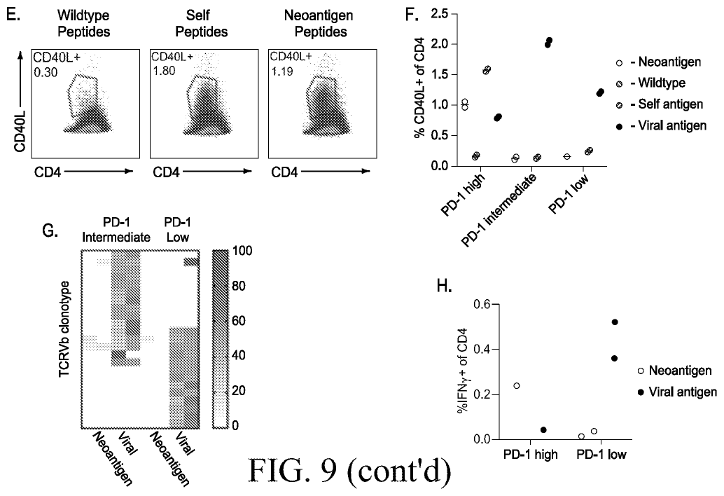

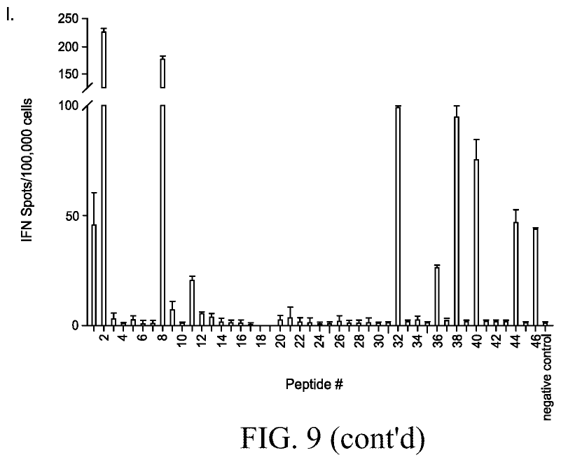

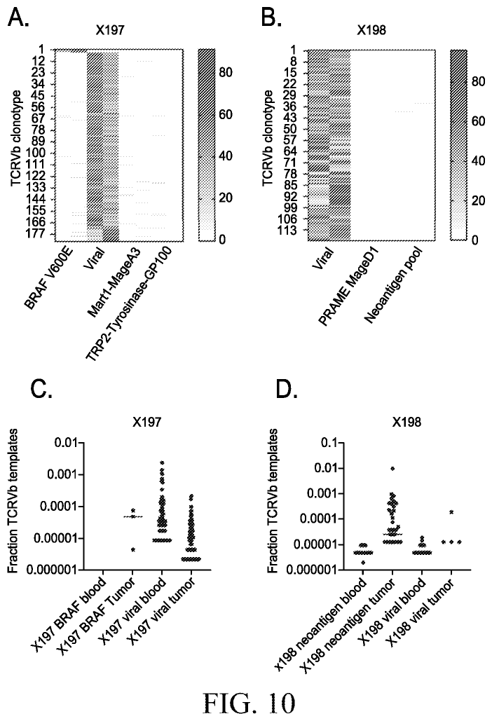

Multiple approaches were used to isolate and identify the TCRVβ sequences of neoantigen-specific CD4+ T cells. PD-1^{high} CD4+ T cells, which have been shown to be enriched for tumor reactivity (Yossef et al., 2018), were sorted from tumor infiltrating lymphocytes (TIL) of patients X205 and X422, expanded in limiting dilution cultures, and screened for IFN-γ secretion against peptide pools containing candidate neoantigens (FIG. 8A). T cell cultures with reactivity for mutant peptides were identified, specificity for the mutant versus wildtype peptide was confirmed, and TCRVβ sequencing was performed to identify unique TCRs (FIG. 9(A-D), FIG. 1(A, B)). Additional tumor-reactive CD4+ T cells were isolated from X205 TIL by stimulating bulk ex-vivo expanded PD-1^{high} T cells in duplicate with peptide pools comprised of tumor associated self-antigens (Tyrosinase, Mart1, TRP2, GP100, and MAGE A3) and with candidate mutant neoantigens and corresponding wild type sequences, and then sorting CD4+ T cells that expressed CD40L after antigen restimulation and performing TCRVβ sequencing (FIG. 9(E)). TCRVβ clonotypes that were present in CD40L-sorted T cells from both replicates were considered antigen-reactive. This approach identified 5/9 previously confirmed TCR clonotypes and an additional 11 TCR clonotypes (FIG. 1C; other data not shown). There was no reactivity after stimulation of PD-1 intermediate and PD-1 low CD4+ T cells from patient X205 with the neoantigen pool, but these T cells exhibited reactivity after stimulation with a panel of viral antigens and 23 viral specific TCRVβ clonotypes were identified in these cultures (FIG. 9 (F,G)). Reactivity to neoantigens was also restricted to the PD-1 high subset of cells in patient X422, with increased reactivity to viral antigens in the PD-1 low subset (FIG. 9(H)). These observations confirmed restriction of tumor antigen specific CD4+ T cells in these patients to the PD-1 high fraction. Patients X197 and X198 had received adoptive immunotherapy with autologous TILs and PBMCs obtained after the TIL infusion were stimulated with a pool of peptides corresponding to candidate neoantigens (FIG. 8B). CD4+ T cells that were specific for BRAF V600E were isolated and 3 different TCRVβ sequences from patient X197 (Veatch et al., 2018) were expressed. CD4+ T cells from patient X198 had reactivity for 7 different mutated peptides and did not recognize the corresponding wild-type peptides (FIG. 1D, FIG. 9(I)). The TCRVβ clonotypes responsible for the neoantigen reactivities in X198 were identified by stimulating PBMC with individual purified mutated peptides and TCRVβ sequences that selectively expanded in replicate cultures were identified as antigen specific (FIG. 1E) (Danilova et al., 2018). To assist with analysis of potential bystander T cells that might be present in TIL, PBMC from patients X197 and X198 were stimulated with viral antigens and TCRVβ clonotypes that expanded in these cultures were identified by sequencing (FIG. 10(A-B)). TCRβ sequencing of tumor and pre-TIL infusion blood samples from X197 and X198 showed enrichment of TCR sequences corresponding to self and neoantigen specific T cells but not virus specific T cells in the tumor relative to blood, (FIG. 10(C-D)). Virus-specific clonotypes were not isolated from patient X422 and no self-antigen specific TCRVβ clonotypes were identified from patients X198, X197 or X422. In total, 66 different TCRVβ clonotypes specific for 14 neoantigens from the 4 patients, 15 TCRVβ clonotypes specific for nonmutated tumor associated antigens from patient X205, and 326 TCRVβ clonotypes specific for viral antigens from patients X197, X198 and X205 (FIG. 1F), were identified.

### Example 2

### Phenotype of Tumor Antigen Specific Cd4+ T Cells in Melanoma

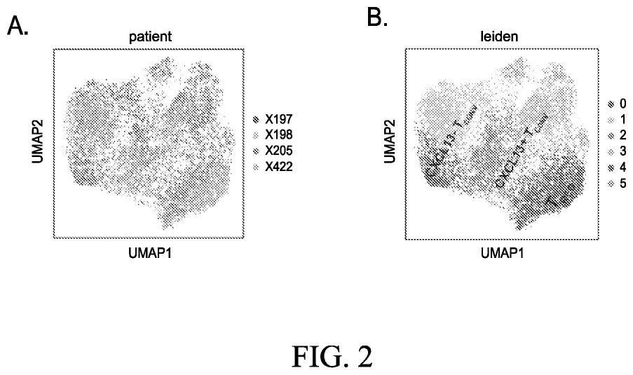

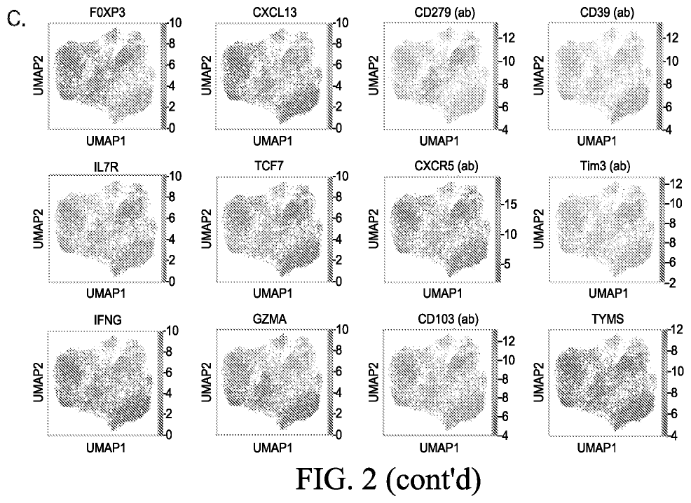

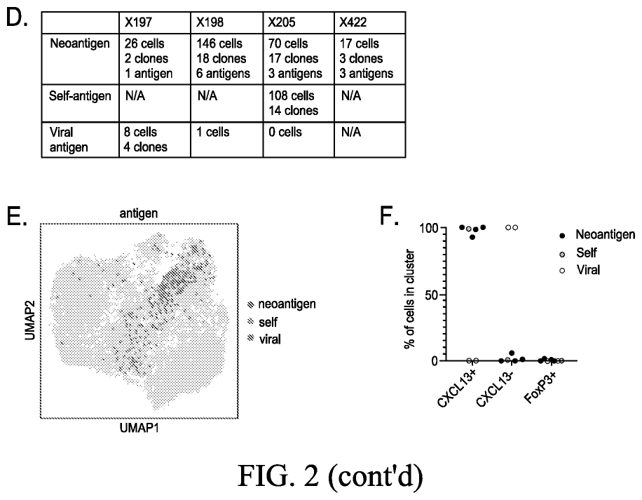

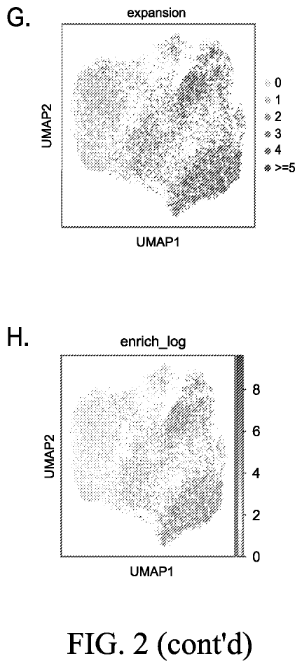

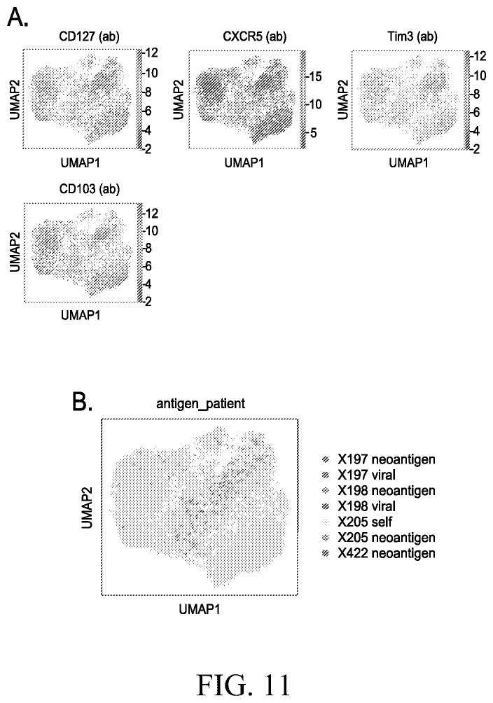

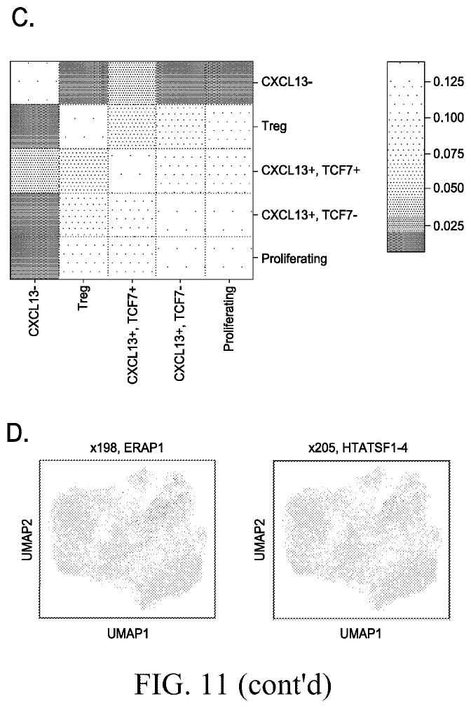

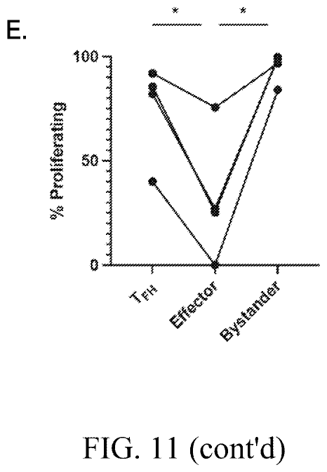

Single cell suspensions from tumor samples of the 4 patients in Example 1 were stained with fluorescent antibodies to CD4 and CD3 and a panel of 34 oligonucleotide labelled antibodies, and CD3+CD4+ T cells were sorted and analyzed by single cell targeted mRNA sequencing of 400 immune response genes and TCR VDJ rearrangements (Ma et al., 2021). BRAF-specific T cells were rare in the tumor of patient X197 and all clones were Vβ 3.1, therefore in this patient 50% of the CD4+ T cells were enriched for Vβ 3.1 prior to sequencing to provide greater coverage of this tumor-specific population. In total, 10,186 tumor infiltrating CD4+ T cells were sequenced from the 4 patients. Unsupervised clustering of these cells by mRNA expression defined a cluster of FoxP3+ regulatory T cells (TREG), 3 clusters of CXCL13+ non-TREG conventional T cells (TCONV) that also expressed surface PD-1, and 2 clusters of CXCL13-TCONV that expressed high levels of IL7R mRNA (FIG. 2(A-C)). 259 cells (17-146 per patient) expressed TCR clonotypes specific for neoantigens, 10⁸ cells from patient X205 expressed TCR clonotypes for self-antigens, and 9 cells from patients X197 and X198 expressed TCR clonotypes for viral antigens (FIG. 2(D)). Strikingly, alignment of TCRVβ sequences placed 97% of neoantigen-specific (range 93-100%) and 99% of self-antigen-specific T cells in the CXCL 13+ clusters, and all 9 virus-specific T cells from 2 patients in the CXCL13-TCONV cluster (FIG. 2(E-F), FIG. 11(B)). TCR clonotypes present in two or more cells in the tumor or enriched in tumor relative to blood were more common in the CXCL13+ TCONV and TREG clusters than in the CXCL13-TCONV cluster (FIG. 2(G-H)). The transcriptional signatures of neoantigen and tumor self-antigen specific cells were shared between each of the 4 patients.

In total, the antigen specificity of 8.8% of the CXCL13+PD-1 high subset of tumor infiltrating cells that were sequenced in 4 different patients was identified; this represents a large increase in the number of CD4+ T cells reactive to tumor antigens that have been described in primary human cancer samples (Cachot et al., 2021). The PD-1 low CXCL 13-subset of Tconv cells contained viral specific T cells from 2 patients, and viral reactivity in the other 2 patients was limited to CD4+ T cells expanded from the PD-1 low fraction (FIG. 9 (F, H)). Previous work has shown TREG cells have specificity for tumor antigens but these cells would not be detected using the present methods which require IFN-γ secretion (Ahmadzadeh et al., 2019). Taken together, these data evidence that conventional CD4+ T cells infiltrating tumors can be divided into populations of clonally expanded, tumor enriched, PD-1 high, CXCL13+ cells that contain tumor antigen specific cells, and clonally diverse PD-1 low cells containing “bystander” cells specific for non-tumor antigens.

### Example 3

### Cxcl13+Cd4+ T Cells in Melanoma Contain Three Distinct Cell States

Unsupervised clustering of CD4+ T cells showed neoantigen-specific cells within each of the three CXCL13+ clusters (FIG. 2(B-F), FIG. 11(B)). One cluster was defined by increased expression of memory genes (TCF7, IL7R) as well as genes (BCL6 and CD200) and surface markers (CXCR5) associated with T follicular helper (T_{FH}) cells. A second TCF7-population expressed coinhibitory (Tim-3 and Lag-3 surface expression), inflammatory (CCL3, CCL4, IFNG mRNA), cytolytic (GZMA/K, PRF1 mRNA) markers, and the tissue resident memory marker CD103 (FIG. 2(A-B, H), FIG. 11(E), other data not shown). A third CXCL13+ population expressed genes involved in proliferation (TYMS, TOP2A, MCM2/4 mRNA) (FIG. 2(C)). There was overlap of TCRVβ clonotypes between each of these CXCL13+ clusters (FIG. 11(C)) and individual neoantigen specific clonotypes were represented in all 3 CXCL13+ clusters (FIG. 11(D)), reminiscent of TCF7+ and TCF7 subsets of CD8+ T cells that respond differently to ICB therapy in murine tumors (Miller et al., 2019; Siddiqui et al., 2019; Wu et al., 2016).

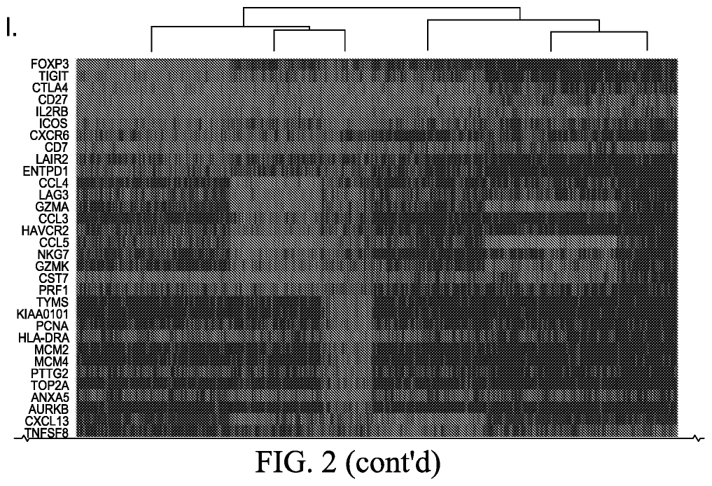

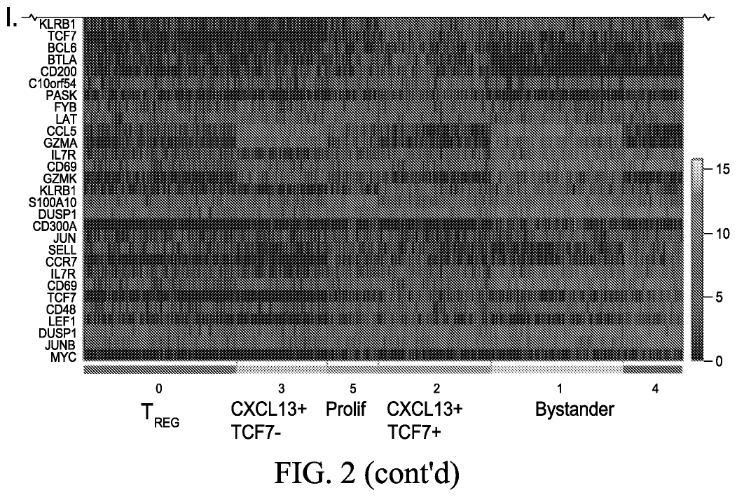

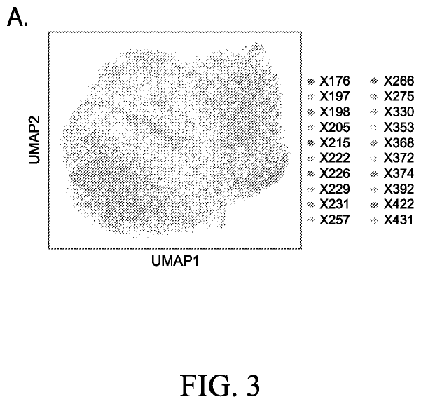

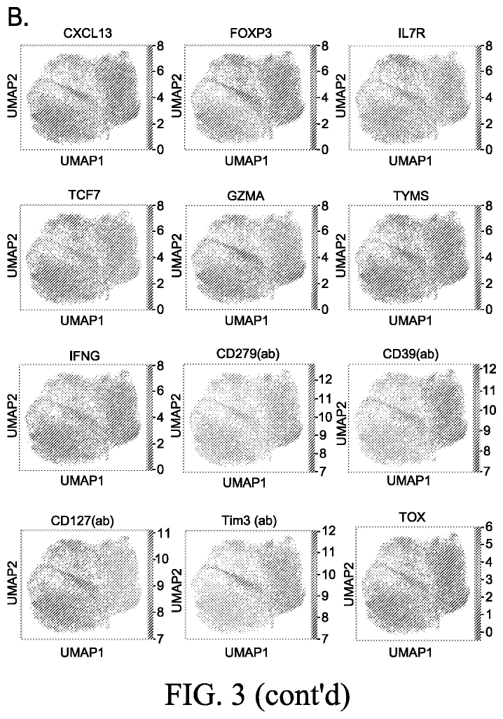

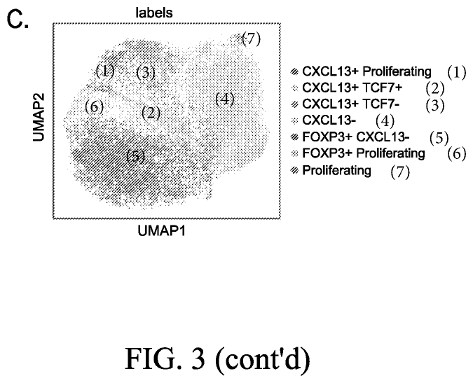

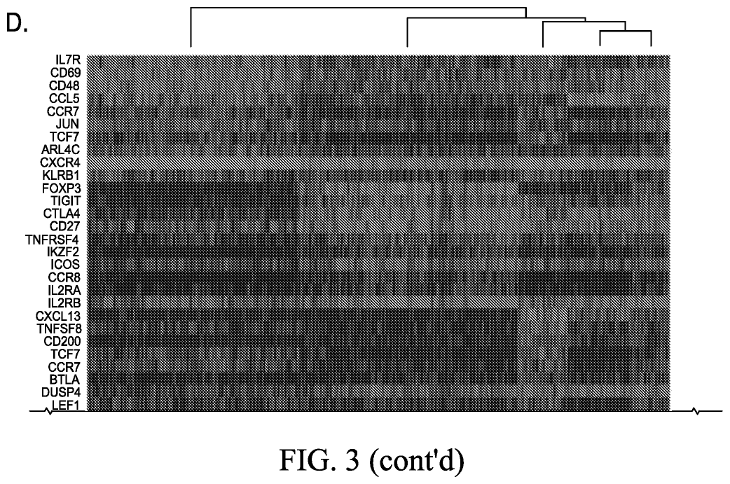

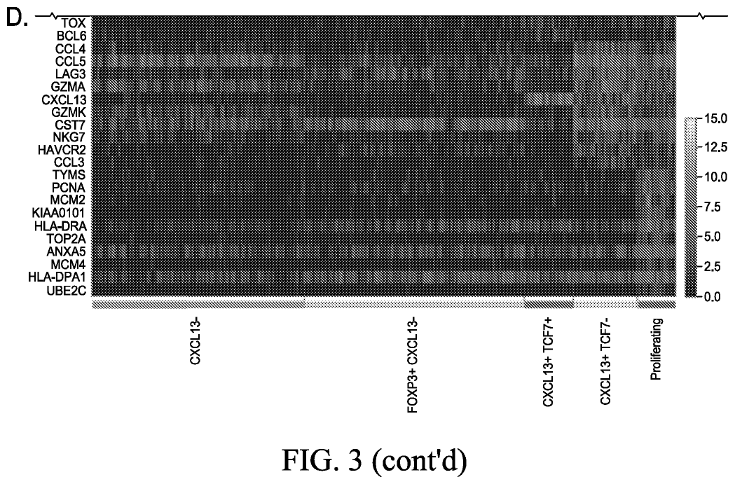

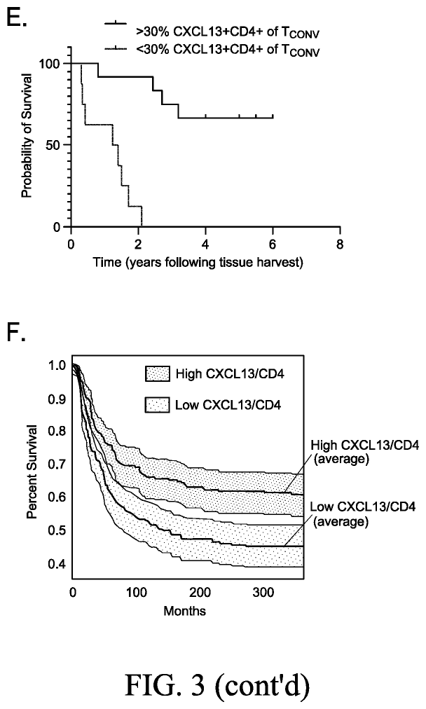

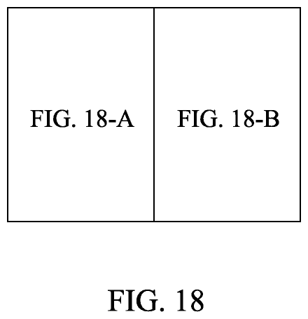

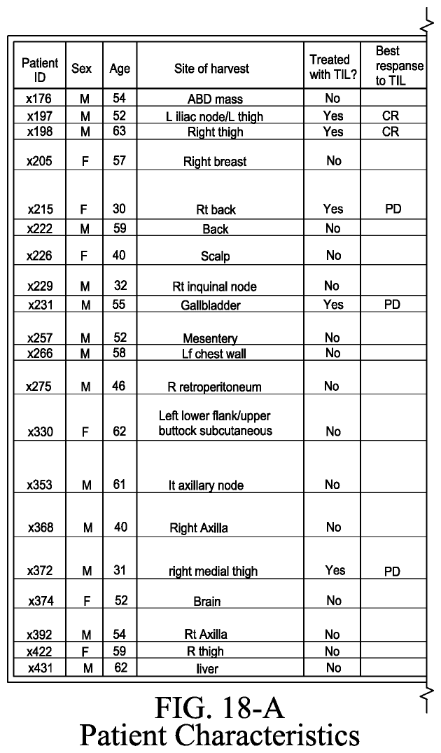

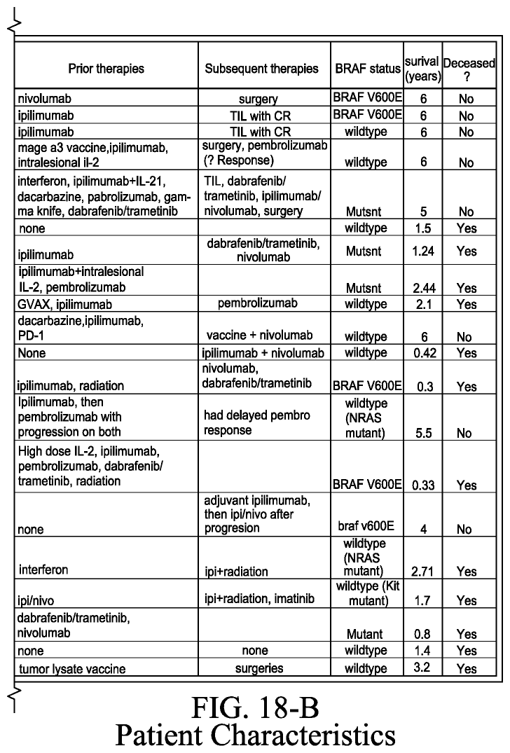

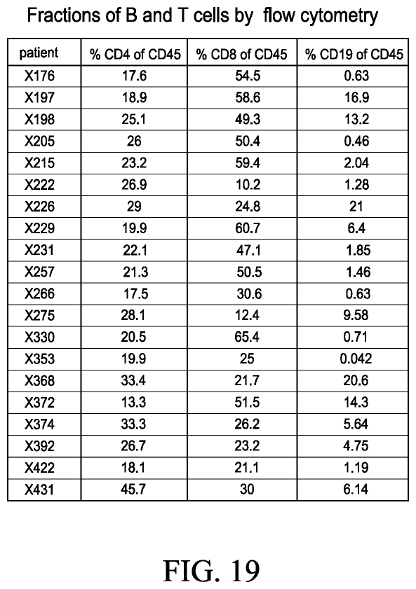

To investigate whether the transcriptional signatures associated with tumor-specific CD4+ T cells were present in a larger melanoma patient cohort (FIGS. 18A-18B), and whether the presence of these CD4+ T cells related to the number and phenotype of other immune cells in the melanoma TME, an extended panel of 53 surface antibodies and targeted mRNA sequencing of 405 genes were used to re-analyze tumor samples from the initial 4 patients and study 16 additional patients. Sequencing of between 422 and 3185 CD4+ T cells from each of these 20 patients followed by unsupervised clustering revealed the populations observed in the initial analysis, including FoxP3+ TREG and CXCL13-TCONV, and 3 clusters of CXCL13+ TCONV, comprised of TCF7+ cells that expressed T_{FH} genes, TCF7 cells that expressed cytolytic genes, IFNG, Tim-3 and Lag-3, and a population that expressed TYMS and other cell proliferation genes (FIG. 3(A-D); FIG. 11(E)). In 4 patients from whom material was available, The T_{FH} population showed an increased ability to proliferate relative to the CXCL13+ TCF7 population, consistent with this being a less differentiated population of cells (FIG. 11(E)). These data identify tumor-infiltrating CXCL13+CD4+ T cells across a large set of melanoma patients that have transcriptional signatures concordant with that of tumor-specific CD4+ T cells.

### Example 4

### The Fraction of Cxcl13+Cd4+ T Cells is Predictive of Overall Survival in Melanoma

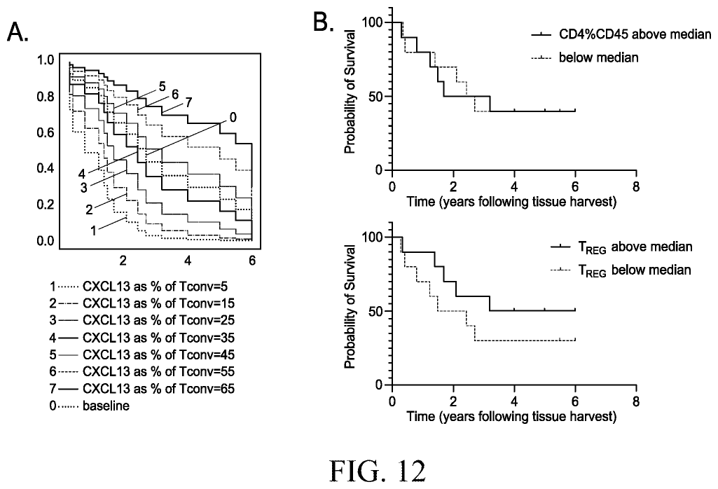

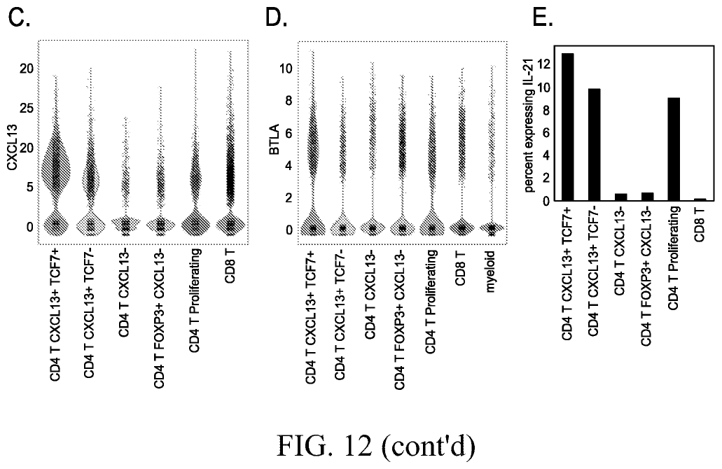

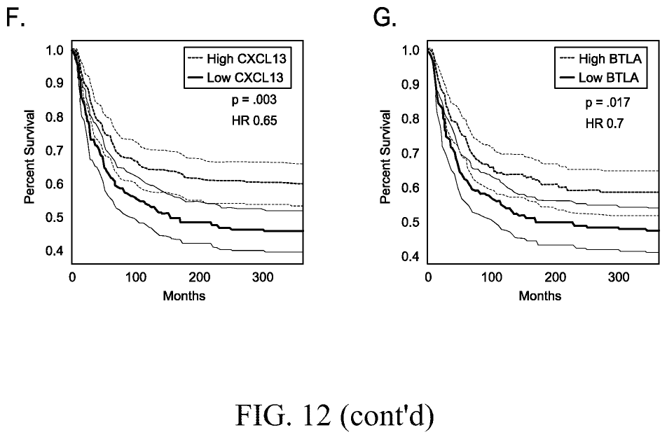

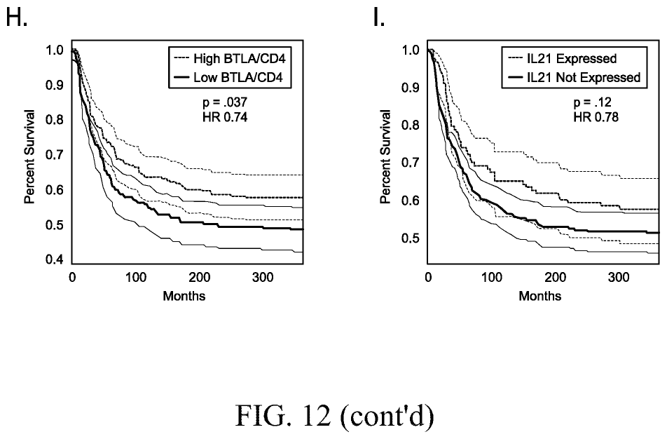

These data demonstrate that CXCL13+CD4+ T cells infiltrating melanoma contain tumor antigen-specific T cells, suggesting that the frequency of such cells relative to TREG and PD-1-low CXCL13-low hypothetical “bystanders” might be associated with a more effective immune response. The fraction of CXCL13+CD4+ TCONV varied between 7% and 55% of total tumor-infiltrating CD4+ T cells in this patient cohort, and those patients with >30% CXCL13+CD4+ T cells as a fraction of TCONV cells exhibited improved survival compared to patients with <30% CXCL13+CD4+ T cells (FIG. 3(E)). This effect was not dependent on an arbitrary threshold, as the association of survival with the proportion of conventional CD4+ T cells belonging to CXCL13+ clusters was observed across various thresholds in a Cox proportional hazards model (FIG. 12(A)). In contrast, the fraction of total CD45+ cells that were CD4+ or the fraction of TREG did not correlate with survival in this cohort (FIG. 12(B)).

Next, supportive evidence that the fraction of CXCL 13+CD4+ T cells is associated with increased survival in melanoma was sought. Genes in the single cell RNA seq data that could act as surrogates for the presence of CXCL13+CD4+ T cells were identified and their expression levels in publicly available bulk RNA sequencing datasets were analyzed. These included CXCL13, which is also expressed in a subset of CD8+ T cells but is at lower levels than CD4+ T cells (FIG. 12(C)), and BTLA and IL21, where expression of these genes is largely restricted to CXCL13+CD4+ T cell subsets (FIG. 12(D-E)). Consistent with the observation that the presence of CXCL13+CD4+ T cells correlated with overall survival in the tested cohort, analysis of TCGA data from 471 melanoma patients showed that increased expression of CXCL13 and BTLA were associated with greater overall survival (corrected p=0.003, 0.017 respectively) (FIG. 12(F-G)). These genes can correlate with total immune infiltrate, however it was found that CXCL13 and BTLA expression also predicted greater survival when normalized to CD4 expression (corrected p=0.001, 0.038 respectively, FIG. 3(F), FIG. 12 (H)). IL-21 was expressed in a minority of melanoma patients in this data set and was associated with a nonsignificant trend toward improved survival (Corrected p=0.12, FIG. 12(I)).

### Example 5

### Phenotype of Cd8+ T Cells in the Tumor Microenvironment

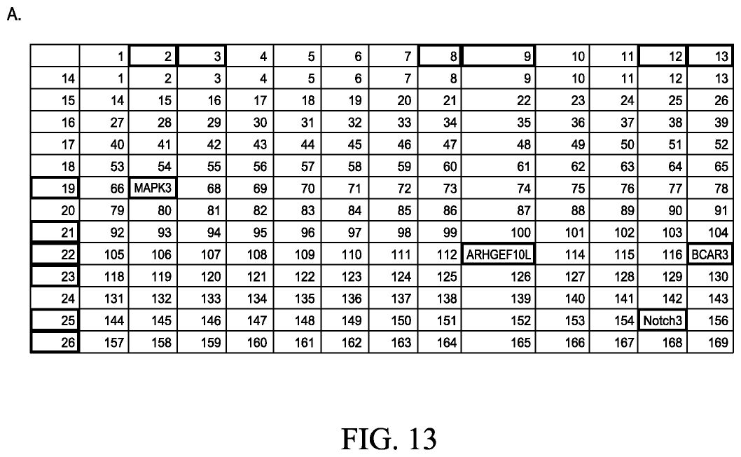

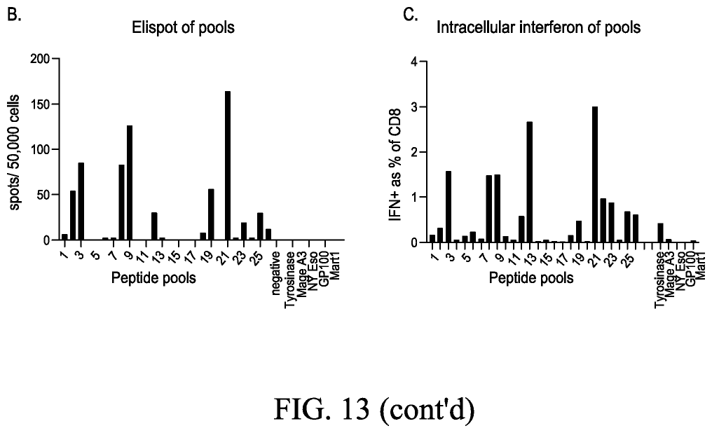

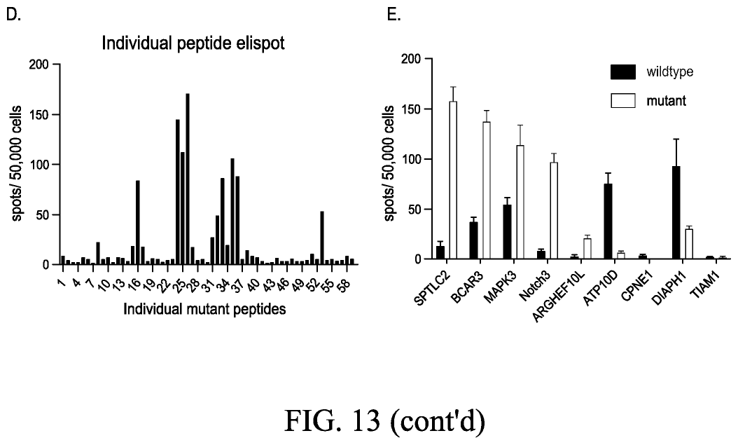

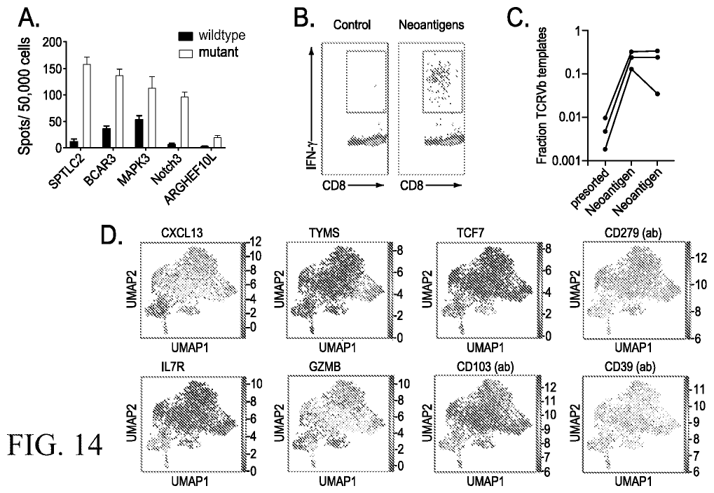

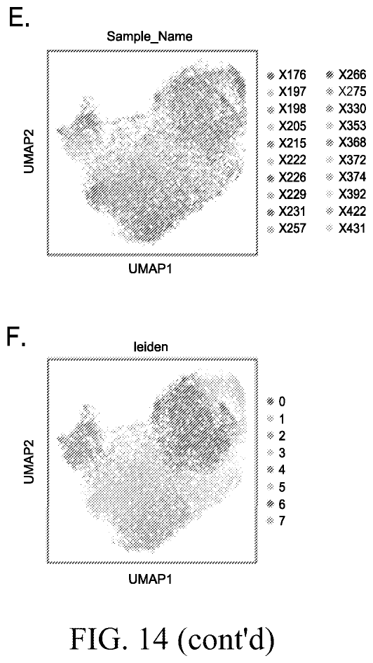

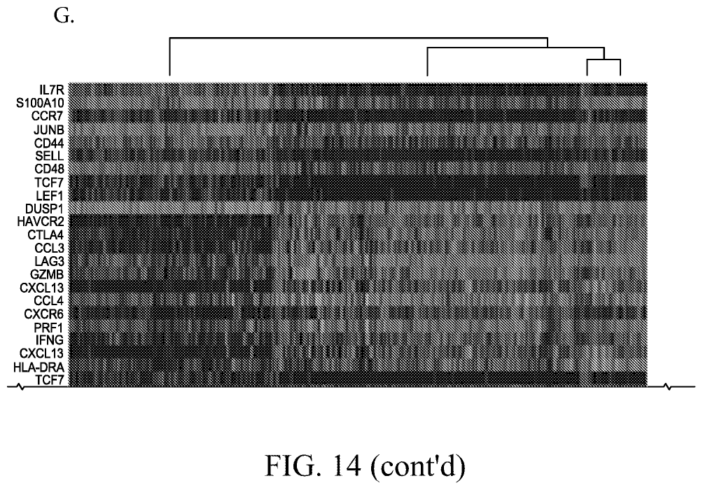

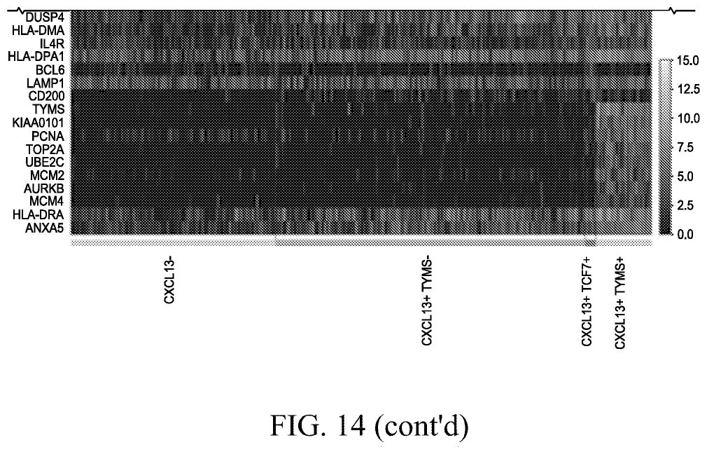

The expression of CXCL13 in subsets of tumor infiltrating CD4+ and CD8+ T cells led to examining other parallels in phenotypes of these T cell populations that may be driven by chronic antigen exposure in the TME. 223 to 2248 CD8+ T cells were analyzed by targeted scRNA seq in tumor samples from each of the 20 patients, including two patients (X197 and X198) in whom CD8+ T cells specific for tumor antigens were identified by culturing tumor fragments in high dose IL-2 and testing for reactivity to peptides encompassing highly expressed mutations and lineage specific antigens. Patient X197 exhibited CD8+ T cell reactivity to tyrosinase, TRP2, and Mart1 and the cancer testes antigen Mage A3, and TCRVβ sequencing of cells sorted based on IFN-γ secretion following incubation with single antigens identified 7 clones specific to these antigens (Veatch et al., 2018). CD8+ T cells specific for self-antigens in Patient X198 were not identified, but 3 TCR clonotypes reactive to mutated neoantigens were identified (FIG. 13, FIG. 14(A)). (FIG. 14(B-C)).

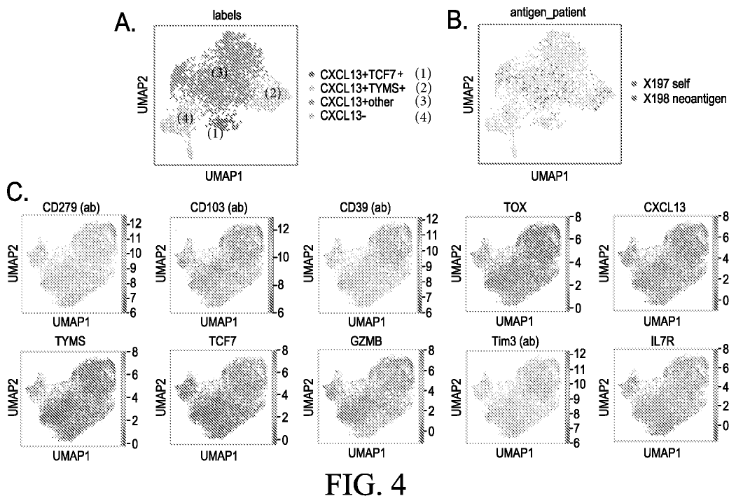

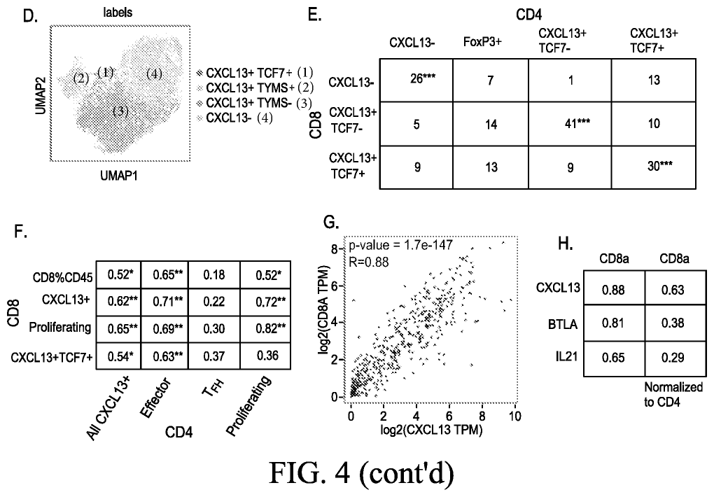

Previous work has indicated that the tumor antigen-specific subset of CD8+ T cells within tumors is characterized by expression of PD-1, CD39, CD103 and CXCL13 (Duhen et al., 2018; Litchfield et al., 2021; Simoni et al., 2018; Thommen et al., 2018). Unsupervised clustering of CD8+ T cells from the two patients in the present study showed that a majority expressed CXCL13, PD-1, and CD39. Within the CXCL13+ cells, distinct clusters that co-expressed CXCL13 and TCF7, or CXCL13, TYMS and other markers of proliferation were identified (FIG. 14(D), FIG. 4(A)). Tumor antigen-specific cells from patients X197 and X198 were found in PD-1+ CXCL 13+ clusters, but not in the cluster lacking expression of CXCL 13 and PD-1 (FIG. 4(B)). Unsupervised clustering of the scRNA seq data from the entire cohort of 20 patients also identified CD8+ T cells that expressed PD-1, CD103, CD39, Tox and CXCL13. As observed in the first 2 patients, a subset of CXCL13+CD8+ T cells expressed TYMS and other markers of cell proliferation, and a minor subset expressed TCF7, consistent with less differentiated cells described by others (FIG. 3(C), FIG. 14(C-D)) (Miller et al., 2019; Siddiqui et al., 2019; Wu et al., 2016).

The sets of genes upregulated greater than 2-fold in individual phenotypic clusters of CD8+ and CD4+ T cells were compared for overlap using a Jaccard index (FIG. 4(E)), and there was a pattern of overlap between the CXCL13+ TCF7+CD4+ and CD8+ populations (30 percent of genes shared), with multiple T_{FH} associated genes such as BCL6 and CD200 upregulated in the CD8+ population. There was also overlap between the CXCL13+ TCF7-populations (41 percent of genes shared), including cytolytic, inflammatory and coinhibitory molecules. As expected, there was also increased overlap in gene expression within the CXCL13⁺ conventional T cell populations (26%).

### Example 6

Cxcl13+Cd4+ T Cells Correlate with Cd8+ T Cell Infiltration and Activation

CD4+ T cells can coordinate the functions of other immune cells in the tumor microenvironment. Studies were performed to examine if the fraction of CXCL13+CD4+ T cells was related to the presence and phenotype of CD8+ T cells, myeloid cells, and B cells. As CD4+ T cells could potentially affect both the number and phenotype of CD8+ T cells and B cells, these efforts compared CD4+ T cell subsets as a fraction of CD4+ T cells to CD8 and B cell subsets as a fraction of CD45+ hematopoietic cells. Across the 20-patient cohort, the fraction of CXCL13+CD4+ T cells correlated with the total fraction of CD8+ T cells (Spearman correlation coefficient R=0.52, corrected p value 0.027) and even more closely with the fraction of CXCL13+CD8+ T cells (R=0.62, p=0.007) and the fraction of proliferating TYMS+ CXCL13+CD8+ T cells (R=0.65, p=0.005). The TCF7-effector (R=0.69, p=0.003) and proliferating CXCL13+CD4+ subsets correlated more closely with proliferating CD8+ T cells (R=0.82 p=0.00013) than with the T_{FH} subset (R=0.30, p=0.14, FIG. 4(F)).

There are currently no public melanoma scRNA seq datasets with sufficient patient numbers to independently validate these observations, therefore the TCGA data was interrogated, using expression of CXCL13, BTLA and IL-21 as surrogates of the presence of CXCL13+CD4+ T cells. Each of these markers correlated with CD8a expression in melanoma (FIG. 4(G-H), p<l-54), and these correlations remained after normalization of CD4 expression (p<l-9). These observations show the fraction of CD4+ T cells with a signature of tumor antigen specificity correlates with infiltration and activation of CD8+ T cells, consistent with such CD4+ T cells providing helper function to tumor-antigen specific CD8+ T cells.

### Example 7

Cxcl13+Cd4+ T Cells Correlate with Macrophage Activation

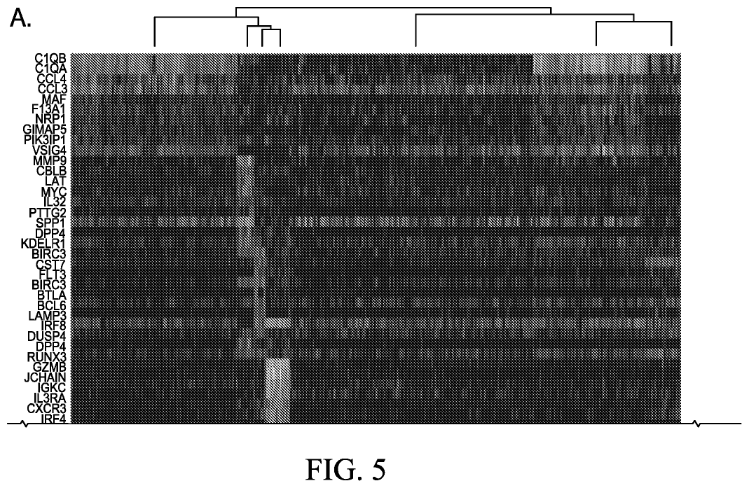

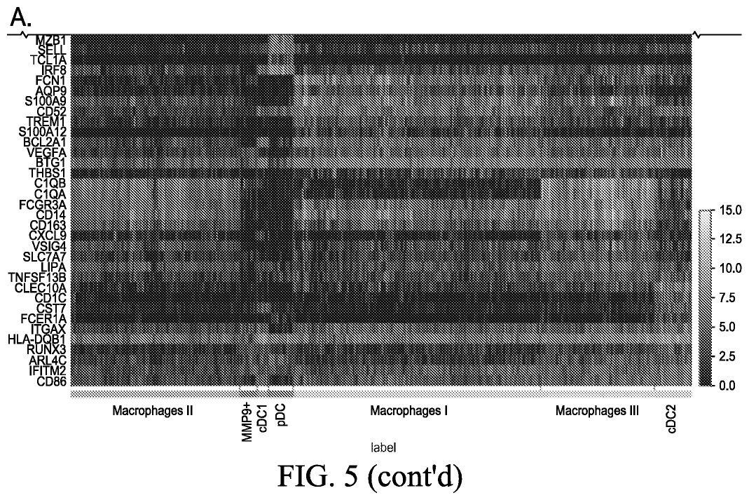

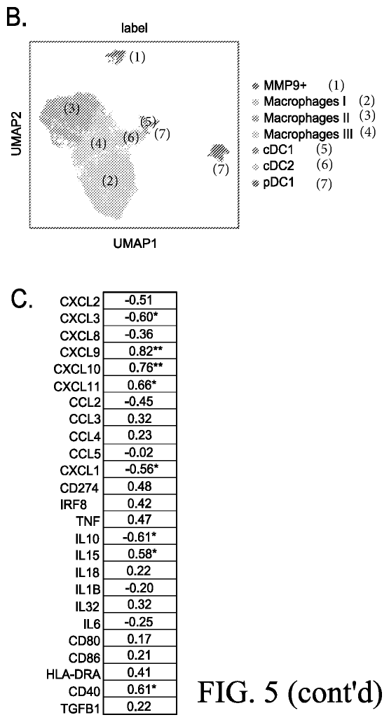

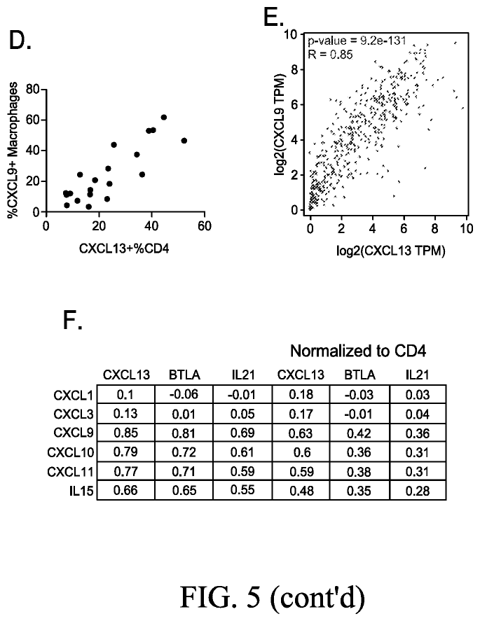

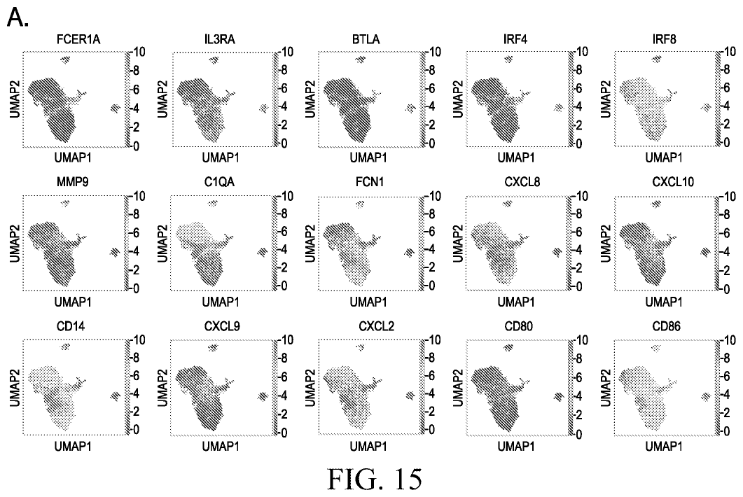

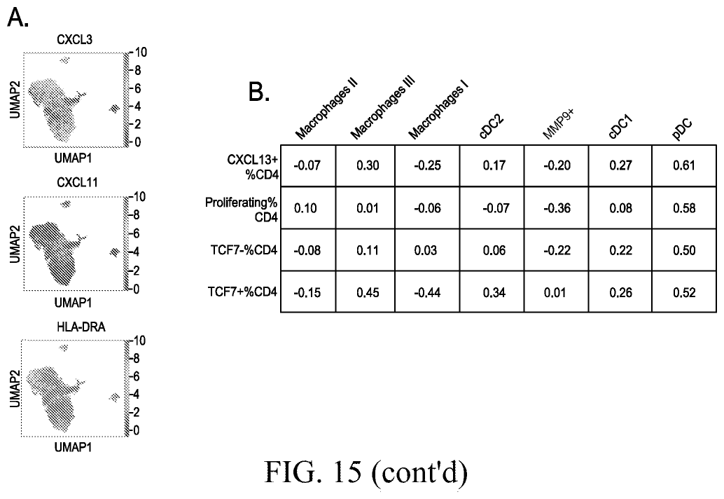

Correlations between CXCL13+CD4+ subsets and myeloid cell populations and phenotypes were examined. Sequencing of between 147 and 2448 CD45+CD3-CD19-cells from each of the 20 tumors identified CD123+CD4+ plasmacytic dendritic cells (pDC); BTLA⁺ LAMP3⁺ type 1 conventional dendritic cells (cDC1), FCER⁺ CD1C⁺ type 2 conventional dendritic cells (cDC2) and a large number of CD14+ macrophages which varied in their expression of C1Qa/b and FCN1 (FIG. 5(A-B), FIG. 15(A)) (Xiong et al., 2020). There were no correlations between the various myeloid subsets and CXCL13+CD4+ T cell fractions that remained significant at an FDR of less than 5% after correction for multiple comparisons (FIG. 15(B)), although in this example, the power to detect these correlations was limited by the small numbers of dendritic cell subsets in the samples.

Macrophages can modulate antitumor immunity by producing proinflammatory or suppressive chemokines and cytokines. CXCL9, CXCL10 and CXCL11 produced in response to IFN-γ mediate CXCR3-dependent lymphocyte recruitment (House et al., 2020), whereas CXCL1 and CXCL3 mediate CXCR2-dependent recruitment of immunosuppressive myeloid cells (Alfaro et al., 2016). TAMs can also produce cytokines such as IL-15 that can support T cell function and IL-10 that is suppressive (Carrero et al., 2019; Ruffell et al., 2014). To evaluate if the presence of CXCL13⁺ CD4⁺ T cells correlated with these phenotypes, the tumor associated macrophages (TAMs) that make up the bulk of the myeloid cells across these tumors were assessed. The expression of 25 immune regulatory genes whose expression varied across TAMs in the sample was examined; the fraction of CXCL13⁺ CD4⁺ T cells positively correlated with the inflammatory chemokines CXCL9, CXCL10 and CXCL11, as well as IL 15 and the activation marker CD40, and negatively correlated with CXCL1, CXCL3 and IL 10 with an FDR of less than 5% (FIG. 5(C-D)).

The association of macrophage activation with the presence of CXCL13+CD4+ T cells was corroborated in the larger TCGA melanoma data set. CXCL13, BTLA and IL-21 expression, which serve as a proxy for the presence of tumor antigen-specific CD4 T cells, correlated with expression of IL-15, CXCL9, CXCL10 and CXCL11 in the TCGA data, but did not correlate with expression of CXCL1 or CXCL3 (FIG. 5(E-F)). These relationships were maintained when expression of these markers in the TCGA data set were normalized to CD4 indicating they were not general features of lymphocyte infiltration.

### Example 8

Cxcl13+Cd4+ T Cells Correlate with B Cell Maturation and Colocalize with B Lineage Cells

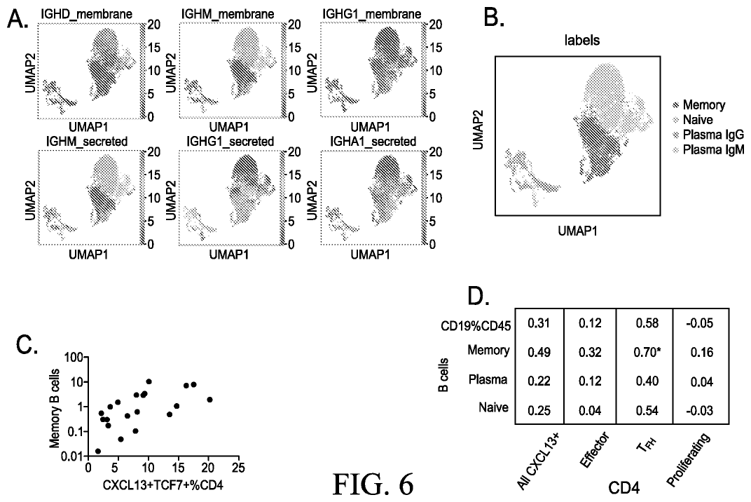

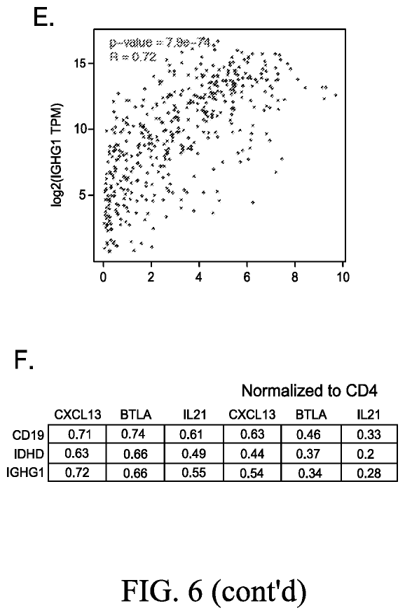

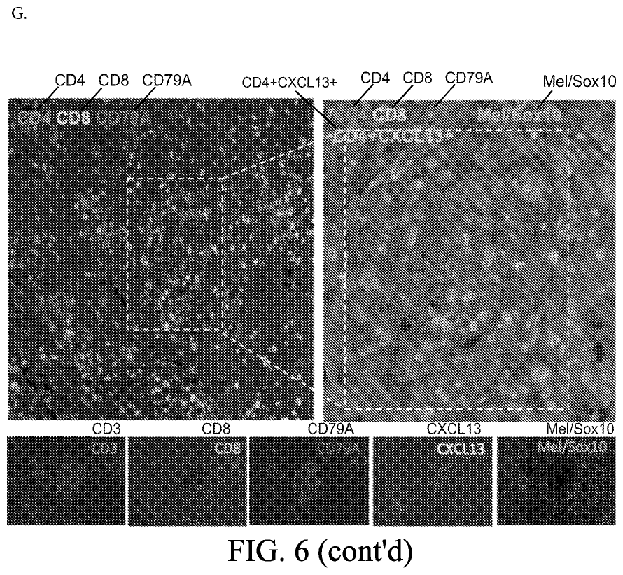

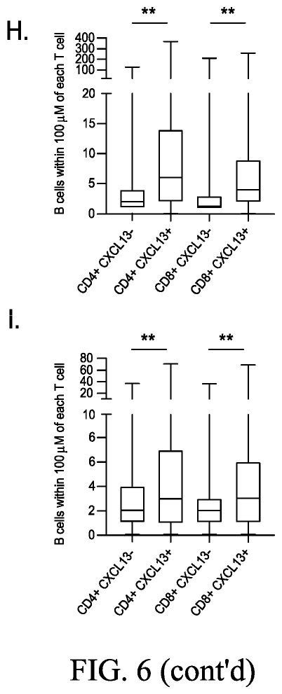

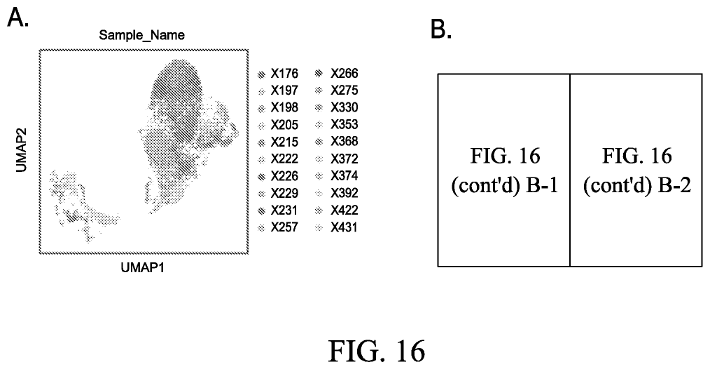

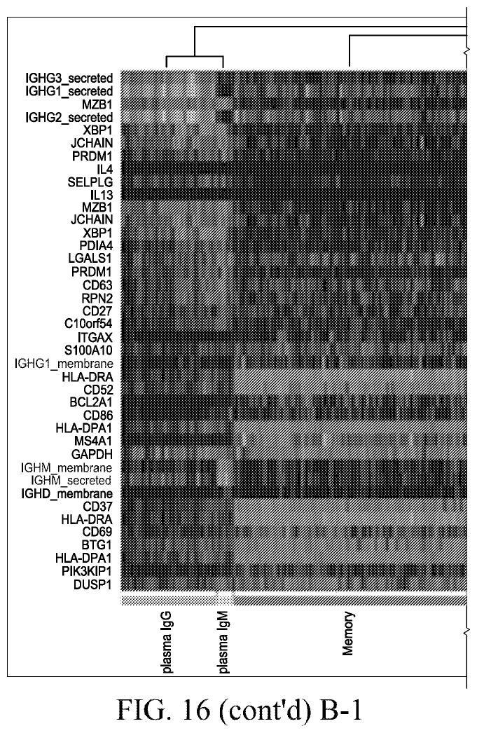

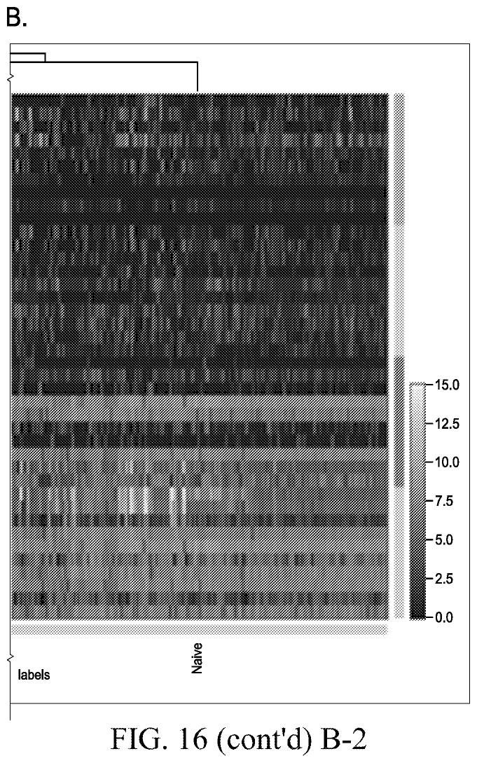

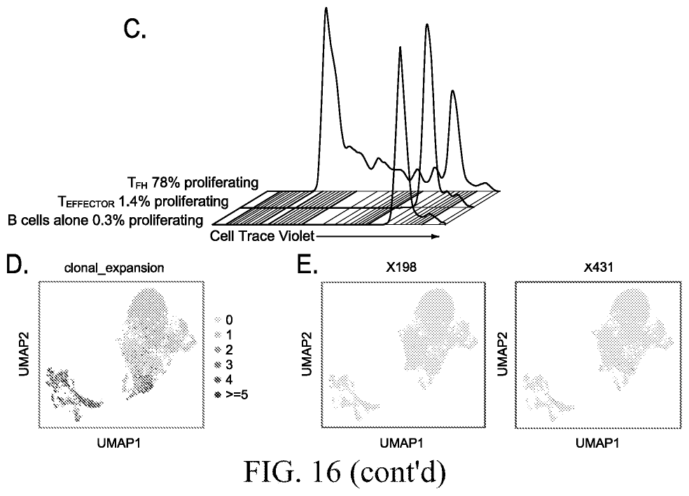

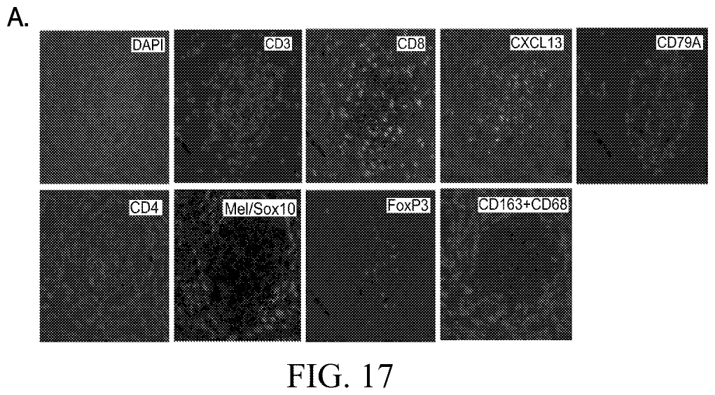

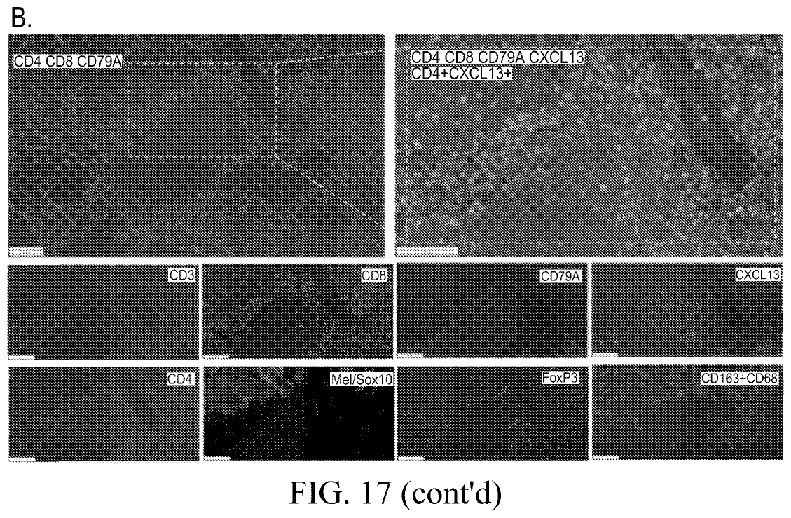

To evaluate whether B cell subsets and maturation stages in TIL related to CXCL13⁺ CD4⁺ T cell infiltration, CD19⁺ B cells were sorted for single cell sequencing. Naïve B cells expressing IGHD and membrane IGHM, memory cells expressing surface IGHG1, and secreted IGHG1⁺ CD20⁺ plasma cells were identified (FIG. 6(A-B), FIG. 16(A-B)) (Horns et al., 2020). The fraction of CXCL13⁺ CD4⁺ T cells did not correlate with the total number of tumor-infiltrating B cells as a fraction of hematopoietic cells, but a trend toward a correlation was seen between the frequency of TCF7⁺ CXCR5⁺ T_{FH} cells and the total number of B cells as a fraction of CD45⁺ hematopoetic cells (Spearman R=0.57, corrected p=0.06) or the number of naïve B cells (Spearman R=0.54, corrected p=0.08) with a significant association with the subset of memory B cells (Spearman R 0.70, corrected p=. 01) (FIG. 6(C-D)). In contrast, there was no correlation between the TCF7⁺ subset of CXCL13⁺ CD4⁺ T cells and any B cell subset. Consistent with a specific role for the T_{FH} subset of CXCL13⁺ CD4⁺ T cells in B cell stimulation, in one patient from whom sufficient quantities of cells could be isolated, T_{FH} cells but not effector cells were able to stimulate B cell proliferation in vitro (FIG. 16(C)). B cell receptor clonotypes were expanded in the memory and plasma cell compartments (FIG. 16(D)), and individual clonotypes were found in both memory and plasma subsets (FIG. 16(E)), suggesting expansion and differentiation of B cell clones in the TME. The presence of T_{FH}-like cells was correlated with B cells in a larger cohort of melanoma patients using CXCL13, BTLA and IL21 expression as surrogates for T_{EH} frequency in these samples. Robust correlations were found between the expression of all 3 genes and a marker of B cells (CD19), a marker of naïve B cells (IGHD) and a marker of memory and plasma b cells (IGHG1), and these correlations persisted when controlled for CD4 expression, arguing against this being a general measure of immune infiltration (FIG. 6(E-F)). In two patients from whom tumor antigen-specific CD4⁺ and CD8⁺ T cells were identified, and also had tertiary lymphoid structures, multiplex immunohistochemistry was used to analyze the localization of CXCL13+CD4⁺ and CD8⁺ T cells relative to TLS (FIG. 6(G), FIG. 17). Analysis of over 10,000 individual CD8⁺ and CD4⁺ T cells across the tumors of these patients revealed that both CD4⁺ CXCL13⁺ and CD8⁺ CXCL13⁺ T cells were more likely to be in close proximity to CD79a⁺ B lineage cells than their CXCL13-counterparts (FIG. 6(H-I)).

### Example 9

Infiltrating Cd4⁺ T Cells in Breast Cancer Treated with Immune Checkpoint Inhibitors Exhibit Similar Phenotypes and Correlate to Activation of Cd8⁺ T Cells and Myeloid Cells

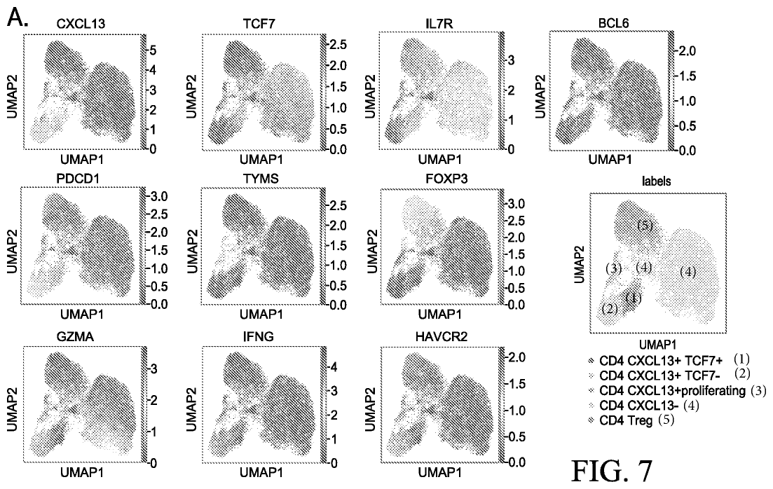

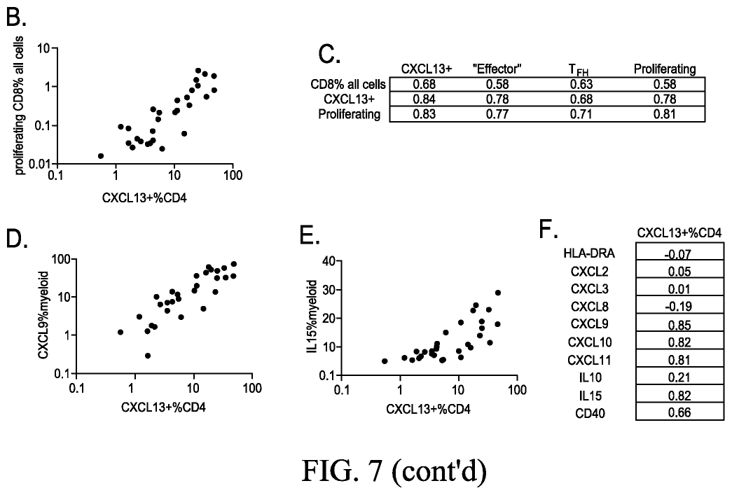

To address whether the population structure of CD4⁺ T cells observed in the single cell sequencing data in melanoma and the associated correlations are similar in another cancer type with distinct tumor biology, a single cell data set from a cohort of 31 breast cancer patients treated with anti PD-1 was utilized (Bassez et al., 2021). CD4⁺ T cells in this cohort showed similar patterns of gene expression, with clusters of CXCL13⁺, PD-1⁺ cells expressing high levels of IL7R, FoxP3⁺ regulatory T cells, and CXCL13⁺ cells comprised of three distinct populations that expressed higher relative levels of TYMS, T_{FH} (Bcl6) and memory markers (TCF7 and IL7R), or HAVCR2 (the gene encoding Tim-3), IFN-γ and GZMA (FIG. 7(A)). These CXCL13+ populations were clonally expanded in tumors and expanded with immune checkpoint inhibitor treatment (Bassez et al., 2021).

This analysis found that CXCL13+CD4+ T cell populations as a fraction of CD4⁺ T cells correlated with CD8+ T cells (Spearman R=0.68, p<104) and more closely with CXCL13⁺ CD8⁺ T cells (R=0.84, p<10⁻⁸) and proliferating CD8⁺ T cells (R=0.83, p<10⁻⁸) as a fraction of tumor infiltrating cells (FIG. 7(B-C)). This result is consistent with the correlation seen in melanoma. CXCL13+CD4⁺ T cells as a fraction of CD4⁺ T cells was also correlated positively with expression of CXCL9, CXCL10, CXCL11, IL15 and CD40 (Spearman R>0.66, p<10⁻⁴), mirroring the present findings in melanoma, but not with expression of CXCL2, CXCL3, or IL10 (FIG. 7(D-F)). The TCF7⁺ CXCL13⁺ subset as a fraction of CD4+ T cells correlated modestly with the fraction of total B cells, naïve B cells, and memory B cells as a fraction of total cells (Spearman R>0.45, p<0.01), but TCF7-CXCL13⁺ CD4⁺ T cells did not, as had been seen with melanoma. Taken together, these data show that similar populations of CD4⁺ T cells exist in a second tumor type with distinct biology from melanoma, and that the fraction of CXCL13⁺ CD4⁺ T cells correlates with activation in the CD8⁺ T cell, myeloid and B cell components of the TME.

### Example 10

### Discussion

The phenotypic states and role of tumor antigen-specific CD4⁺ T cells in the tumor microenvironment is poorly understood. The present disclosure identifies neoantigen and tumor self-antigen-specific CD4⁺ T cells in human melanoma and shows that their phenotypes and transcriptional signatures are distinct from other infiltrating CD4⁺ T cell populations. Unlike clonally diverse CD4⁺ T cells that include virus-specific cells, CD4⁺ T cell clusters containing tumor antigen-specific cells are frequently clonally expanded and characterized by the expression of CXCL13, PD-1 and Tox. 350 tumor-specific CD4⁺ T cells were identified from primary tumor samples of 4 different patients, which represents a large increase in the number of neoantigen-specific CD4⁺ T cells compared to previous studies (Balança et al., 2021; Cachot et al., 2021), and the vast majority of these resided in the CXCL13⁺ clusters. CXCL13⁺ CD4⁺ T cells could be subdivided into three distinct clusters defined by: a first cluster with expression of markers of proliferation, a second with expression of TCF7 and T_{FH} markers, and a third TCF7, Tim3⁺, CD103⁺, GZMA⁺, IFNG⁺ population. While the finding of the presence of tumor antigen-specific T cells exclusively within the PD-1-high CXCL13-high subset in TIL suggests that this is a signature of tumor antigen-specificity, the possibility that some tumor antigen-specific T cells reside in the clonally diverse PD-1-negative TCONv compartment or that other CD4⁺ T cells with this phenotype are not tumor antigen-specific cannot be excluded.

CXCL13⁺ T_{FH}-like CD4⁺ T cells and more generally PD-1-high conventional CD4⁺ T cells have been identified in non-small cell lung, head and neck, colorectal and triple negative breast cancers, however their antigen specificity has not been defined and it has been controversial whether such cells are stimulatory or suppressive of antitumor immunity (Bonnal et al., 2021; Cui et al., 2020; Gu-Trantien et al., 2013; Hollern et al., 2019; Ruffin et al., 2021; Singh et al., 2020; Zappasodi et al., 2018). The present disclosure shows that the presence of CXCL13⁺ CD4⁺ T cells, and that CXCL13 expression more broadly, correlated with overall survival in the study cohort despite diverse subsequent therapy, supporting a potential role of these cells in antitumor immunity. This is supported by the observation that CXCL13 expression predicts response to immune checkpoint inhibition independent of CD8⁺ T cell infiltration in a meta-analysis of multiple cancer types (Litchfield et al., 2021), and that secretion of CXCL13 by cultured tumor fragments also predicted checkpoint inhibitor response (Voabil et al., 2021). CXCL13 is also produced by tumor antigen-specific CD8⁺ T cell subsets, but the present data and that of others suggest that CD4⁺ T cells are a major source of CXCL13 within tumors (Gu-Trantien et al., 2013). Furthermore, it was recently shown that greater numbers of PD-1-high CD4⁺ T cells within tumors predicted immune checkpoint inhibitor responsiveness better than overall immune infiltration or infiltration of PD-1-high CD8⁺ T cells (Voabil et al., 2021).

Although CD4⁺ T cells can mediate direct lysis of class II MHC-positive tumor cells (Oh et al., 2020), a significant fraction of tumor cells from only 5 out of the 20 patients in the present cohort showed evidence of class II MHC expression, consistent with prior work showing the majority of melanoma cells are class II MHC-negative (Rodig et al., 2018). CD4⁺ T cells can also coordinate antitumor immunity through several other mechanisms, which have been demonstrated in studies of murine models. These include local support of CD8⁺ T cell responses (Alspach et al., 2019), activation of innate immune cells such as macrophages (Mumberg et al., 1999; Tveita et al., 2016), and activation of B cells (Hollern et al., 2019). In the study cohort of 20 patients, the abundance of CD4⁺ T cells with the signature of tumor antigen reactivity correlated with local activation of CD8⁺ T cells, activation of an immune stimulatory phenotype in macrophages, and the presence and differentiation of B cells, suggesting diverse functional roles of tumor antigen-specific CD4⁺ T cells in shaping the melanoma TME. While phenotyping data alone do not prove that tumor antigen recognition by CD4⁺ T cells mediates these changes, it is plausible that tumor antigen specific interactions with MHC class II positive B cells and myeloid cells could mediate each of these effects. Recent work in a murine model showing CD4+ T cells that were activated by tumor antigen specific B cells could stimulate antitumor CD8⁺ T cell responses through IL-21 secretion could point to one potential mechanism (Cui et al., 2020; Zander et al., 2019). These findings with a sample size of 20 patients are supported by correlations of the associated markers CXCL13, BTLA, and IL-21 identified in the study with markers of CD8⁺ T cell and B cell infiltration and macrophage activation in a larger cohort of melanoma patients, and by analysis of an independent scRNA seq data from breast cancer. A further test of whether tumor antigen-specific CD4⁺ T cells have a causal role in modifying the tumor microenvironment in the direction of successful antitumor immunity is whether therapeutic interventions that enhance the frequency and function of these cells such as by the adoptive transfer of tumor antigen-specific CD4⁺ T cells are capable of mediating these changes. Prospectively identifying tumor antigen-specific CD4⁺ T cells by phenotype may allow the isolation of such cells or their tumor-reactive TCRs for use in adoptive T cell therapy approaches to activate the TME and potentially induce or augment clinical responses to ICB. The existence of such cells across different tumor types suggests that such a therapeutic approach may be broadly applicable in immunogenic solid tumors.

### Example 11

### Experimental Model and Subject Details

### Patient Samples:

Twenty patients were enrolled in a clinical protocol approved by the Institutional Review Board of Fred Hutchinson Cancer Research Center (FHCRC 2643; NCT01807182) to provide tumor samples for research and for production of therapeutic tumor infiltrating lymphocyte (TIL) products. Melanoma patients with stage IV or stage III disease unlikely to be cured by surgery, >18 years of age, with an ECOG</=1, and with a site of metastatic disease that could be safely resected or biopsied, were eligible for enrollment. Leukapheresis products were obtained under a separate IRB-reviewed clinical protocol from patients X197 and X198 after the patients received TIL infusion for progressive disease. All patients provided informed consent for enrollment on these protocols.

### Method Details

### Nucleic Acid Preparation

Tumor single cell suspensions for whole exome sequencing and bulk RNA sequencing were thawed and depleted of hematopoietic cells using the EasySep™ Human CD45 Depletion Kit (StemCell) and DNA and RNA were extracted using the AllPrep DNA/RNA Mini Kit (Qiagen). Patient X198 tumor DNA and RNA was isolated without depletion of hematopoietic cells. DNA extraction from T cells for TCRVβ sequencing, and from peripheral blood mononuclear cells (PBMC) for TCRVβ sequencing or exome sequencing was performed using the the DNEasy kit (Qiagen) or the QIAamp DNA Micro Kit (Qiagen) if cellular input was below 500,000 cells. DNA following stimulation of PBMC was extracted following enrichment of CD4⁺ T cells using Easy Sep™ Human CD4+ T Cell Isolation Kit (StemCell).

### Whole Exome Sequencing:

Exome sequencing libraries were prepared using the Agilent SureSelectXT Reagent Kit and exon targets isolated using the Agilent All Human Exon v6 (Agilent Technologies, Santa Clara, CA, USA). 200 ng of genomic DNA was fragmented using a Covaris LE220 focused-ultrasonicator (Covaris, Inc., Woburn, MA, USA) and libraries prepared and captured on a Sciclone NGSx Workstation (PerkinElmer, Waltham, MA, USA). Library size distributions were validated using an Agilent 2200 TapeStation. Additional library QC, blending of pooled indexed libraries, and cluster optimization was performed using Life Technologies' Invitrogen Qubit® 2.0 Fluorometer.

The resulting libraries were sequenced on an Illumina HiSeq 2500 using a paired-end 100 bp (PE100) strategy. Image analysis and base calling was performed using Illumina's Real Time Analysis v1.18 software, followed by “demultiplexing” of indexed reads and generation of FASTQ files using Illumina's bcl2fastq Conversion Software v1.8.4 (http://support.illumina.com/downloads/bcl2fastq_conversion_software_184.html). Read pairs passing standard Illumina quality filters were retained for further analysis. Paired reads were aligned to the human genome reference (GRCh37/hg19) with the BWA-MEM short-read aligner (Li, 2013; Li and Durbin, 2009). The resulting alignment files, in standard BAM format, were processed by Picard 2.0.1 and GATK 3.5 (McKenna et al., 2010) for quality score recalibration, indel realignment, and duplicate removal according to recommended best practices (Van der Auwera et al., 2013).

To call somatic mutations from the analysis-ready tumor and normal BAM files, three independent software packages were used: MuTect 1.1.7 (Cibulskis et al., 2013) and Strelka 1.0.14 (Saunders et al., 2012). Variant calls from both tools, in VCF format, were annotated with Oncotator (Ramos et al., 2015). Annotated missense somatic variants were combined into a single summary for each sample as follows. First, any mutation annotated as “somatic” but present in dbSNP was removed if it was not also present in COSMIC or its minor allele frequency was greater than 1% (according to the UCSC Genome Browser snp150Common table). Variants supported by both variant callers were retained, and those supported by only one variant caller were subject to manual inspection.

### RNA-Seq Data Processing:

An RNA-seq library was prepared from total RNA using the TruSeq RNA Sample Prep v2 Kit (Illumina, Inc., San Diego, CA, USA) and a Sciclone NGSx Workstation (PerkinElmer, Waltham, MA, USA). Library size distributions were validated using an Agilent 2200 TapeStation (Agilent Technologies, Santa Clara, CA, USA). Additional library QC, blending of pooled indexed libraries, and cluster optimization was performed using Life Technologies' Invitrogen Qubit® 2.0 Fluorometer (Life Technologies-Invitrogen, Carlsbad, CA, USA). The library was sequenced on an Illumina HiSeq 2500 to generate 61M read pairs (two 50nt reads per pair). Reads were aligned to a human RefSeq derived reference transcriptome with RSEM 1.2.19 (Li and Dewey, 2011) to derive abundances for each gene in transcript-per-million (TPM) units.

### Selection of Mutations for Screening:

Screening of mutations from patient X197 for T cell reactivity was described previously. (Veatch et al., 2018) Patient X198 had a large number of mutations (>1100) and variants called by both MuTect and Strelka were filtered for variant allele frequency (VAF) of greater than 20% and only the top 46 mutations by total number variant reads in the RNAseq were selected for screening. Patient X205 had 164 SNVs identified. Variants called by both MuTect and Strelka were filtered for VAF greater than 30% and the top 30 mutations were selected with a mRNA expression level of >14TPM. Patient X422 had 299 identified SNVs, which were filtered for a VAF greater than 25% and the top 33 mutations with mRNA expression above 20 TPM were selected for screening. There were few insertions or deletions resulting in frameshifts that would need be subject to nonsense mediated decay and while these were examined manually, none were selected for screening.

For identifying CD8 epitopes, SNVs from patient X198 mutations that were called by both mutect and strelka were filtered for VAF>0.1 and detection of the variant in at least 5 reads from RNA sequencing. NetMHC version 4.0 was used to select 169 different 9 and 10 amino acid peptides predicted to bind to HLA-A0101, HLA-A2301 and HLA-B0801 which were then used in screening.

### Peptides

Two peptides spanning each mutation with the mutated residue at position +7 or +13 of the 20 amino acid sequence were synthesized by Elim Biopharma and used for initial stimulation and screening to detect CD4+ T cell responses. Subsequent experiments to confirm T cell reactivity only to the mutant and not the wild type peptides were performed with >80% purity 27-mer peptides with the mutant or wildtype amino acid at position +13.

### Antigen Presenting Cells

Autologous B cells were isolated from fresh or thawed PBMC by positive selection using human CD19 microbeads (Miltenyi, cat #130-050-301) according to the manufacturer's instructions. Isolated B cells were cultured with irradiated (5000Gy) NIH 3T3 cells expressing human CD40L for 7 days in B cell medium supplemented with 200U/ml human IL-4 (Peprotech) as described. (Tran et al., 2014) B cells were subsequently harvested and restimulated with 3T3 CD40L and fresh medium every 3 days. B cells were pulsed with peptides and used in assays at day +3 of stimulation 2 or 3.

### T Cell Culture:

For production of clinical tumor infiltrating lymphocyte (TIL) products used to detect CD8⁺ T cell responses from patients X197 and X198, TIL were expanded from tumor fragments in 6,000 IU/ml recombinant IL-2 (Proleukin; Novartis), using methodologies developed at the Surgery Branch of the National Cancer Institute (Dudley et al., 2001). TIL cultures were selected based on cell growth and autologous tumor reactivity as determined by IFN-g secretion after coculture with autologous tumor cells. The TIL were cryopreserved until needed for use, then thawed and further expanded using a rapid expansion protocol, as previously-described (Riddell and Greenberg, 1990).

For isolation of CD4+ T cells from tumor samples, cryopreserved tumor single cell suspensions made from tumor tissue that remained after TIL generation were thawed and incubated overnight in RPMI media with L-glutamine and HEPES (Gibco) supplemented with 10% human serum, 50 μM beta-mercaptoethanol, penicillin and streptomycin, 4 mM L-glutamine (termed CTL media) and 2 ng/ml recombinant human IL-7 (Peprotech). The cell suspensions were then stained with fluorescently labelled antibodies to human CD4, CD3 and PD-1 and the top tertile of PD-1 expressing CD3+CD4+ T cells were sorted, and plated at 10 cells per well in a 96 well plates with 100,000 irradiated allogeneic PBMC in CTL media supplemented with 3,000 U/ml of recombinant human IL-2 and 50 ng/ml anti-CD3 (OKT3). (Yossef et al., 2018) Cytokines were added every 7 days and colonies of cells were screened for reactivity against mutant paptides at day 28 of culture. Between 3 and 10% of wells formed T cell colonies large enough to screen. These T cell lines from patients X422 and X205 were screened for antigen reactivity by incubation with a pools of all 20-mer crude mutant peptide pairs (2 ug/ml/peptide) from that patient in the presence of brefeldin A, followed by staining with antibodies for human CD4 and CD19, fixing and permeabilizing using the BD Cytofix/Cytoperm kit (BD cat #554714) using the manufacturer's instructions, and staining of intracellular IFN-g (#B27). Quantitation of IFN-g secreting cells by FACS was compared to an unstimulated aliquot of T cells. Cell lines with >50% IFN-g secretion were then expanded using a rapid expansion protocol and cryopreserved (Riddell and Greenberg, 1990). Cryopreserved T cells were thawed and rested overnight in CTL media supplemented with 10 U/ml IL-2 prior to ELISA after stimulation with overlapping peptide pools to determine the individual target mutation, and then stimulation with purified 27-mer peptides with either the wildtype or mutant amino acid at position +14 to confirm antigen specificities.

Ten thousand of the top tertile of PD-1 expressing CD4+CD3+ from patient X205 were also polyclonally expanded in 4 ml CTL in a 12 well plate by stimulation with anti-CD3 (OKT3, 50 ng/ml) in cultures supplemented with 6×10⁶ irradiated (5000 rad) allogeneic PBMC and 3,000 U/ml IL-2. After 21 days, the T cells were incubated at a 1:1 ratio with autologous B cells and neoantigen peptide pools (2 ug/ml each peptide) in the presence of mononesin, anti-CD40, anti-CD28, anti-CD49a, and fluorescently labelled antiCD40L for 16 hours as described (Chattopadhyay et al., 2006). CD40L expressing CD4+ T cells were sorted by FACS and DNA was prepared for TCRVβ sequencing to identify additional neoantigen reactive TCRVβ clonotypes.

Cryopreserved PBMC from the two patients that received TIL infusion were thawed and rested overnight in CTL and 2 ng/ml recombinant human IL-7. The following morning PBMC were washed and 10×10⁶ cells were plated in individual wells of a 6 well plate in 5 ml CTL media containing a pool of lug/ml of each candidate peptide supplemented with IL-21 (30 ng/ml). Recombinant IL-7 (5 ng/ml), IL-15 (1 ng/ml) and IL-2 10 U/ml (Peprotech) was added on day +3, and half media changes with supplemental IL-2, IL-7 and IL-15 were performed on days +3, +6, and +9. On day +13, cells from individual wells were harvested and T cells assayed by ELISA, ELISpot, or cytokine staining assays. Cultures that were evaluated by TCRVβ sequencing for expansion of TCRVβ clones were enriched for CD4+ T cells by negative magnetic selection with the human CD4 isolation kit (StemCell) prior to DNA isolation and TCRVβ sequencing.

### Antigen Specificity Assays

ELISpot assays were performed by incubating 50,000 T cells from cultures with 100,000 peptide pulsed (10 mg/ml or otherwise indicated) and control autologous B cells as APC overnight using the human interferon gamma ELISpot kit (Mabtech) following the manufacturer's instructions. ELISA was performed by incubating 50,000 T cells with 100,000 peptide pulsed and control autologous B cells and IFN-g release in the supernatant was quantitated using the human interferon gamma ELISA kit (Thermo fisher).

### Ex Vivo T Cell Assays

T cell populations were sorted from tumor single cell suspensions and For T cell stimulation assays, rested cells were labelled with cell trace violet (thermo fisher) combined with antiCD3/antiCD28 dynabeads (thermo fisher) at a 3:1 ratio in CTL. 24 hours later 10U/ml IL-2 was added. Cells were assayed for proliferation by flow cytometry on day 5 after stimulation. For B cell stimulation assays, allogeneic B cells were isolated from the peripheral blood of a healthy donor using the human B cell positive selection release kit (StemCell) and rested overnight in B cell medium prior to labelling with cell trace violet (thermo fisher) and coculture of with 1:1 rested CD4+ T cells. B cell proliferation was measured at day 5 by flow cytometry.

### B Cell Proliferation/TFH Assay

Tumor single cell suspensions were thawed and labelled with LIVE/DEAD™ Fixable Aqua Dead Cell Stain Kit (Thermofisher Scientific, cat #L34957) as per manufacturer's protocol. The cells were incubated in Fc block at RT for 5 mins, then stained with fluorescently labelled antibodies antibodies CD3, CD4, PD-1, CD25, CD127, CD200, CXCR6 ( ) at 4degC for 30 mins. Next, CD3+CD4+ T cells were sorted into a CD127-CD25+ Treg population, and conventional T cells were separated into PD-1 high and PD-1 low CD127+ high populations (termed “bystanders”) and PD-1 high cells were separated into CXCR6-TFH cells and CXCR6+ “effector” cells using a symphony S6 cell sorter. Sorted cells were rested overnight in CTL+50U/mL IL-2 and 5 ng/ml IL-7 prior to assays. On the same day, B cells were isolated from thawed PBMCs of a healthy donor using. Isolated B cells were rested overnight in IMDM supplemented with 10% human serum, penicillin-streptomycin, and L-glutamine (termed BCM). After overnight culture, B cells were labelled with CellTrace Violet Cell Proliferation Kit (Thermofisher Scientific, cat #C34571) per manufacturer's protocol, then cocultured with each of the different T cell subsets in the presence of SEB 100 ng/ml. Proliferation was analyzed after 5 days by flow cytometry.

### T Cell Proliferation Assay

Tumor single cell suspensions from were thawed and CD45⁺ cells were enriched using Easy Sep™ Release Human CD45 Positive Selection Kit (StemCell Technologies, cat #100-0105) based on manufacturer's protocol. Fc block was added to the isolated cells and cells were incubated RT for 5 mins, then stained with fluorescently labelled antibodies against CD3, CD4, PD-1, CD25, CD127, CD200, CXCR6 at 4degC for 30 mins. Next, the different CD4 T cell subsets (Effectors, TFH, Bystanders) were sorted using BD FACSymphony S6 System and sorted cells were cultured overnight in CTL media supplemented with IL-2 50U/ml and IL-7 5 ng/ml. After resting the cells overnight, T cells were labelled using CellTrace Violet Cell Proliferation Kit (Thermofisher Scientific, cat #C34571) per manufacturer's recommendations. Next, CD3/CD28 Dynabeads were added to the labelled cells at 3:1 bead to cell ratio in CTL, and cultured cells at 37degC. After 24 hours, IL-2 10U/ml was added to the cells and cultured for 4 days. On day 4, the Dynabeads were removed and cells were stained with antibodies CD3 BV711, CD4 BUV395, CD19 PE and proliferation was analyzed using flow cytometry.

### TCRVβ Sequencing

DNA from tumors, sorted T cells, PBMC, or T cell cultures was analyzed for TCRVβ repertoire using the human TCRβ kit (Adaptive Biotechnologies) and analyzed using company software. A single PCR reaction was used for analyzing oligoclonal cell lines and small populations of sorted cells, 2 PCR reactions (survey depth) were used for PBMC cultures, and 4 PCR reactions per sample were used for analysis of PBMC and primary tumor samples. TCRVβ clonotypes identified as antigen specific had to meet the following criteria: 1) detected with at least 2 templates in both replicates of a specific stimulation and 2) both replicates of the specific stimulation had to be greater than the sum of all other stimulations. All primary TCRVβ sequencing data will be made available at the Adaptive Biotechnologies website https://clients.adaptivebiotech.com/immuneaccess.

Single-Cell Capture and cDNA Library Preparation for Single Cell RNA Sequencing

Single-cell libraries were prepared using the BD Rhapsody Express system (BD Biosciences, #633707) and Targeted mRNA and AbSeq Reagent Kit-4 pack (BD Biosciences, #633771) according to the manufacturer's protocol (BD Biosciences). Briefly, tumor samples from each donor were thawed and labelled with sample tags using BD Single-Cell Multiplexing Kit (BD Biosciences, #633781). Cells from each donor were labelled with a unique sample tag. Cells were then washed in BD Stain buffer (BD Biosciences, #554656), pooled together and incubated in Fc block followed by labelling cells with BD AbSeq Ab-Oligos master mix (3 uL per Ab-Oligo). For flow sorting various lymphoid and myeloid cell populations, cells were stained with a mixture of fluorescently labelled antibodies (BD Biosciences or BioLegend): anti-human CD3 (#OKT3), CD4 (#RPA-T4), CD8 (#RPA-T8), CD19 (#HIB19), CD45 (#2D1), for 30 mins at 4° C. After sorting, cells were counted and pooled such that there were 20,000 cells resuspended in 620 mL of BD Sample buffer. The BD Rhapsody cartridges were primed, and the pooled cells were loaded onto the cartridges and incubated at room temperature. Next, Cell Capture beads were washed & loaded onto the cartridge. After incubation, cartridges were washed twice with BD Sample Buffer. This was followed by lysis of cells and retrieval of beads. Reverse transcription was performed immediately after the retrieved Cell Capture beads were washed. For the subsequent steps, the BD Rhapsody system VDJ CDR3, Sample Tag, and BD AbSeq library protocol (BD Biosciences) were used to generate TCR and BCR libraries in addition to mRNA, AbSeq, Sample Tag libraries. Poly-T Template Switching Oligo (TSO) was added during reverse transcription reaction to allow identification of VDJ recombination events in B and T cells. This was followed by denaturation, hybridization, Klenow extension (New England Biolabs, #M0212L) and treatment with Exonuclease-I.

cDNA Libraries were prepared in a two-step nested PCR reaction followed by Index PCR using BD Rhapsody Targeted mRNA and AbSeq Amplification Kit (BD Biosciences, #633774) and BD Rhapsody™ Immune Response Panel Hs (BD Biosciences, #633750). We included BD Rhapsody Supplemental Panel (BD Biosciences, #633742) to look at additional genes GATA3, MAF, CCR6, CD40L, TOX, IL6R, IL9R and IL 10. After the first PCR, longer PCR products (mRNA, TCR, BCR) were separated from shorter products (AbSeq & Sample Tag) based on double-sided size selection using Agencourt AMPure XP beads (Beckman Coulter, #A63880). The concentration of libraries was estimated using Qubit™ dsDNA HS Assay Kit (Thermo Fisher Scientific, #Q32851). The products from second PCR were diluted as per protocol's recommendations for Index PCR. The quality check and quantification of Index libraries was performed using Agilent 2200 TapeStation with High Sensitivity D5000 ScreenTape (Agilent).

Sequencing was performed on a Novaseq SP flow cell at 75×225 and after demultiplexing, sample tags, mRNA, and antibody sequencing reads were trimmed to 75nt and TCR and BCR VDJ libraries were kept at 225nt and fastq files were uploaded to the BD Rhapsody analysis pipeline (https://www.sevenbridges.com/bdgenomics/) with refined cell calling disabled. For the initial analysis of 4 patients, CD4+ T cells were sorted and sequenced. For the analysis of the full cohort of 20 patients, CD19+ and CD3+ cell populations were labelled and sequenced separately from CD45+CD19-CD3-myeloid populations to facilitate greater cell detection and sequencing depth of the lymphoid cells.

### Immunohistochemistry

Formalin-fixed paraffin-embedded tissues were sectioned at 4 microns onto positively-charged slides and baked for 1 hour at 60° C. The slides were then dewaxed and stained on a Leica BOND Rx stainer (Leica, Buffalo Grove, IL) using Leica Bond reagents for dewaxing (Dewax Solution), antigen retrieval/antibody stripping (Epitope Retrieval Solution 2), and rinsing after each step (Bond Wash Solution). Antigen retrieval and antibody stripping steps were performed at 100° C. with all other steps at ambient temperature.

TABLE 2
Immunohistochemistry antibodies

| | | | Manufacturer/ | Dilution/ | | OOpal |
|---|---|---|---|---|---|---|
| Position | Antibody | Clone/Host | Catalog # | Concentration | Secondary | Dye |
| 1 | CD3 | SP7/ | Invitrogen/ | 1:400 | PowerVision | OOpal |
| | | Rabbit | MAB190582 | | Rabbit HRP | 480 |
| 2 | CD8 | 144B/ | DAKO/ | 1:500 | PowerVision | OOpal |
| | | Mouse | M7103 | (0.314 mg/ml) | Mouse HRP | 540 |
| 3 | FoxP3 | 236A/E7/ | eBioscience/ | 1:100 | PowerVision | OOpal |
| | | Mouse | 14-4777-82 | (5 mg/ml) | Mouse HRP | 650 |
| 4 | CD163 + | ERP324/ | BioSB/ | 1:800 | OPAL Ms + Rbt | OOpal |
| | CD68 | Rabbit | BSB3276 | (0.125 mg/ml) | HRP | 690 |
| | | PG-M1/ | Dako/M0876 | 1:800 | | |
| | | Mouse | | (0.038 mg/ml) | | |
| 5 | Mel + | M2-7C10; M2- | Novus/NBP2- | 1:2000 | OPAL Ms + Rbt | OOpal |
| | Sox10 | 9E3, T311, | 34337 | (0.05 mg/ml) | HRP | 520 |
| | | HMB45/Ms | CellMarque/ | 1:400 | | |
| | | Polyclonal/ | 383A-74 | (0.67 mg/ml) | | |
| | | Rabbit | | | | |
| 6 | CXCL13 | Polyclonal/ | R&D/AF801 | 1:200 | ImmPress Goat | OOpal |
| | | Goat | | (1 mg/ml) | HRP | 570 |
| 7 | CD4 | EP204/ | Cell Marque/ | 1:50 | PowerVision | OOpal |
| | | Rabbit | 104R-25 | (0.196 mg/ml) | Rabbit HRP | 620 |
| 8 | CD79a | JCB117/ | BioSB/ | 1:500 | PowerVision | OOpal |
| | | Mouse | BSB5305 | 0.2 mg/ml | Mouse HRP | 780 |

Endogenous peroxidase was blocked with 3% H₂O₂ for 5 minutes followed by protein blocking with TCT buffer (0.05M Tris, 0.15M NaCl, 0.25% Casein, 0.1% Tween 20, 0.05% ProClin300 pH 7.6) for 10 minutes. The first primary antibody (position 1) was applied for 60 minutes followed by the secondary application for 10 minutes and the application of the tertiary TSA-amplification reagent (PerkinElmer OPAL fluor) for 10 minutes. A high stringency wash was performed after the secondary and tertiary applications using high-salt TBST solution (0.05M Tris, 0.3M NaCl, and 0.1% Tween-20, pH 7.2-7.6). Species specific HRP polymer was used for all secondary applications.

The primary and secondary antibodies were stripped with retrieval solution for 20 minutes before repeating the process with the second primary antibody (position 2) starting with a new application of 3% H2O2. The process was repeated until seven positions were completed. For the eighth position, following the secondary antibody application, Opal TSA-DIG was applied for 10 minutes, followed by the 20 minute stripping step in retrieval solution and application of Opal 780 fluor for 10 minutes with high stringency washes performed after the secondary, TSA-DIG, and Opal 780 fluor applications. The stripping step was not performed after the final position.

Slides were removed from the stainer and stained with DAPI for 5 minutes, rinsed for 5 minutes, and coverslipped with Prolong Gold Antifade reagent (Invitrogen/Life Technologies, Grand Island, NY).

Slides were cured overnight at room temperature, then whole slide images were acquired on the Vectra Polaris Quantitative Pathology Imaging System (Akoya Biosciences, Marlborough, MA). The entire tissue was selected for imaging using Phenochart and multispectral image tiles were acquired using the Polaris. Images were spectrally unmixed using Phenoptics inForm sotware and exported as multi-image TIF files, which were analyzed with HALO image analysis software (Indica Labs, Cooales, NM). Cellular analysis of the images was performed by first identifying cells based on nuclear recognition (DAPI stain), then measuring fluorescence intensity of the estimated cytoplasmic areas of each cell. A mean intensity threshold above background was used to determine positivity for each fluorochrome within the cytoplasm, thereby, defining cells as either positive or negative for each marker. The positive cell data was then used to define colocalized populations.

### Quantitation and Statistical Analysis

### Single-Cell Data Preprocessing and Analysis

Data preprocessing and analysis was performed primarily using the Scanpy toolkit (Wolf et al., 2018) in Python. Low quality cells with fewer than 100 total counts and 10 expressed genes were removed. Doublets were first removed with Scrublet, (Wolock et al., 2019) using its automatic doublet detection threshold. CPM normalization was performed with the Scanpy ‘normalize_total’ function, and the data was then log-transformed. Sample tags were extracted from BD Rhapsody analysis pipeline (https://www.sevenbridges.com/bdgenomics/), and cells that had been identified as multiplets or that were labeled as ‘undetermined’ were removed. Batch correction to correct for variability among patients was performed on mRNA data using ComBat (Johnson et al., 2007; Tangherloni et al., 2021) implemented in Scanpy.

Dimension reduction was first performed by principal component analysis and then by UMAP after construction of a nearest neighbors graph using the first 20 principal components. This was performed on mRNA data alone. Unsupervised clustering was performed using the Leiden algorithm, implemented in Scanpy. The resulting clusters were manually labeled according to top differentially expressed genes in each cluster; clusters containing tumor cells or evident doublets not captured by Scrublet were discarded. Differential expression between clusters was assessed using MAST (Finak et al., 2015) with a threshold of FDR of 0.05. Heatmaps were generated from the top ten differentially expressed genes from each cluster as calculated with MAST.

TCRVβ analysis was performed using some functionality from Scirpy (Sturm et al., 2020) to assess clonal expansion and calculate clonotype size. The Scirpy ‘repertoire_overlap’ function was used to calculate the Jaccard similarity coefficients based on clonotype representation between clusters.

### TCGA Analysis

Gene expression data was extracted from the TCGA-SKCM (Skin Cutaneous Melanoma) data set using the TCGAbiolinks package (Colaprico et al., 2015). Relevant genes were extracted and normalized to CD4. Spearman correlations and corresponding p-values were calculated between genes of interest. Correlation plots were generated using Gene Expression Profiling Interactive Analysis (GEPIA, http://gepia.cancer-pku.cn/) (Tang et al., 2017)

### Breast Cancer Data Analysis

Single cell RNA-seq data was downloaded from cohort 1 of (Bassez et al., 2021). The provided cell metadata was used to identify B cells, CD4 and CD8 T cells, and myeloid cells. For each broad cell type, data was processed with the standard Scanpy (Wolf et al., 2018) pipeline (log-normalization, highly variable genes extraction, PCA, and UMAP transform). Leiden clustering was performed, and differential expression heatmaps (generated with the ‘rank genes_groups’ function in Scanpy) were used to identify cell subtypes from the Leiden clusters.

### Statistics

Differential expression of genes and surface proteins from single cell sequencing were determined using MAST as above. Other statistical testing was done using Prism software (Graphpad). Survival analysis was conducted using the log rank test. Correlation analyses were performed using spearman correlation and for multiple testing a false discovery rate was determined by the Benjamini-Hochberg procedure. T cell proliferation between different cell populations from different patients was performed with a paired T test. Overlap of gene sets from CD4 and CD8 T cell populations was determined using a fishers exact test, and comparisons between proximity of T cell populations to B cells were done with the Kruskal-Wallis test with Dunn's correction.

### Proximity Analysis of Immunohistochemistry

Images were analyzed using digital image analysis software (HALO v3.1, Indica Labs, Corrales, NM). Briefly, specific area(s) within the image were annotated for analysis; for X197 lymph node metastasis section, a layer with total classified area of 29.53 mm² was annotated based on Mel-Sox10 staining, and for X198 tumor section, a randomly selected layer with total classified area of 42.77 mm² was annotated for analysis. For analysis (algorithm: Indica Labs-HighPlex FL v3.2.1), settings for each stain/marker were tuned to identify cell phenotypes defined using one or more markers (e.g., CD4+ CXCL13+ defined as CD3+CD8-CXCL13+). Randomly selected regions were screened across the image for positive cells for a given phenotype using real-time tuning. Finally, the analysis algorithm was run on the annotation layer to obtain the number of cells corresponding to each of the defined phenotypes. For proximity analysis, the Spatial Analysis Module in HALO was used to quantify the number of cells within 100 um distance of another cell type.

### Example 12

### Further Experiments

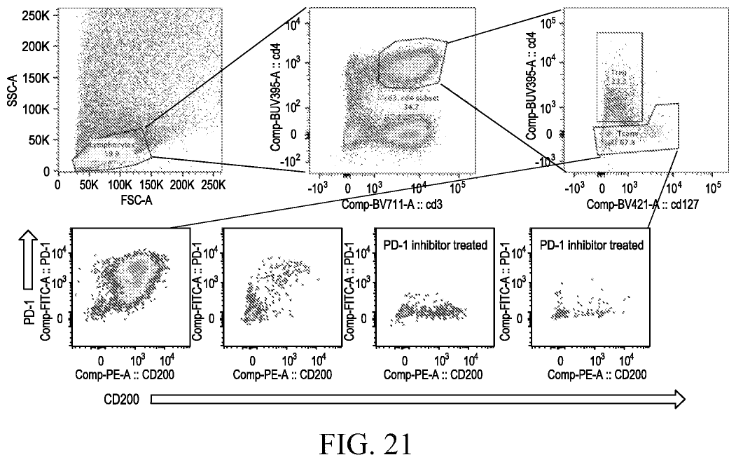

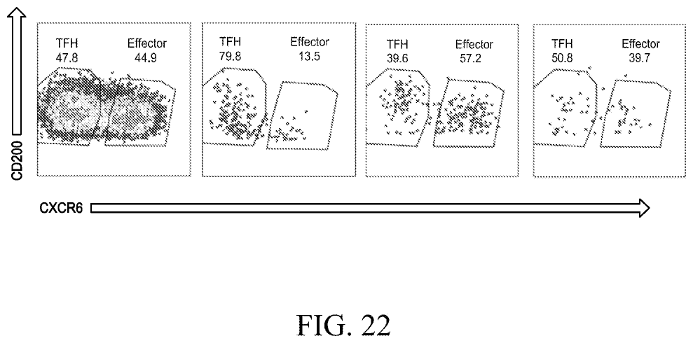

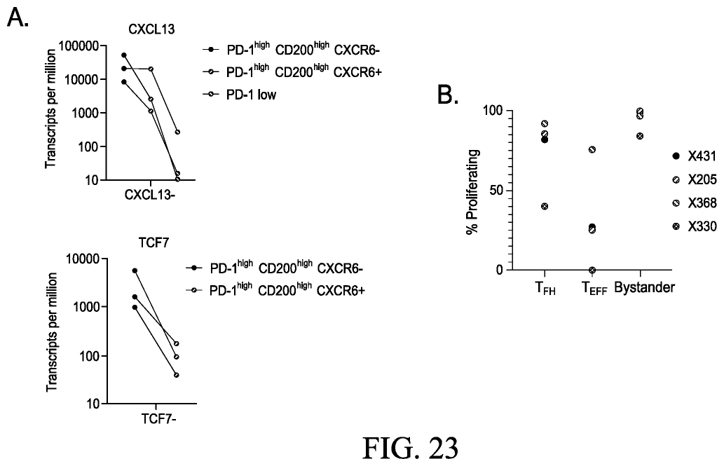

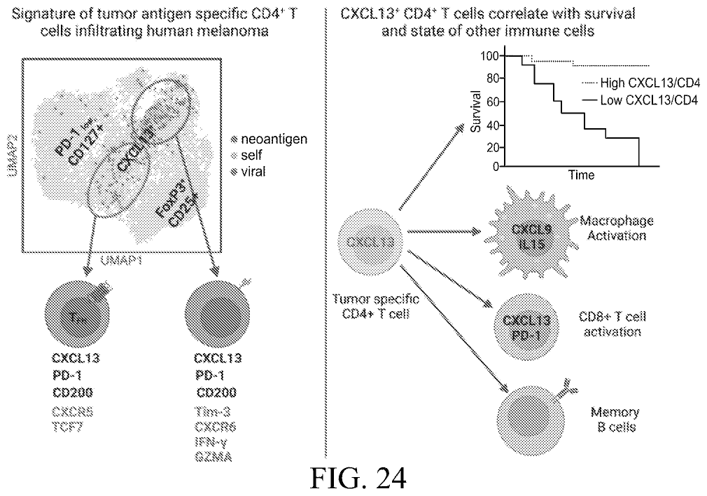

Further experiments were performed, with results shown and described for FIGS. 20-24. Briefly, these data show: that tumor antigen antigen-specific CD4+ T cells are lost during conventional TIL culture (FIG. 20); schema of a flow cytometry panel of the present disclosure for isolating CXCL13+ conventional CD4+ T cells (FIG. 21); flow sorting of CD25low PD-1high CD200high cells from tumors of 4 patients based on CXCR6 expression (FIG. 22); results from RNA sequencing of CD25low PD-1high CD200high cells sorted into CXCR6+ and CXCR6-fractions and TCF7 and CXCL13 expression, indicating that that CD200 and PD-1 sorting isolates CXCL13+ cells from CXCL13-cells, and that CXCR6-negative cells define the TCF7+ population (FIG. 23A); sorting of CD25low PD-1high CD200high cells into CXCR6+ (“TEFF”), CXCR6-(“T_{FH}”) and PD-1low CD200low (“bystander”) fractions, suggesting that the TCF7+ population isolated by CXCR6-negative staining is the proliferative stem/progenitor population (FIG. 23B); and schematic diagrams showing (left) certain marker signatures of CD4+ T cells infiltrating human melanoma and (right) correlation of CXCL13+CD4+ T cells with survival and state of other immune cells (FIG. 24).

### REFERENCES

Ahmadzadeh, M., Pasetto, A., Jia, L., Deniger, D. C., Stevanović, S., Robbins, P. F., and Rosenberg, S. A. (2019). Tumor-infiltrating human CD4⁺ regulatory T cells display a distinct TCR repertoire and exhibit tumor and neoantigen reactivity. Science immunology 4, eaao4310.
Alfaro, C., Teijeira, A., Oñate, C., Pérez, G., Sanmamed, M. F., Andueza, M. P., Alignani, D., Labiano, S., Azpilikueta, A., and Rodriguez-Paulete, A. (2016). Tumor-produced interleukin-8 attracts human myeloid-derived suppressor cells and elicits extrusion of neutrophil extracellular traps (NETs). Clinical Cancer Research 22, 3924-3936.
Alspach, E., Lussier, D. M., Miceli, A. P., Kizhvatov, I., DuPage, M., Luoma, A. M., Meng, W., Lichti, C. F., Esaulova, E., and Vomund, A. N. (2019). MHC-II neoantigens shape tumour immunity and response to immunotherapy. Nature, 1-6.
Balança, C.-C., Salvioni, A., Scarlata, C.-M., Michelas, M., Martinez-Gomez, C., Gomez-Roca, C., Sarradin, V., Tosolini, M., Valle, C., and Pont, F. (2021). PD-1 blockade restores helper activity of tumor-infiltrating, exhausted PD-1hiCD39⁺ CD4 T cells. JCI insight 6.
Bassez, A., Vos, H., Van Dyck, L., Floris, G., Arijs, I., Desmedt, C., Boeckx, B., Bempt, M. V., Nevelsteen, I., and Lambein, K. (2021). A single-cell map of intratumoral changes during anti-PD1 treatment of patients with breast cancer. Nature Medicine 27, 820-832.
Bonnal, R. J., Rossetti, G., Lugli, E., De Simone, M., Gruarin, P., Brummelman, J., Drufuca, L., Passaro, M., Bason, R., and Gervasoni, F. (2021). Clonally expanded EOMES+Tr1-like cells in primary and metastatic tumors are associated with disease progression. Nature Immunology 22, 735-745.
Cabrita, R., Lauss, M., Sanna, A., Donia, M., Larsen, M. S., Mitra, S., Johansson, I., Phung, B., Harbst, K., and Vallon-Christersson, J. (2020). Tertiary lymphoid structures improve immunotherapy and survival in melanoma. Nature 577, 561-565.
Cachot, A., Bilous, M., Liu, Y.-C., Li, X., Saillard, M., Cenerenti, M., Rockinger, G. A., Wyss, T., Guillaume, P., and Schmidt, J. (2021). Tumor-specific cytolytic CD4 T cells mediate immunity against human cancer. Science Advances 7, eabe3348.
Cafri, G., Yossef, R., Pasetto, A., Deniger, D. C., Lu, Y.-C., Parkhurst, M., Gartner, J. J., Jia, L., Ray, S., Ngo, L. T., et al. (2019). Memory T cells targeting oncogenic mutations detected in peripheral blood of epithelial cancer patients. Nature Communications 10, 449.
Carrero, R. M. S., Beceren-Braun, F., Rivas, S. C., Hegde, S. M., Gangadharan, A., Plote, D., Pham, G., Anthony, S. M., and Schluns, K. S. (2019). IL-15 is a component of the inflammatory milieu in the tumor microenvironment promoting antitumor responses. Proceedings of the National Academy of Sciences 116, 599-608.
Caushi, J. X., Zhang, J., Ji, Z., Vaghasia, A., Zhang, B., Hsiue, E. H.-C., Mog, B. J., Hou, W., Justesen, S., and Blosser, R. (2021). Transcriptional programs of neoantigen-specific TIL in anti-PD-1-treated lung cancers. Nature, 1-7.
Chattopadhyay, P. K., Yu, J., and Roederer, M. (2006). Live-cell assay to detect antigen-specific CD4⁺ T-cell responses by CD154 expression. Nature protocols 1, 1-6.
Cibulskis, K., Lawrence, M. S., Carter, S. L., Sivachenko, A., Jaffe, D., Sougnez, C., Gabriel, S., Meyerson, M., Lander, E. S., and Getz, G. (2013). Sensitive detection of somatic point mutations in impure and heterogeneous cancer samples. Nature biotechnology 31, 213.
Cui, C., Wang, J., Chen, P.-M., Connolly, K. A., Damo, M., Fagerberg, E., Chen, S., Eisenbarth, S. C., Zhao, H., and Craft, J. (2020). Neoantigen driven B cell and CD4⁺ T follicular helper cell collaboration promotes robust anti-tumor CD8⁺ T cell responses. bioRxiv.
Danilova, L., Anagnostou, V., Caushi, J. X., Sidhom, J.-W., Guo, H., Chan, H. Y., Suri, P., Tam, A., Zhang, J., and El Asmar, M. (2018). The mutation-associated neoantigen functional expansion of specific T cells (MANAFEST) assay: a sensitive platform for monitoring antitumor immunity. Cancer immunology research 6, 888-899.
Dudley, M. E., Wunderlich, J., Nishimura, M. I., Yu, D., Yang, J. C., Topalian, S. L., Schwartzentruber, D. J., Hwu, P., Marincola, F. M., and Sherry, R. (2001). Adoptive transfer of cloned melanoma-reactive T lymphocytes for the treatment of patients with metastatic melanoma. Journal of immunotherapy 24, 363-373.
Duhen, T., Duhen, R., Montler, R., Moses, J., Moudgil, T., de Miranda, N. F., Goodall, C. P., Blair, T. C., Fox, B. A., and McDermott, J. E. (2018). Co-expression of CD39 and CD103 identifies tumor-reactive CD8 T cells in human solid tumors. Nature communications 9, 1-13.
Finak, G., McDavid, A., Yajima, M., Deng, J., Gersuk, V., Shalek, A. K., Slichter, C. K., Miller, H. W., McElrath, M. J., and Prlic, M. (2015). MAST: a flexible statistical framework for assessing transcriptional changes and characterizing heterogeneity in single-cell RNA sequencing data. Genome biology 16, 1-13.
Gu-Trantien, C., Loi, S., Garaud, S., Equeter, C., Libin, M., De Wind, A., Ravoet, M., Le Buanec, H., Sibille, C., and Manfouo-Foutsop, G. (2013). CD4⁺ follicular helper T cell infiltration predicts breast cancer survival. The Journal of clinical investigation 123, 2873-2892.
Gubin, M. M., Zhang, X., Schuster, H., Caron, E., Ward, J. P., Noguchi, T., Ivanova, Y., Hundal, J., Arthur, C. D., and Krebber, W.-J. (2014). Checkpoint blockade cancer immunotherapy targets tumour-specific mutant antigens. Nature 515, 577-581.
Helmink, B. A., Reddy, S. M., Gao, J., Zhang, S., Basar, R., Thakur, R., Yizhak, K., Sade-Feldman, M., Blando, J., and Han, G. (2020). B cells and tertiary lymphoid structures promote immunotherapy response. Nature 577, 549-555.
Hollern, D. P., Xu, N., Thennavan, A., Glodowski, C., Garcia-Recio, S., Mott, K. R., He, X., Garay, J. P., Carey-Ewend, K., and Marron, D. (2019). B cells and T follicular helper cells mediate response to checkpoint inhibitors in high mutation burden mouse models of breast cancer. Cell 179, 1191-1206. e1121.
Horns, F., Dekker, C. L., and Quake, S. R. (2020). Memory B cell activation, broad anti-influenza antibodies, and bystander activation revealed by single-cell transcriptomics. Cell reports 30, 905-913. e906.
House, I. G., Savas, P., Lai, J., Chen, A. X., Oliver, A. J., Teo, Z. L., Todd, K. L., Henderson, M. A., Giuffrida, L., and Petley, E. V. (2020). Macrophage-derived CXCL9 and CXCL10 are required for antitumor immune responses following immune checkpoint blockade. Clinical Cancer Research 26, 487-504.
Hunder, N. N., Wallen, H., Cao, J., Hendricks, D. W., Reilly, J. Z., Rodmyre, R., Jungbluth, A., Gnjatic, S., Thompson, J. A., and Yee, C. (2008). Treatment of metastatic melanoma with autologous CD4⁺ T cells against NY-ESO-1. New England Journal of Medicine 358, 2698-2703.
Johnson, W. E., Li, C., and Rabinovic, A. (2007). Adjusting batch effects in microarray expression data using empirical Bayes methods. Biostatistics 8, 118-127.
Kortekaas, K. E., Santegoets, S., Sturm, G., Ehsan, I., van Egmond, S. L., Finotello, F., Trajanoski, Z., Welters, M. J., van Poelgeest, M. I., and van der Burg, S. H. (2020). CD39 identifies the CD4⁺ tumor-specific T-cell population in human cancer. Cancer immunology research 8, 1311-1321.
Le, D. T., Uram, J. N., Wang, H., Bartlett, B. R., Kemberling, H., Eyring, A. D., Skora, A. D., Luber, B. S., Azad, N. S., and Laheru, D. (2015). PD-1 blockade in tumors with mismatch-repair deficiency. New England Journal of Medicine 372, 2509-2520.
Li, B., and Dewey, C. N. (2011). RSEM: accurate transcript quantification from RNA-Seq data with or without a reference genome. BMC bioinformatics 12, 323.
Li, H. (2013). Aligning sequence reads, clone sequences and assembly contigs with BWA-MEM. arXiv preprint arXiv: 13033997.
Li, H., and Durbin, R. (2009). Fast and accurate short read alignment with Burrows-Wheeler transform. Bioinformatics 25, 1754-1760.
Linnemann, C., Van Buuren, M. M., Bies, L., Verdegaal, E. M., Schotte, R., Calis, J. J., Behjati, S., Velds, A., Hilkmann, H., and El Atmioui, D. (2015). High-throughput epitope discovery reveals frequent recognition of neo-antigens by CD4⁺ T cells in human melanoma. Nature medicine 21, 81.
Litchfield, K., Reading, J. L., Puttick, C., Thakkar, K., Abbosh, C., Bentham, R., Watkins, T. B., Rosenthal, R., Biswas, D., and Rowan, A. (2021). Meta-analysis of tumor-and T cell-intrinsic mechanisms of sensitization to checkpoint inhibition. Cell 184, 596-614. e514.
Ma, K.-Y., Schonnesen, A. A., He, C., Xia, A. Y., Sun, E., Chen, E., Sebastian, K. R., Balderas, R., Kulkarni-Date, M., and Jiang, N. (2021). High-Throughput and High-Dimensional Single Cell Analysis of Antigen-Specific CD8+ T cells. bioRxiv.
McKenna, A., Hanna, M., Banks, E., Sivachenko, A., Cibulskis, K., Kernytsky, A., Garimella, K., Altshuler, D., Gabriel, S., and Daly, M. (2010). The Genome Analysis Toolkit: a MapReduce framework for analyzing next-generation DNA sequencing data. Genome research 20, 1297-1303.
Miller, B. C., Sen, D. R., Al Abosy, R., Bi, K., Virkud, Y. V., LaFleur, M. W., Yates, K. B., Lako, A., Felt, K., and Naik, G. S. (2019). Subsets of exhausted CD8+ T cells differentially mediate tumor control and respond to checkpoint blockade. Nature immunology 20, 326-336.
Mumberg, D., Monach, P. A., Wanderling, S., Philip, M., Toledano, A. Y., Schreiber, R. D., and Schreiber, H. (1999). CD4+ T cells eliminate MHC class II-negative cancer cells in vivo by indirect effects of IFN-γ. Proceedings of the National Academy of Sciences 96, 8633-8638.
Oh, D. Y., Kwek, S. S., Raju, S. S., Li, T., McCarthy, E., Chow, E., Aran, D., Ilano, A., Pai, C.-C. S., and Rancan, C. (2020). Intratumoral CD4⁺ T cells mediate anti-tumor cytotoxicity in human bladder cancer. Cell 181, 1612-1625. e1613.
Oliveira, G., Stromhaug, K., Klaeger, S., Kula, T., Frederick, D. T., Le, P. M., Forman, J., Huang, T., Li, S., and Zhang, W. (2021). Phenotype, specificity and avidity of antitumour CD8+ T cells in melanoma. Nature, 1-7.
Ossendorp, F., Mengedé, E., Camps, M., Filius, R., and Melief, C. J. (1998). Specific T helper cell requirement for optimal induction of cytotoxic T lymphocytes against major histocompatibility complex class II negative tumors. Journal of Experimental Medicine 187,
Ott, P. A., Hu, Z., Keskin, D. B., Shukla, S. A., Sun, J., Bozym, D. J., Zhang, W., Luoma, A., Giobbie-Hurder, A., and Peter, L. (2017). An immunogenic personal neoantigen vaccine for patients with melanoma. Nature 547, 217.
Quezada, S. A., Simpson, T. R., Peggs, K. S., Merghoub, T., Vider, J., Fan, X., Blasberg, R., Yagita, H., Muranski, P., and Antony, P. A. (2010). Tumor-reactive CD4⁺ T cells develop cytotoxic activity and eradicate large established melanoma after transfer into lymphopenic hosts. Journal of Experimental Medicine 207, 637-650. 693-702.
Ramos, A. H., Lichtenstein, L., Gupta, M., Lawrence, M. S., Pugh, T. J., Saksena, G., Meyerson, M., and Getz, G. (2015). Oncotator: cancer variant annotation tool. Human mutation 36.
Riddell, S. R., and Greenberg, P. D. (1990). The use of anti-CD3 and anti-CD28 monoclonal antibodies to clone and expand human antigen-specific T cells. Journal of immunological methods 128, 189-201.
Rizvi, N. A., Hellmann, M. D., Snyder, A., Kvistborg, P., Makarov, V., Havel, J. J., Lee, W., Yuan, J., Wong, P., Ho, T. S., et al. (2015). Cancer immunology. Mutational landscape determines sensitivity to PD-1 blockade in non-small cell lung cancer. Science 348, 124-128.
Rodig, S., Gusenleitner, D., Jackson, D. G., Gjini, E., Giobbie-Hurder, A., Jin, C., Chang, H., Lovitch, S. B., Horak, C., and Weber, J. S. (2018). MHC proteins confer differential sensitivity to CTLA-4 and PD-1 blockade in untreated metastatic melanoma. Science translational medicine 10.
Ruffell, B., Chang-Strachan, D., Chan, V., Rosenbusch, A., Ho, C. M., Pryer, N., Daniel, D., Hwang, E. S., Rugo, H. S., and Coussens, L. M. (2014). Macrophage IL-10 blocks CD8⁺ T cell-dependent responses to chemotherapy by suppressing IL-12 expression in intratumoral dendritic cells. Cancer cell 26, 623-637.
Ruffin, A. T., Cillo, A. R., Tabib, T., Liu, A., Onkar, S., Kunning, S. R., Lampenfeld, C., Atiya, H. I., Abecassis, I., and Kürten, C. H. (2021). B cell signatures and tertiary lymphoid structures contribute to outcome in head and neck squamous cell carcinoma. Nature communications 12, 1-16.
Sahin, U., Derhovanessian, E., Miller, M., Kloke, B.-P., Simon, P., Löwer, M., Bukur, V., Tadmor, A. D., Luxemburger, U., and Schrörs, B. (2017). Personalized RNA mutanome vaccines mobilize poly-specific therapeutic immunity against cancer. Nature 547, 222.
Saunders, C. T., Wong, W. S., Swamy, S., Becq, J., Murray, L. J., and Cheetham, R. K. (2012). Strelka: accurate somatic small-variant calling from sequenced tumor-normal sample pairs. Bioinformatics 28, 1811-1817.
Scheper, W., Kelderman, S., Fanchi, L. F., Linnemann, C., Bendle, G., de Rooij, M. A., Hirt, C., Mezzadra, R., Slagter, M., and Dijkstra, K. (2019). Low and variable tumor reactivity of the intratumoral TCR repertoire in human cancers. Nature medicine 25, 89.
Siddiqui, I., Schaeuble, K., Chennupati, V., Marraco, S. A. F., Calderon-Copete, S., Ferreira, D. P., Carmona, S., Scarpellino, L., Gfeller, D., and Pradervand, S. (2019). Intratumoral Tcf1⁺ PD-1⁺ CD8⁺ T cells with stem-like properties promote tumor control in response to vaccination and checkpoint blockade immunotherapy. Immunity 50, 195-211. e110.
Simoni, Y., Becht, E., Fehlings, M., Loh, C. Y., Koo, S.-L., Teng, K. W. W., Yeong, J. P. S., Nahar, R., Zhang, T., and Kared, H. (2018). Bystander CD8⁺ T cells are abundant and phenotypically distinct in human tumour infiltrates. Nature 557, 575.
Singh, D., Ganesan, A. P., Panwar, B., Eschweiler, S., Hanley, C., Madrigal, A., Suastegui, C. R., Wang, A., Clarke, J., and Wood, O. (2020). CD4⁺ follicular helper-like T cells are key players in anti-tumor immunity. bioRxiv.
Sturm, G., Szabo, T., Fotakis, G., Haider, M., Rieder, D., Trajanoski, Z., and Finotello, F. (2020). Scirpy: a Scanpy extension for analyzing single-cell T-cell receptor-sequencing data. Bioinformatics 36, 4817-4818.
Tang, Z., Li, C., Kang, B., Gao, G., Li, C., and Zhang, Z. (2017). GEPIA: a web server for cancer and normal gene expression profiling and interactive analyses. Nucleic acids research 45, W98-W102.
Tangherloni, A., Ricciuti, F., Besozzi, D., Liò, P., and Cvejic, A. (2021). Analysis of single-cell RNA sequencing data based on autoencoders. bioRxiv, 727867.
Thommen, D. S., Koelzer, V. H., Herzig, P., Roller, A., Trefny, M., Dimeloe, S., Kiialainen, A., Hanhart, J., Schill, C., and Hess, C. (2018). A transcriptionally and functionally distinct PD-1⁺ CD8⁺ T cell pool with predictive potential in non-small-cell lung cancer treated with PD-1 blockade. Nature medicine 24, 994-1004.
Tran, E., Turcotte, S., Gros, A., Robbins, P. F., Lu, Y.-C., Dudley, M. E., Wunderlich, J. R., Somerville, R. P., Hogan, K., and Hinrichs, C. S. (2014). Cancer immunotherapy based on mutation-specific CD4⁺ T cells in a patient with epithelial cancer. Science 344, 641-645.
Tveita, A., Fauskanger, M., Bogen, B., and Haabeth, O. A. W. (2016). Tumor-specific CD4⁺ T cells eradicate myeloma cells genetically deficient in MHC class II display. Oncotarget 7,67175.
Van der Auwera, G. A., Carneiro, M. O., Hartl, C., Poplin, R., Del Angel, G., Levy-Moonshine, A., Jordan, T., Shakir, K., Roazen, D., and Thibault, J. (2013). From FastQ data to high-confidence variant calls: the genome analysis toolkit best practices pipeline. Current protocols in bioinformatics, 11.10. 11-11.10. 33.
Veatch, J. R., Jesernig, B. L., Kargl, J., Fitzgibbon, M., Lee, S. M., Baik, C., Martins, R., Houghton, A. M., and Riddell, S. R. (2019). Endogenous CD4⁺ T cells recognize neoantigens in lung cancer patients, including recurrent oncogenic KRAS and ERBB2 (Her2) driver mutations. Cancer immunology research 7, 910-922.
Veatch, J. R., Lee, S. M., Fitzgibbon, M., Chow, I.-T., Jesernig, B., Schmitt, T., Kong, Y. Y., Kargl, J., Houghton, A., and Thompson, J. A. (2018). Tumor infiltrating BRAFV600E-specific CD4 T cells correlated with complete clinical response in melanoma. The Journal of clinical investigation.
Voabil, P., de Bruijn, M., Roelofsen, L. M., Hendriks, S. H., Brokamp, S., van den Braber, M., Broeks, A., Sanders, J., Herzig, P., and Zippelius, A. (2021). An ex vivo tumor fragment platform to dissect response to PD-1 blockade in cancer. Nature Medicine, 1-12.
Wolf, F. A., Angerer, P., and Theis, F. J. (2018). SCANPY: large-scale single-cell gene expression data analysis. Genome biology 19, 1-5.
Wolock, S. L., Lopez, R., and Klein, A. M. (2019). Scrublet: computational identification of cell doublets in single-cell transcriptomic data. Cell systems 8, 281-291. e289.
Wu, T., Ji, Y., Moseman, E. A., Xu, H. C., Manglani, M., Kirby, M., Anderson, S. M., Handon, R., Kenyon, E., and Elkahloun, A. (2016). The TCF1-Bcl6 axis counteracts type I interferon to repress exhaustion and maintain T cell stemness. Science immunology 1.
Xiong, D., Wang, Y., and You, M. (2020). A gene expression signature of TREM2 hi macrophages and yd T cells predicts immunotherapy response. Nature communications 11, 1-12.
Yossef, R., Tran, E., Deniger, D. C., Gros, A., Pasetto, A., Parkhurst, M. R., Gartner, J. J., Prickett, T. D., Cafri, G., and Robbins, P. F. (2018). Enhanced detection of neoantigen-reactive T cells targeting unique and shared oncogenes for personalized cancer immunotherapy. JCI insight 3.
Zander, R., Schauder, D., Xin, G., Nguyen, C., Wu, X., Zajac, A., and Cui, W. (2019). CD4⁺ T cell help is required for the formation of a cytolytic CD8⁺ T cell subset that protects against chronic infection and cancer. Immunity 51, 1028-1042. e1024.
Zappasodi, R., Budhu, S., Hellmann, M. D., Postow, M. A., Senbabaoglu, Y., Manne, S., Gasmi, B., Liu, C., Zhong, H., and Li, Y. (2018). Non-conventional inhibitory CD4⁺ Foxp3-PD-lhi T cells as a biomarker of immune checkpoint blockade activity. Cancer cell 33, 1017-1032. e1017.
Colaprico A, Silva TC, Olsen C, Garofano L, Cava C, Garolini D, Sabedot T, Malta™, Pagnotta S M, Castiglioni I, Ceccarelli M, Bontempi G, Noushmehr H (2015). “TCGAbiolinks: An R/Bioconductor package for integrative analysis of TCGA data.” Nucleic Acids Research. doi: 10.1093/nar/gkv1507, http://doi.org/10.1093/nar/gkv1507.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 63/314,294, filed on Feb. 25, 2022, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method comprising identifying, selecting, and/or sorting, from a sample comprising CD4+ T cells, one or more CD4+ T cells positive for expression of CD200, wherein, optionally, the sample is a tumor sample.

2. A method comprising identifying, selecting, and/or sorting, from a sample comprising CD4+ T cells, CD4+ T cells that:
(i) express CD200, wherein, optionally, the sample comprises a tumor sample; or
(ii) have increased expression of CD200 relative to one or more other CD4+ T cells of the sample, wherein, optionally, the sample comprises a tumor sample.

3. A method comprising identifying, selecting, and/or sorting, from a sample comprising CD4+ T cells, one or more CD4+ T cells positive for expression of CXCL13, wherein, optionally, the sample is a tumor sample.

4. A method comprising identifying, selecting, and/or sorting, from a sample comprising CD4+ T cells, CD4+ T cells that:
(i) express CXCL13, wherein, optionally, the sample comprises a tumor sample; or
(ii) have increased expression of CXCL13 relative to one or more other CD4+ T cells of the sample, wherein, optionally, the sample comprises a tumor sample.

5. The method of any one of claims 1-4, wherein:
(i) the sample comprising CD4+ T cells comprises blood and/or tumor; and/or
(ii) the sample comprising CD4+ T cells is from a subject having, or having previously been diagnosed with, a cancer, such as a solid cancer (e.g., melanoma or breast cancer); and/or
(iii) the sample comprising CD4+ T cells comprises tumor-infiltrating lymphocytes and/or non-tumor-infiltrating lymphocytes; and/or
(iv) the sample comprising CD4+ T cells is from or comprises tumor infiltrated by lymphocytes; and/or
(v) the sample comprising CD4+ T cells is from a subject that has previously been administered an anti-PD-1 antibody or antigen-binding fragment thereof, optionally nivolumab or pembrolizumab; and/or
(vi) the sample comprising CD4+ T cells has previously been exposed to an anti-PD-1 antibody or antigen-binding fragment thereof, optionally nivolumab or pembrolizumab.

6. The method of any one of claims 1-5, wherein:
(i) identifying, selecting, and/or sorting the one or more CD4+ T cells positive for expression of CXCL13 or expressing CXCL13 or having increased expression of CXCL13 further comprises identifying, selecting, and/or sorting one or more of the identified, selected, and/or sorted CD4+ T cells positive for expression of PD-1 or expressing PD-1 or having increased expression of PD-1 relative to one or more other CD4+ T cells of the sample; and/or
(ii) identifying, selecting, and/or sorting the one or more CD4+ T cells positive for expression of CD200 or expressing CD200 or having increased expression of CD200 further comprises identifying, selecting, and/or sorting one or more of the of the identified, selected, and/or sorted CD4+ T cells positive for expression of PD-1 or expressing PD-1 or having increased expression of PD-1 relative to one or more other CD4+ T cells of the sample; and/or
(iii) the sample comprising CD4+ T cells has an increased frequency of CD4+ T cells positive for expression of PD-1 or expressing PD-1 or having increased expression of PD-1, relative a sample from the subject having an equivalent number of cells as compared to the sample.

7. A method comprising identifying, selecting, and/or sorting, from a sample comprising CD4+ T cells:
(i) one or more CD4+ T cells positive for expression of CXCL13 and PD-1;
(ii) one or more CD4+ T cells expressing CXCL13 and PD-1; or
(iii) one or more CD4+ T cells that have increased expression of CXCL13 and PD-1 relative to one or more other CD4+ T cells of the sample.

8. A method comprising identifying, selecting, and/or sorting, from a sample comprising CD4+ T cells:
(i) one or more CD4+ T cells expressing CD200 and PD-1;
(ii) one or more CD4+ T cells that have increased expression of CD200 and PD-1 relative to one or more other CD4+ T cells of the sample; or
(iii) one or more CD4+ T cells positive for expression of CD200 and PD-1.

9. A method comprising identifying, selecting, and/or sorting, from a sample comprising CD4+ T cells, one or more CD4+ T cells positive for expression of CD200 or that express CD200 or that have increased expression of CD200 relative to one or more other CD4+ T cells of the sample,
wherein the sample is from a subject that has previously been administered an anti-PD-1 antibody or antigen-binding fragment thereof and/or the sample has been exposed to an anti-PD-1 antibody or antigen-binding fragment thereof.

10. The method of any one of claims 1-9, wherein:
(i) the sample comprises blood and/or tumor, wherein, optionally, the sample comprises blood from a subject that has, or has previously been diagnosed with, a cancer, such as a solid cancer (e.g., melanoma or breast cancer), and has further optionally previously been administered an anti-PD-1 antibody or antigen-binding fragment thereof; and/or
(ii) the other cells of the sample consist essentially of, or consist of CD4+ T cells; and/or
iii) the sample comprises, consists essentially of, or consists of tumor-infiltrating lymphocytes.

11. The method of any one of claims 1-10, wherein the one or more identified, selected, and/or sorted CD4+ T cells:
(i) are positive for expression, express, or have increased expression relative to one or more other CD4+ T cells from the sample, and optionally have intermediate or high expression of, (1) one or more memory gene and/or (2) one or more gene or surface marker associated with T follicular helper (TFH) cells; and/or
(ii)(1) are negative for TCF7 expression, do not express TCF7, or have reduced TCF7 expression as compared to other CD4+ T cells in the sample; and/or (2) are positive for expression of, express, or have increased expression as compared to one or more other CD4+ T cells from the sample, of one or more coinhibitory marker, one or more inflammatory marker, one or more cytolytic marker, and/or one or more tissue resident memory marker; and/or
(iii) are positive for expression of, express, or have increased expression as compared to one or more other CD4+ T cell from the sample, of one or more gene involved in proliferation.

12. The method of claim 11, wherein:
(i) the one or more memory gene comprises TCF7, IL7-R, or both; and/or
(ii) the one or more gene or surface marker associated with T follicular helper (TFH) cells comprises BCL6, CD200, CXCR5, or any combination thereof; and/or
(iii) the one or more coinhibitory marker comprises TIM-3, LAG-3, or both; and/or
(iv) the one or more inflammatory marker comprises CCL3, CCL4, IFNγ, IFN-γ mRNA, or any combination thereof; and/or
(v) the one or more cytolytic marker comprises GZMA/K, PRF1 mRNA, or both; and/or
(vi) the one or more gene involved in proliferation comprises TYMS, TOP2A, MCM2/4 mRNA, or any combination thereof; and/or
(vii) the one or more tissue resident memory marker comprises CD103.

13. The method of claim 11 or 12, wherein the one or more identified, selected, and/or sorted CD4+ T cells:
(i) are positive for expression of, express, or have increased expression relative to one or more other CD4+ T cells of the sample, of any one or more (i.e. any one, any two, any three, any four, any five, or all six) of CXCL13, TCF7, IL7R, BCL6, CD200, and CXCR5, and optionally have high expression of any one or more of CXCL13, TCF7, IL7R, BCL6, CD200, and CXCR5;
(ii) (a) are positive for expression of, express, or have increased expression relative to one or more other CD4+ T cells of the sample, of CXCL13, and optionally have high expression of CXCL13, (b) are negative for TCF7 expression or do not express TCF7 expression or have reduced TCF7 expression as compared to one or more other CD4+ T cells of the sample, and (c) are positive for expression of, express, or have increased expression relative to one or more other CD4+ T cells from the sample, of TIM-3, LAG-3, IFN-γ mRNA, GZMA/K, PRF1 mRNA, and CD103; or
(iii) (a) are positive for expression of, express, or have increased expression relative to one or more other CD4+ T cells of the sample, of CXCL13, and optionally have high expression of CXCL13, and (b) are positive for expression of, express, or have increased expression relative to one or more other CD4+ T cells of the sample, of TYMS, TOP2A, and MCM2/4 mRNA,
and, optionally, (c) express, are positive for expression of, or have increased expression relative to one or more other CD4+ T cells of the sample, BTLA and/or IL-21.

14. The method of any one of claims 1-13, comprising identifying, selecting, and/or sorting one or more CD4+ T cells (a) positive for expression of, expressing, or having increased expression relative to one or more other CD4+ T cells of the sample, of CXCL13, CD200, or both, optionally (b) positive for expression of, expressing, or have increased expression of BTLA relative to one or more other CD4+ T cells of the sample, and:
(c) (i) positive for expression of, expressing, or having increased expression relative to one or more other CD4+ T cells of the sample, of (1) one or more memory gene and/or (2) one or more gene or surface marker associated with T follicular helper (TFH) cells; and/or
(c) (ii) (1) negative for TCF7 expression, not expressing TCF7, or having reduced TCF7 expression, relative to other CD4+ T cells of the sample; and/or (2) are positive for expression of, express, or have increased expression relative to one or more other CD4+ T cells of the sample, of one or more coinhibitory marker, one or more inflammatory marker, one or more cytolytic marker, and/or one or more tissue resident memory marker; and/or
(c) (iii) positive for expression of, expressing, or having increased expression relative to one or more other CD4+ T cells of the sample, of one or more gene involved in proliferation.

15. The method of claim 14, wherein:
(i) the one or more memory gene comprises TCF7, IL7-R, or both; and/or
(ii) the one or more gene or surface marker associated with T follicular helper (TFH) cells comprises BCL6, CD200, CXCR5, or any combination thereof; and/or
(iii) the one or more coinhibitory marker comprises TIM-3, LAG-3, or both; and/or
(iv) the one or more inflammatory marker comprises CCL3, CCL4, IFNγ, IFN-γ mRNA, or any combination thereof; and/or
(v) the one or more cytolytic marker comprises GZMA/K, PRF1 mRNA, or both; and/or
(vi) the one or more gene involved in proliferation comprises TYMS, TOP2A, MCM2/4 mRNA, or any combination thereof; and/or
(vii) the one or more tissue resident memory marker comprises CD103.

16. The method of claim 15, wherein:
(i) the one or more memory gene comprises TCF7, IL7-R, or both;
(ii) the one or more gene or surface marker associated with T follicular helper (TFH) cells comprises BCL6, CD200, CXCR5, or any combination thereof;
(iii) the one or more coinhibitory marker comprises TIM-3, LAG-3, or both;
(iv) the one or more inflammatory marker comprises CCL3, CCL4, IFNγ, IFN-γ mRNA, or any combination thereof;
(v) the one or more cytolytic marker comprises GZMA/K, PRF1 mRNA, or both;
(vi) the one or more gene involved in proliferation comprises TYMS, TOP2A, MCM2/4 mRNA, or any combination thereof; and
(vii) the one or more tissue resident memory marker comprises CD103.

17. The method of claim 15 or 16, comprising identifying, selecting, and/or sorting one or more CD4+ T cells that:
(i) have high expression of CXCL13 and/or CD200 and any one or more of TCF7, IL7R, and BCL6;
(ii) have high expression of CXCL13, are negative for TCF7 expression or have reduced TCF7 expression as compared to one or more other CD4+ T cells in the sample, and are positive for expression of, express, or have increased expression relative to one or more other CD4+ T cells of the sample, any one or more of TIM-3, LAG-3, IFN-γ mRNA, GZMA/K, PRF1 mRNA, and CD103; or
(iii) have high expression of CXCL13 and are positive for expression, express, or have increased expression relative to one or more other CD4+ T cells of the sample, of any one or more of TYMS, TOP2A, and MCM2/4 mRNA, and,
optionally, are positive for expression of BTLA, express BTLA, or have increased expression of BTLA relative to one or more other CD4+ T cells of the sample.

18. The method of any one of claims 1-17, further comprising identifying, selecting, and/or sorting from the one or more identified, selected, and/or sorted CD4+ T cells, (a) one or more CD4+ T cells positive for expression of, expressing, or having increased expression relative to one or more other CD4+ T cells of the sample, of TCF7 and/or (b) one or more CD4+ T cells that are negative for expression of TCF7 or that do not express TCF7 or that have reduced expression of TCF7 as compared to one or more other CD4+ T cells from the sample and/or as compared to other of the one or more identified, selected, and/or sorted CD4+ T cells.

19. The method of any one of claims 1-18, wherein: (1) identifying, selecting, and/or sorting one or more CD4+ T cells positive for expression of CXCL13 or expressing CXCL13 comprises identifying, selecting, and/or sorting one or more CD4+ T cells positive for expression of CD200 or expressing CD200, and optionally identifying, selecting, and/or sorting one or more CD4+ T cells that have high expression of CD200 or that have increased expression of CD200 relative to one or more other CD4+ T cells from the sample and/or as compared to other of the one or more identified, selected, and/or sorted CD4+ T cells; and/or (2) identifying, selecting, and/or sorting one or more CD4+ T cells positive for expression of CXCL13 or expressing CXCL13 further comprises identifying, selecting, and/or sorting one or more CD4+ T cells that express PD-1 or that are positive for expression of PD-1 or that have high expression of PD-1 or that have increased expression of PD-1 relative to one or more other CD4+ T cells from the sample and/or relative to other of the one or more identified, selected, and/or sorted CD4+ T cells.

20. The method of any one of claims 1-19:
(i) further comprising identifying, selecting, and/or sorting, from the one or more identified, selected, and/or sorted CD4+ T cells, one or more CD4+ T cells positive for expression of CXCR6, expressing CXCR6, or having increased expression of CXCR6 relative to one or more other CD4+ T cells in the sample and/or relative to other of the one or more identified, selected, and/or sorted CD4+ T cells; and/or
(ii) further comprising identifying, selecting, and/or sorting, from the one or more identified, selected, and/or sorted CD4+ T cells, one or more CD4+ T cells that are negative for expression of CXCR6 or that do not express CXCR6 or that have reduced expression of CXCR6 relative to other CD4+ T cells of the sample and/or as relative to other of the one or more identified, selected, and/or sorted CD4+ T cells; and/or
iii) wherein the one or more identified, selected, and/or sorted CD4+ T cells are negative for CD25 expression or do not express CD25 or have reduced expression of CD25 relative to one or more other CD4+ T cells of the sample and/or as compared to other of the one or more identified, selected, and/or sorted CD4+ T cells.

21. A method comprising identifying, selecting, and/or sorting, from a sample comprising CD4+ T cells, one or more CD4+ T cells that:
(i) optionally are negative for expression of CD25 or do not express CD25 or have reduced expression of CD25 relative to one or more other CD4+ T cells in the sample;
(ii) are positive for expression of PD-1 and CD200 or express PD-1 and CD200 or have increased expression relative to one or more other CD4+ T cells in the sample and optionally have high expression of PD-1 and/or CD200; and
(iii) are positive for expression of CXCR6 or express CXCR6 and optionally have high expression of CXCR6 or increased expression of CXCR6 as compared to one or more other CD4+ T cells in the sample,
wherein, optionally,
(a) the sample comprises blood and/or tumor from a subject; and/or
(b) the sample is from a subject having, or having previously been diagnosed with, a cancer, such as a solid cancer (e.g., melanoma or breast cancer).

22. The method of any one of claims 1-21, wherein identifying, selecting, and/or sorting comprises use of flow cytometry.

23. The method of any one of claims 1-22, further comprising isolating the one or more identified, selected, and/or sorted CD4+ T cells, wherein, optionally, isolating the one or more identified, selected, and/or CD4+ T cells comprises removing from the sample:
(i) the one or more sorted CD4+ T cells; and/or
(ii) CD4+ T cells positive for expression of CD25, expressing CD25, or having increased expression of CD25 relative to other CD4+ T cells of the sample; and/or
(iii) CD4+ T cells negative for expression of CD127, do not expressing CD127, or having reduced expression of CD127 relative to other CD4+ T cells of the sample; and/or
(iii) CD4+ T cells negative for expression of, not expressing, or having reduced expression relative to one or more other CD4+ T cells of the sample, of any one or more of CXCL13, PD-1 and CD200.

24. The method of any one of claims 1-23, comprising sorting the one or more identified, selected, and/or sorted CD4+ T cells away from CD4+ T cells negative for expression of, not expressing, or having reduced expression as compared to one or more other CD4+ T cells of the sample, of any one or more of CXCL13, PD-1, and CD200.

25. The method of any one of claims 1-24, further comprising culturing and/or expanding the one or more one or more identified, selected, sorted, and/or isolated CD4+ T cells.

26. The method of any one of claims 1-25, further comprising administering the one or more of the one or more identified, selected, sorted, and/or isolated CD4+ T cells to a subject having a cancer, optionally a solid cancer (e.g., melanoma or breast cancer), wherein, optionally, the subject having a cancer is the subject from which the sample was sourced.

27. The method of any one of claims 1-26, further comprising sequencing a TRBV gene segment, a TRBD gene segment, a TRBJ gene segment, a TRAV gene segment, a TRAJ gene segment, or any combination thereof, from one or more of the one or more identified, selected, sorted, and/or isolated CD4+ T cells.

28. The method of claim 27, further comprising introducing:
(1) a polynucleotide that encodes a TCR Vβ from an identified, selected, sorted, and/or isolated CD4+ T cell; and/or
(2) a polynucleotide that encodes a TCR Vα from an identified, selected, sorted, and/or isolated CD4+ T cell; and/or
(3) a polynucleotide that encodes a TCR Vβ and a TCR Vα, wherein the TCR Vβ comprises CDR1β, CDR2β, and/or CDR3β from ane identified, selected, sorted, and/or isolated CD4+ T cell, and/or wherein the TCR Vα comprises CDR1, CDR2α, and/or CDR3α from an/the identified, selected, sorted, and/or isolated CD4+ T cell,
into one or more host cells, optionally comprising one or more T cells.

29. The method of claim 28, wherein the one or more host T cells comprise CD4+ T cells and/or CD8+ T cells.

30. A CD4+ T cell or a population of CD4+ T cells or a host cell:
(i) identified, selected, sorted, isolated, cultured, expanded, and/or activated by the method of any one of claims 1-25; or
(ii) made by the method of claim 28 or 29.

31. A method comprising expanding:
(1) one or more CD4+ T cells obtained, selected, or isolated from a sample comprising a plurality of CD4+ T cells, wherein the one or more CD4+ T cells: (i) is/are positive for expression of CXCL13 or expresses CXCL13 or has increased expression of CXCL13 relative to one or more other CD4+ T cells of the plurality; (ii) is/are positive for expression of, expresses, or has increased expression relative to one or more other CD4+ T cells of the plurality, of PD-1 and CXCL13; (iii) is/are positive for expression of CD200, expresses CD200 or has increased expression of CD200 relative to one or more other CD4+ T cells of the plurality; or (iv) is/are positive for expression of PD-1 and CD200, expresses PD-1 and CD200, or has increased expression of PD-1 and/or of CD200 as relative to one or more other CD4+ T cells of the plurality; or
(2) one or more CD4+ T cells that: (i) express CXCL13; (ii) express PD-1 and CXCL13; (iii) express CD200; and/or (iv) express PD-1 and CD200, and, optionally, have increased expression of CXCL13, PD-1 and CXCL13, CD200, and/or PD-1 and CD200 as compared to other CD4+ T cells of a sample from which the one or more CD4+ T cells that are expanded was/were derived.

32. The method of claim 31, wherein the CD4+ T cell or the one or more CD4+ T cells:
(i) express or has/have increased expression of (1) one or more memory gene and/or (2) one or more gene or surface marker associated with T follicular helper (TFH) cells, as compared to other CD4+ T cells in the sample;
(ii) (1) is/are negative for TCF7 expression or has/have reduced TCF7 expression, as compared to other CD4+ T cells in the sample; and/or (2) express one or more coinhibitory marker, one or more inflammatory marker, one or more cytolytic marker, and/or one or more tissue resident memory marker; and/or
(iii) express one or more gene involved in proliferation.

33. The method of claim 32, wherein:
(i) the one or more memory gene comprises TCF7, IL7-R, or both; and/or
(ii) the one or more gene or surface marker associated with T follicular helper (TFH) cells comprises BCL6, CD200, CXCR5, or any combination thereof; and/or
(iii) the one or more coinhibitory marker comprises TIM-3, LAG-3, or both; and/or
(iv) the one or more inflammatory marker comprises CCL3, CCL4, IFNγ, IFN-γ mRNA, or any combination thereof; and/or
(v) the one or more cytolytic marker comprises GZMA/K, PRF1 mRNA, or both; and/or
(vi) the one or more gene involved in proliferation comprises TYMS, TOP2A, MCM2/4 mRNA, or any combination thereof; and/or
(vii) the one or more tissue resident memory marker comprises CD103.

34. The method of any one of claims 31-33, wherein the CD4+ T cell or the one or more CD4+ T cells:
(i) is/are positive for expression, express, or has/have increased expression of CXCL13 and any one or more of TCF7, IL7R, BCL6, and CD200, relative to one or more other CD4+ T cells from the sample;
(ii) is/are positive for expression, express, or has/have increased expression of CXCL13 relative to other CD4+ T cells from the sample, are negative for TCF7 expression or have reduced TCF7 expression, relative to one or more other CD4+ T cells from the sample, and express one or more of TIM-3, LAG-3, IFN-γ mRNA, GZMA/K, PRF1 mRNA, and CD103; or
(iii) is/are positive for expression of CXCL13, express CXCL13, or has/have increased expression of CXCL13 relative to one or more other CD4+ T cells from the sample and express one or more of TYMS, TOP2A, and MCM2/4 mRNA, and
optionally, express BTLA.

35. A CD4+ T cell expressing: (i) CXCL13; (ii) PD-1 and CXCL13; (iii) CD200; and/or (iv) PD-1 and CD200.

36. A composition comprising a plurality of CD4+ T cells, wherein 5% or more, 6% or more, 7% or more, 8% or more, 9% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, or 100% of the CD4+ T cells in the composition are according to claim 35.

37. The composition of claim 36, further comprising a pharmaceutically acceptable carrier, excipient, or diluent.

38. The composition of claim 36 or 37, wherein the CD4+ T cell:
(i) further expresses (1) one or more memory gene and/or (2) one or more gene or surface marker associated with T follicular helper (TFH) cells;
(ii) (1) does not express TCF7 or is negative for TCF7 expression; and/or (2) expresses one or more coinhibitory marker, one or more inflammatory marker, one or more cytolytic marker, and/or one or more tissue resident memory marker;
(iii) expresses one or more gene involved in proliferation;
(iv) expresses BTLA;
(v) expresses CXCR6; and/or
(vi) does not express CXCR6 or is negative for expression of CXCR6,
and, optionally, does not express CD25 or is negative for CD25 expression.

39. A CD4+ T cell from a sample comprising a plurality of CD4+ T cells, wherein the CD4+ T cell: (i) expresses CXCL13, optionally having increased expression of CXCL13 as compared to one or more other CD4+ T cells of the plurality; (ii) expresses PD-1 and CXCL13, optionally having increased expression of PD-1 and/or of CXCL13 as compared to one or more other CD4+ T cells of the plurality; (iii) expresses CD200, optionally having increased expression of CD200 as compared to one or more other CD4+ T cells of the plurality; and/or (iv) expresses PD-1 and CD200, optionally having increased expression of PD-1 and/or of CXCL13 as compared to one or more other CD4+ T cells of the plurality,
wherein, optionally, the CD4+ T cell:
(i) is negative for CD25 expression or has reduced expression of CD25 as compared to one or more other CD4+ T cells of the plurality; and/or
(j) expresses CXCR6, optionally at an increased level as compared to one or more other CD4+ T cells in the plurality; and/or
(k) is negative for expression of CXCR6 or has reduced expression of CXCR6 as compared to one or more other CD4+ T cells of the plurality; and/or
(l) is from a sample from a subject who had previously been administered an anti-PD-1 antibody or antigen-binding fragment thereof.

40. The CD4+ T cell of claim 39, wherein the CD4+ T cell:
(i) has increased expression of (1) one or more memory gene and/or (2) one or more gene or surface marker associated with T follicular helper (TFH) cells, as compared to other CD4+ T cells in the sample;
(ii) (1) is/are negative for TCF7 expression or has/have reduced TCF7 expression, as compared to other CD4+ T cells in the sample; and/or (2) expresses/express one or more coinhibitory marker, one or more inflammatory marker, one or more cytolytic marker, and or one or more tissue resident memory marker; and/or
(iii) expresses/express one or more gene involved in proliferation.

41. The CD4+ T cell of claim 39 or 40, wherein:
(i) the one or more memory gene comprises TCF7, IL-7R, or both;
(ii) the one or more gene or surface marker associated with T follicular helper (TFH) cells comprises BCL6, CD200, CXCR5, or any combination thereof; and/or
(iii) the one or more coinhibitory marker comprises TIM-3, LAG-3, or both; and/or
(iv) the one or more inflammatory marker comprises CCL3, CCL4, IFNγ, IFN-γ mRNA, or any combination thereof; and/or
(v) the one or more cytolytic marker comprises GZMA/K, PRF1 mRNA, or both; and/or
(vi) the one or more gene involved in proliferation comprises TYMS, TOP2A, MCM2/4 mRNA, or any combination thereof; and/or
(vii) the one or more tissue resident memory marker comprises CD103.

42. The CD4+ T cell of claim 41, wherein the CD4+ T cell expresses (i) CXCL13, (ii) PD-1 and CXCL13, (iii) CD200; and/or (iv) PD-1 and CD200, and further expresses:
(a) TCF7, IL7R, BCL6, CD200, CXCR5, or any combination thereof;
(b) TIM-3, LAG-3, IFN-γ mRNA, GZMA/K, PRF1 mRNA, CD103, or any combination thereof;
(c) TYMS, TOP1A, MCM2/4 mRNA, or any combination thereof; and/or
(d) BTLA.

43. The CD4+ T cell of claim 41 or 42, wherein the CD4+ T cell:
(i) has increased expression of CXCL13 and any one or more of TCF7, IL7R, BCL6, and CD200, as compared to other CD4+ T cells from the sample;
(ii) has increased expression of CXCL13 as compared to other CD4+ T cells from the sample, are negative for TCF7 expression or have reduced TCF7 expression, as compared to other CD4+ T cells in the sample, and express one or more of TIM-3, LAG-3, IFN-γ mRNA, GZMA/K, PRF1 mRNA, and CD103; or
(iii) has increased expression of CXCL13 as compared to other CD4+ T cells from the sample and express one or more of TYMS, TOP2A, and MCM2/4 mRNA, and
optionally, expresses BTLA.

44. The CD4+ T cell of any one of claims 35-43, which was obtained from a sample:
(i) comprising tumor, tumor infiltrated by lymphocytes, tumor infiltrating lymphocytes, and/or blood; and/or
(ii) from a subject having, or having previously been diagnosed with, a cancer, such as a solid cancer (e.g., melanoma or breast cancer); and/or
(iii) from a subject who had previously been administered an anti-PD-1 antibody or antigen-binding fragment thereof.

45. The CD4+ T cell of any one of claims 30, 35, and 39-44, which is specific for tumor antigen or a tumor neoantigen, optionally an antigen or neoantigen from a solid tumor.

46. A composition comprising a plurality of the CD4+ T cell of any one of claims 30, 35, and 39-45, and optionally one or both of:
(i) a plurality of tumor antigen-specific or tumor neoantigen-specific CD8+ T cells; and
(ii) a pharmaceutically acceptable carrier, excipient, or diluent.

47. A population of CD4+ T cells or a composition comprising CD4+ T cells, wherein 5% or more, 6% or more, 7% or more, 8% or more, 9% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, or 100% of the CD4+ T cells in the population or composition, respectively, are CD4+ T cells according to any one of claims 30, 35, and 39-45.

48. A cell population comprising a plurality of CD4+ T cells according to any one of claims 30, 35, and 39-45, wherein, optionally, the population comprises the CD4+ T cells in an amount comprising from about 2-fold to about 20-fold (e.g., about 2-, about 3-, about 4-, about 5-, about 6, about 7, about 8-, about 9-, about 10-, about 11-, about 12-, about 13-, about 14-, about 15-, about 16-, about 17-, about 18-, about 19-, or about 20-fold) higher than the amount of such CD4+ T cells present in a subject sample (e.g. comprising tumor, such as a solid tumor sample, and/or blood) comprising an equivalent number of cells as the population.

49. A method of treating a cancer in a subject, comprising administering to the subject an effective amount of:
(i) the CD4+ T cell or CD4+ T cell population of any one of claims 30, 35, 39-45, 47 and 48, respectively; and/or
(ii) the composition of any one of claims 36-38 and 46; and/or
(iii) a host cell made by the method of claim 28, wherein optionally the host cell comprises a T cell, optionally a CD4+ T cell.

50. The CD4+ T cell or CD4+ T cell population of any one of claims 30, 35, 39-45, 47, and 48, respectively, the composition of any one of claims 36-38 and 46, and/or a host cell made by the method of claim 28, wherein, optionally, the host cell comprises a T cell, further optionally a CD4+ T cell, for use in a method of treating cancer, optionally a solid cancer, in a subject.

51. The CD4+ T cell or CD4+ T cell population of any one of claims 30, 35, 39-45, 47, and 48, respectively, the composition of claim any one of claims 36-38 and 46, and/or a host cell made by the method of claim 28, wherein, optionally, the host cell comprises a T cell, further optionally a CD4+ T cell,
for use in the manufacture of a medicament for treating cancer, optionally a solid cancer, in a subject.

52. The method of any one of claims 5-29 and 50, the use of claim 51, the CD4+ T cell or CD4+ T cell population of any one of claims 30, 39-45, 47, and 48, and/or the composition of claim 46 or 47, wherein the solid cancer is selected from: melanoma; breast cancer; a cancer of the head or neck; pancreatic cancer; cholangiocarcinoma; hepatocellular cancer; breast cancer including triple-negative breast cancer (TNBC); gastric cancer; non-small-cell lung cancer; prostate cancer; esophageal cancer; mesothelioma; small-cell lung cancer; colorectal cancer; glioblastoma; carcinoma; sarscoma; chondrosarcoma; fibrosarcoma (fibroblastic sarcoma); Dermatofibrosarcoma protuberans (DFSP); osteosarcoma; rhabdomyosarcoma; Ewing's sarcoma; a gastrointestinal stromal tumor; Leiomyosarcoma; angiosarcoma (vascular sarcoma); Kaposi's sarcoma; liposarcoma; pleomorphic sarcoma; synovial sarcoma; a lung carcinoma (e.g., Adenocarcinoma, Squamous Cell Carcinoma (Epidermoid Carcinoma); Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carciroma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma); an ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, or an adenocarcinoma of the prostate.

53. The method of claim 52, the use of claim 52, the CD4+ T cell or CD4+ T cell population of claim 52, and/or the composition of claim 52, wherein the solid cancer comprises a melanoma, a breast cancer, or both.

54. A method for:
(1) identifying a subject as being at increased risk of relapse and/or progression of a solid cancer, the method comprising identifying a subject for whom less than 30% of CD4+ T cells present in a tumor sample of the solid cancer express CXCL13, CXCL13 and PD-1, CD200, or CD200 and PD-1, wherein, optionally, expression of CD200 and/or PD-1 is determined using flow cytometry comprising an isotype control antibody or antigen-binding fragment thereof; or
(2) identifying a subject as having a positive prognosis (e.g. increased likelihood of longer survival, longer disease-free survival, longer progression-free survival, or disease remission) for and/or as being at a reduced risk for progression or relapse of a solid cancer, the method comprising identifying a subject for whom 30% or more of CD4+ T cells present in a tumor sample of the solid cancer express CXCL13, CXCL13 and PD-1, CD200, or CD200 and PD-1, thereby identifying the subject as having a positive prognosis and/or as being at reduced risk for progression or relapse of a solid cancer as compared to a subject in whom less than 30% of CD4+ T cells that infiltrate a tumor of the solid cancer express CXCL13, CXCL13 and PD-1, CD200, or CD200 and PD-1.

55. A method for treating or for reducing a risk of a subject experiencing relapse and/or progression of a solid cancer, the method comprising administering, to a subject for whom less than 30% of CD4+ T cells present in a tumor sample of the solid cancer express CXCL13, CXCL13 and PD-1, CD200, or CD200 and PD-1, one or more therapies for the solid cancer, wherein, optionally, the one or more therapies comprise surgery, local radiation therapy, systemic radiation therapy, proton therapy, immunotherapy (e.g. comprising a cytokine, an antibody or antigen-binding fragment thereof, a fusion protein, antigen-specific T cells, antigen-specific NK cells, antigen-specific phagocytic cells, or any combination thereof), or any combination thereof.

56. The method of claim 54 or 55, wherein the CD4+ T cells that express CXCL13, CXCL13 and PD-1, CD200, or CD200 and PD-1 have increased expression of CXCL13, CXCL13 and PD-1, CD200, or CD200 and PD-1, respectively, as compared to CD4+ T cells of the subject that do not infiltrate a (optionally the) tumor of the solid cancer and/or as compared to one or more other CD4+ T cells present in the tumor sample.

57. The method of any one of claims 54-56, wherein the CD4+ T cells that express CXCL13, CXCL13 and PD-1, CD200, or CD200 and PD-1 also:
(i) express (1) one or more memory gene and/or (2) one or more gene or surface marker associated with T follicular helper (TFH) cells;
(ii) (1) are negative for TCF7 expression; and/or (2) express one or more coinhibitory marker, one or more inflammatory marker, one or more cytolytic marker, and/or one or more tissue resident memory marker; and/or
(iii) express one or more gene involved in proliferation.

58. The method of claim 57, wherein:
(i) the one or more memory gene comprises TCF7, IL-7R, or both;
(ii) the one or more gene or surface marker associated with T follicular helper (TFH) cells comprises BCL6, CD200, CXCR5, or any combination thereof; and/or
(iii) the one or more coinhibitory marker comprises TIM-3, LAG-3, or both; and/or
(iv) the one or more inflammatory marker comprises CCL3, CCL4, IFNγ, IFN-γ mRNA, or any combination thereof; and/or
(v) the one or more cytolytic marker comprises GZMA/K, PRF1 mRNA, or both; and/or
(vi) the one or more gene involved in proliferation comprises TYMS, TOP2A, MCM2/4 mRNA, or any combination thereof; and/or
(vii) the one or more tissue resident memory marker comprises CD103.

59. The method of claim 58, wherein the CD4+ T cells that express CXCL13, CXCL13 and PD-1, CD200, or CD200 and PD-1 also express:
(a) TCF7, IL7R, BCL6, CD200, CXCR5, or any combination thereof, optionally at a level that is increased as compared to the level(s) expressed by CD4+ T cells that do not infiltrate a (optionally the) tumor of the solid cancer and/or as compared to one or more other CD4+ T cells present in the tumor sample;
(b) TIM-3, LAG-3, IFN-γ mRNA, GZMA/K, PRF1 mRNA, CD103, or any combination thereof, optionally at a level that is increased as compared to the level(s) expressed by CD4+ T cells that do not infiltrate a (optionally the) tumor of the solid cancer; and/or as compared to one or more other CD4+ T cells present in the tumor sample
(c) TYMS, TOP1A, MCM2/4 mRNA, or any combination thereof, optionally at a level that is increased as compared to the level(s) expressed by CD4+ T cells that do not infiltrate a tumor of the solid cancer and/or as compared to one or more other CD4+ T cells present in the tumor sample; and/or
(d) BTLA, optionally at a level that is increased as compared to the level(s) expressed by CD4+ T cells that do not infiltrate a tumor of the solid cancer and/or as compared to one or more other CD4+ T cells present in the tumor sample.

60. The method of any one of claims 55-59, wherein the CD4+ T cells that express CXCL13, CXCL13 and PD-1, CD200, or CD200 and PD-1 also express CXCR6.

61. The method of any one of claims 55-60, wherein the CD4+ T cells that express CXCL13, CXCL13 and PD-1, CD200, or CD200 and PD-1 are negative for expression of CXCR6 or have reduced expression of CXCR6 as compared to one or more other CD4+ T cells from the subject, optionally that infiltrate a tumor of the solid cancer.

62. A method for identifying a population of CD4+ T cells from a sample, the method comprising:
(1) identifying CD4+ T cells that;
(i) express PD-1 and CD200, optionally at increased levels as compared to other CD4+ T cells in the sample, and, optionally, do not express or have reduced expression of CD25 as compared to one or more other CD4+ T cells from the sample; and
(ii) (a) express CXCR6, optionally at an increased level as compared to other CD4+ T cells from the sample, wherein the identified population comprises a T effector cell phenotype and has reduced proliferative capacity; or (b) do not express CXCR6 or have reduced expression of CXCR6 as compared to one or more other CD4+ T cells from the sample, wherein the identified population comprises a T follicular helper cell phenotype and/or comprises stem and/or progenitor CD4+ T cells with proliferative capacity; or
(2) identifying CD4+ T cells that;
(i) express PD-1 and CD200, optionally at increased levels as compared to other CD4+ T cells from the sample, and, optionally, do not express or have reduced expression of CD25 as compared to one or more other CD4+ T cells from the sample; and
(ii) (a) express TCF7, optionally at an increased level as compared to one or more other CD4+ T cells from the sample, wherein the identified population comprises a T follicular helper cell phenotype and/or comprises stem and/or progenitor CD4+ T cells with proliferative capacity; or (b) do not express TCF7 or have reduced expression of TCF7 as compared to one or more other CD4+ T cells from sample, wherein the identified population comprises a T effector cell phenotype and has reduced proliferative capacity; or
(3) identifying CD4+ T cells that;
(i) express CXCL13 and optionally PD-1, optionally at increased levels as compared to one or more other CD4+ T cells from the sample, and, optionally, do not express or have reduced expression of CD25 as compared to one or more other CD4+ T cells from the sample; and
(ii) (a) express CXCR6, optionally at an increased level as compared to one or more other CD4+ T cells from the sample, wherein the identified population comprises a T effector cell phenotype and has reduced proliferative capacity; or (b) do not express CXCR6 or have reduced expression of CXCR6 as compared to one or more other CD4+ T cells from the sample, wherein the identified population comprises a T follicular helper cell phenotype and/or comprises stem and/or progenitor CD4+ T cells with proliferative capacity; or
(4) identifying CD4+ T cells that;
(i) express CXCL13 and optionally PD-1, optionally at increased levels as compared to one or more other CD4+ T cells from the sample, and, optionally, do not express or have reduced expression of CD25 as compared to one or more other CD4+ T cells from the sample; and
(ii) (a) express CXCR6, optionally at an increased level as compared to one or more other CD4+ T cells from the sample, wherein the identified population comprises a T effector cell phenotype and has reduced proliferative capacity; or (b) do not express CXCR6 or have reduced expression of CXCR6 as compared to one or more other CD4+ T cells from the sample, wherein the identified population comprises a T follicular helper cell phenotype and/or comprises stem and/or progenitor CD4+ T cells with proliferative capacity.

63. A method for identifying a CD4+ T cell, such as for use in adoptive cell therapy, or for identifying a CD4+ T cell having and/or capable of contributing to an antitumor effect, the method comprising identifying, from a sample comprising CD4+ T cells, one or more CD4+ T cells that:
(i) express CXCL13;
(ii) have increased expression of CXCL13 as compared to one or more other CD4+ T cells from the sample;
(iii) express CD200; and/or
(iv) have increased expression of CD200 as compared to one or more other CD4+ T cells from the sample.

64. The method of claim 63,
(i) wherein the identified CD4+ T cells express PD-1 and/or have increased expression of PD-1 as compared to one or more other CD4+ T cells from the sample; and/or
(ii) further comprising identifying, from the one or more identified CD4+ T cells, CD4+ T cells that are negative for expression of CXCR6 or that have reduced expression of CXCR6 as compared to one or more other of the one or more identified CD4+ T cells; and/or
iii) further comprising identifying, from the one or more identified CD4+ T cells, CD4+ T cells that express CXCR6 or that have increased expression of CXCR6 as compared to one or more other of the one or more identified CD4+ T cells; and/or
(iv) further comprising identifying, from the one or more identified CD4+ T cells, CD4+ T cells that are negative for expression of TCF7 or that have reduced expression of TCF7 as compared to one or more other of the one or more identified CD4+ T cells; and/or
(v) further comprising identifying, from the one or more identified CD4+ T cells, CD4+ T cells that express TCF7 or that have increased expression of TCF7 as compared to one or more other of the one or more identified CD4+ T cells; and/or
(vi) further comprising sorting the one or more identified CD4+ T cells away from other cells; and/or
(vii) further comprising expanding the one or more identified or sorted CD4+ T cells; and/or
(viii) wherein the sample comprises tumor infiltrated by lymphocytes and/or tumor infiltrating lymphocytes; and/or
(ix) further comprising exposing the identified, sorted, and/or expanded CD4+ T cells to:
(1) one or more peptides that comprise a tumor antigen or tumor neoantigen, optionally wherein the tumor antigen or tumor neoantigen is present in the subject and/or against which the one or more identified, selected, sorted, and/or isolated CD4+ T cells are reactive; and/or
(2) antigen-presenting cells that present a tumor antigen or tumor neoantigen, optionally against which the one or more identified, selected, sorted, and/or isolated CD4+ T cells are known to be reactive; and/or
(3) one or more activating cytokine; and/or
(4) one or more agent that binds to a stimulatory or costimulatory protein expressed on the cell surface of the one or more identified, sorted, and/or expanded CD4+ T cells, wherein binding by the one or more agent to the stimulatory or costimulatory protein stimulates the one or more identified, selected, sorted, and/or isolated CD4+ T cells, wherein, optionally, the one or more agent that binds to a stimulatory or costimulatory protein comprises an antibody, or an antigen-binding fragment thereof, that binds to CD3, CD28, CD27, 4-1BB, OX40, ICOS, GITR, or any combination thereof.

65. A method for identifying a T cell receptor, or one or more variable domains thereof, or one or more complementarity determining regions thereof, for use in cellular immunotherapy, the method comprising
sequencing a TRAV gene segment, a TRAJ gene segment, a TRBV gene segment, a TRBD gene segment, and a TRBJ gene segment from one or more CD4+ T cells obtained from a sample, wherein the one or more CD4+ T cells;
(i) express CXCL13;
(ii) have increased expression of CXCL13 as compared to one or more other CD4+ T cells from the sample;
(iii) express CD200; and/or
(iv) have increased expression of CD200 as compared to one or more other CD4+ T cells from the sample.

66. The method of claim 65, wherein the one or more CD4+ T cells:
(i) express PD-1 and/or have increased expression of PD-1 as compared to one or more other CD4+ T cells from the sample; and/or
(ii) (a) are negative for expression of CXCR6 or have reduced expression of CXCR6 as compared to one or more other CD4+ T cells from the sample or (b) express CXCR6 or have increased expression of CXCR6 as compared to one or more other CD4+ T cells from the sample; and/or
(iii) (a) express TCF7 or have increased expression of TCF7 as compared to one or more other CD4+ T cells from the sample, or (b) are negative for expression of TCF7 or have reduced expression of TCF7 as compared to one or more other CD4+ T cells from the sample.

67. The method of claim 65 or 66, wherein the sample comprises tumor infiltrated by lymphocytes and/or tumor infiltrating lymphocytes.

68. A population of CD4+ T cells or a composition comprising CD4+ T cells, wherein 5% or more, 6% or more, 7% or more, 8% or more, 9% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, or 100% of the CD4+ T cells in the population or composition are: (i) CXCL13+; (ii) CXCL13+PD-1+; (iii) CD200+; (iv) CD200+PD-1+; (v) CD200+PD-1+ CXCR6-; (vi) CD200+PD-1+ CXCR6+; (vii) TCF7+; (vii) TCF7-; or (viii) any combination of (i)-(viii).

69. The population or composition of claim 68, wherein the CD4+ T cells were obtained from a sample and have increased expression of CXCL13, PD-1, and/or CD200 as compared to one or more other CD4+ T cells from the sample.

70. A kit for sorting or for identifying CD4+ T cells, comprising:
(i) a reagent for detecting expression of CXCL13; and/or
(ii) a reagent for detecting expression of CD200; and, optionally,
(iii) instructions for using the reagent of (i) and/or the reagent of (ii) to detect expression of CXCL13 or CD200, respectively, on a cell of interest, such as a T cell, such as a CD4+ T cell.

71. The kit of claim 70, further comprising:
(iii) a reagent for detecting expression of PD-1 or for detecting an anti-PD-1 antibody or antigen-binding fragment thereof.

72. The kit of claim 70 or 71, further comprising:
(iv) a reagent for detecting expression of CXCR6; and/or
(v) a reagent for detecting expression of TCF7.
